SEQUENCE LISTING (18 sequences)

SEQ ID NO: 1 - protein, 337 aa, E. coli
STLRLLISDSYDPWFNLAVEECIFRQMPATQRVLFLWRNADTVVIGRAQNPWKECNTRRMEEDNVRLARRSSGGGAVFHDLGNTCFTFMAGKPEYDKTISTSIVLNALNALGVSAEASGRNDLVVKTVEGDRKVSGSAYRETKDRGFHHGTLLLNADLSRLANYLNPDKKKLAAKGITSVRSRVTNLTELLPGITHEQVCEAITEAFFAHYGERVEAEIISPNKTPDLPNFAETFARQSSWEWNFGQAPAFSHLLDERFTWGGVELHFDVEKGHITRAQVFTDSLNPAPLEALAGRLQGCLYRADMLQQECEALLVDFPEQEKELRELSAWMAGAVR

SEQ ID NO: 2 - protein, 10 aa, Artificial Sequence
  Synthetic Polypeptide
XXXXKXXXXX

SEQ ID NO: 3 - protein, 22 aa, Artificial Sequence
  Synthetic Polypeptide
DEVLVEIETDKAVLEVPGGEEE

SEQ ID NO: 4 - protein, 13 aa, Artificial Sequence
  Synthetic Polypeptide
GFEIDKVWYDLDA

SEQ ID NO: 5 - protein, 13 aa, Artificial Sequence
  Synthetic Polypeptide
GFEIDKVWHDFPA

SEQ ID NO: 6 - protein, 13 aa, Artificial Sequence
  Synthetic Polypeptide
GFEIDKVFYDLDA

SEQ ID NO: 7 - protein, 8 aa, Artificial Sequence
  Synthetic Polypeptide
DYKDDDDK

SEQ ID NO: 8 - protein, 9 aa, Artificial Sequence
  Synthetic Polypeptide
YPYDVPDYA

SEQ ID NO: 9 - DNA, 70 bp, Artificial Sequence
  Synthetic Polynucleotide
aaaacatatgggattcgagatcgacaaggtgtggtacgacctggacgccggtgctgaagctggtatcacc

SEQ ID NO: 10 - DNA, 25 bp, Artificial Sequence
  Synthetic Polynucleotide
gtgcggccgcaagcttttattaatg

SEQ ID NO: 11 - DNA, 77 bp, Artificial Sequence
  Synthetic Polynucleotide
tcgacatggactacaaggatgacgacgataagggcttcgagatcgacaaggtgtggtacgacctggacgccggagct

SEQ ID NO: 12 - DNA, 69 bp, Artificial Sequence
  Synthetic Polynucleotide
ccggcgtccaggtcgtaccacaccttgtcgatctcgaagcccttatcgtcgtcatccttgtagtccatg

SEQ ID NO: 13 - protein, 17 aa, C. coli
GDTLCIVEADKASMEIP

SEQ ID NO: 14 - protein, 17 aa, B. stearoth.
DDVLCEVQNDKAVVEIP

SEQ ID NO: 15 - protein, 17 aa, E. coli
DEVLVEIDTDKVVLEVP

SEQ ID NO: 16 - protein, 6 aa, Artificial Sequence
  Synthetic Polypeptide
HHHHHH

SEQ ID NO: 17 - protein, 10 aa, Artificial Sequence
  Synthetic Polypeptide
EQKLISEEDL

SEQ ID NO: 18 - protein, 17 aa, E. coli
DEVLVEIETDKAVLEVP

(19) United States
(12) Patent Application Publication
(10) Pub. No.: US 2015/0125904 A1
(43) Pub. Date: May 7, 2015

(54) PROBE INCORPORATION MEDIATED BY ENZYMES

(71) Applicant: Massachusetts Institute of Technology, Cambridge, MA (US)

(72) Inventors: Alice Y. Ting, Allston, MA (US); Daniel Shao-Chen Liu, Cambridge, MA (US)

(73) Assignee: Massachusetts Institute of Technology, Cambridge, MA (US)

(21) Appl. No.: 14/389,499

(22) PCT Filed: Mar. 13, 2013
(86) PCT No.: PCT/US2013/030774

§ 371 Date: Sep. 30, 2014

Related U.S. Application Data
(60) Provisional application No. 61/617,808, filed on Mar. 30, 2012.

(51) Int. Cl. C12P 21/00

(52) U.S. Cl. CPC C12P 21/00
USPC 435/68.1

(57) ABSTRACT

Compositions (e.g., lipoic acid ligase polypeptides and lipoic acid analogs) and uses thereof in the Probe Incorporation Mediated By Enzymes (PRIME) methods both in vitro and in vivo. Also described herein are kits for performing the PRIME method and vectors/kits for expressing the lipoic acid ligases.

### RELATED APPLICATION

This PCT application claims the priority to U.S. Provisional Application No. 61/617,808, filed Mar. 30, 2012, the entire content of which is herein incorporated by reference.

### BACKGROUND OF THE INVENTION

Biophysical probes such as fluorophores, spin labels, and photoaffinity tags have greatly improved the understanding of protein structure and function in vitro, and there is great interest in using them inside cells to study proteins within their native context. The major bottleneck to using such probes inside cells, however, is the difficulty of targeting the probes with very high specificity to particular proteins of interest, given the chemical heterogeneity of the cell interior. The most prominent method for labeling cellular proteins is to genetically encode green fluorescent protein (GFP) or one of its variants as a fusion to the protein of interest. Because GFPs are genetically encoded, their labeling is absolutely specific and GFP variants have proven extremely useful for in vivo studies of protein localization, however, they still have severe limitations such as their large size (˜235 amino acids), which can perturb the function of the protein of interest, and the fact that they are not very bright and only amenable to optical microscopy. For example, the best of the previously described methods, the FlAsH labeling method uses an extremely small tetracysteine motif to direct a biarsenical-containing probe. This method has yielded exciting new biological information, but suffers from poor specificity, and cell toxicity. Most other methods such as the SNAP/AGT, Halotag, DHFR, FKBP (Gama et al., Methods Mol. Biol. 182:77-83, 2002), and single-chain antibody methods use protein rather than peptide-based targeting sequences, raising concerns about steric interference with receptor function. Peptide-based targeting methods include FlAsH, His₆-tag labeling, phosphopantetheinyl transferase labeling, transglutaminase labeling, and keto/biotin ligase labeling. His₆ labeling and FlAsH suffer from probe dissociation, whereas ketone/biotin lipase and transglutaminase are restricted to labeling at the cell surface.

### SUMMARY OF THE INVENTION

In one aspect, the present disclosure provides a method for preparing a protein conjugate via an enzymatic reaction catalyzed by a lipoic acid ligase. The method comprises contacting a fusion protein with a lipoic acid analog in the presence of a lipoic acid ligase polypeptide to produce a protein conjugate in which the lipoic acid analog is linked to the fusion protein. The lipoic acid analog is a substrate of the lipoic acid ligase polypeptide and has the following Formula:

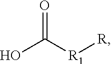

or an ester thereof, wherein R₁ is a branched or unbranched, substituted or unsubstituted C₂-C₁₄ alkyl or alkene, and R is a moiety that comprises a functional group handle, or a directly detectable group. In some examples, the directly detectable label is not a moiety of aryl azide, diazirine, benzophenone, chloroalkane, fluorobenzoic derivative, coumarin, resorufin, xanthene-type fluorophore, fluorescein, or metal-binding ligand. Optionally, the detectable label is not 7-aminocoumarin and/or hydroxycoumarin. In other examples, when R₁ is a C₅-C₁₀ alkyl or alkene, the functional group handle is not an azide; when R₁ is a C₄-C₈ alkyl or alkene, the functional group handle is not an alkyne; when R₁ is C₈-C₁₁ alkyl or alkene, the functional group handle is not a halide; or when R₁ is a C₃-C₄ alkyl, the directly detectable group is not aryl azide, a tetrafluorobenzoic derivative, benzophenone, coumarin, or Pacific blue. In some examples, when R₁ is a C₃-C₄ alkyl, the directly detectable group is not 7-aminocoumarin or 7-hydroxycoumarin, and/or the functional group handle is not cyclooctene or trans-cyclooctene.

The acceptor polypeptide can comprise the amino acid sequence

P⁻⁴P⁻³P⁻²P⁻¹P⁰P⁺¹P⁺²P⁺³P⁺⁴P⁺⁵ (SEQ ID NO:2), in which P⁴ is a hydrophobic amino acid residue (e.g., I, V, L, or F), P⁻³ is E or D, P⁻² is any amino acid residue (e.g., I), P⁻¹ is D, N, E, Y, A, or V, P⁰ is K, P⁺¹ is a hydrophobic amino acid residue (e.g., A or V), P⁺² is a hydrophobic amino acid residue (e.g., an aromatic residue) or S, P⁺³ is a hydrophobic amino acid residue (e.g., an aliphatic hydrophobic residue or an aromatic hydrophobic residue), P⁺⁴ is E or D, and P⁺⁵ is a hydrophobic amino acid residue (e.g., an aliphatic hydrophobic residue). Exemplary acceptor polypeptides include, but are not limited to, DEVLVEIETDKAVLEVPGGEEE (LAP1; SEQ ID NO:3), GFEIDKVWYDLDA (LAP2; SEQ ID NO:4), GFEIDKVWHDFPA (LAP4.2; SEQ ID NO:5) and GFEIDKVFYDLDA (LAP2-F; SEQ ID NO:6).

In some embodiments, R in the lipoic acid analog described herein is a moiety comprising a functional group handle selected from the group consisting of cyclooctene, trans-cyclooctene, azide, picolyl azide, alkyne, tetrazine, aldehyde, hydrazine, hydrozide, ketone, hydrozylamine, quadricyclane, alkene, diaryltetrazole, phosphine, diene, haloalkane, thiol, allyl sulfide, ether, thiophene, thioether, and alkyl amine.

When a lipoic acid analog used in the method described herein comprises a functional group handle, the method can further comprise contacting the protein conjugate that contains the lipoic acid analog with a compound that contains a detectable label to produce a labeled protein conjugate. Examples of the detectable label include, but are not limited to, benzophenone, diazirine, aryl azide, coumarin, unbelliferone, pacific blue, resorufin, BODIPYs, cyanine, AlexaFluor, ATTO dye, NBD, rhodamine, tetramethylrhodamine, Texas red, Lucifer yellow, Cascade yellow, dansyl, Rose Bengal, and erosin.

In other embodiments, R in the lipoic acid analog described herein comprises a directly detectable group, e.g. benzophenone, diazirine, aryl azide, coumarin, unbelliferone, pacific blue, resorufin, BODIPYs, cyanine, AlexaFluor, ATTO dye, NBD, rhodamine, tetramethylrhodamine, Texas red, Lucifer yellow, Cascade yellow, dansyl, Rose Bengal, and erosin.

The lipoic acid ligase polypeptide used in the method described herein can be a wild-type lipoic acid ligase, a functional fragment thereof, or a functional variant thereof. In some embodiments, the lipoic acid ligase polypeptide is a functional variant of a wild-type lipoic acid ligase (e.g., E. coli LplA) that comprises at least one amino acid substitution at a position corresponding to W37 in SEQ ID NO:1. Examples of E. coli LplA functional variants include, but are not limited to, W37V, W37S, W37I, W37L, W37A, W37G, E20G/W37T, and E20A/F147A/H149G.

In another aspect, the present disclosure provides a method for preparing a protein conjugate, the method comprising contacting a fusion protein with a lipoic acid analog in the presence of a lipoic acid ligase polypeptide as described above to produce a protein conjugate in which the lipoic acid analog is linked to the fusion protein. In some examples, the lipoic acid analog is a substrate of the lipoic acid ligase polypeptide and has the following Formula:

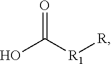

or an ester thereof, in which R₁ is a branched or unbranched, substituted or unsubstituted C₉-C₁₄ alkyl or alkene (e.g. C₁₁-C₁₄ alkyl or alkene), and R is a moiety that comprises a functional group handle or a directly detectable group. The fusion protein comprises the target protein and an acceptor polypeptide, which can be any of the acceptor polypeptides described herein.

In some embodiments, R in the lipoic acid analogs comprises a functional group handle, e.g., cyclooctene, trans-cyclooctene, azide, picolyl azide, alkyne, tetrazine, aldehyde, hydrazine, hydrozide, ketone, hydrozylamine, quadricyclane, alkene, diaryltetrazole, phosphine, diene, haloalkane, thiol, allyl sulfide, ether, thiophene, thioether, and alkyl amine. The method can further comprise contacting the protein conjugate that contains the just-described lipoic acid analog with a compound that comprises a detectable label (e.g., benzophenone, diazirine, aryl azide, coumarin, unbelliferone, pacific blue, resorufin, BODIPYs, cyanine, AlexaFluor, ATTO dye, NBD, rhodamine, tetramethylrhodamine, Texas red, Lucifer yellow, Cascade yellow, dansyl, Rose Bengal, and erosin) to produce a labeled protein conjugate.

In other embodiments, R in the lipoic acid analogs comprises a directly detectable group, which can be benzophenone, diazirine, aryl azide, coumarin, unbelliferone, pacific blue, resorufin, BODIPYs, cyanine, AlexaFluor, ATTO dye, NBD, rhodamine, tetramethylrhodamine, Texas red, Lucifer yellow, Cascade yellow, dansyl, Rose Bengal, or erosin.

Also within the scope of this disclosure is a method for preparing a protein conjugate, the method comprising contacting a fusion protein with a lipoic acid analog in the presence of a lipoic acid ligase polypeptide to produce a protein conjugate in which the lipoic acid analog is linked to the fusion protein. The lipoic acid analog can be a substrate of the lipoic acid ligase polypeptide and has the following Formula:

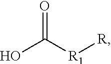

or an ester thereof, wherein R₁ is a branched or unbranched, substituted or unsubstituted C₂-C₁₄ alkyl or alkene, and R is a moiety that comprises a functional group handle (e.g., those described herein) or a directly detectable group (e.g., those described herein). The fusion protein comprises the target protein and an acceptor polypeptide, e.g., any of the acceptor polypeptide described herein. The lipoic acid ligase polypeptide to be used in this method is a truncated mutant of a wild-type lipoic acid ligase, the mutant having a deletion of a C-terminal fragment up to a position corresponding to E256 in SEQ ID NO:1 as compared to the wild-type lipoic acid ligase. The truncated mutant can contain further mutations at one or more positions, e.g., W37 in SEQ ID NO:1, as described herein.

When the lipoic acid analog comprises a functional group handle, the protein conjugate that contains such a lipoic acid analog can further react with a compound carrying a detectable label (e.g., those described herein) to produce a labeled protein.

Any of the lipoic acid analogs, lipoic acid ligase polypeptides, nucleic acids encoding same, vectors (e.g., expression vectors) comprising the nucleic acids, host cells containing the vectors, and kits containing such vectors/host cells for expressing the lipoic acid ligase polypeptides are also within the scope of this disclosure.

Also disclosed herein are kits for performing the methods for preparing protein conjugates as described above. These kits can comprise (a) any of the lipoic acid ligase polypeptide disclosed herein or an expression vector for expressing the polypeptide, (b) a lipoic acid analog recognizable by the lipoic acid ligase polypeptide, and (c) an expression vector designed for producing a fusion protein comprising a target protein and an acceptor polypeptide disclosed herein. The expression vector can comprise a first nucleotide acid sequence coding for the acceptor polypeptide and a cloning site for insertion of nucleotide sequence coding for a target protein.

The details of one or more embodiments of the invention are set forth in the description below. Other features or advantages of the present invention will be apparent from the following drawings and detailed description of several embodiments, and also from the appending claims.

### BRIEF DESCRIPTION OF THE DRAWINGS

The drawings are first described.

FIG. 1 is a schematic illustration showing the Probe Incorporation Mediated By Enzymes (PRIME) technology.

FIG. 2 is a diagram showing structures of exemplary lipoic acid analogs for use in PRIME.

FIG. 3 is a diagram showing chelation-assisted CuI-catalyzed click for site-specific and metabolic labeling of biomolecules. A: Generic reaction scheme for CuI-catalyzed, picolyl azide-alkyne cycloaddition (chelation-assisted CuAAC). B: Site-specific probe targeting to cell surface proteins via LplA-mediated picolyl azide ligation and chelation-assisted CuAAC. An engineered PRIME ligase (Trp→Val LplA) first ligated a picolyl azide derivative, called picolyl azide 8, onto LplA Acceptor Peptide (LAP), which was genetically fused to a protein of interest (POI). Picolyl azide-modified proteins were then derivatized with a terminal alkyne-probe conjugate, via live cell-compatible chelation-assisted CuAAC. BTTAA and THPTA are Cu(I) tris-triazole ligands. C: Labeling of newly synthesized RNAs (top) and proteins (bottom) in cells via alkynyl metabolites and chelation-assisted CuAAC. Besanceney-Webler, et al., Angewandte Chemie-International Edition 50:8051-8056 (2011) and Hong, et al., Bioconjugate Chemistry, 21:1912-1916 (2010). EU is a uridine surrogate and Hpg is a methionine surrogate. Jao et al, PNAS, 105:15779-15784 (2008); and Beatty et al., JACS, 127:14150-14151 (2005). Alkyne-labeled RNAs and proteins were derivatized after cell fixation with picolyl azide-fluorophore conjugates.

FIG. 4 is a graph illustrating in vitro analysis of CuAAC rates with chelating azides. A: A fluorogenic click reaction with 7-ethynyl coumarin was used to quantify CuAAC reaction progress. Zhou et al., JACS, 126:8862-8863 (2004). B: Various chelating azide structures tested and their CuAAC reaction yields after 10 min and 30 min. Reactions were run with 10 μM CuSO4 and no ligand (THPTA or BTTAA). C: Kinetic comparison of chelating azide 4 and its non-chelating benzyl counterpart 3 at different copper concentrations. CuAAC product was quantified using the assay in A), at 100, 40, and 10 μM CuSO4, both in the absence and presence of Cu(I) ligand THPTA. Measurements were performed in triplicate. Error bars, ±s.d.

FIG. 5 is a graph showing CuAAC time courses for azide compounds shown in FIG. 4B. Fluorescence was converted to coumarin triazole product quantity by comparison to standard curves, individually generated for each azide-coumarin alkyne adduct. Entries with less than 1% reaction yield (azides 1 and 3) are omitted from the plot. Measurements were performed in triplicate. Error bars, ±s.d.

FIG. 6 is a diagram showing comparison of protein labeling signals on live cells using PRIME and CuAAC, with and without chelating azides. Two-step site-specific protein labeling was performed as in FIG. 3B above and 9 below, on HEK cells expressing LAP-tagged cyan fluorescent protein fused to the transmembrane domain of the PDGF receptor (LAP-CFP-TM). In the first step, either W37VLplA was used to target picolyl azide 8 to LAP, or wild-type LplA was used to ligate non-chelating 8-azidooctanoic acid. The efficiencies of these two ligation reactions are compared in FIG. S5. In the second step, CuAAC was performed for 5 min with Alexa Fluor® 647-alkyne and CuSO4 (10, 40, or 100 μM) in combination with either THPTA or BTTAA ligand (provided in 5-fold excess relative to the CuSO4 concentration). Cells were imaged live immediately and representative images are shown in FIG. S4. To quantify labeling signals, the mean Alexa Fluor® 647 and mean CFP intensities were calculated for >90 cells for each condition, ratioed to normalize for variations in LAP-CFP-TM expression level, and averaged. Error bars, ±s.e.m.

FIG. 7 is a schematic illustration showing synthesis of PRIME ligase substrate, picolyl azide 8. TsCl: p-toluenesulfonyl chloride; TEA: triethylamine; DSC: disuccinimidyl carbonate.

FIG. 8 is a diagram showing in vitro characterization of W37VLplA-catalyzed ligation of picolyl azide 8. A: Reverse-phase HPLC traces showing LAP peptide conversion to LAP-picolyl azide 8 adduct, catalyzed by W37VLplA. For the red trace, the reaction was performed for 30 min with 1 mM ATP. In black are shown negative controls with ATP omitted or W37VLplA replaced by wild-type LplA. B: Mass-spectrometric analysis of the starred peak in (A). Calculated mass for the LAP-picolyl azide 8 adduct is 1829.28 g/mol; 1829.20 g/mol was detected.

FIG. 9 shows comparison of protein labeling signals on live cells using PRIME and CuAAC, with and without the benefit of chelation assistance. A: Two-step site-specific cell surface protein labeling protocol. In the first step, HEK cells expressing LAP-CFP-TM (TM is the transmembrane helix of the PDGF receptor) were labeled with picolyl azide 8 using W37VLplA and ATP added to the cell medium for 20 min. Alternatively, LAP-CFP-TM was labeled with non-chelating azide 8-azidooctanoic acid using wild-type LplA. In the second step, CuAAC was performed for 5 min using Alexa Fluor® 647-alkyne, various concentrations of CuSO4 (10, 40, or 100 μM), and either THPTA or BTTAA ligand added in 4-fold excess of the CuSO4. B: Representative confocal cell images for twelve different conditions (three CuSO4 concentrations, either THPTA or BTTAA ligand, and either alkyl azide or picolyl azide). For each condition, the Alexa Fluor® 647 labeling channel and the CFP channel, overlaid on DIC, are shown. Insets show the Alexa Fluor® 647 channel at higher contrast. Quantitation of this data is provided in FIG. 3. Scale bars, 10 μm.

FIG. 10 shows enzyme-catalyzed azide ligation efficiencies at the cell surface. A: Labeling protocol. HEK cells expressing LAP-CFP-TM were labeled with picolyl azide 8 and W37VLplA, or 8-azidooctanoic acid and wild-type LplA, using the same exact conditions as in FIGS. 6 and 9. Thereafter, cells were washed and any remaining unmodified LAP sites were labeled under forcing conditions with lipoic acid (200 μM lipoic acid, 1 mM ATP, and 20 μM wild-type LplA for 20 min). Anti-lipoic acid antibody staining was used to quantify the extent of lipoylation, and CuAAC was performed thereafter with 20 μM Alexa Fluor® 647-alkyne, 100 μM CuSO4, and 500 μM BTTAA ligand for 5 min. Cells were imaged live. B: Representative confocal images. Results obtained using picolyl azide 8 (condition 2) are shown below results with 8-azidooctanoic acid (condition 1). A negative control with neither azide added during the LplA step is shown in the bottom row (condition 3). The Alexa Fluor® 647 channel reflects CuAAC labeling. The Alexa Fluor® 568 channel reflects anti-lipoic acid antibody labeling. The CFP channel showing LAP-CFP-TM expression is overlaid on DIC. Scale bars, 10 μm. C: Quantitation of data in (B). The mean intensities in all three channels were collected for >90 single cells for each condition. To compare the extents of lipoylation, the Alexa Fluor® 568/CFP ratios were calculated (to normalize for variations in LAP expression level), averaged, and plotted on the graph. CuAAC labeling extent was quantified in a similar way. Error bars, ±s.e.m. Due to the forcing conditions of the LplA-catalyzed lipoylation, we set condition 3 to represent 100% lipoylation extent for the cell surface LAP-CFP-TM population. By comparison, lipoylation after picolyl azide 8 labeling proceeds to 19% that of condition. Lipoylation after 8-azidooctanoic acid labeling proceeds to 37% that of condition 3. Based on these, we can indirectly estimate that picolyl azide 8 ligation proceeds to 81%, and 8-azidooctanoic acid ligation proceeds to 63%, under these conditions.

FIG. 11 is a photo showing site-specific labeling of cell surface proteins with an engineered picolyl azide ligase and chelation-assisted CuAAC. A: Labeling of LAP-neurexin-1β on live HEK cells using PRIME and CuAAC. First, picolyl azide 8 was ligated to LAP using 10 μM W37VLplA and 1 mM ATP for 20 min. Second, the cell media was replaced with 20 μM Alexa Fluor® 647-alkyne, 50 μM CuSO4, and 250 μM THPTA for 5 min. Negative controls are shown with ATP omitted from the first step, or wild-type LplA used in place of W37VLplA. Histone2B-YFP was used as a transfection marker. B: Labeling of LAP-neuroligin-1 on the surface of living hippocampal neurons. 11 day-old cultures of rat hippocampal neurons expressing LAP-neuroligin-1 and GFP-Homer1b were labeled with picolyl azide 8 via W37VLplA, then Alexa Fluor® 647-alkyne via chelation-assisted CuAAC, and imaged live after brief rinsing. Labeling conditions were the same as in B. except: 1) higher [CuSO4] of 300 μM was used for the bottom row; 2) a radical scavenger Tempol (50 μM) was added to the CuAAC labeling solution; and 3) a biocompatible copper chelator bathocuproine sulfonate (500 μM) was used during the first rinse to immediately quench the click reaction. Alexa Fluor® 647 images in the second column correspond to the boxed regions 1 and 2, shown at higher zoom. White arrows denote regions of focal swelling when 300 μM CuSO4 is used. Confocal images are shown for both A) and B). Scale bars for all images, 10 μm.

FIG. 12 shows site-specific labeling of cell surface proteins with an alkyne ligase, followed by chelation-assisted CuAAC with a picolyl azide-probe conjugate (the inverse reaction compared to FIGS. 1B, 3, and 4). Six LplA W37 mutants—G, A, V, I, L, S—were screened for ligation activity with 6-heptynoic acid and 10-undecynoic acid. The combination of 10-undecynoic acid and W37VLplA gave the greatest product in a 30-minute assay. A: Labeling scheme. W37VLplA first ligates 10-undecynoic acid onto a LAP-tagged fusion protein. Ligated alkynes are then derivatized with a picolyl azide-probe conjugate via chelation-assisted CuAAC. B: HPLC analysis of W37VLplA-catalyzed ligation of 10-undecynoic acid onto LAP peptide. A negative control with ATP omitted is shown. C: ESI-mass spectrometric analysis of 10-undecynoic acid-LAP conjugate (starred peak in (B)). D: Fluorescent labeling of LAP-neurexin-1β on the surface of live HEK cells following the scheme in (A). The first step was performed with 200 μM 10-undecynoic acid, 10 μM purified W37VLplA, 1 mM ATP, and 5 mM Mg(OAc)₂ for 20 min. The second step was performed with 20 μM Alexa Fluor® 647-picolyl azide, 50 μM CuSO4, 250 μM THPTA, and 2.5 mM sodium ascorbate in DPBS for 5 min. Negative controls are shown with ATP omitted (second row) or wild-type LplA in place of W37VLplA (third row). H2B-YFP was used as a nuclear-localized transfection marker. Scale bars, 10 μm.

FIG. 13 shows comparison of cell-surface labeling efficiencies for four different LplA-CuAAC labeling schemes. LplA labeling was performed with picolyl azide 8,8-azidooctanoic acid, or 10-undecynoic acid. CuAAC was performed with either alkyne, picolyl azide, or alkyl azide conjugates to Alexa Fluor® 647. A: Representative images showing labeling of LAP-CFP-TM on the surface of live HEK cells under four different conditions. CFP channels are shown, along with Alexa Fluor® 647 labeling channels normalized to the same intensity range (bottom) or not normalized (middle). LplA labeling protocol for all four conditions: 200 μM azide or alkyne substrate, 10 μM LplA (wild-type or mutant), 1 mM ATP, and 5 mM Mg(OAc)₂ in cell culture medium for 20 min. CuAAC labeling protocol for all four conditions: 20 μM click probe, 100 μM CuSO4, 500 μM THPTA, and 2.5 mM sodium ascorbate in DPBS for 5 min. B: Quantitation of data in (A). Average Alexa Fluor® 647/CFP intensity ratios were calculated for ˜50 single cells from each condition. Error bars, ±s.d.

FIG. 14 shows comparison of chelation-assisted CuAAC and strain-promoted azide-alkyne cycloaddition. A: HEK cells expressing LAP-tagged neurexin-1β were labeled by W37VLplA with picolyl azide 8, then derivatized with either Alexa Fluor® 647-alkyne via chelation-assisted CuAAC (top row), or Alexa Fluor® 647-dibenzocyclooctyne (DIBO; bottom row) via strain-promoted cycloaddition. Live-cell anti-c-myc immunostaining, with a secondary antibody conjugated to Alexa Fluor® 568, shows c-myc-tagged LAP-neurexin expression on the cell surface. LplA labeling conditions: 200 μM picolyl azide 8, 10 μM W37VLplA, 1 mM ATP, and 5 mM Mg(OAc)2 in cell culture medium for 20 min. CuAAC labeling conditions: 25 μM Alexa Fluor® 647-alkyne, 50 μM CuSO4, 250 μM THPTA, 2.5 mM sodium ascorbate in DPBS for 5 min. Strain-promoted cycloaddition labeling conditions: 25 μM Alexa Fluor® 647-DIBO in 3% w/v bovine serum albumin in DPBS for 5 min. Confocal images are shown. Scale bars, 10 μm. B: CellTiter-Glo cell viability assay to test the cytotoxicity of various labeling conditions. HeLa cells transfected with LAP-neuroligin-1 plasmid were labeled using CuAAC or strain-promoted cycloaddition as indicated for 5 min. In the last row, cells were subjected to toxic treatment with 600 μM CuSO4 for 10 min. Values are normalized to that of untransfected, unlabeled cells (first entry), which is set to 100% cell viability. Measurements were performed in triplicate. Errors, ±s.d.

FIG. 15 is a schematic illustration showing application of PRIME in studying protein-protein interaction.

FIG. 16 shows metabolic labeling of cellular RNAs and proteins, and detection by chelation-assisted CuAAC. A: RNA labeling and imaging as shown in FIG. 3C. Left: A375 cells were incubated with 200 μM 5-ethynyl uridine (EU) for 90 min, then fixed. Detection was performed with either Alexa Fluor 647®-picolyl azide (first column) or Alexa Fluor® 647-alkyl azide (second column). 2 mM CuSO4 and 8 mM THPTA were used. Thereafter, cellular DNA was stained with Hoechst 33342. A negative control with EU omitted is shown (third column). Right: Graph showing mean Alexa Fluor® 647 intensities, for >3500 single cells for each condition. B: Same as A, except that instead of RNA, proteins were metabolically labeled with 50 μM homopropargylglycine (Hpg) for 90 min, before fixation and detection with Alexa Fluor 647® (picolyl azide or alkyl azide conjugate). Error bars, ±s.e.m.

FIG. 17 is a schematic illustration showing synthesis of trans-cyclooctenes and Tz2. (A) Synthesis of trans-cyclooctene substrates for LplA. (B) Synthesis of Tz2. DIPEA, diisopropylethylamine; DMF, dimethylformamide; HATU, (2-(7-Aza-1H-benzotriazole-1-yl)-1,1,3,3-tetramethyluronium hexafluorophosphate); TFA, trifluoroacetic acid; DCM, dichloromethane.

FIG. 18 shows comparison of Diels-Alder tetrazine-trans-cyclooctene cycloaddition, copper catalyzed azide-alkyne cycloaddition (CuAAC), and strain-promoted azide-alkyne cycloaddition for cell surface fluorescence labeling. (A) HEK cells expressing LAP-LDL receptor and a nuclear cyan fluorescent protein transfection marker (shown in cyan, overlaid with DIC) were labeled in two steps, using three methodologies, as indicated by the scheme: Diels-Alder cycloaddition (left), CuAAC (middle), and strain-promoted cycloaddition (right). Fernandez-Suarez et al., Nature Biotechnology 2007, 25, 1483-1487. For the latter two, LAP was first derivatized with 8-azidooctanoic acid under conditions known to give quantitative yield. DIBO is dibenzylcyclooctyne. In all three cases, the second step was performed for 3 min., using the indicated Alexa 647 conjugates at the three indicated concentrations. Cells were imaged live after brief rinsing. Specific fluorescence staining with 1 μM DIBO-Alexa 647 was detectable (shown with enhanced contrast in inset). (B) Comparing cell viability after cell surface fluorescence labeling. Chinese hamster ovary cells expressing LAP-LDL receptor were labeled using Diels-Alder cycloaddition or CuAAC under the indicated conditions. Cell viability was then measured in triplicate, with untransfected and untreated cells defined as 100% viable. The tris(benzyltriazolylmethyl)amine (TBTA) ligand (Chan et al., Organic Letters 2004, 6, 2853-2855) was used at 100 μM. The tris(hydroxypropyltriazolyl)methylamine (THPTA) ligand4 was used at 250 μM. Error bars, 2 s.d.

FIG. 19 shows two-step, site-specific fluorescence labeling of proteins using lipoic acid ligase (LplA) and Diels-Alder cy-cloaddition. (A) Optimized labeling scheme. In the first step, the Trp37→Val mutant of LplA ligates trans-cyclooctene TCO2 onto LplA acceptor peptide (LAP), which is fused to the protein of interest. In the second step, ligated trans-cyclooctene is chemoselectively derivatized with a fluorophore conjugated to Tz1 tetrazine. (B) Three trans-cyclooctenes synthesized and evaluated in this study. (C) Two tetrazines used in this study.

FIG. 20 shows fluorophore targeting via LplA-catalyzed azide ligation followed by strain-promoted azide-alkyne cycloaddition. (A) Top: natural ligation of lipoic acid catalyzed by wild-type LplA. Cronan, Adv. Micro. Phys., 50, 103-146 (2005). Bottom: two-step fluorophore targeting used in this work. First, the ^{W37I}LplA mutant ligates 10-azidodecanoic acid (“azide 9”) onto the 13-amino acid LplA acceptor peptide (LAP). Puthenveetil et al., JACS, 131, 16430-16438 (2009). Second, the azido moiety is chemoselectively derivatized using a cyclooctyne-fluorophore conjugate, via strain-promoted, copper-free [3+2] cycloaddition. Sletten et al., Accounts of Chemical Research null (2011). The red circle represents any fluorophore or probe. (B) Screening to identify the best LplA mutant/azide substrate pair. The table shows relative conversions (normalized to that of the ^{W37V}LplA/azide 9 pair, which is set to 100%) of LAP to the LAP-azide product conjugate. Wild-type LplA and six W37 point mutants were screened against four azidoalkanoic acid substrates of various lengths. N.D. indicates that product was not detected. Screening was performed with 100 nM ligase, 600 μM LAP and 20 μM azide substrate for 20 min at 30° C. Conversions were measured in duplicate. Note that ^{W37S}LplA was active with the natural substrate, lipoic acid, despite being inactive with all the azide substrates. The starred combinations in the table were evaluated.

FIG. 21 shows evaluation of various cyclooctyne structures for site-specific intracellular protein labeling. Top: labeling protocol for HEK cells co-expressing ^{W37I}LplA and nuclear-localized LAP-BFP (LAP-BFP-NLS). After labeling with azide 9 for 1 hr and washing for 1 hr, cells were treated with the indicated cyclooctyne, conjugated to fluorescein diacetate (R, grey circle; structure shown in box), for 10 min. Cells were washed again for 2.5 hr to remove excess unconjugated fluorophore, except for the case of MOFO, in which cells required only 1.5 hr of washing. Bottom: images of labeled HEK cells. The LAP-BFP-NLS image is overlaid on the DIC image. Fluorescein signal intensity and specificity can be compared in the first two columns, which show the fluorescein images at lower contrast (left) and higher contrast (middle). Cyclooctyne structures are shown at right, and second-order rate constants (with reference below) are given on the left. ADIBO, aza-dibenzocyclooctyne; DIBO, 4-dibenzocyclooctynol; MOFO, monofluorinated cyclooctyne; DIMAC, 6,7-dimethoxyazacyclooct-4-yne; DIFO, difluorinated cyclooctyne. All scale bars, 10 μm.

FIG. 22 shows identification of the best LplA mutant/azide substrate pair for intracellular protein labeling. For each condition, the mean fluorescein intensity was plotted against the mean BFP intensity, for >100 single cells. Fluorescein ligation yield is highest for the ^{W37I}LplA/azide 9 combination.

FIG. 23 shows application of PRIME methods for site-specific labeling of proteins of interest (POIs) with coumarin fluorophores. A: Labeling scheme. Coumarin ligase is the W37V mutant of E. coli lipoic acid ligase (LplA). LAP2 is a 13-amino acid recognition sequence for LplA. B: Coumarin substrates for coumarin ligase. 7-Hydroxycoumarin and Pacific Blue substrates have been previously described. 7-Aminocoumarin was synthesized and characterized in this work.

FIG. 24 is a schematic illustration showing synthesis of the 7-aminocoumarin substrate for coumarin ligase.

FIG. 25 shows engineering a Pacific Blue (PB) ligase. (A) Fluorophore ligations catalyzed by mutants of lipoic acid ligase (LplA). The top row shows ligation of 7-hydroxycoumarin (HC) by ^{W37V}LplA onto a LAP (LplA Acceptor Peptide) fusion protein, demonstrated in previous work.² The bottom row shows ligation of PB by ^{E20G/W37T}LplA, demonstrated in this work. (B) Cut-away view of wild-type LplA in complex with lipoyl-AMP ester, the intermediate of the natural ligation reaction. Adapted from PDB ID 3A7R. W37 and E20 sidechains are highlighted. (C) Modeled structure of ^{E20G/W37T}LplA in complex with PB-AMP ester. The PB-AMP conformation was energetically-minimized using Avogadro.

FIG. 26 shows screening of LplA mutants for Pacific Blue ligation activity. (A) Relative product conversions measured for nineteen LplA single and double mutants with two hydroxycoumarin (HC) probes and two Pacific Blue (PB) probes. HC3 and PB3 have n=3 linkers, and HC4 and PB4 have n=4 linkers. To generate these grids, ligation reactions were performed under both forcing conditions (12 hrs, 500 μM probe) and milder conditions (2 hrs, 50 μM probe), and analyzed by Ultra Performance Liquid Chromatography, as described in the Methods. Sample traces are shown in FIG. S2. The activity grid was generated with the following tiers: no activity, <25% conversion in a 12 hr reaction, 25-50% conversion in a 12 hr reaction, <25% conversion in 2 hr reaction, 25-50% conversion in 2 hr reaction, >50% conversion in 2 hr reaction. (B) Quantitative product yields for the top five PB ligases in (A), after 45 min reaction with 500 μM of each probe. N.D. indicates not detected. The best LplA mutants for PB3, HC3, and HC4 are highlighted. Errors are reported as standard errors of the mean. (C) HPLC trace showing formation of LAP-PB3 conjugate, catalyzed by our best PB ligase, ^{E20G/W37T}LplA. The identity of the LAP-PB3 peak was confirmed by mass spectrometry. Traces below show negative control reactions with ATP omitted (red) or ^{E20G/W37T}LplA replaced by wild-type LplA (black).

FIG. 27 shows a site-specific PRIME labeling method using lipoic acid analogs comprising aldehyde or hydrazine moieties via lipoic acid ligase-catalyzed reactions. A: a schematic illustration showing a two-step PRIME labeling method. B: tables showing conversion efficiencies using wild-type and mutant LplA. C: a chart showing conjugation of the above-described lipoic acid analogs onto LAP.

FIG. 28 shows site-specific fluorophore conjugation to (A) LAP-alkaline phosphatase, and (B) E2p protein. E2p is a domain of pyruvate dehydrogenase, one of LplA's natural protein substrates. E2p or crude LAP-alkaline phosphatase in periplasmic extract was labeled with W37ILplA and Ald substrate, then fluorescein-hydrazide (lanes 1 and 2). Similarly, E2p was labeled with W37ILplA and Hyd substrate, then fluorescein-aldehyde in lanes 3 and 4. Coomassie-stained gels are shown beside fluorescence images to show fluorescein-labeled bands. In both gels, even numbered lanes are negative controls with ATP omitted from the ligation reaction. The crude LAP-alkaline phosphatase periplasmic extract was generated as previously described. See Jewett et al., J. Am. Chem. Soc. 2010, 132:3688.

### DETAILED DESCRIPTION OF THE INVENTION

Prior attempts to label specific proteins have been frustrated by a lack of reagents with sufficient specificity. The methods described herein aims at overcoming this lack of specificity, relying on the specificity of the enzymatic reactions catalyzed by lipoic acid ligases.

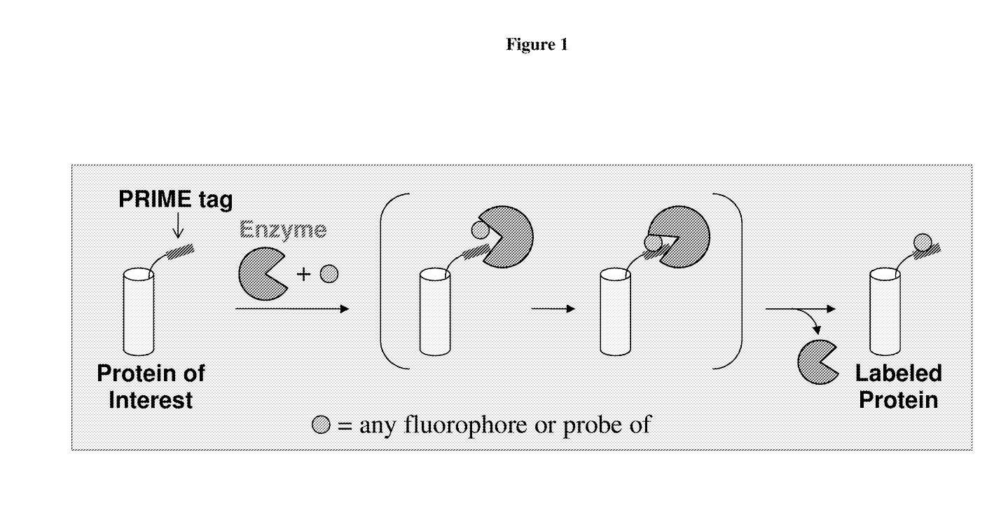

Lipoic acid ligase is an enzyme that catalyzes the ATP-dependent ligation of the small molecule lipoic acid to a specific lysine sidechain within one of three natural acceptor proteins E2p, E2o, and H-protein. The reaction between a wild-type lipoic acid ligase and its substrates is referred to as orthogonal. This means that neither the ligase nor its substrate react with any other enzyme or molecule when present either in their native environment (i.e., a bacterial cell) or in a non-native environment (e.g., a mammalian cell). Accordingly, the present disclosure takes advantage of the high degree of specificity that has evolved between wild-type lipoic acid ligase and its substrate. The natural reaction of LplA has now been redirected such that unnatural structures, dissimilar to lipoic acid, can be ligated to either the natural protein substrates or engineered peptide substrates. A schematic illustration of the technology described herein (Probe Incorporation Mediated By Enzymes or PRIME) is provided in FIG. 1.

The present disclosure is based on the unexpected discovery that lipoic acid ligases, including both wild-type enzymes and modified version, can conjugate designed lipoic acid analogs (e.g., non-naturally occurring analogs of lipoic acid) to designed acceptor polypeptides (e.g., non-naturally peptide substrates of a lipoic acid ligase), which can be fused with a protein of interest. Accordingly, described herein are methods for preparing protein conjugates via enzymatic reactions catalyzed by lipoic acid ligase polypeptides to conjugate a lipoic acid analog with an acceptor polypeptide, which is fused with a target protein. The ligation interactions of the methods described herein may or may not be orthogonal ligation reactions.

### Lipoic Acid Ligase Polypeptides

The lipoic acid ligase polypeptides used in the methods described herein are proteins possessing lipoic acid ligase activity, i.e., capable of catalyzing an ATP-dependent ligation of a small molecule lipoic acid analog to a specific lysine sidechain within an acceptor polypeptide. The lipoic acid ligase polypeptides, which are also within the scope of this disclosure, can be either wild-type enzymes or functional variants thereof, which preferably have altered substrate specificity as compared with their wild-type counterparts.

### (i) Wild-type Lipoic Acid Ligases

The lipoic acid ligase polypeptides used in the method described herein can be naturally-occurring (i.e., wild-type) lipoic acid ligases, which are well known in the art.

In some embodiments, a wild-type lipoic acid ligase is an E. coli lipoic acid ligase, such as LplA. In one example, an E. coli LpLA has the amino acid sequence SEQ ID NO:1 shown below:

```
Ser Thr Leu Arg Leu Leu Ile Ser Asp Ser Tyr Asp Pro Trp Phe Asn
1 5 10 15
Leu Ala Val Glu Glu Cys Ile Phe Arg Gln Met Pro Ala Thr Gln Arg
                        20 25 30
Val Leu Phe Leu Trp Arg Asn Ala Asp Thr Val Val Ile Gly Arg Ala
                35 40 45
Gln Asn Pro Trp Lys Glu Cys Asn Thr Arg Arg Met Glu Glu Asp Asn
        50 55 60
Val Arg Leu Ala Arg Arg Ser Ser Gly Gly Gly Ala Val Phe His Asp
65 70 75 80
Leu Gly Asn Thr Cys Phe Thr Phe Met Ala Gly Lys Pro Glu Tyr Asp
                                85 90 95
Lys Thr Ile Ser Thr Ser Ile Val Leu Asn Ala Leu Asn Ala Leu Gly
                        100 105 110
Val Ser Ala Glu Ala Ser Gly Arg Asn Asp Leu Val Val Lys Thr Val
                115 120 125
Glu Gly Asp Arg Lys Val Ser Gly Ser Ala Tyr Arg Glu Thr Lys Asp
        130 135 140
Arg Gly Phe His His Gly Thr Leu Leu Leu Asn Ala Asp Leu Ser Arg
145 150 155 160
Leu Ala Asn Tyr Leu Asn Pro Asp Lys Lys Lys Leu Ala Ala Lys Gly
                                165 170 175
Ile Thr Ser Val Arg Ser Arg Val Thr Asn Leu Thr Glu Leu Leu Pro
                        180 185 190
Gly Ile Thr His Glu Gln Val Cys Glu Ala Ile Thr Glu Ala Phe Phe
                195 200 205
Ala His Tyr Gly Glu Arg Val Glu Ala Glu Ile Ile Ser Pro Asn Lys
        210 215 220
Thr Pro Asp Leu Pro Asn Phe Ala Glu Thr Phe Ala Arg Gln Ser Ser
225 230 235 240
Trp Glu Trp Asn Phe Gly Gln Ala Pro Ala Phe Ser His Leu Leu Asp
                                245 250 255
Glu Arg Phe Thr Trp Gly Gly Val Glu Leu His Phe Asp Val Glu Lys
                        260 265 270
Gly His Ile Thr Arg Ala Gln Val Phe Thr Asp Ser Leu Asn Pro Ala
                275 280 285
Pro Leu Glu Ala Leu Ala Gly Arg Leu Gln Gly Cys Leu Tyr Arg Ala
        290 295 300
Asp Met Leu Gln Gln Glu Cys Glu Ala Leu Leu Val Asp Phe Pro Glu
305 310 315 320
Gln Glu Lys Glu Leu Arg Glu Leu Ser Ala Trp Met Ala Gly Ala Val
                                325 330 335
Arg
```

SEQ ID NO:1 differs from the GenBank sequence set forth as Accession No. AAA21740 in one aspect, i.e., the first amino-acid (methionine) in AAA21740 is not included in SEQ ID NO:1. See also U.S. Pat. No. 8,137,925, which is herein incorporated by reference.

In other embodiments, wild-type lipoic acid ligases can be homologs of the E. coli LplA described above. Examples include, but are not limited to: Thermoplasma acidophilum LplA; Plasmodium falciparum LipL1, or LipL2; Oryza Sativa LplA (rice); Streptococcus pneumoniae LplA; and homologs from Pyrococcus horikoshii; Saccharomyces cerevisiae, Trypanosoma cruzi, Bacillus subtilis, Leuconostoc mesenteroides, E. coli (e.g., GenBank accession nos. YP_{—}002394530.1 and EFZ57048.1), Shigella dysenteriae (e.g., GenBank accession no. ZP_{—}03066442.1), Salmonella enterica (e.g., GenBank accession no. ZP_{—}03218054.1), Citrobacter youngae (e.g., GenBank accession no. ZP_{—}06354791.1), Enterobacter hormaechei (e.g., GenBank accession no. ZP_{—}08497578.1), and Klebsiella pneumoniae (e.g., GenBank accession no. AEJ96389.1).

Other homologs of E. coli LplA can be retrieved from any gene database via methods known in the art, for example, using the LpLA sequence (amino acid sequence or gene sequence), or a conservative fragment thereof, as a search query.

### (ii) Functional Mutants of Lipoic Acid Ligases

Functional mutants of wild-type lipoic acid ligases preserve the enzymatic activity to catalyze an ATP-dependent ligation of a lipoic acid or lipoic acid analog to a specific lysine sidechain within an acceptor polypeptide. In preferred embodiments, a functional lipoic acid ligase mutant has altered substrate specificity as compared to its wild-type counterpart such that it can conjugate an unnatural compound substrate (a lipoic acid analog) to an unnatural peptide substrate.

A functional lipoic acid ligase mutant may retain some level of activity for lipoic acid or an analog thereof. Its binding affinity for lipoic acid or an analog thereof may be similar to that of wild-type lipoic acid ligase. Preferably, the mutant has higher binding affinity for a lipoic acid analog than it does for lipoic acid. Consequently, lipoic acid conjugation to an acceptor peptide would be lower in the presence of a lipoic acid analog. In still other embodiments, the lipoic acid ligase mutant has no binding affinity for lipoic acid.

Lipoic acid ligase is a well-characterized enzyme family with its structure/function correlation known in the art. See, e.g., Fujiwara et al., J Biol Chem. 2005, 280(39):33645-51; and Fujiwara et al., J. Biol. Chem., 2010, 285(13):9971-9980. Based on the knowledge in the art and disclosed herein, one of ordinary skill in the art will recognize how to identify suitable lipoic acid ligases and how to modify lipoic acid ligases of the invention to prepare additional lipoic acid ligases that are useful in methods described herein.

The functional mutants of lipoic acid ligases described can be designed based on the structure/function correlation of lipoic acid ligases as known in the art and/or described herein, using the E. coli LpLA having the amino acid sequence of SEQ ID NO:1 as an example. Table 1 below lists the functional amino acid residues in SEQ ID NO:1:

TABLE 1
Functional amino acid residues in SEQ ID NO: 1

| Function | Involved Amino Acid Residues |
|---|---|
| Lipoate binding loop | R70, S71, S72, G73, G74, G75, A76, |
| | V77, F78, H79 |
| Interaction with phosphate | N121, D122 |
| and magnesium | |
| 2^{nd} side of lipoate binding | K133, V133, S135, G136, S137, A138 |
| tunnel | |
| H-protein interaction loop | Y139, R140, E141, T142, K143, D144 |
| 3^{rd} side of lipoate binding | H149, G150, T151, L152, L153 |
| tunnel | |
| Adenosine binding loop | T178, S179, V180, R181, S182, R183, |
| | V184 |

The 36 amino acid residues listed in Table 1 above play at least one role in the enzymatic activity of E. coli LplA. Thus, at least 70%, 75%, 80%, 85%, 90%, 95%, 97%, 98%, or 99% of these 36 residues should not be mutated in the functional mutants of lipoic acid ligase described herein. In some embodiments, only conservative mutations are introduced into positions corresponding to these 36 residues within the tolerable range. In some examples, none of the 36 positions is mutated in the functional mutants described herein. In other embodiments, 1, 2, 3, 4, 5, 10, 15, 20, 25, or 30 of the involved amino acids include a conservative mutation.

As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Conservative amino-acid substitutions in the amino acid sequence of lipoic acid ligase mutants to produce functionally equivalent variants typically are made by alteration of a nucleic acid encoding the mutant. Such substitutions can be made by a variety of methods known to one of ordinary skill in the art. For example, amino acid substitutions may be made by PCR-directed mutation, site-directed mutagenesis according to the method of Kunkel (Kunkel, PNAS 82: 488-492, 1985), or by chemical synthesis of a nucleic acid molecule encoding a lipoic acid ligase mutant.

Further, truncation of a C-terminal fragment (e.g., residues 256-337) was found not to abolish the enzymatic activity of E. coli LplA, indicating that the C-terminal fragment can be deleted without affecting lipoic acid ligase activity. As such, the functional mutants described herein can contain C-terminal truncations (e.g., up to T185 or E256 in SEQ ID NO:1) as compared to their wild-type counterparts. In some examples, the truncated mutants encompass all of the 36 functional residues listed above. The truncated mutants can further contain additional mutations at positions corresponding to, e.g., one or more non-functional amino acid residues, or one or more residues noted below that are involved in determination of substrate specificity.

Functional mutants having altered compound substrate specificity as compared to their wild-type counterparts can be developed based on an analysis of the lipoic acid binding site of wild-type lipoic acid ligase. Residues in SEQ ID NO:1 that appear important in the interaction with lipoic acid include: N16, L17, V19, E20, E21, W37, F35, N41, R70, S71, S72, H79, C85, T87, R140, F147, and H149. For example, mutations at positions E20, F147, and/or H149 might enlarge the lipoic acid-binding pocket, thereby resulting in lipoic acid ligase mutant reactive to lipoic acid analog carrying relative large moieties (e.g., coumarin, resorufin, and Pacific blue). This has been demonstrated by the crystal structure of a resorufin-specific lipoic acid ligase comprising the triple mutant E20A/F147A/H149G of SEQ ID NO:1 (see U.S. patent application Ser. No. 13/267,761).

Briefly, the resorufin-specific lipoic acid ligase with an N-terminal hexahistidine tag followed by a tobacco etch virus (TEV) protease cleavage site was overexpressed in E. coli and then purified by immobilized metal affinity chromatography. The hexahistidine tag was cleaved using TEV protease (AcTEV, Invitrogen) and the resulting tag-less ligase purified by size-exclusion chromatography on a Superdex S75 column developed in 20 mM Tris-HCl, pH 7.5 supplemented with 30 mM NaCl and 1 mM dithiothreitol (Buffer A). To generate and cryopreservate of protein crystals, 1 uL of 5.5 mg/mL the ligase in Buffer A was supplemented with 2.5 molar equivalence of resorufin sulfamoyl adenosine and mixed with 1 uL of precipitant (0.15 M MES:NaOH, pH 6.5 containing 11% (w/v) PEG 20,000) in a hanging drop vapor diffusion setup, stored at 4 degrees Celsius. Pink-colored crystal plate clusters were observed after 24 hours. Single crystal plates in the hanging drop buffer supplemented with 15% (v/v) glycerol were flash frozen in liquid nitrogen. Diffraction data were collected at Beamline 24-IDE at the Advanced Photon Source (Argonne, Ill.) and were processed with HKL2000. The structure was phased using a previously solved wild-type LplA structure with lipoyl-AMP bound (PDB ID 3A7R). Iterative rounds of model building and refinement were done using the COOT software. The results obtained from this study demonstrate that, as predicted, the mutated ligase has an enlarged lipoic acid-binding pocket that fit the resorufin moiety. Thus, mutations at one or more residues involved in binding to the lipoic acid compound substrate would result in lipoic acid ligase mutants reactive to lipoic acid analogs having relatively large moieties, such as resorufin and coumarin.

Accordingly, mutations can be introduced into one or more of the above listed positions to produce functional mutants that recognize lipoic acid analogs. See also U.S. Pat. No. 8,137,925 and U.S. patent application Ser. No. 13/267,761, which is herein incorporated by references. Specific examples of the functional mutants described herein include, but are not limited to, proteins having at least one of the amino acid substitution that corresponds to: N16A, L17A, V19A, E20A, E21A, W37A, W37G, W37S, W37V, W37A+S71A, W37A+E20A, W37L, W37I, W37T, W37N, W37V+E20G, W37V+F35A, W37V+E20A, F35A, N41A, R70A, S71A, S72A, H79A, C85A, T87A, R140A, F147A, H149A, and H149V of wild-type E. coli lipoic acid ligase set forth as SEQ ID NO:1. Of particular importance in some embodiments are functional mutants that harbor amino acid substitutions at positions that correspond to E20, F35, W37, S71, H79, F147 and H149 of SEQ ID NO:1. Examples include but are not limited to substitutions that correspond to E20A, W37A, W37G, W37S, W37V, W37L, W37N, W37I, W37T, W37V+E20G, W37V+E20A and W37V+F35A of SEQ ID NO:1.

To obtain functional mutants that can accommodate relatively larger compound substrates, amino acid residue substitutions can be introduced into one or more positions corresponding to residues E20, W37, and F147 in SEQ ID NO:1.

In some embodiments, a functional mutant of lipoic acid ligase described herein comprises an amino acid sequence at least 75% (e.g., 85%, 90%, 95%, 97%, or 99%) identical to residues 1-256 of SEQ ID NO:1. In other examples, a functional mutant described herein comprises an amino acid sequence at least 70% (e.g., 75%, 80%, 85%, 90%, 95%, 97%, or 99% identical to SEQ ID NO:1.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

Lipoic acid ligase mutants can be generated in any number of ways, including in vitro compartmentalization, genetic selections, yeast display, or FACS in mammalian cells, described in greater detail herein, all of which are standard methods understood and routinely practiced by those of ordinary skill in the art.

Table 2 below listed a number of exemplary functional mutants of E. coli LpLA and the lipoic acid analogs recognizable by these mutants:

TABLE 2
E. coli LpLA Mutants and Lipoic Acid Analogs Recognizable Thereby

| | Lipoic Acid Analog 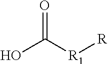 | | Acceptor | |
|---|---|---|---|---|
| Lipoic Acid Ligase | R1 | R | Polypeptide | References |
| LplA | (CH2)ₙ, n: | Azide | LAP1 | Fernandez-Suarez et al., |
| | 5-10 | | | Nature Biotechnology, |
| | | | | 2007, 25(12): 1483-1487 |
| LplA | (CH2)ₙ, n: | Alkyne | LAP1 | Fernandez-Suarez et al., |
| | 4-8 | | | Nature Biotechnology, |
| | | | | 2007, 25(12): 1483-1487 |
| W37V LplA; | (CH2)ₙ, | Aryl azide | LAP1 | Baruah et al., Angew |
| W37S LplA | n: 4 | | | Chem Int. Ed. Engl., 2008, |
| | | | | 47(37): 7018-7021 |
| W37V LplA | (CH2)ₙ, | Courmarin, | LAP2 | Uttamapinant et al., PNAS, |
| W37I LplA | n: 4 | 7-hydroxycoumarin | | 2010, 107(24): 10914- |
| W37L LplA | | | | 10919 |
| W37A LplA | | | | |
| E20G/W37T LplA | (CH2)ₙ, | Pacific blue | LAP2 | Cohen et al., Biochemistry, |
| | n: 3 | fluorophore | | 2011, 50(38): 8221-8225 |
| W37V LplA | (CH2)ₙ, | 7-aminocourmarin | LAP2 | Jin et al., ChemBioChem, |
| | n: 4 | | | 2011, 12(1): 65-70 |
| W37I LplA, | (CH2)ₙ, | Azide | LAP2 | Yao et al., J. Am. Chem. |
| W37V LpLA | n: 9 or 10 | | | Soc. 2012, 134(8): 3720- |
| | | | | 3728. |
| E20A/F147A/H149G | (CH2)ₙ, | resorufin | LAP2, LAP2- | USSN 13/267,761 |
| | n: 4 | | F | |
| W37V LplA, | (CH2)ₙ, | Trans-cyclooctene | LAP2 | Liu et al., J. Am. Chem. |
| W37G LplA, | n: 4 | | | Soc. 2012, 134(2): 792-795 |
| W37I LplA | | | | |
| W37I LplA | (CH2)ₙ, | Aldehyde, | LAP2 | Cohen et al., |
| W37V LplA | n: 3 | Hydrazine | | Chembiochem, 2012. |
| W37T LplA | | | | |
| W37L LplA | | | | |
| W37C LplA | | | | |
| LplA | (CH2)ₙ, | Azide, Coumarin | LAP1, LAP2 | Slavoff et al., J. Am. |
| W37V LplA | n: 4 | | | Chem. Soc., 133: 19769- |
| | | | | 19776 |

(iii) Preparation of Lipoic Acid Ligase Polypeptides

Any of the lipoic acid ligase polypeptides described above can be either isolated from a nature source via routine protein purification technology or prepared by routine recombinant technology.

Various assays can be used to test the specificity and functionality of a lipoic acid ligase polypeptide and its suitability for mammalian cell labeling applications. A non-limiting example of a method for identifying a lipoic acid ligase includes contacting a lipoic acid or lipoic acid analog with an acceptor polypeptide in the presence of a candidate lipoic acid ligase molecule, and detecting a lipoic acid or lipoic acid analog that is bound to the acceptor polypeptide, wherein the presence of a lipoic acid or lipoic acid analog bound to an acceptor polypeptide indicates that the candidate lipoic acid ligase molecule is a lipoic acid ligase that has specificity for the lipoic acid or lipoic acid analog.

Any of the isolated lipoic acid ligase polypeptides described herein, their encoding nucleic acids (in isolated form), vectors (e.g., expression vectors) comprising such nucleic acids, and host cells comprising the vectors are within the scope of this disclosure.

Also within the scope of this disclosure are methods of making any of the lipoic acid ligase polypeptides, comprising culturing the host cells noted above under suitable conditions known in the art to allow expression of the polypeptides, and collecting the cells thus obtained for isolation and purification of the polypeptides.

As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art.

### Lipoic Acid Analogs

The lipoic acid analogs described herein are compound substrates of lipoic acid ligases. Like the compound substrate of naturally-occurring lipoic acid ligases, lipoic acid, the lipoic acid analogs all contain an aliphatic carboxylic acid moiety or an ester thereof, e.g., an AMP ester. In some embodiments, lipoic acid analog described herein has the structure of CO₂H—CH₂-L-X, in which L is a linear string of 1-13 atoms, such as (CH₂)n, n being 1-13, and X is a chemical moiety. L can be branched or unbranched, substituted, or not substituted. In some embodiments, X is a chemical moiety having a dimension not exceeding 1.6 nm×0.9 nm×0.8 nm. The 3-D dimension of a chemical moiety can be determined via methods known in the art, for example, Maestro and viewing the crystal structure in Pymol and measuring distances using that software.

In some embodiments, a lipoic acid analog described herein has the structure of

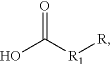

or an ester thereof, e.g., an AMP ester, wherein R₁ is a branched or unbranched, substituted or unsubstituted C₂-C₁₄ alkyl or alkene (e.g., C₂-C₈, C₄-C₈, C₈-C₁₄, or C₁₁-C₁₄), and R is a chemical moiety having the dimension as set forth above. Examples of substituents include, but are not limited to, halo, hydroxy, amino, cyano, nitro, mercapto, alkoxycarbonyl, amido, alkanesulfonyl, alkylcarbonyl, carbamido, carbamyl, carboxy, thioureido, thiocyanato, sulfonamido, alkyl, alkenyl, alkynyl, alkyloxy, aryl, heteroaryl, cyclyl, and heterocyclyl.

In the above structure, R can comprise a functional group handle or a directly detectable group. When R₁ is a C₅-C₁₀ alkyl or alkene, the functional group handle is not an azide, when R₁ is a C₄-C₈ alkyl or alkene, the functional group handle is not an alkyne, when R₁ is C₈-C₁₁ alkyl or alkene, the functional group handle is not a halide, and when R₁ is a C₃-C₄ alkyl, the directly detectable group is not a moiety selected from the group consisting of an aryl azide, a tetrafluorobenzoic derivative, benzophenone, coumarin, or Pacific blue.

A functional group handle is a moiety (e.g., an azide group) capable of reacting with another chemical moiety to form a bond (e.g. a covalent bond) such that the other chemical moiety is conjugated to the functional group handle. Incorporation of a “functional group handle” in a lipoic acid analog described herein can be more feasible due to the small size of the lipoate binding pocket in a lipoic acid ligase. This approach provides greater versatility for subsequent incorporation of probes of any structure.

Functional group handles have been widely used in chemical biology, including ketones, organic azides, and alkynes (Prescher, J. A. & Bertozzi, C. R. 2005 Nat. Chem. Biol. 1, 13-21). Organic azides are suitable for live cell applications, because the azide group is both abiotic and non-toxic in animals and can be selectively derivatized under physiological conditions (without any added metals or cofactors) with cyclooctynes, which are also unnatural (Agard, N. J., et. al., 2006 ACS Chem. Biol. 1, 644-648). Methods of using functional group handles such as azides and alkynes are well known in the art and methods and procedures for the use of such functional group handles in combination with a cyclooctyne reaction a partner are understood and can be practiced by those of ordinary skill in the art using routine techniques.

Other functional group handles for use in the lipoic acid analogs described herein include, but are not limited to, cyclooctene, trans-cyclooctene, azide, picolyl azide, alkyne, tetrazine, aldehyde, hydrazine, hydrozide, ketone, hydrozylamine, quadricyclane, alkene, diaryltetrazole, phosphine, diene, haloalkane, thiol, allyl sulfide, ether, thiophene, thioether, and alkyl amine.

A directly detectable group is a chemical moiety (e.g., a photoaffinity probe or a fluorophore) that has the ability to emit and/or absorb light of a particular wavelength and can be directly detected by a variety of methods including fluorescence, electrical conductivity, radioactivity, size, and the like. Such a group can be a fluorescent molecule, a chemiluminescent molecule (e.g., chemiluminescent substrates), a phosphorescent molecule, a radioisotope, a chromogenic substrate, a contrast agent, or a phosphorescent label. Examples of directly detectable group include, but are not limited to, benzophenone, diazirine, aryl azide, coumarin, unbelliferone, pacific blue, resorufin, BODIPYs, cyanine, AlexaFluor, ATTO dye, NBD, rhodamine, tetramethylrhodamine, Texas red, Lucifer yellow, Cascade yellow, dansyl, Rose Bengal, and erosin. Others include fluorophores such as fluorescein isothiocyanate (“FITC”), Texas Red®, tetramethylrhodamine isothiocyanate (“TRITC”), 4,4-difluoro-4-bora-3a, and 4a-diaza-s-indacene (“BODIPY”), Cy-3, Cy-5, Cy-7, Cy-Chrome™, R-phycoerythrin (R-PE), PerCP, allophycocyanin (APC), PharRed™, Mauna Blue, Alexa™ 350 and other Alexa™ dyes, and Cascade Blue®.

In some examples, the directly detectable group is a positron emission tomography (PET) label such as 99m technetium and 18FDG. In other examples, it is an singlet oxygen radical generator including but not limited to resorufin, malachite green, fluorescein, benzidine and its analogs including 2-aminobiphenyl, 4-aminobiphenyl, 3,3′-diaminobenzidine, 3,3′-dichlorobenzidine, 3,3′-dimethoxybenzidine, and 3,3′-dimethylbenzidine. These molecules are useful in EM staining and can also be used to induce localized toxicity.

In yet other examples, the directly detectable group is a heavy atom carrier, which would be particularly useful for X-ray crystallographic study of the target protein. Heavy atoms used in X-ray crystallography include but are not limited to Au, Pt and Hg. An example of a heavy atom carrier is iodine.

In still other examples, the directly detectable group is a photoactivatable cross-linker, which is a cross linker that becomes reactive following exposure to radiation (e.g., a ultraviolet radiation, visible light, etc.). Examples include benzophenones, aziridines, a photoprobe analog of geranylgeranyl diphosphate (2-diazo-3,3,3-trifluoropropionyloxy-farnesyl diphosphate or DATFP-FPP) (Quellhorst et al. J Biol Chem. 2001 Nov. 2; 276(44):40727-33), a DNA analogue 5-[N-(p-azidobenzoyl)-3-aminoallyl]-dUTP (N(3)RdUTP), sulfosuccinimidyl-2(7-azido-4-methylcoumarin-3-acetamido)-ethyl-1,3′-dithiopropionate (SAED) and 1-[N-(2-hydroxy-5-azidobenzoyl)-2-aminoethyl]-4-(N-hydroxysuccinimidyl)-succinate.

Alternatively, the directly detectable group is a photoswitch label, which is a molecule that undergoes a conformational change in response to radiation. For example, the molecule may change its conformation from cis to trans and back again in response to radiation. The wavelength required to induce the conformational switch will depend upon the particular photoswitch label. Examples of photoswitch labels include azobenzene, 3-nitro-2-naphthalenemethanol. Examples of photoswitches are also described in van Delden et al. Chemistry. 2004 Jan. 5; 10(1):61-70; van Delden et al. Chemistry. 2003 Jun. 16; 9(12):2845-53; Zhang et al. Bioconjug Chem. 2003 July-August; 14(4):824-9; Irie et al. Nature. 2002 December 19-26; 420(6917):759-60; as well as many others.

A directly detectable group can also be a photolabile protecting group, including a nitrobenzyl group, a dimethoxy nitrobenzyl group, nitroveratryloxycarbonyl (NVOC), 2-(dimethylamino)-5-nitrophenyl (DANP), Bis(o-nitrophenyl)ethanediol, brominated hydroxyquinoline, and coumarin-4-ylmethyl derivative. Photolabile protecting groups are useful for photocaging reactive functional groups.

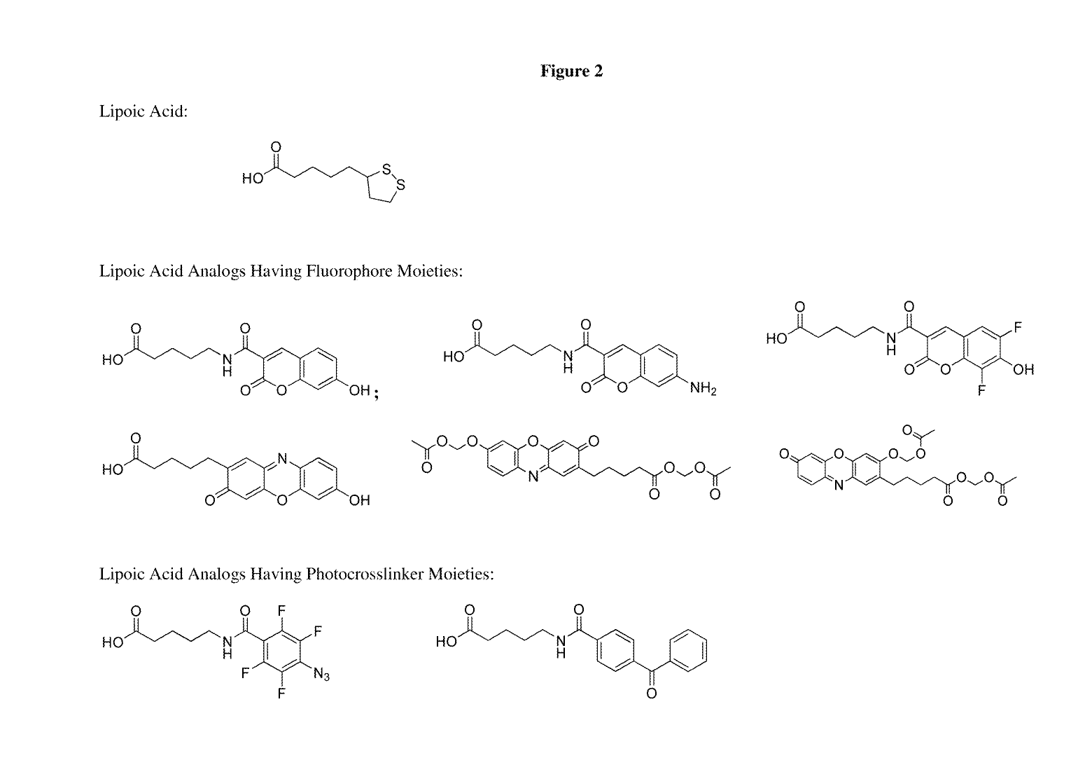

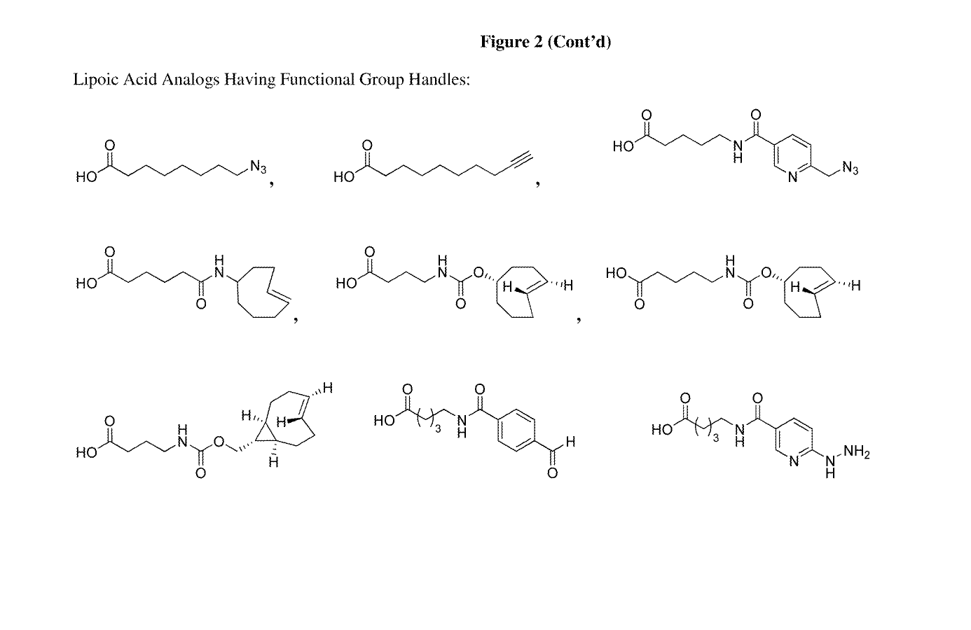

Exemplary lipoic acid analogs for use in the methods described herein include, but are not limited to, those shown below and those listed in FIG. 2.

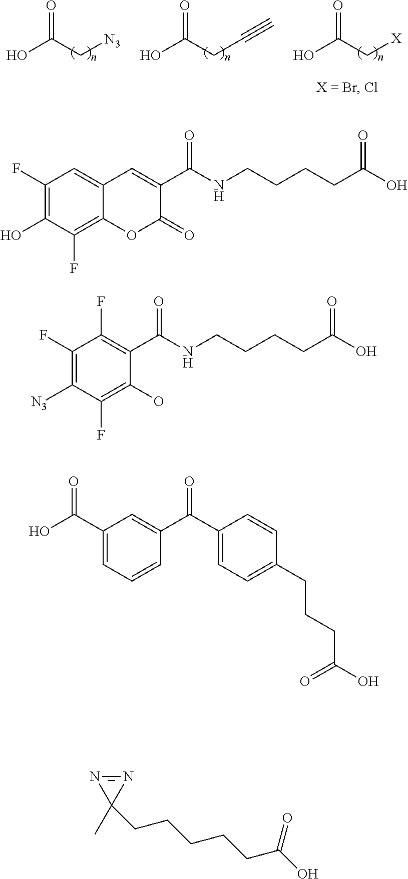

In some embodiments, a lipoic acid analog for use in the methods described herein is not one of the compounds shown directly above. In some embodiments, a lipoic acid analog for use in the methods described herein is not one of the compounds shown in FIG. 2. In some embodiments, when R¹ is C₅ alkyl, R does not comprise a diaziridine.

Any of the lipoic acid analogs can be synthesized by chemistry transformations (including protecting group methodologies), e.g., those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof. Exemplary synthetic schemes for preparing a number of lipoic acid analogs are provided in U.S. Pat. No. 8,137,925 and U.S. patent application Ser. No. 13/267,761, and also in the references listed in Table 2 above, all of which are herein incorporated by reference.

Further, one of ordinary skill in the art will recognize how to modify lipoic acid analogs to prepare additional lipoic acid analogs that are useful in methods described herein. Various assays can be used to test the substrate specificity of a lipoic acid ligase polypeptide, and the suitability of various lipoic acid analogs and acceptor polypeptides for mammalian cell labeling applications. A non-limiting example of a method for identifying a lipoic acid analog having specificity for a lipoic acid ligase polypeptide includes combining an acceptor polypeptide with a candidate lipoic acid analog molecule in the presence of a lipoic acid ligase or mutant thereof and determining the presence of lipoic acid analog incorporation, wherein lipoic acid analog incorporation is indicative of a candidate lipoic acid analog having specificity for a lipoic acid ligase or mutant thereof. Additional exemplary assays and methods of determining the presence of lipoic acid incorporation are provided in the Examples section herein.

Any of the lipoic acid analogs, in isolated form, are also within the scope of this disclosure. Isolated lipoic acid analogs similarly are analogs that have been substantially separated from either their native environment (if it exists in nature) or their synthesis environment. Accordingly, the lipoic acid analogs are substantially separated from any or all reagents present in their synthesis reaction that would be toxic or otherwise detrimental to the target protein, the acceptor peptide, the lipoic acid ligase mutant, or the labeling reaction. Isolated lipoic acid analogs, for example, include compositions that comprise less than 25% contamination, less than 20% contamination, less than 15% contamination, less than 10% contamination, less than 5% contamination, or less than 1% contamination (w/w).

### Acceptor Polypeptides

Native protein substrates of lipoic acid ligase (e.g., E2o, E2p, or H-protein) contain a 12-17 amino acid minimal substrate sequence that encompasses a lysine lipoylation site at the tip of a sharp β-turn. For example in E. coli E2o, the lysine at the tip of a sharp β-turn is the lysine that is in position 44 of E. coli E2o, see GenBank Accession No. AAA23898. In each of the three lipoyl domains of E. coli E2p, the lysines at the tip of the sharp β-turn are the lysine lipoylation sites (e.g., the lysine in position of the lipoyl hybrid domain, see ProteinDataBank Accession No. 1QJO). In E. coli H-protein, the lysine at the tip of a sharp β-turn is the lysine that is in position 65 of E. coli H-protein, see GenBank Accession No. CAA52145. Testing has shown that although accurate positioning of the target lysine within the β-turn is important for LplA recognition, the residues flanking the lysine can be varied.

Acceptor polypeptides are peptide substrates of a lipoic acid ligase, which can be designed based on the structure of a native lipoic acid ligase peptide substrate. Typically, an acceptor polypeptide has a length of 8-22 amino acid residues (e.g., 8-13 amino acid residues), forms a β-turn structure, and has a lysine residue at the tip of the β-turn, this lysine residue being reactive to a lipoic acid analog as catalyzed by a lipoic acid ligase polypeptide.

In some embodiments, the acceptor polypeptides described herein each comprises the P⁻⁴P⁻³P⁻²P⁻¹P⁰P⁺¹P⁺²P⁺³P⁺⁴P⁺⁵ (SEQ ID NO:2), in which P⁻⁴ is a hydrophobic amino acid residue (e.g., I, V, L, and F), P⁻³ is E or D, P⁻² is any amino acid residue (e.g., I), P⁻¹ is D, N, E, Y, A, or V, P⁰ is K, P⁺¹ is a hydrophobic amino acid residue (e.g., A, I, V, or L), P⁺² is a hydrophobic amino acid residue (e.g., an aromatic residue such as W, F and Y) or S, P⁺³ is a hydrophobic amino acid residue (e.g., an aliphatic hydrophobic residue such as L or V or an aromatic hydrophobic residue such as W, F, or Y), P⁺⁴ is E or D, and P⁺⁵ is a hydrophobic amino acid residue (e.g., an aliphatic hydrophobic residue such as L and V). Exemplary acceptor polypeptides include, but are not limited to DEVLVEIETDKAVLEVPGGEEE (LAP1; SEQ ID NO:3), GFEIDKVWYDLDA (LAP2; SEQ ID NO:4), GFEIDKVWHDFPA (LAP4.2; SEQ ID NO:5), or GFEIDKVFYDLDA (LAP2-F; SEQ ID NO:6). Additional acceptor polypeptides were disclosed in U.S. Pat. No. 8,137,925 and US 20110130348, which is incorporated by reference herein.

In one example, an acceptor polypeptide can derive from a native protein substrate of a lipoic acid ligase, for example, GDTLCIVEADKASMEIP (from C. coli BCCP), DDVLCEVQNDKAVVEIP (from B. stearoth. E2p), DEVLVEIDTDKVVLEVP (from E. coli E2o), DEVLVEIETDKAVLEVP (from E. coli E2o). U.S. Pat. No. 8,137,925. In another example, an acceptor polypeptide can be a high affinity peptide substrate of a lipoic acid ligase polypeptide identified by a screening method known in the art, e.g., screening a peptide-display library (see e.g., US 20110130348 and Puthenveetil et al., J. Am. Chem. Soc. 2009, 131:16430-16438). Such a high affinity acceptor polypeptides can have a k_{cat} value in the range of 0.001 s⁻¹-1.0 s⁻¹ (e.g., approximately 0.22±0.01 s⁻¹) and/or a Kₘ value in the range of 1 μM-500 μM (e.g., approximately 13.32±1.78 μM), and/or a k_{cat}/Kₘ ratio in the range of 0.0001-10 μM⁻¹ min⁻¹. High affinity acceptor polypeptides can have a length ranging from 8-13 amino acids.

One of ordinary skill in the art will recognize how to identify acceptor polypeptides and how to modify acceptor polypeptides to prepare additional acceptor polypeptides that are useful in the methods described herein. Various assays can be used to test the sequence specificity of acceptor polypeptides and their suitability for mammalian cell labeling applications. A non-limiting example of a method for identifying an acceptor polypeptide includes combining a candidate acceptor polypeptide with a labeled lipoic acid or analog thereof in the presence of a lipoic acid ligase or mutant thereof and determining a level of lipoic acid or lipoic acid analog incorporation, wherein lipoic acid or lipoic acid analog incorporation is indicative of a candidate acceptor polypeptide having specificity for a lipoic acid ligase or mutant thereof.

Any of the acceptor peptides described herein can be tagged to a target protein to be labeled by a lipoic acid analog catalyzed by a lipoic acid ligase polypeptide. The acceptor peptide and target protein may be fused to each other either at the nucleic acid or amino acid level. Recombinant DNA technology for generating fusion nucleic acids that encode both the target protein and the acceptor peptide are well known in the art. Additionally, the acceptor peptide may be fused to the target protein post-translationally. Such linkages may include cleavable linkers or bonds which can be cleaved once the desired labeling is achieved. Such bonds may be cleaved by exposure to a particular pH, or energy of a certain wavelength, and the like. Cleavable linkers are known in the art. Examples include thiol-cleavable cross-linker 3,3′-dithiobis(succinimidyl proprionate), amine-cleavable linkers, and succinyl-glycine spontaneously cleavable linkers.

The acceptor peptide can be fused to the target protein at any position. In some instances, it is preferred that the fusion not interfere with the activity of the target protein, accordingly, the acceptor peptide is fused to the protein at positions that do not interfere with the activity of the protein. Generally, the acceptor peptides can be C- or N-terminally fused to the target proteins. In still other instances, the acceptor peptide is fused to the target protein at an internal position (e.g., a flexible internal loop). These proteins are then susceptible to specific tagging by lipoic acid ligase and/or mutants thereof in vivo and in vitro. This specificity is possible because neither lipoic acid ligase nor the acceptor peptide react with any other enzymes or peptides in a cell.

### Methods for Preparing Protein Conjugates

To conjugate a lipoic acid analog as described above to a protein of interest, the analog is in contact with a fusion protein containing a protein of interest and any suitable acceptor polypeptide described above in the presence of a suitable lipoic acid ligase polypeptide, which is also described above, under conditions allowing a lipoic acid ligase reaction to take place.

In one example, this conjugation reaction is carried out in vitro. Conditions for in vitro lipoic acid ligase reactions are well known in the art, e.g., those described in the U.S. Pat. No. 8,137,925 and U.S. patent application Ser. No. 13/267,761, as well as in the references listed in Table 2 above, and in Examples below. Lipoic acid analog incorporation can be measured using ³H-lipoic acid and measuring incorporation of radioisotope in the peptide. Conjugation of the lipoic acid analog to an acceptor peptide can be assayed by various methods including, but not limited to, HPLC or mass-spec assays, as described herein and as shown in the figures herein.

Alternatively, the conjugation reaction can be carried out in vivo. Briefly, expression vectors for producing the above-noted fusion protein and the lipoic acid ligase polypeptide are introduced into cells via routine recombinant technology. The transformed cells are cultured under suitable conditions in the presence of the lipoic acid analog, which preferably can be detected directly, e.g., containing a fluorescent moiety such as the coumarin and resorufin analogs described herein. The cells are then washed to remove free lipoic acid analogs. Conjugation of the lipoic acid analog to the fusion protein can then be examined via routine technology, e.g., fluorescent microscopy. U.S. Pat. No. 8,137,925 and U.S. patent application Ser. No. 13/267,761, as well as in the references listed in Table 2 above, and in Examples below.

Virtually any cells, prokaryotic or eukaryotic, which can be transformed with heterologous DNA or RNA and which can be grown or maintained in culture, may be used in the in vivo methods described above. Examples include bacterial cells such as E. coli, mammalian cells such as mouse, hamster, pig, goat, primate, etc., and other eukaryotic cells such as Xenopus cells, Drosophila cells, Zebrafish cells, C. elegans cells, and the like. They may be of a wide variety of tissue types, including mast cells, fibroblasts, oocytes and lymphocytes, and they may be primary cells or cell lines. Specific examples include CHO cells, COS cells, and 293T cells. Cell-free transcription systems also may be used in lieu of cells.

As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids, phagemids and virus genomes. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase.

An expression vector is one into which a desired DNA sequence may be inserted by restriction and ligation such that it is operably joined to regulatory sequences and may be expressed as an RNA transcript. Vectors may further contain one or more marker sequences (i.e., reporter sequences) suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., beta-galactosidase or alkaline phosphatase), and genes which visibly affect the phenotype of transformed or transfected cells, hosts, colonies or plaques. Preferred vectors are those capable of autonomous replication and expression of the structural gene products present in the DNA segments to which they are operably joined.

As used herein, a marker or coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CCAAT sequence, and the like. Especially, such 5′ non-transcribed regulatory sequences will include a promoter region which includes a promoter sequence for transcriptional control of the operably joined coding sequence. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous nucleic acid, usually DNA, molecules, encoding a lipoic acid ligase mutant. The heterologous nucleic acid molecules are placed under operable control of transcriptional elements to permit the expression of the heterologous nucleic acid molecules in the host cell.

Preferred systems for mRNA expression in mammalian cells are those such as pcDNA3.1 (available from Invitrogen, Carlsbad, Calif.) that contain a selectable marker such as a gene that confers G418 resistance (which facilitates the selection of stably transfected cell lines) and the human cytomegalovirus (CMV) enhancer-promoter sequences. Additionally, suitable for expression in primate or canine cell lines is the pCEP4 vector (Invitrogen, Carlsbad, Calif.), which contains an Epstein Barr virus (EBV) origin of replication, facilitating the maintenance of plasmid as a multicopy extrachromosomal element. Another expression vector is the pEF-BOS plasmid containing the promoter of polypeptide Elongation Factor 1α, which stimulates efficiently transcription in vitro. The plasmid is described by Mishizuma and Nagata (Nuc. Acids Res. 18:5322, 1990), and its use in transfection experiments is disclosed by, for example, Demoulin (Mol. Cell. Biol. 16:4710-4716, 1996). Still another preferred expression vector is an adenovirus, described by Stratford-Perricaudet, which is defective for E1 and E3 proteins (J. Clin. Invest. 90:626-630, 1992). The use of the adenovirus as an Adeno.P1A recombinant is disclosed by Warnier et al., in intradermal injection in mice for immunization against P1A (Int. J. Cancer, 67:303-310, 1996).

The present disclosure also embraces so-called expression kits, which allow the artisan to prepare a desired expression vector or vectors. Such expression kits include at least separate portions of each of the previously discussed coding sequences (e.g., a coding sequence for a lipoic acid ligase polypeptide and a coding sequence for a fusion protein containing a protein of interest and an acceptor polypeptide. Other components may be added, as desired, as long as the previously mentioned sequences, which are required, are included.

It will also be recognized that the invention embraces the use of the above described, lipoic acid ligase mutant encoding nucleic acid containing expression vectors, to transfect host cells and cell lines, be these prokaryotic (e.g., E. coli), or eukaryotic (e.g., rodent cells such as CHO cells, primate cells such as COS cells, Drosophila cells, Zebrafish cells, Xenopus cells, C. elegans cells, yeast expression systems and recombinant baculovirus expression in insect cells). Especially useful are mammalian cells such as human, mouse, hamster, pig, goat, primate, etc., from a wide variety of tissue types including primary cells and established cell lines.

Various methods of the invention also require expression of fusion proteins in vivo. The fusion proteins are generally recombinantly produced proteins that comprise the lipoic acid ligase acceptor peptides. Such fusions can be made from virtually any protein and those of ordinary skill in the art will be familiar with such methods. Further conjugation methodology is also provided in U.S. Pat. Nos. 5,932,433; 5,874,239 and 5,723,584.

In some instances, it may be desirable to place the lipoic acid ligase polypeptide and possibly the fusion protein under the control of an inducible promoter. An inducible promoter is one that is active in the presence (or absence) of a particular moiety. Accordingly, it is not constitutively active. Examples of inducible promoters are known in the art and include the tetracycline responsive promoters and regulatory sequences such as tetracycline-inducible T7 promoter system, and hypoxia inducible systems (Hu et al. Mol Cell Biol. 2003 December; 23(24):9361-74). Other mechanisms for controlling expression from a particular locus include the use of synthetic short interfering RNAs (siRNAs).

Alternatively, it may be desirable to insert into the lipoic acid ligase polypeptide and possibly the fusion protein a subcellular localization signaling peptide such that the expressed lipoic acid ligase polypeptide and/or the fusion protein are localized in a desired subcellular compartment, e.g., mitochondria or the Golgi apparatus. Such signaling peptides are well known in the art.

In some embodiments, the method for preparing a protein conjugate described above is a one-step method for labeling a protein of interest, using a lipoic acid analog that comprises a directly detectable group. Following any of the in vitro and in vivo preparation methods described above, the lipoic acid analog is conjugated to a protein of interest, thereby labeling that protein.

In other embodiments, the methods described above involve two steps to label a protein of interest. In the first step, a lipoic acid analog comprising a functional group handle is conjugated to a protein of interest fused with an acceptor polypeptide in the presence of a suitable lipoic acid ligase polypeptide to form a first protein conjugate. In the second step, the first protein conjugate is in contact with a compound comprising a functional group that is reactive to the functional group handle in the first protein conjugate and a detectable (directly detectable or indirectly detectable) label. Upon reaction between the functional group handle in the first protein conjugate and the functional group in the compound, the detectable label is linked to the protein of interest.

When the functional group handle in a lipoic acid analog is a trans-cyclooctene compound, such as those described in Liu et al., J. Am. Chem. Soc. 2012, 134(2):792-795, a protein conjugate containing such a lipoic acid analog can further react to a tetrazine conjugate containing a detectable label via the diels-alder cycloaddition reaction. Exemplary tetrazine compounds to be used in the second reactive step include, but are not listed to, Tz1 and Tz2 shown below:

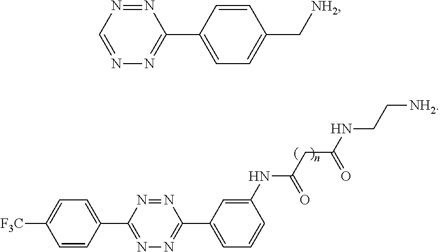

In some embodiments, the labeled compound used in the second step contains a phosphine group and a lipoic acid analog (e.g., an azide) may be reacted with the phosphine group in a Staudinger reaction. Azides and aryl phosphines generally have no cellular counterparts. As a result, the reaction is quite specific. Azide variants with improved stability against hydrolysis in water at pH 6-8 are also useful in the methods of the invention. The alkyne/azide [3+2] cycloaddition chemistry, based on Click chemistry (Wang et al. J. Am. Chem. Soc. 125:11164-11165, 2003), is also specific, in part because the two reactive partners do not have cellular counterparts (i.e., the two functional groups are non-naturally occurring). Nonlimiting examples of fluorophores that may be conjugated to a cyclooctyne are Alexa Fluor 568 and Cy3.

Other examples of functional groups include, but are not limited to, (functional group: reactive group of light emissive compound) activated ester:amines or anilines; acyl azide:amines or anilines; acyl halide:amines, anilines, alcohols or phenols; acyl nitrile: alcohols or phenols; aldehyde:amines or anilines; alkyl halide:amines, anilines, alcohols, phenols or thiols; alkyl sulfonate:thiols, alcohols or phenols; anhydride:alcohols, phenols, amines or anilines; aryl halide:thiols; aziridine:thiols or thioethers; carboxylic acid:amines, anilines, alcohols or alkyl halides; diazoalkane:carboxylic acids; epoxide:thiols; haloacetamide:thiols; halotriazine:amines, anilines or phenols; hydrazine:aldehydes or ketones; hydroxyamine:aldehydes or ketones; imido ester:amines or anilines; isocyanate:amines or anilines; and isothiocyanate:amines or anilines.

A “detectable label” as used herein is a molecule or compound that can be detected by a variety of methods including fluorescence, electrical conductivity, radioactivity, size, and the like. The label may be of a chemical (e.g., carbohydrate, lipid, etc.), peptide or nucleic acid nature although it is not so limited. The label may be directly or indirectly detectable. The label can be detected directly for example by its ability to emit and/or absorb light of a particular wavelength. A label can be detected indirectly by its ability to bind, recruit and, in some cases, cleave (or be cleaved by) another compound, thereby emitting or absorbing energy. An example of indirect detection is the use of an enzyme label that cleaves a substrate into visible products.

The type of label used will depend on a variety of factors, such as but not limited to the nature of the protein ultimately being labeled. The label should be sterically and chemically compatible with the lipoic acid analog, the acceptor peptide and the target protein. In most instances, the label should not interfere with the activity of the target protein.

Generally, the label can be selected from the group consisting of a fluorescent molecule, a chemiluminescent molecule (e.g., chemiluminescent substrates), a phosphorescent molecule, a radioisotope, an enzyme, an enzyme substrate, an affinity molecule, a ligand, an antigen, a hapten, an antibody, an antibody fragment, a chromogenic substrate, a contrast agent, an MRI contrast agent, a PET label, a phosphorescent label, and the like.

Specific examples of labels include radioactive isotopes such as ³²P or ³H; haptens such as digoxigenin and dintrophenyl; affinity tags such as a FLAG tag, an HA tag, a histidine tag, a GST tag; enzyme tags such as alkaline phosphatase, horseradish peroxidase, beta-galactosidase, etc. Other labels include fluorophores such as fluorescein isothiocyanate (“FITC”), Texas Red®, tetramethylrhodamine isothiocyanate (“TRITC”), 4,4-difluoro-4-bora-3a, and 4a-diaza-s-indacene (“BODIPY”), Cy-3, Cy-5, Cy-7, Cy-Chrome™, R-phycoerythrin (R-PE), PerCP, allophycocyanin (APC), PharRed™, Mauna Blue, Alexa™ 350 and other Alexa™ dyes, and Cascade Blue®.

The labels can also be antibodies or antibody fragments or their corresponding antigen, epitope or hapten binding partners. Detection of such bound antibodies and proteins or peptides is accomplished by techniques well known to those skilled in the art. Antibody/antigen complexes which form in response to hapten conjugates are easily detected by linking a label to the hapten or to antibodies which recognize the hapten and then observing the site of the label. Alternatively, the antibodies can be visualized using secondary antibodies or fragments thereof that are specific for the primary antibody used. Polyclonal and monoclonal antibodies may be used. Antibody fragments include Fab, F(ab)₂, Fd and antibody fragments which include a CDR3 region. The conjugates can also be labeled using dual specificity antibodies.

The label can be a positron emission tomography (PET) label such as 99m technetium and 18FDG.

The label can also be an singlet oxygen radical generator including but not limited to resorufin, malachite green, fluorescein, benzidine and its analogs including 2-aminobiphenyl, 4-aminobiphenyl, 3,3′-diaminobenzidine, 3,3′-dichlorobenzidine, 3,3′-dimethoxybenzidine, and 3,3′-dimethylbenzidine. These molecules are useful in EM staining and can also be used to induce localized toxicity.

The label can also be an analyte-binding group such as but not limited to a metal chelator (e.g., a copper chelator). Examples of metal chelators include EDTA, EGTA, and molecules having pyridinium substituents, imidazole substituents, and/or thiol substituents. These labels can be used to analyze local environment of the target protein (e.g., Ca²⁺ concentration).

The label can also be a heavy atom carrier. Such labels would be particularly useful for X-ray crystallographic study of the target protein. Heavy atoms used in X-ray crystallography include but are not limited to Au, Pt and Hg. An example of a heavy atom carrier is iodine.

The label may also be a photoactivatable cross-linker. A photoactivable cross linker is a cross linker that becomes reactive following exposure to radiation (e.g., an ultraviolet radiation, visible light, etc.). Examples include benzophenones, aziridines, a photoprobe analog of geranylgeranyl diphosphate (2-diazo-3,3,3-trifluoropropionyloxy-farnesyl diphosphate or DATFP-FPP) (Quellhorst et al. J Biol Chem. 2001 Nov. 2; 276(44):40727-33), a DNA analogue 5-[N-(p-azidobenzoyl)-3-aminoallyl]-dUTP (N(3)RdUTP), sulfosuccinimidyl-2(7-azido-4-methylcoumarin-3-acetamido)-ethyl-1,3′-dithiopropionate (SAED) and 1-[N-(2-hydroxy-5-azidobenzoyl)-2-aminoethyl]-4-(N-hydroxysuccinimidyl)-succinate.

The label may also be a photoswitch label. A photoswitch label is a molecule that undergoes a conformational change in response to radiation. For example, the molecule may change its conformation from cis to trans and back again in response to radiation. The wavelength required to induce the conformational switch will depend upon the particular photoswitch label. Examples of photoswitch labels include azobenzene, 3-nitro-2-naphthalenemethanol. Examples of photoswitches are also described in van Delden et al. Chemistry. 2004 Jan. 5; 10(1):61-70; van Delden et al. Chemistry. 2003 Jun. 16; 9(12):2845-53; Zhang et al. Bioconjug Chem. 2003 July-August; 14(4):824-9; Irie et al. Nature. 2002 December 19-26; 420(6917):759-60; as well as many others.

The label may also be a photolabile protecting group. Examples of photolabile protecting group include a nitrobenzyl group, a dimethoxy nitrobenzyl group, nitroveratryloxycarbonyl (NVOC), 2-(dimethylamino)-5-nitrophenyl (DANP), Bis(o-nitrophenyl)ethanediol, brominated hydroxyquinoline, and coumarin-4-ylmethyl derivative. Photolabile protecting groups are useful for photocaging reactive functional groups.

The label may comprise non-naturally occurring amino acids. Examples of non-naturally occurring amino acids include for glutamine (Glu) or glutamic acid residues: α-aminoadipate molecules; for tyrosine (Tyr) residues: phenylalanine (Phe), 4-carboxymethyl-Phe, pentafluoro phenylalanine (PfPhe), 4-carboxymethyl-L-phenylalanine (cmPhe), 4-carboxydifluoromethyl-L-phenylalanine (F₂ cmPhe), 4-phosphonomethyl-phenylalanine (Pmp), (difluorophosphonomethyl)phenylalanine (F₂Pmp), O-malonyl-L-tyrosine (malTyr or OMT), and fluoro-O-malonyltyrosine (FOMT); for proline residues: 2-azetidinecarboxylic acid or pipecolic acid (which have 6-membered, and 4-membered ring structures respectively); 1-aminocyclohexylcarboxylic acid (Ac₆c); 3-(2-hydroxynaphtalen-1-yl)-propyl; S-ethylisothiourea; 2-NH₂-thiazoline; 2-NH₂-thiazole; asparagine residues substituted with 3-indolyl-propyl at the C terminal carboxyl group. Modifications of cysteines, histidines, lysines, arginines, tyrosines, glutamines, asparagines, prolines, and carboxyl groups are known in the art and are described in U.S. Pat. No. 6,037,134. These types of labels can be used to study enzyme structure and function.

The label may be an enzyme or an enzyme substrate. Examples of these include (enzyme (substrate)): Alkaline Phosphatase (4-Methylumbelliferyl phosphate Disodium salt; 3-Phenylumbelliferyl phosphate Hemipyridine salt); Aminopeptidase (L-Alanine-4-methyl-7-coumarinylamide trifluoroacetate; Z-L-arginine-4-methyl-7-coumarinylamide hydrochloride; Z-glycyl-L-proline-4-methyl-7-coumarinylamide); Aminopeptidase B (L-Leucine-4-methyl-7-coumarinylamide hydrochloride); Aminopeptidase M (L-Phenylalanine 4-methyl-7-coumarinylamide trifluoroacetate); Butyrate esterase (4-Methylumbelliferyl butyrate); Cellulase (2-Chloro-4-nitrophenyl-beta-D-cellobioside); Cholinesterase (7-Acetoxy-1-methylquinolinium iodide; Resorufin butyrate); alpha-Chymotrypsin, (Glutaryl-L-phenylalanine 4-methyl-7-coumarinylamide); N—(N-Glutaryl-L-phenylalanyl)-2-aminoacridone; N—(N-Succinyl-L-phenylalanyl)-2-aminoacridone); Cytochrome P450 2B6 (7-Ethoxycoumarin); Cytosolic Aldehyde Dehydrogenase (Esterase Activity) (Resorufin acetate); Dealkylase (O⁷-Pentylresorufin); Dopamine beta-hydroxylase (Tyramine); Esterase (8-Acetoxypyrene-1,3,6-trisulfonic acid Trisodium salt; 3-(2 Benzoxazolyl)umbelliferyl acetate; 8-Butyryloxypyrene-1,3,6-trisulfonicacid Trisodium salt; 2′,7′-Dichlorofluorescin diacetate; Fluorescein dibutyrate; Fluorescein dilaurate; 4-Methylumbelliferyl acetate; 4-Methylumbelliferyl butyrate; 8-Octanoyloxypyrene-1,3,6-trisulfonic acid Trisodium salt; 8-Oleoyloxypyrene-1,3,6-trisulfonic acid Trisodium salt; Resorufin acetate); Factor X Activated (Xa) (4-Methylumbelliferyl 4-guanidinobenzoate hydrochloride Monohydrate); Fucosidase, alpha-L-(4-Methylumbelliferyl-alpha-L-fucopyranoside); Galactosidase, alpha-(4-Methylumbelliferyl-alpha-D galactopyranoside); Galactosidase, beta-(6,8-Difluoro-4-methylumbelliferyl-beta-D-galactopyranoside; Fluorescein di(beta-D-galactopyranoside); 4-Methylumbelliferyl-alpha-D-galactopyranoside; 4-Methylumbelliferyl-beta-D-lactoside: Resorufin-beta-D-galactopyranoside; 4-(Trifluoromethyl)umbelliferyl-beta-D-galactopyranoside; 2-Chloro-4-nitrophenyl-beta-D-lactoside); Glucosaminidase, N-acetyl-beta-(4-Methylumbelliferyl-N-acetyl-beta-D-glucosaminide Dihydrate); Glucosidase, alpha-(4-Methylumbelliferyl-alpha-D-glucopyranoside); Glucosidase, beta-(2-Chloro-4-nitrophenyl-beta-D-glucopyranoside; 6,8-Difluoro-4-methylumbelliferyl-beta-D-glucopyranoside; 4-Methylumbelliferyl-beta-D-glucopyranoside; Resorufin-beta-D-glucopyranoside; 4-(Trifluoromethyl)umbelliferyl-beta-D-glucopyranoside); Glucuronidase, beta-(6,8-Difluoro-4-methylumbelliferyl-beta-D-glucuronide Lithium salt; 4-Methylumbelliferyl-beta-D-glucuronide Trihydrate); Leucine aminopeptidase(L-Leucine-4-methyl-7-coumarinylamide hydrochloride); Lipase (Fluorescein dibutyrate; Fluorescein dilaurate; 4-Methylumbelliferyl butyrate; 4-Methylumbelliferyl enanthate; 4-Methylumbelliferyl oleate; 4-Methylumbelliferyl palmitate; Resorufin butyrate); Lysozyme (4-Methylumbelliferyl-N,N′,N″-triacetyl-beta-chitotrioside); Mannosidase, alpha-(4-Methylumbelliferyl-alpha-D-mannopyranoside); Monoamine oxidase (Tyramine); Monooxygenase (7-Ethoxycoumarin); Neuraminidase (4-Methylumbelliferyl-N-acetyl-alpha-D-neuraminic acid Sodium salt Dihydrate); Papain (Z-L-arginine-4-methyl-7-coumarinylamide hydrochloride); Peroxidase (Dihydrorhodamine 123); Phosphodiesterase (1-Naphthyl 4-phenylazophenyl phosphate; 2-Naphthyl 4-phenylazophenyl phosphate); Prolyl endopeptidase (Z-glycyl-L-proline-4-methyl-7-coumarinylamide; Z-glycyl-L-proline-2-naphthylamide; Z-glycyl-L-proline-4-nitroanilide); Sulfatase (4-Methylumbelliferyl sulfate Potassium salt); Thrombin (4-Methylumbelliferyl 4-guanidinobenzoate hydrochloride Monohydrate); Trypsin (Z-L-arginine-4-methyl-7-coumarinylamide hydrochloride; 4-Methylumbelliferyl 4-guanidinobenzoate hydrochloride Monohydrate); Tyramine dehydrogenase (Tyramine).

Labels can be attached to a functional group to prepare the compounds to be used in the second step of the methods described herein by any mechanism known in the art.

The labels are detected using a detection system. The nature of such detection systems will depend upon the nature of the detectable label. The detection system can be selected from any number of detection systems known in the art. These include a fluorescent detection system, a photographic film detection system, a chemiluminescent detection system, an enzyme detection system, an atomic force microscopy (AFM) detection system, a scanning tunneling microscopy (STM) detection system, an optical detection system, a nuclear magnetic resonance (NMR) detection system, a near field detection system, and a total internal reflection (TIR) detection system.

### Study Protein-Protein Interaction

Also described herein is a method for imaging protein-protein interaction (PPI) via a reaction catalyzed by a lipoic acid ligase polypeptide. FIG. 15 provides an example of how this imaging method is performed. In this method, A and B are two proteins whose interaction is to be studied. A lipoic acid ligase polypeptide as described herein is fused to protein A, and an acceptor polypeptide (e.g., a low affinity acceptor polypeptide as described above) is fused to protein B. If A and B interact, the ligase attaches a probe, which is a lipoic acid analog as described herein, to the acceptor polypeptide. If A and B do not interact, the enzyme and peptide do not associate and no labeling occurs. See also Slavoff et al., J. Am. Chem. Soc. 2011, 133:19769-19776, which is herein incorporated by reference.

The system is engineered to provide high labeling sensitivity when an interaction occurs and low background in the absence of an interaction. This is achieved by treating the interaction as a kinetic switch: when no interaction occurs, the rate of peptide labeling by the enzyme is undetectably slow, but when an interaction does occur, the labeling rate is maximally fast. Such switching depends on the kinetic parameters of our system. In the absence of a PPI, the protein concentrations in the cell are far below the ligase-acceptor polypeptide Kₘ, and the bimolecular reaction rate will be governed by kcat/Km. In the presence of a PPI, on the other hand, when the local concentration of the acceptor polypeptide with respect to the ligase is very high, the pseudo-zero-order reaction rate is governed by kcat. Therefore, by engineer-ing high Km, background labeling can be minimized, and by engineering high kcat, signal in the presence of a PPI can be maximized.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1

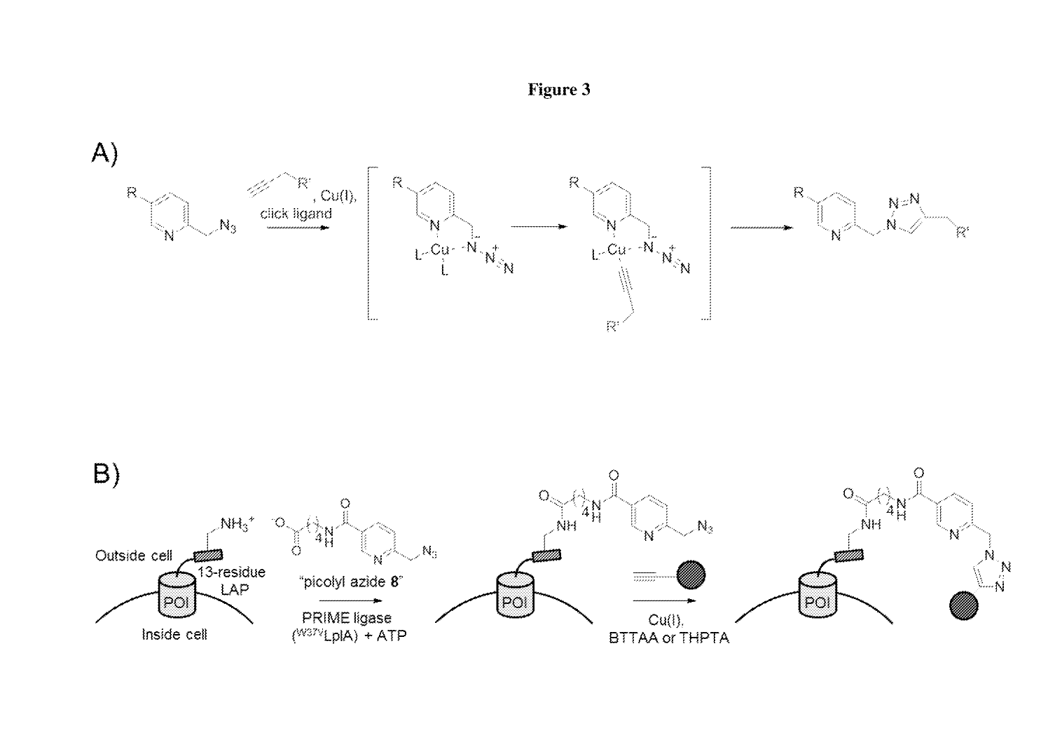

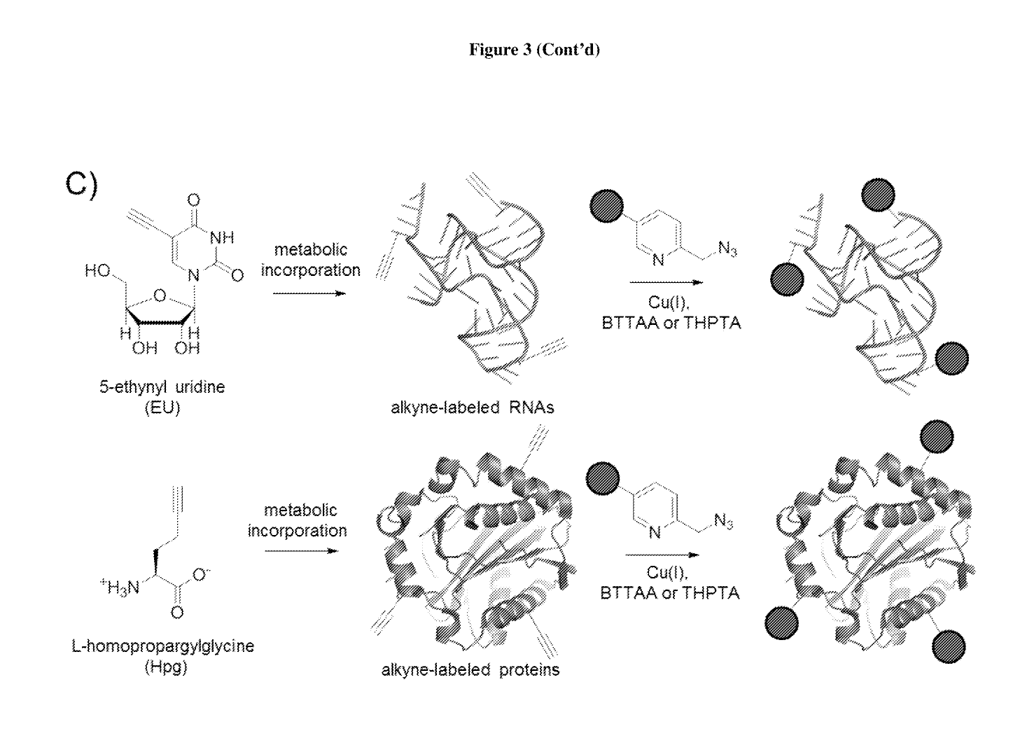

Fast Cell-Compatible Click Chemistry with Copper-Chelating Azides for Studies Disclosed in this Example Aim at Improving the Cell-Compatibility of CuAAC via introducing an internal copper chelating moiety into the azide or alkyne reaction partner without sacrificing reaction rate. FIG. 3A. The goal was to extend and optimize this concept for aqueous CuAAC reactions, under conditions relevant for biomolecular labeling.

In these studies, azides were found to be capable of copper-chelation undergo much faster “Click chemistry” (copper-accelerated azide-alkyne cycloaddition, or CuAAC) than non-chelating azides under a variety of biocompatible conditions. This kinetic enhancement allowed for performing site-specific protein labeling on the surface of living cells with only 10-40 μM CuI/II and much higher signal than could be obtained using the best previously-reported live-cell compatible CuAAC labeling conditions. Detection sensitivity was also greatly increased for CuAAC detection of metabolic labeling of total RNA and proteins in cells.

### Methods

### Kinetic Analysis of the CuAAC Reaction

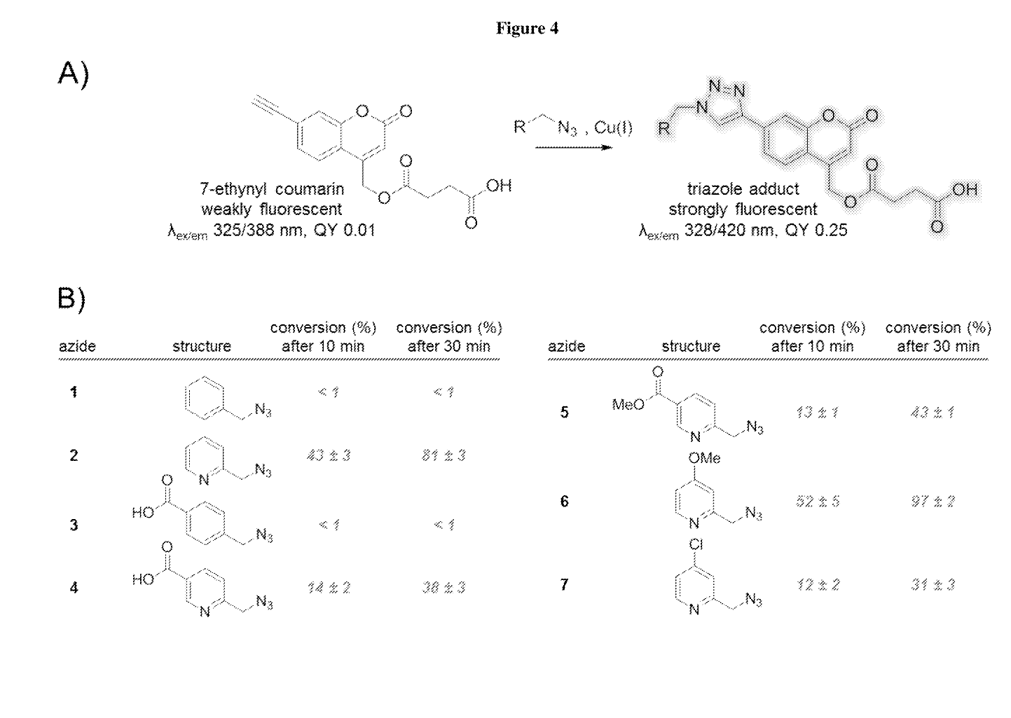

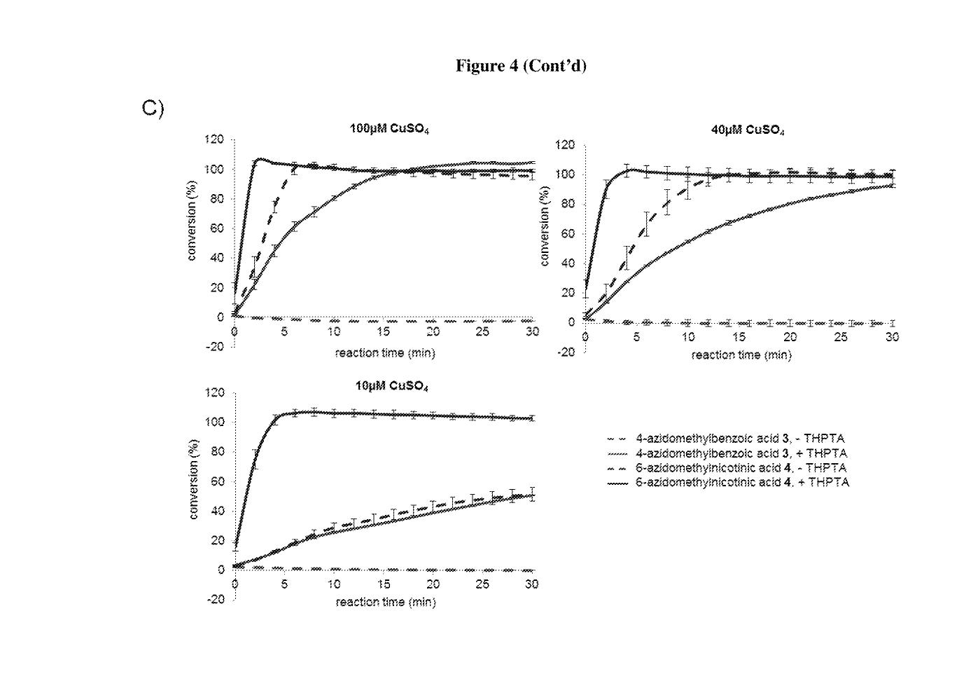

General reaction conditions: 20 μM azide, 40 μM 7-ethynyl coumarin (A), and 4 mM sodium ascorbate in 100 mM sodium phosphate buffer at pH 7.4 at 25±1° C. 100 μM Tempol was added to each reaction to minimize Cu-dependent fluorescence quenching of 7-ethynyl coumarin and coumarin-triazoles. FIG. 4A.

Reactions were initiated by the addition of CuSO₄: 10 μM for the azide compounds shown in FIG. 4B, and 10, 40, or 100 μM for the compounds shown in FIG. 4C. In FIG. 4C when THPTA was included, the THPTA:copper ratio was fixed at a 4:1 molar ratio. Coumarin fluorescence was recorded on a Tecan SAFIRE microplate reader at 2-min intervals for 30 min with excitation at 320 nm and emission detection at 430 nm. For each azide, the turn-on fluorescence of coumarin was correlated to % conversion to product using a calibration curve made from a mixture of known concentrations of 7-ethynyl coumarin and coumarin-triazole adduct of each azide, as follows:

| [7-ethynyl coumarin], | [coumarin-triazole], | % conversion to product |
|---|---|---|
| μM | μM | represented |
| 40 | 0 | 0 |
| 37.5 | 2.5 | 12.5 |
| 35 | 5 | 25 |
| 30 | 10 | 50 |
| 25 | 15 | 75 |
| 20 | 20 | 100 |

Coumarin-triazole standards for azide 1, 2, 5, 6, and 7 (FIG. 4B) were generated from reacting 120 μM of each azide with 100 μM 7-ethynyl coumarin until 7-ethynyl coumarin was fully converted to the triazole adduct, using 100 μM CuSO₄, 400 μM THPTA, and 4 mM sodium ascorbate. Complete conversion of 7-ethynyl coumarin to coumarin-triazole was achieved in 30 min for all azides, and was confirmed by thin-layer chromatography, and by monitoring for saturation of turn-on fluorescence levels of coumarin. Such reaction mixture, now representing coumarin-triazole of a known concentration (100 μM), was then mixed with 7-ethynyl coumarin in defined ratios in the presence of 20-fold molar excess of EDTA relative to CuSO₄, (which was carried over from the triazole generation reaction), to generate the calibration curve above.

Coumarin-triazole standards for azide 3 and 4 were generated from purified coumarin-triazole adducts for each azide (synthetic methods described below). Calibration curves were generated for azide 3 and 4 using coumarin-triazoles from crude reaction mixtures as described above, and found them to perform similarly to calibration curves generated from purified triazoles. FIG. 4C.

### Mammalian and Neuronal Cell Culture

Human embryonic kidney (HEK) and HeLa were cultured in minimal essential medium (MEM, Mediatech) supplemented with 10% v/v fetal bovine serum (PAA Laboratories). Human malignant melanoma (A375) cells expressing Erk2-GFP (Life Technologies) were cultured in L-glutamine-containing Dulbecco's modified Eagle Medium (Life Technologies) supplemented with 10% v/v fetal bovine serum (Life Technologies), non-essential amino acids (Life Technologies), and 5 μg/mL blasticidin. All cells were maintained at 37° C. under 5% CO₂. For imaging, HEK cells were plated as a monolayer on glass coverslips, while A375 cells were plated directly onto 96-well plates. Adherence of HEK cells was promoted by pre-coating the coverslip with 50 μg/mL fibronectin (Millipore).

For hippocampal neuron cultures, Spague Dawley rat pups were sacrificed at embryonic day 18. Hippocampal tissue was digested with papain (Worthington) and DNaseI (Roche) and plated on glass coverslips pretreated with poly-D-lysine (Sigma) and mouse laminin (Life Technologies) in L-glutamine-containing MEM (Sigma) supplemented with 10% v/v fetal bovine serum (PAA Laboratories) and B27 (Life Technologies). At 3 days in vitro, half of the growth medium was replaced with Neurobasal medium (Life Technologies) supplemented with B27 and GlutaMAX (Life Technologies).

General Protocol for Cell-Surface Protein Labeling with PRIME Followed by Chelation-Assisted CuAAC

HEK cells were transfected at ˜80% confluency with expression plasmids for LAP-tagged neurexin-1β (400 ng for a 0.95 cm² dish) and yellow fluorescent protein-tagged histone 2B protein (H2B-YFP; 100 ng) using lipofectamine 2000 (Invitrogen). 24 hr after transfection, cells were treated with 10 μM purified ^{W37V}LplA, 200 μM picolyl azide 8, 1 mM ATP, and 5 mM Mg(OAc)₂ in cell growth medium for 20 min at room temperature. After excess LplA labeling reagents had been removed by quickly replacing the medium 2-3 times, cells were further labeled with 20 μM Alexa Fluor® 647-alkyne, 50 μM CuSO₄, 250 μM THPTA (or BTTAA), and 2.5 mM sodium ascorbate in DPBS for 5 min at room temperature. Cells were immediately imaged after excess CuAAC labeling reagents were removed by 2-3 quick washes with fresh growth medium.

Labeling of LAP-Neuroligin-1 in Live Dissociated Neurons with PRIME Followed by Chelation-Assisted CuAAC

Neurons were transfected at 5 days in vitro with expression plasmids for LAP-tagged neuroligin-1 (500 ng for a 1.9 cm² dish) and green fluorescent protein-tagged Homer1b (Homer-GFP; 100 ng for a 1.9 cm² dish) using Lipofectamine 2000, using half the amount of the manufacturer's recommended reagent quantity. Neurons were labeled at 11 days in vitro with 10 μM purified ^{W37V}LplA, 200 μM picolyl azide 8, 1 mM ATP, and 5 mM Mg(OAc)₂ in preconditioned supplemented Neurobasal medium for 20 min at 37° C. After brief rinsing in supplemented preconditioned medium, neurons were further labeled with 20 μM Alexa Fluor® 647-alkyne, 50 μM Tempol, 50 μM CuSO₄, 250 μM THPTA (or BTTAA), and 2.5 mM sodium ascorbate in Tyrode's buffer for 5 min at room temperature. The labeling solution was then replaced with supplemented Neurobasal medium containing 500 μM bathocuproin sulfonate, which was incubated with neurons for 30 sec. Neurons were imaged live in Tyrode's buffer after 2 further washes with supplemented Neurobasal medium.

Metabolic Labeling of Proteins and Ribonucleic Acids with Chelation-Assisted CuAAC

A375 cells were plated at a density of ˜5000 cells per 0.3 cm² well and cultured in complete culture medium overnight. For labeling of nascent RNA transcripts, cells were incubated with culture medium containing 200 μM 5-ethynyl uridine (Life Technologies) for 90 min. For labeling of newly-synthesized proteins, cells were incubated with culture medium containing 50 μM L-homopropargylglycine (Hpg) for 90 min. Prior to incubation with Hpg-containing medium, cells were washed once with DPBS with calcium and magnesium, then grown in methionine-free DMEM (Life Technologies) for 30 min. Cells were fixed with 4% formaldehyde in PBS pH 7.4 (Life Technologies) and permeabilized with 0.5% Triton® X-100 in PBS (Sigma). CuAAC labeling was performed for 1 hr in the dark with 5 μM Alexa Fluor® 647-picolyl azide, 2 mM CuSO₄, 8 mM THPTA, and 10 mM sodium ascorbate in PBS at room temperature. After washing cells twice with 3% w/v bovine serum albumin in PBS, Hoechst 33342 staining (10 μg/mL) was performed in PBS for 30 min at room temperature. Cells were washed 3 times with PBS before imaging.

### General Synthetic Methods

Chemicals were purchased from Sigma-Aldrich, Alfa Aesar, TCI America, Fisher Scientific, Adesis Inc, or EMD unless specified otherwise. Analytical thin-layer chromatography was performed using 0.25 mm silica gel 60 F₂₅₄ plates and visualized with 254 nm UV light or with bromocresol green. ¹H NMR spectra were recorded on a Bruker Avance 400 MHz or a Varian Inova 500 MHz spectrometer. All samples were dissolved in CDCl₃, CD₃OD, D₂O, or d₆-DMSO and chemical shifts (δ) are expressed in parts per million relative to residual solvent peak as an internal standard. Abbreviations are: s, singlet; d, doublet; t, triplet; q, quartet; m, multiplet; br, broad. Coupling constants (J) are reported in hertz (Hz). Mass analyses of peptides were recorded using electrospray ionization (ESI) on an Applied Biosystems 200 QTRAP mass spectrometer or an Agilent 1100 MSD ion trap mass spectrometer. Absorbance and fluorescence properties for selected compounds were determined on a Perkin Elmer LS50B Luminescence Spectrometer in HPLC-grade methanol.

High-resolution mass spectrometric data was obtained using Waters SYNAPT-HDMS mass spectrometer equipped with Waters ACQUITY UPLC and a BEH C18 column (1.7 μm particle size, 2.1×50 mm dimension). For positive ion detection mode, the gradient used was 5-95% acetonitrile in water with 0.1% formic acid, at a 0.3 mL/min flow rate over 10 minutes. The mass spectrometry for each chromatogram was re-calibrated relative to the internal standards' accurate mass: reduced glutathione (m/z 308.0916); oxidized glutathione (m/z 613.1598); and Leu-enkephalin (m/z 556.2771-positive ion). Each azide or click-chemistry product compound's mass was centered for accurate mass and chemical formula calculated using Mass Lynx V4.1 software.

### (a) Synthesis of Organic Azides (Structures in FIG. 4B)

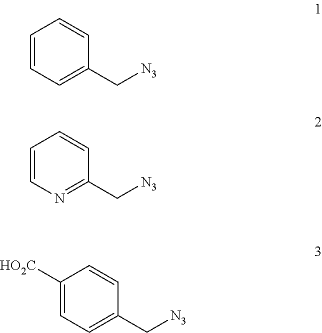

Benzyl azide (1) is commercially available.

Azide 2 (2-azidomethylpyridine) was prepared according to Brotherton, et al., Organic Letters, 11:4954-4957 (2009). ¹H NMR (400 MHz, CDCl₃): 8.57 (dd, 1H, J=4.9, 1.8 Hz), 7.69 (dt, 1H, J=7.8, 1.8 Hz), 7.31 (d, 1H, J=7.8 Hz), 7.22 (dd, 1H, J=7.8, 4.9 Hz). ¹³C NMR (100 MHz, CDCl₃): 115.8, 149.7, 137.1, 123.0, 122.0, 55.7. HR-ESI-MS: [M+H]⁺ m/z 135.0671 calculated, 135.0667 observed.

Azide 3 (4-azidomethylbenzoic acid) was prepared according to WO2010009062. ¹H NMR (400 MHz, CD₃OD): 8.03 (d, 2H, J=8.4 Hz), 7.45 (d, 2H, J=8.4 Hz), 4.91 (br, 1H), 4.46 (s, 2H). ¹³C NMR (100 MHz, CD₃OD): 169.4, 142.4, 131.7, 131.2, 129.2, 55.0. HR-ESI-MS: [M+H]⁺ m/z 176.0460 calculated, 176.0467 observed.

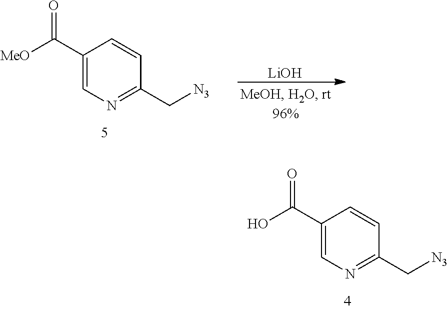 [SMILES: COC(=O)c1ccc(CN=[N+]=[N-])nc1 ; "LiOH" ; [N-]=[N+]=NCc1ccc(C(=O)O)cn1]

Azide 4 (6-Azidomethylnicotinic acid). Methyl 5-(azidomethyl)nicotinate 5 (114 mg, 0.59 mmol) was dissolved in methanol (2.5 mL). A 1.0 M solution of LiOH in water (1.78 mL, 1.78 mmol) was then added and the mixture was stirred for 25 minutes, at which time acetic acid (60 μL) was added and the mixture was loaded directly onto a silica gel column equilibrated with ethyl acetate+1% acetic acid and chromatographed with ethyl acetate+1% acetic acid to 4% acetonitrile/ethyl acetate+1% acetic acid to provide 101 mg (96%) of 4 as a yellow solid. R_{f}=0.35 (ethyl acetate+1% acetic acid, 254 nm UV). ¹H NMR (400 MHz, CD₃OD): 9.10 (dd, J=2.1, 0.8 Hz), 8.39 (dd, 1H, J=8.1, 2.1 Hz), 7.57 (dd, 1H, J=8.1, 0.8 Hz), 4.59 (s, 2H). ¹³C NMR (100 MHz, CD₃OD): 167.7, 161.3, 151.5, 139.9, 127.6, 123.3, 56.0. HR-ESI-MS: [M+H]⁺ m/z 179.0569 calculated, 179.0563 observed.

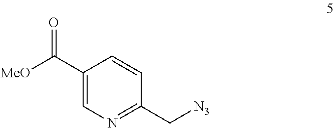 [SMILES: COC(=O)c1ccc(CN=[N+]=[N-])nc1]

Azide 5 (Methyl 5-(azidomethyl)nicotinate) was prepared according to EP Patent 127992. ¹H NMR (500 MHz, CDCl₃): 9.18 (d, 1H, J=2.0 Hz), 8.32 (dd, 1H, J=8.5, 2.0 Hz), 7.44 (d, 1H, J=8.5 Hz), 4.56 (s, 2H), 3.95 (s, 3H). ¹³C NMR (125 MHz, CDCl₃): 165.7, 160.3, 151.6, 138.4, 125.5, 121.6, 55.7, 52.7. HR-ESI-MS: [M+H]⁺ m/z 193.0726 calculated, 193.0733 observed.

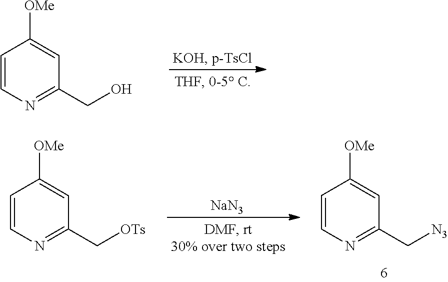

Azide 6 (2-Azidomethyl-4-methoxypyridine). 2-Hydroxymethyl-4-methoxypyridine (278 mg, 2.0 mmol) was dissolved in tetrahydrofuran (15 mL) in a 50 mL round-bottomed flask under argon. The flask was cooled to 0-5° C. with an ice/water bath for 10 minutes at which time, powdered KOH (157 mg, 2.8 mmol) was added followed by para-toluenesulfonyl chloride (p-TsCl). The reaction was stirred for 12 hours, at which time diethyl ether (30 mL) was added. The mixture was transferred to a separatory funnel, and a saturated solution of NaHCO₃ (40 mL) was added. The organic layer was dried with MgSO₄, filtered, and concentrated to a residue, which was chromatographed on a silica gel column with a 10% to 50% gradient of ethyl acetate/hexanes. R_{f}=0.69 (ethyl acetate, 254 nm UV). This material was then dissolved in N,N-dimethylformamide (5 mL), and sodium azide (266 mg, 4.09 mmol) was added and the reaction was stirred at ambient temperature for 16 hours, at which time the reaction mixture was diluted with diethyl ether (30 mL) and washed with a saturated solution of NaHCO₃ (3×30 mL), then with brine (25 mL), dried with MgSO₄, filtered and concentrated in vacuo. The resulting residue was chromatographed over silica gel with a 15% to 50% gradient of ethyl acetate/hexanes to furnish 100 mg (30% yield) of 6 as a light yellow oil. R_{f}=0.68 (ethyl acetate, 254 nm UV). ¹H NMR (400 MHz, CDCl₃): 8.38 (d, 1H, J=5.8 Hz), 6.85 (d, 1H, J=2.4 Hz), 6.74 (dd, 1H, J=5.8, 2.4 Hz), 4.42 (s, 2H), 3.85 (s, 3H). ¹³C NMR (100 MHz, CDCl₃): 166.6, 157.5, 151.0, 109.1, 108.1, 55.8, 55.3. HR-ESI-MS: [M+H]⁺ m/z 165.0776 calculated, 165.0777 observed.

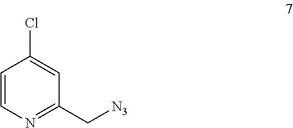 [SMILES: [N-]=[N+]=NCc1cc(Cl)ccn1]

Azide 7 (2-Azidomethyl-4-chloropyridine) was prepared according to Fernandez-Suarez, et al., Nature Biotechnology, 25:1483-1487 (2007). ¹H NMR (400 MHz, CDCl₃): 8.44 (d, 1H, J=5.3 Hz), 7.33 (d, 1H, J=2.0 Hz), 7.21 (dd, 1H, J=5.3, 2.0 Hz), 4.46 (s, 2H), 4.44 (s, 2H). ¹³C NMR (100 MHz, CDCl₃): 157.5, 150.5, 145.1, 123.3, 122.2, 55.1. HR-ESI-MS: [M+H]⁺ m/z 169.0281 calculated, 169.0279 observed.

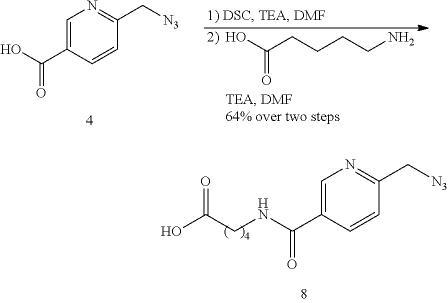

Picolyl azide 8 (5-(6-(Azidomethyl)nicotinamido)pentanoic acid). To a solution of 6-azidomethylnicotinic acid 4 (30 mg, 0.168 mmol) in anhydrous DMF (500 μL) was added disuccinimidyl carbonate (DSC; 65 mg, 0.253 mmol) and triethylamine (TEA; 120 μL, 0.840 mmol). The reaction was allowed to proceed for 3 hours at ambient temperature. The reaction mixture was diluted with chloroform and water. Layers were separated, and the aqueous layer was extracted with chloroform three times. The combined organic layer was washed with brine, dried over MgSO₄, and concentrated in vacuo. The residual mixture was purified by silica chromatography (1:1 hexanes:ethyl acetate) to afford the succinimidyl ester of 6-azidomethylnicotinic acid. R_{f}=0.67 in 9:1 chloroform:methanol.

To a solution of 5-azidomethylnicotinic acid succinimidyl ester (15 mg, 0.055 mmol) in anhydrous DMF (500 μL) was added 5-aminovaleric acid (32 mg, 0.273 mmol) and TEA (38 μL, 0.273 mmol). The reaction proceeded for 12 hours at ambient temperature. TEA and DMF were then removed in vacuo, and the resulting residue was dissolved in water and subjected to purification by preparative-scale HPLC. For this purification, we used Varian Prostar 210 HPLC equipped with Agilent 325 UV/Vis dual-wavelength detector, Agilent 440-LC fraction collector, and a Microsorb C18 column (Varian, 5 μm particle size, 21 mm×250 mm dimension). The gradient used was 0-10% acetonitrile in water at a 10 mL/min flow rate over 30 min. Picolyl azide 8 eluted at 29-30 minutes. After collecting desired fractions, acetonitrile was removed in vacuo, and the resulting solution was flash-frozen and lyophilized to yield the final product as white powder. Rf=0.58 in 90:5:5 ethyl acetate: methanol: acetic acid. ¹H NMR (500 MHz, D₂O): 8.83 (s, 1H), 8.18 (d, 1H, J=8.5 Hz), 7.59 (d, 1H, J=8 Hz), 4.62 (s, 2H), 3.42 (m, 2H), 2.32 (m, 2H), 1.65 (m, 4H). ¹³C NMR (100 MHz, CD₃OD): 167.3, 161.4, 158.2, 149.3, 137.7, 131.2, 123.3, 55.9, 42.4, 40.8, 32.0, 29.9. HR-ESI-MS: [M+H]⁺ m/z 278. 1248 calculated, 278. 1264 observed.

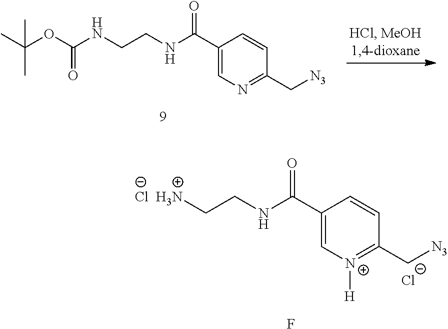

### (b) Preparation of N-(2-aminoethyl)-6-(azidomethyl)nicotinamide (F)

To a solution of 9 (16.9 mg, 0.053 mmol) in methanol (0.5 mL) was added a 4M HCl/dioxane solution (132 μL, 0.264 mmol hydrogen chloride). The reaction mixture was stirred for 1 hour and 40 min under ambient temperature, at which time the mixture was concentrated under a stream of nitrogen to provide 7.6 mg of F, which was used in the next step without further purification.

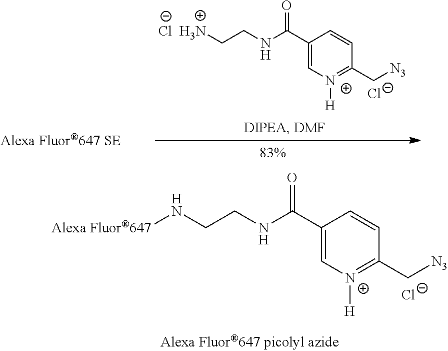

### (c) Alexa Fluor® 647-Picolyl Azide Conjugate

To a solution of F (5.5 mg, 0.019 mmol) in DMF (0.95 mL) was added DIPEA (100 μL) and Alexa Fluor® 647 succinimidyl ester (Alexa Fluor® 647-SE; 20 mg, 0.016 mmol). After stirring at ambient temperature for 10 hours, the reaction mixture was concentrated and directly purified by preparative-scale HPLC. For this purification, we used Waters 600 HPLC equipped with Waters 996 diode array detector, Waters 717 plus autosampler, and a Luna C18 column (Phenomenex, 5 μm particle size, 4.6 mm×250 mm dimension). The gradient used was 5-95% 10 mM NH₄OAc/MeOH at a 1 mL/min flow rate over 30 min. Fractions containing the product were combined and concentrated in vacuo. The residual was then dissolved in water (10 mL), flash-frozen, then lyophilized to yield 13.6 mg of Alexa Fluor® 647-picolyl azide a bright blue powder (83%). Tᵣ=20.8 min at 647 nm. MS (ESI+): 1061.3 (M+H⁺; 2%), 531.2, 6%); (ESI−): 1060.3 (Zwitterion, 17%), 540.3 (52%), 529.3 (M²⁻, 100%). HPLC: >99% purity at 254 nm and 644 nm.

### (d) Characterization of Triazole Adducts

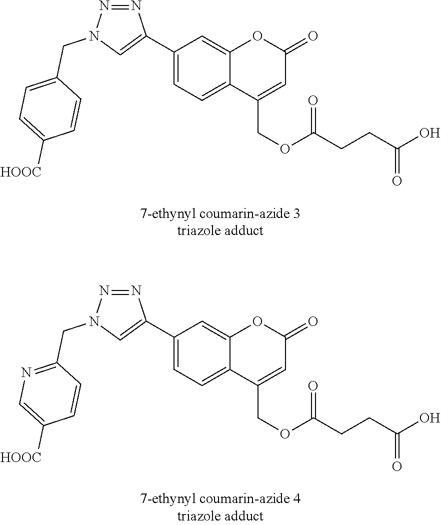 [SMILES: O=C(O)CCC(=O)OCc1cc(=O)oc2cc(-c3cn(Cc4ccc(C(=O)O)cc4)nn3)ccc12 ; O=C(O)CCC(=O)OCc1cc(=O)oc2cc(-c3cn(Cc4ccc(C(=O)O)cn4)nn3)ccc12]

7-ethynylcoumarin was synthesized and characterized as previously reported. Brotherton, et al., Organic Letters, 11:4954-4957 (2009).

To prepare the triazole adduct between 7-ethynyl coumarin and 4-azidomethylbenzoic acid (azide 3), 7-ethynyl coumarin (20 mg, 0.067 mmol) and 3 (20 mg, 0.11 mmol) were dissolved in tetrahydrofuran (4 mL). Sodium ascorbate (0.5M solution in water, 59 μL, 0.029 mmol) and copper(II) sulfate (0.25M solution in water, 30 μL, 0.007 mmol) were then added, and the reaction was heated to reflux overnight. After the solvent was removed in vacuo, the resulting residue was washed three times with methanol, and the remaining solid dried in vacuo. Pure product was obtained as white powder. ¹H NMR (400 MHz, DMSO-d6): 8.88 (s, 1H, 7.96 (br, 2H), 7.88 (m, 2H), 7.82 (m, 1H), 7.47 (br, 2H), 6.47 (s, 1H), 5.78 (s, 2H). 5.43 (s, 2H), 2.71 (br, 2H), 2.57 (br, 2H). HR-ESI-MS: [M+H]⁺ m/z 478.1250 calculated, 478.1239 observed.

To prepare the triazole adduct between 7-ethynyl coumarin and 6-azidomethylnicotinic acid (azide 4), 7-ethynyl coumarin (20 mg, 0.067 mmol) and 4 (20 mg, 0.11 mmol) were dissolved in DMSO (4 mL). Sodium ascorbate (0.5M solution in water, 59 μL, 0.029 mmol) and copper(II) sulfate (0.25M solution in water, 30 μL, 0.007 mmol) were then added, and the reaction was stirred for 1 hour. After the solvent was removed in vacuo, The resulting residue was taken up in methanol and loaded directly onto a preparative TLC plate (0.25 mm thickness) and the plate was developed with 95:5 acetonitrile:H₂O. The product-containing silica was collected and sonicated in chloroform (30 mL) for 3 minutes and filtered. The filtrate was concentrated to deliver the triazole adduct as a tan solid. ¹H NMR (400 MHz, DMSO-d6): 8.91 (s, 1H), 8.77 (s, 1H), 8.17 (d, 1H, J=8.0 Hz), 7.86-7.75 (m, 3H), 7.34 (d, 1H, J=8.0 Hz), 6.41 (s, 1H), 5.76 (s, 1H), 5.35 (s, 2H), 2.64 (t, 1H, J=6.2 Hz), 2.50-2.45 (m, 2H), 1.86 (s, 1H). ¹³C NMR (100 MHz, DMSO-d6): 175.0, 173.7, 172.8, 160.4, 155.4, 153.88, 150.7, 150.6, 145.45, 138.4, 134.6, 125.9, 124.3, 122.1, 121.9, 116.7, 113.0, 112.4, 61.5, 54.9, 48.9, 30.0, 29.5, 21.9. HR-ESI-MS: [M+H]⁺ m/z 479.1203 calculated, 479.1210 observed.

### (e) Other Chemicals

8-azidooctanoic acid, tris(3-hydroxypropyltriazolylmethyl)amine (THPTA) and bis(tert-butyltriazoylmethyl)-2-carboxy methyltriazoylmethylamine (BTTAA) were synthesized and characterized according to methods known in the art. See, e.g., Fernandez-Suarez, et al., Nature Biotechnology, 25:1483-1487 (2007); Hong, et al., Angew. Chem., Int. Ed., 48:9879-9883 (2009), and Besanceney-Webler, et al., Angew. Chem., Int. Ed., 50:8051-8056 (2011). 10-undecynoic acid is commercially available.

### Genetic Constructs.

Complete nucleotide sequences of the following constructs can be found at stellar.mit.edu/S/project/tinglabreagents/r02/materials.html: LplA variants in pYFJ16 for expression in E. coli; LAP-CFP in pDisplay; LAP-neurexin-1β in pECFP-N1; and LAP-neuroligin-1 in pNICE.

### Fluorescence Imaging.

Cells were imaged in Tyrode's buffer or DPBS in epifluorescence or confocal modes. For epifluorescence imaging, we used a Zeiss AxioObserver inverted microscope with a 40× oil-immersion objective. CFP (420/20 excitation, 425 dichroic, 475/40 emission), Alexa Fluor® 647 (630/20 excitation, 660 dichroic, 680/30 emission) and differential interference contrast (DIC) images were collected and analyzed using Slidebook software (Intelligent Imaging Innovations). For confocal imaging, we used a Zeiss Axiovert 200M inverted microscope with a 40× oil-immersion objective. The microscope was equipped with a Yokogawa spinning disk confocal head, a Quad-band notch dichroic mirror (405/488/568/647), and 491 (DPSS), 561 nm (DPSS), 640 nm (DPSS) lasers (all 50 mW). YFP/Alexa Fluor® 488 (491 laser excitation, 528/38 emission), Alexa Fluor® 568 (561 laser excitation, 617/73 emission), Alexa Fluor® 647 (640 laser excitation, 680/30 emission), and DIC images were collected using Slidebook software. Fluorescence images in each experiment were normalized to the same intensity ranges. Acquisition times ranged from 10-1000 milliseconds.

Automated image acquisition and analysis were performed on ArrayScan® VTI platform (ThermoFisher Cellomics) using MeanCircAveInten algorithm to determine channel signal intensity. Images were acquired with a Nikon Eclipse 200 inverted fluorescence microscope using a 20× objective. We used the following Semrock Brightline® filters for imaging: DAPI 5060B for DAPI; FITC 3540B for Alexa Fluor® 488; TxRed 4040B for Alexa Fluor® 594; and Cy5 4040A for Alexa Fluor® 647. Acquisition times ranged from 10-2000 milliseconds.

### In Vitro LplA-Catalyzed Picolyl Azide and Alkyne Ligation

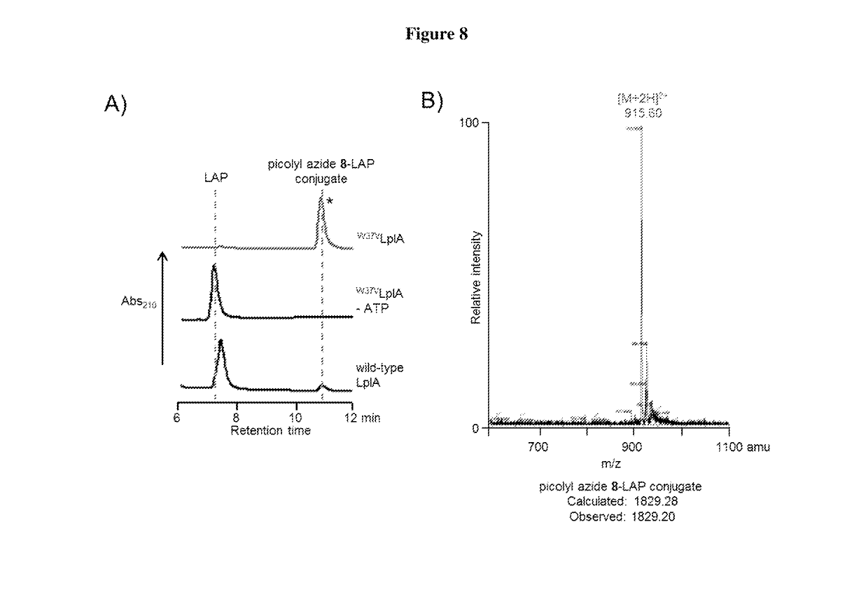

For picolyl azide 8 ligation (FIG. 8), the enzymatic reaction was assembled as follows: 150 μM LAP (amino acid sequence: GFEIDKVWYDLDA; SEQ ID NO:4), 5 μM ^{W37V}LplA, 500 μM picolyl azide 8, 1 mM ATP, and 5 mM Mg(OAc)₂ in 20% v/v glycerol in Dulbecco's phosphate-buffered saline (DPBS) at 30° C. for 30 min. The reaction was quenched with EDTA (final concentration 50 mM) and analyzed on a Varian Prostar HPLC using a reverse phase C18 Microsorb-MV100 column (250×4.6 mm). Chromatograms were recorded at 210 nm. We used a 10-min gradient of 30-60% acetonitrile in water with 0.1% trifluoroacetic acid at a flow rate of 1 mL/min. LAP had a retention time of 7.5 min; after ligation to picolyl azide 8, the retention time increased to 11 min.

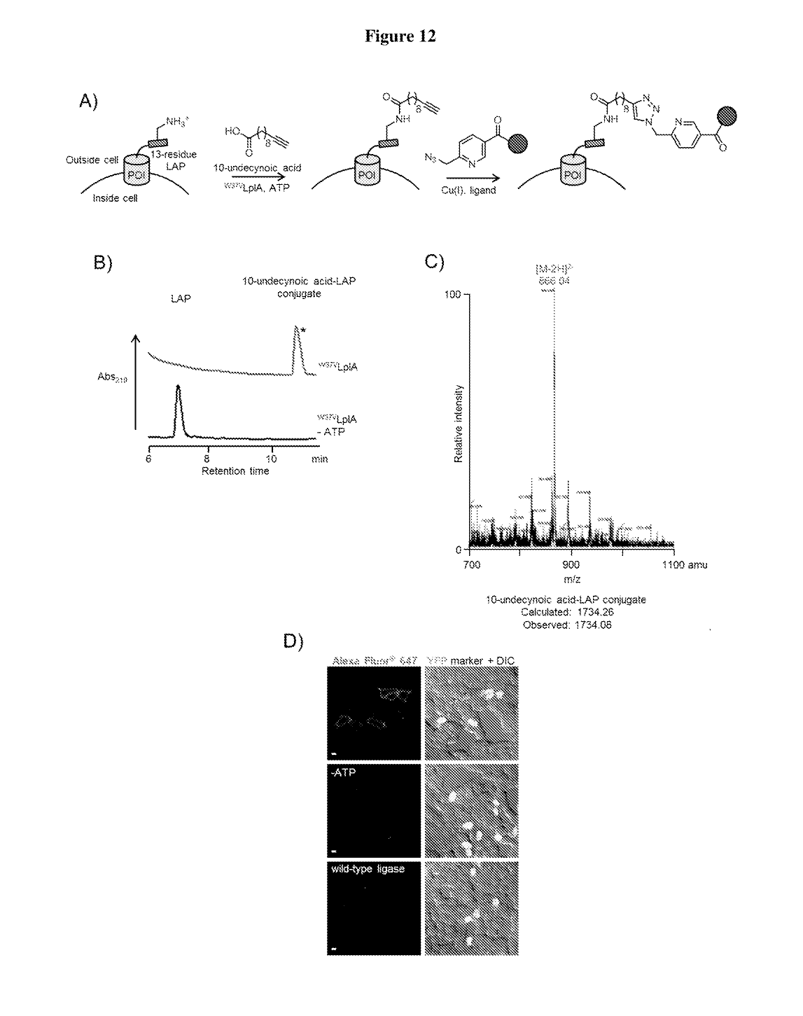

For 10-undecynoic acid ligation (FIG. 12B), the enzymatic reaction was as follows: 150 μM LAP, 5 μM ^{W37V}LplA, 500 μM 10-undecynoic acid, 1 mM ATP, and 5 mM Mg(OAc)₂ in 20% v/v glycerol in Dulbecco's phosphate-buffered saline (DPBS) at 30° C. for 30 min. The reaction was quenched with EDTA (final concentration 50 mM) and analyzed as described for picolyl azide ligation in the main methods. The retention time of 10-undecynoic acid-LAP adduct is 11 min.

### Mass Spectrometric Analysis of LAP-Probe Conjugates

To characterize LAP-picolyl azide 8 adduct (FIG. 8B), the starred peak from FIG. 8A was manually collected and injected into an Applied Biosystems 200 QTRAP mass spectrometer. The flow rate was 3 mL/min, and mass spectra were recorded under the positive-enhanced multicharge mode. To characterize 10-undecynoic acid-LAP adduct (FIG. 12C), the starred peak from FIG. 12B was similarly collected and injected into the mass spectrometer under 3 mL/min flow rate. Its mass spectra were recorded under the negative-enhanced multicharge mode.

Live-Cell Immunostaining with Anti-Lipoic Acid Antibody

Live HEK cells were incubated with rabbit anti-lipoic acid antibody (Calbiochem) in cell growth medium at 1:300 dilution for 10 min at room temperature, followed by two washes with cell growth medium. Thereafter, cells were incubated with anti-rabbit secondary antibody conjugated to Alexa Fluor® 568 (Life Technologies) in cell growth medium at 1:300 dilution for 10 min at room temperature, followed by two washes with cell growth medium.

Cell Surface Labeling with an Alkyne Ligase and Alexa Fluor® 647-Picolyl Azide

HEK cells were transfected with expression plasmids for LAP-tagged neurexin-1β (400 ng and H2B-YFP using lipofectamine 2000. 24 hr after transfection, cells were treated with 10 μM purified ^{W37V}LplA, 200 μM 10-undecynoic acid, 1 mM ATP, and 5 mM Mg(OAc)₂ in cell growth medium for 20 min at room temperature. After brief rinsing, cells were further labeled with 20 μM Alexa Fluor® 647-picolyl azide, 50 μM CuSO₄, 250 μM THPTA, and 2.5 mM sodium ascorbate in DPBS for 5 min at room temperature. Cells were imaged after brief rinsing.

Analysis of Cytotoxicity after Chelation-Assisted CuAAC HeLa cells were analyzed in 96-well plates. Transfected cells expressing LAP-tagged neuroligin-1 were labeled 24 hours after transfection as described in the figure legend. Thereafter, 100 μL of premised CellTiter-Glo reagent (Promega) was added into each well. The plate was shaken at 30° C. for 10 min, and the luminescence from each well was recorded with a SPECTRAmax dual-scanning microplate spectrofluorometer. Measurements were performed in triplicate.

### Analysis of Protective Effects of THPTA Ligand on Phalloidin Staining on Microfilaments

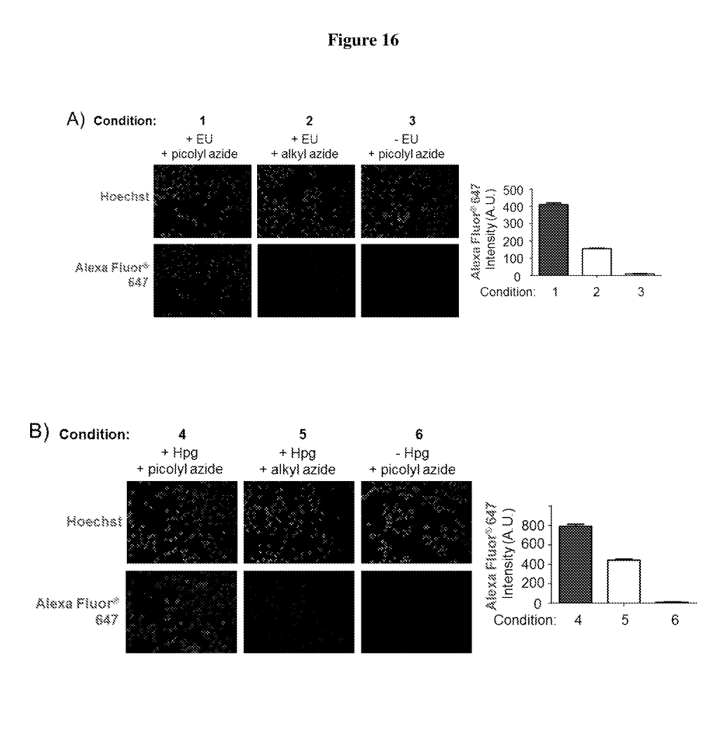

A375 cells stably expressing GFP-Erk2 were metabolically labeled with EU and derivatized with Alexa Fluor® 647-picolyl azide as described for FIG. 16. After CuAAC labeling, cells were stained with phalloidin-Alexa Fluor® 594 conjugate (170 nM; 5 U/mL) in PBS for 30 min, then further stained with Hoechst 33342 as described.

### Results

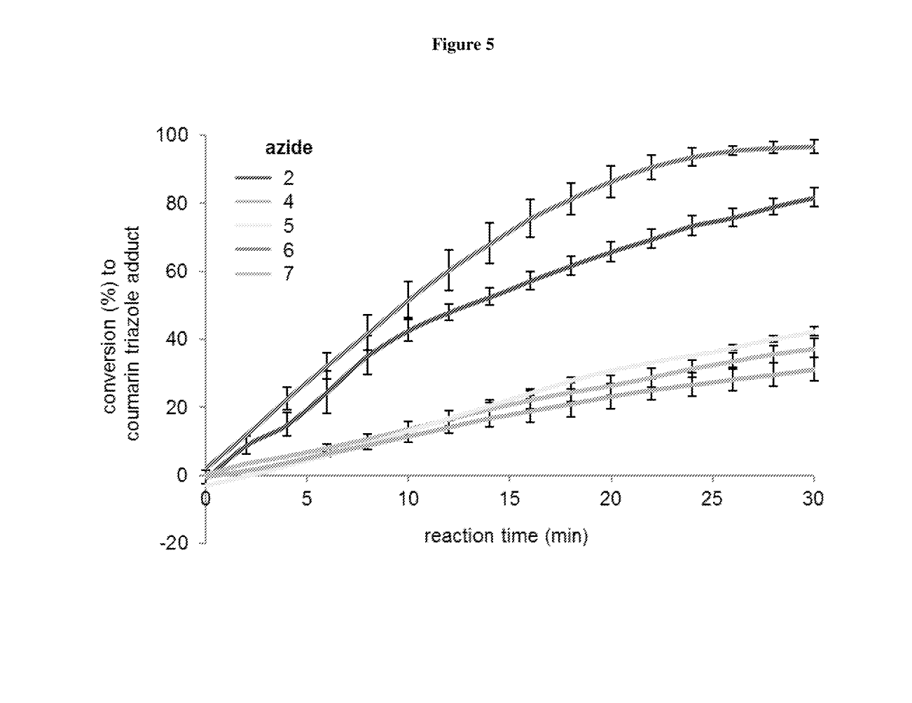

The rate-determining step of CuAAC is postulated to be the metallacycle formation between the CuI-acetylide and the organic azide. Himo, et al., J. Am. Chem. Soc., 127:210-216 (2005). To examine CuAAC rates of azides with Cu-coordinating motifs, 2-picolyl azide 2 and 6-(azidomethyl)nicotinic acid 4, both bearing an sp2-hybridized ring nitrogen, were prepared for binding to CuI/II, and compared their CuAAC rates to their carbocyclic analogs 1 and 3, respectively (FIG. 4). Relative CuAAC rates were evaluated with 7-ethynylcoumarin, whose fluorescence quantum yield increases from 1% to 25% upon reaction with azide4 (FIG. 4A). Assays were performed with 10 μM CuSO4 and no accelerating ligand such as THPTA or BTTAA. FIG. 5 shows the product conversion vs. time profiles, while FIG. 4B summarizes the calculated percent conversion to product after 10 min and 30 min, for each azide structure. It was found that picolyl azides 2 and 4 are much faster reactants than 1 and 3, giving 43-fold and 14-fold improvements in initial CuAAC rates, respectively. Substitution of the aromatic ring with an electron-donating methoxy group (azide 6) further accelerated the CuAAC reaction, while an electron-withdrawing chloride substituent (azide 7) dampened the accelerating effect, consistent with the proposed mechanism of copper chelation.

Picolyl azide 4 was further investigated, since it is the building block of the LplA substrate and fluorophore conjugates described later in this work. FIG. 4C. Time courses for reaction with 7-ethynylcoumarin are shown at three different Cu concentrations, with and without the CuI ligand THPTA. As has previously been shown, addition of THPTA has a large effect. For the non-chelating carbocyclic analog of 4, azide 3, product is undetectable after 30 min in the absence of THPTA (consistent with FIG. 4B), whereas the reactions at the two higher copper concentrations (100 and 40 uM) proceed to completion within 30 min when THPTA is added. It is consistent with our understanding of the cycloaddition mechanism that reduction of Cu concentration reduces the reaction rate.

Dramatic rate enhancements were seen for all 6 conditions when azide 3 was substituted by the chelation-competent azide 4. First, product can be detected and the reactions even proceed to completion within 30 min for the two higher Cu concentrations (100 and 40 uM), when THPTA is absent, in striking contrast to azide 3. Second, when THPTA is added, azide 4 reacts to completion within 5 min at all three copper concentrations. In other words, the use of chelating azide 4 far offsets the reduction in CuAAC reaction rate caused by lowering Cu concentration. The effect is so strong that the reaction rate of chelating azide 4 at the lowest Cu concentration of 10 uM exceeds the reaction rate of the non-chelating azide 3 at the highest Cu concentration (100 uM). It is also noteworthy that the use of picolyl azide 4 over the conventional azide 3 can more than offset the effect of omitting the accelerating ligand THPTA. FIG. 4C shows that the reaction rates with picolyl azide 4 at all three Cu concentrations in the absence of THPTA are at least as high as the reaction rates of conventional azide 3 in the presence of THPTA.

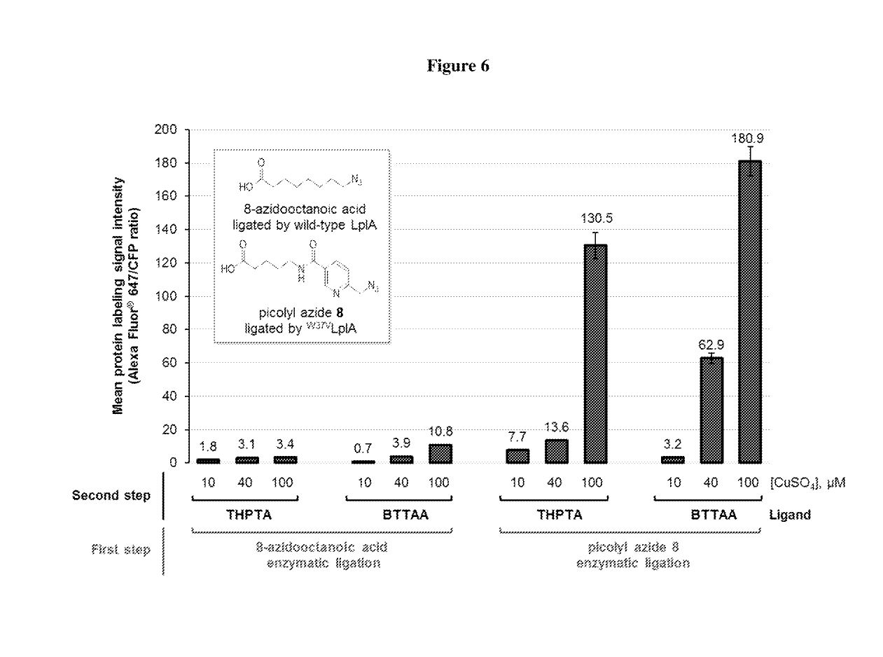

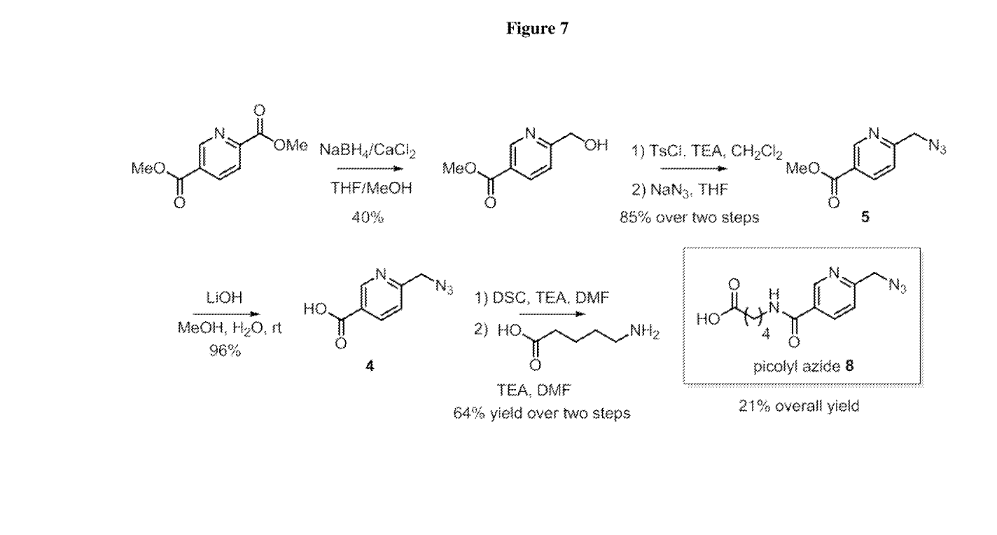

Based on these promising in vitro observations, the utility of picolyl azide in the cellular setting was tested. To develop a method to target the picolyl azide moiety to specific cellular proteins of interest, the PRIME (Probe Incorporation Mediated by Enzymes protein labeling platform as described herein was explored. Utamapinant, et al., Proc. Natl. Acad. Sci. U.S.A., 107:10914-10919 (2010). A panel of E. coli lipoic acid ligase (LplA) mutants was prepared, each with a mutation at the gatekeeper residue, Trp37. Utamapinant, et al., Proc. Natl. Acad. Sci. U.S.A., 107:10914-10919 (2010); Baruah, et al., Angew. Chem. Int. Ed. Engl., 47:7018-7021 (2008); and Baruah, et al., Angew. Chem. Int. Ed. Engl., 47:7018-7021 (2008). A picolyl azide derivative was synthesized that matches the substrate requirements for LplA, i.e., carboxylic acid joined by a 3-4 methylene linker to the picolyl azide moiety (picolyl azide 8; structure in FIGS. 3B and 6; synthesis in FIG. 7). In vitro screening using HPLC revealed that among six LplA mutants (W37G, A, V, I, L, S), W37VLplA was most efficient at recognizing picolyl azide 8 and catalyzing its covalent and ATP-dependent ligation to LplA's 13 amino acid recognition sequence, LAP (LplA acceptor peptide) FIG. 8. See also Puthenveetil, et al., J. Am. Chem. Soc., 131:16430-16438 (2009).

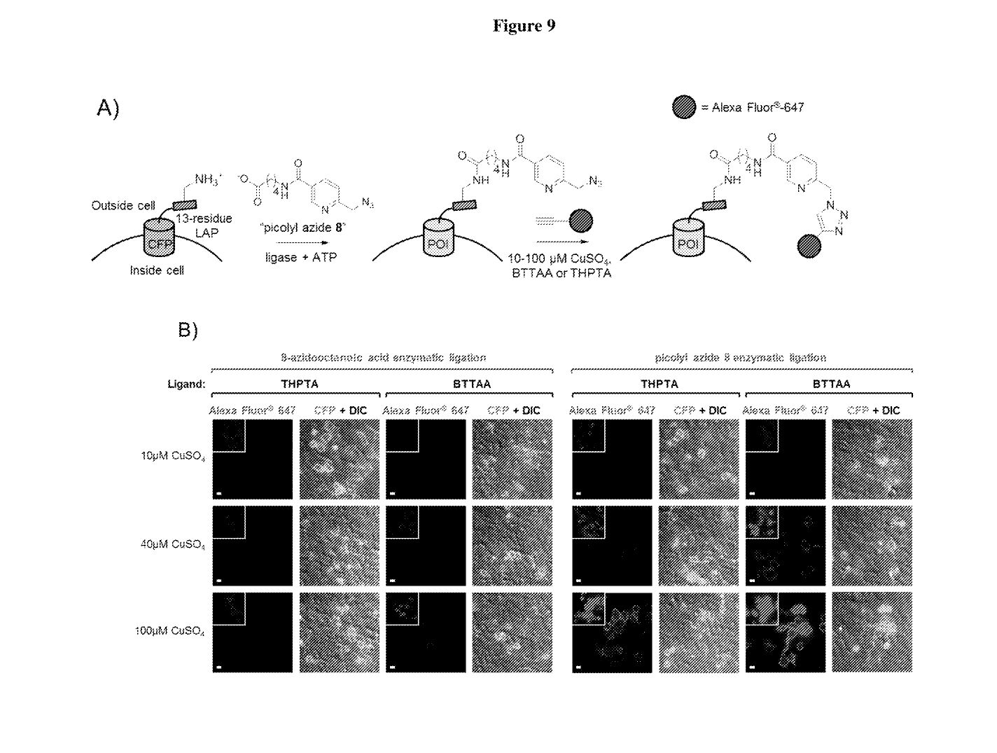

To test enzyme-catalyzed picolyl azide ligation on cells, HEK cells expressing a cell surface LAP fusion protein-LAP-CFP-TM were prepared, CFP being cyan fluorescent protein and TM is the transmembrane helix of the PDGF receptor. Picolyl azide 8 and W37VLplA were added to cells for 20 min. Thereafter, ligated picolyl azide was detected by CuAAC with Alexa Fluor® 647-alkyne. Labeling was easily detectable and specific to transfected cells (FIGS. 6 and 9). However, to systematically evaluate the effect of chelation assistance at different Cu concentrations, multiple labeling conditions were compared in parallel. Furthermore, new and improved cell-compatible CuAAC ligands have been developed since the initial report of THPTA. Besanceney-Webler, et al., Angew. Chem. Int. Ed. Engl., 50:8051-8056 (2011)); del Amo, et al, J. Am. Chem. Soc., 132:16893-16899 (2010), and Hong, et al., Bioconjugate Chemistry, 21:912-1916 (2010). BTTAA has been shown to be the best in terms of reaction-accelerating and cell-protective effects by Wu et al. and so this ligand was synthesized and tested it alongside THPTA in our multi-condition comparison shown in FIGS. 6 and 9.

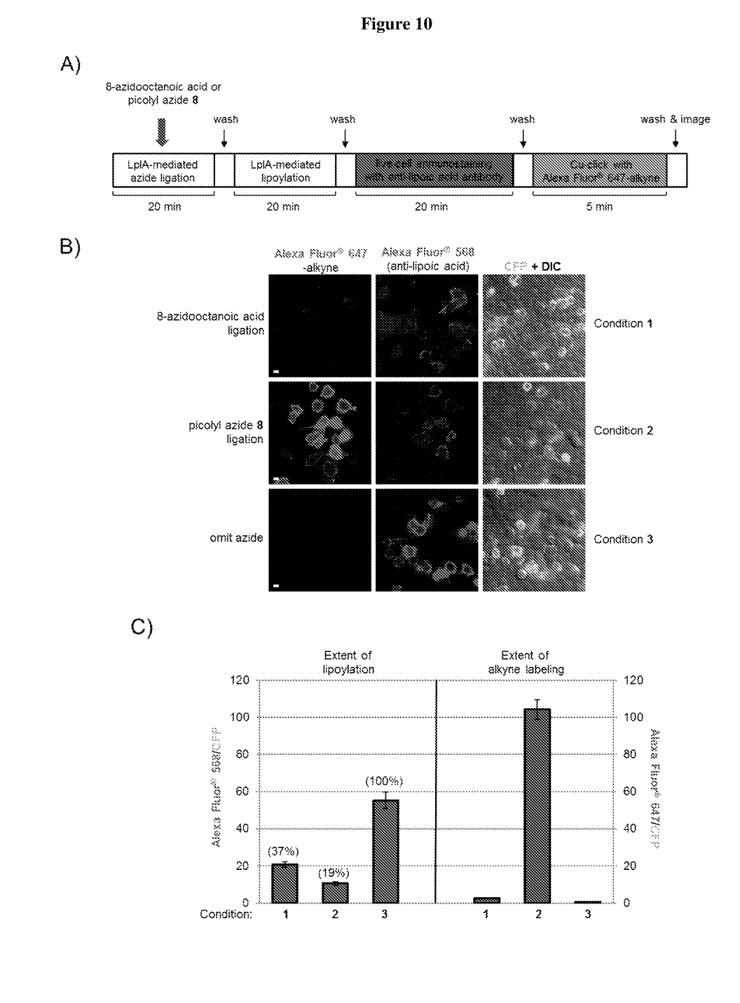

In these figures, three Cu concentrations were tested (10, 40, and 100 μM, same as FIG. 4C). Both THPTA and BTTAA ligands were tested. To evaluate the contribution of chelation assistance, we tested picolyl azide ligation to LAP versus alkyl azide (8-azidooctanoic acid) ligation to LAP, catalyzed by wild-type LplA[20]. FIG. 10 shows the labeling extent for these two enzyme-catalyzed ligations, and though picolyl azide ligation proceeds to a greater extent under the 20 min labeling conditions, the difference is at most 1.5-fold over 8-azidooctanoic acid ligation. Representative images of two-step labeling of LAP-CFP-TM on cells with Alexa Fluor® 647-alkyne are shown in FIG. 9; quantitation of this data in shown in FIG. 6.

Several trends are apparent. First, for the non-chelating azide 8-azidooctanoic acid, reduction of Cu concentration reduces the cell labeling signal, as expected. Second, BTTAA does indeed give higher signals than THPTA, but not as much as previously reported[8], and not at the lowest Cu concentration of 10 μM. Third, replacement of 8-azidooctanoic acid on LAP with the chelation-competent picolyl azide 8 boosts cell signal across the board 4- to 38-fold, or 2.7- to 25-fold when differences in picolyl azide versus alkyl azide enzymatic ligation efficiencies are taken into account (FIG. 10). The signal enhancements were greatest at the higher Cu concentrations of 40 and 100 μM. Like the in vitro data shown in FIG. 4C, the signal enhancement caused by picolyl azide more than offsets the decrease in CuAAC rate caused by lowering the Cu concentration. For instance, the signal with picolyl azide at 10 μM Cu (+THPTA) was still 1.6-fold (corrected value) greater than the signal with alkyl azide at 100 μM Cu (+THPTA). Comparisons in the presence of BTTAA showed that picolyl azide at 40 μM Cu gave 3.9-fold (corrected value) greater signal than alkyl azide at 100 μM Cu. This experiment also showed that the rate enhancement caused by picolyl azide (compared to non-chelating alkyl azide) was much greater than the rate enhancement due to switching from a previous-generation ligand (THPTA) to a newest-generation ligand (BTTAA). Overall, the best cell labeling results were obtained using picolyl azide in combination with BTTAA ligand and either 40 or 100 μM CuSO4.

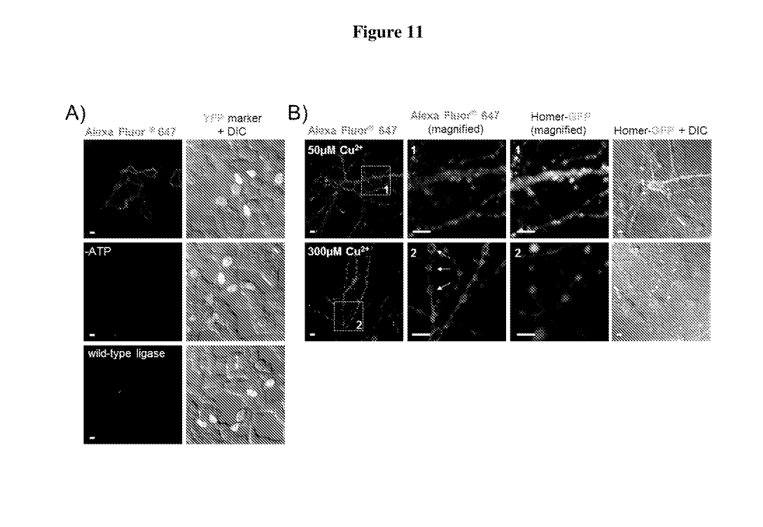

Site-specificity of cell surface protein labeling was tested using LplA and CuAAC. In FIG. 11, HEK cells expressing LAP-neurexin-1β were labeled first with W37VLplA and picolyl azide 8, followed by CuAAC with Alexa Fluor® 647-alkyne and 50 μM CuSO4. Transfected cells (expressing the nuclear YFP marker) were strongly labeled with a ring of Alexa Fluor® 647 fluorescence, whereas neighboring untransfected cells were not labeled. Negative controls with ATP omitted or with wild-type LplA replacing W37VLplA eliminated Alexa Fluor® 647 labeling. The use of the picolyl azide ligase in combination with chelation-assisted CuAAC thus seems clearly advantageous, dramatically increasing signal without sacrificing specificity.

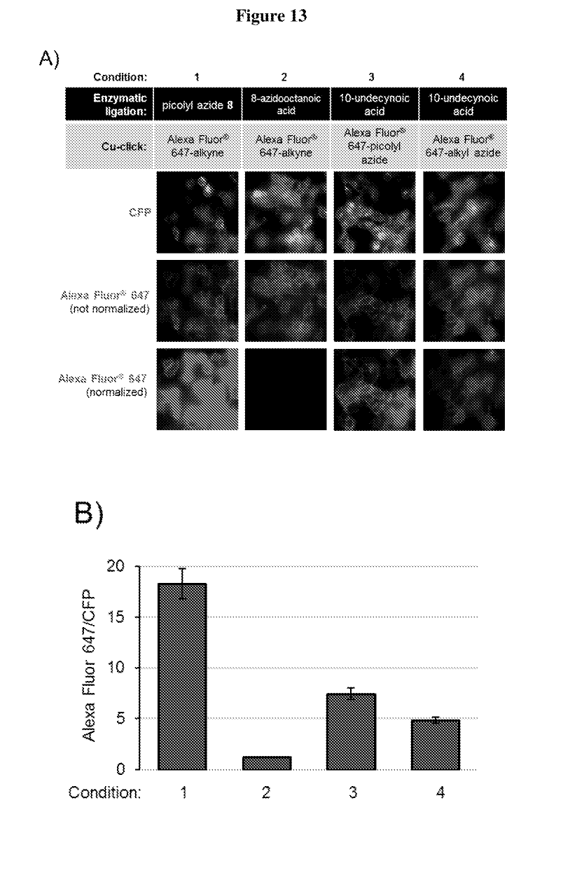

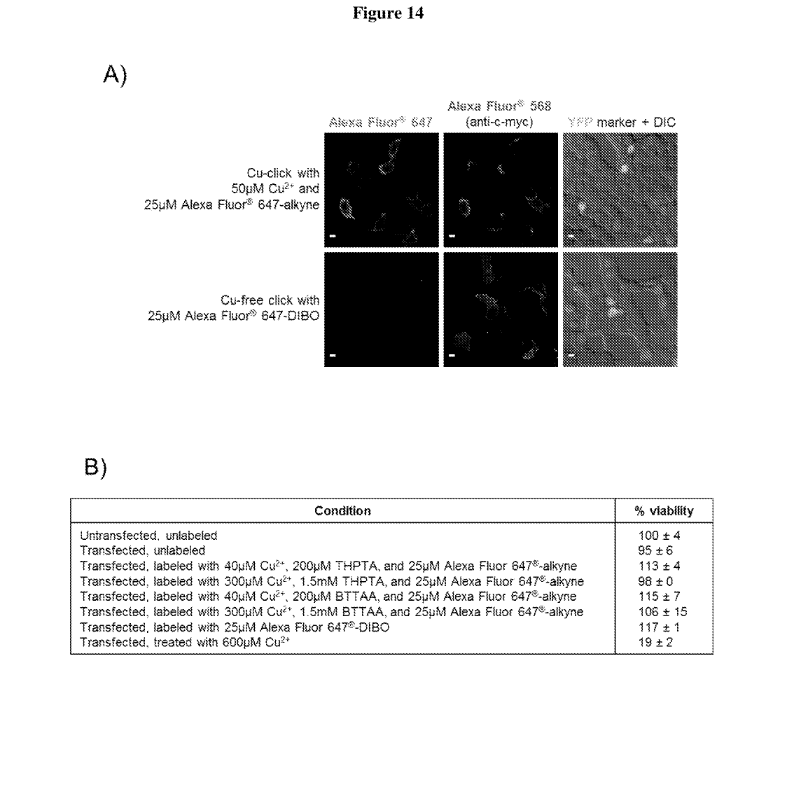

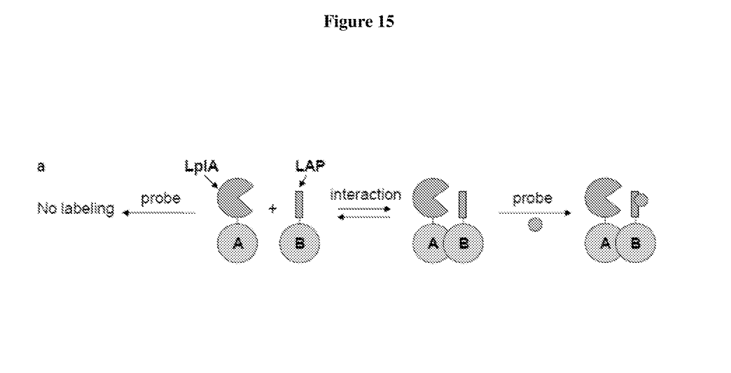

For maximum versatility, an analogous enzymatic alkyne ligation was developed for 10-undecynoic acid, demonstrated and characterized in FIG. 12. An analogous two step labeling experiment with enzymatic ligation of 10-undecynoic acid, followed by chelation-assisted CuAAC with Alexa Fluor® 647-picolyl azide, is shown in FIGS. 12A and 12D. The two labeling schemes involving picolyl azide, either as an LplA substrate or as a fluorophore conjugate, were compared side by side in FIG. 13. Picolyl azide ligation, followed by fluorophore-alkyne, gave ˜2.4-fold greater signal on average than alkyne ligation followed by fluorophore-picolyl azide. This may be due to enhanced chelation effect in one orientation compared to the other, or it may also reflect higher efficiency for the enzymatic ligation of picolyl azide 8 versus enzymatic ligation of 10-undecynoic acid. These two labeling schemes with picolyl azide nevertheless gave 1.5- to 9-fold greater signal on average than their counterpart schemes with an alkyl azide. One example to use PRIME and chelation-assisted CuAAC in combination is to use LplA to ligate the picolyl azide substrate, and then derivatize with a fluorophore-alkyne.

As a further benchmark, a side-by-side comparison of this two-step labeling (at 50 μM CuSO4) with picolyl azide ligation was performed followed by strain-promoted cycloaddition. FIG. 14 shows that picolyl azide ligation followed by chelation-assisted CuAAC is a much more sensitive labeling method than alkyl azide ligation followed by dibenzocyclooctyne-fluorophore. Ning et al., Angewandte Chemie-International Edition 47: 2253-2255 (2008). A more sensitive, biocompatible CuAAC labeling protocol is also beneficial in the detection of biomolecules in other contexts. To illustrate the general utility, we also used chelation-assisted CuAAC to image cellular RNAs and proteins metabolically labeled with 5-ethynyl uridine (EU) and L-homopropargylglycine (Hpg), respectively (FIG. 5). Jao, et al., Proc. Natl. Acad. Sci. U.S.A., 105:15779-15784 (2008) and Beatty, et al., J. Am. Chem. Soc., 127:14150-14151 (2005). Detection of these alkynes on fixed cells with Alexa Fluor® 647-picolyl azide gave ˜2.7-fold higher signal on average than detection with the alkyl azide counterpart.

In summary, the use of copper-chelating azides dramatically accelerates the CuAAC reaction under conditions relevant to biomolecular labeling. This advance is complementary to advances in ligand design, which have led to CuAAC rate acceleration and reduced cell toxicity. Hong, et al., Bioconjugate Chemistry, 21:912-1916 (2010); and Besanceney-Webler, et al., Angew. Chem. Int. Ed. Engl., 50:8051-8056 (2011). The in vitro data show that the picolyl azide effect is so strong that it more than compensates for the effect of omitting THPTA ligand, or reducing the Cu concentration 10-fold from 100 μM to 10 μM. On living cells, our experiments showed that use of picolyl azide instead of a conventional non-chelating azide increased specific protein signal by as much as 25-fold.

By engineering a lipoic acid ligase mutant capable of ligating picolyl azide 8 to LAP fusion proteins, it was straightforward to use chelation-assisted CuAAC to tag specific cell surface proteins with bright and photostable fluorophores such as the Alexa Fluors. The utility of picolyl azide for highly sensitive detection of metabolically labelled proteins and RNAs in cells was also demonstrated. In summary, the CuAAC protocol reported here, utilizing a copper-chelating azide, a newest-generation CuI ligand (BTTAA), and low Cu concentrations (10-100 μM) may represent the fastest and most biocompatible version of CuAAC to date.

### Example 2

### Diels-Alder Cycloaddition for Fluorophore Targeting to Specific Proteins Inside Living Cells

The inverse-electron-demand Diels-Alder cycloaddition between trans-cyclooctenes and tetrazines is biocompatible and exceptionally fast. This chemistry was utilized for site-specific fluorescence labeling of proteins on the cell surface and inside living mammalian cells by a two-step protocol. E. coli lipoic acid ligase site-specifically ligates a trans-cyclooctene derivative onto a protein of interest in the first step, followed by chemoselective derivatization with a tetrazine-fluorophore conjugate in the second step. On the cell surface, this labeling was fluorogenic and highly sensitive. Inside the cell, specific labeling of cytoskeletal proteins with green and red fluorophores was achieved. By incorporating the Diels-Alder cycloaddition, the panel of fluorophores that can be targeted by lipoic acid ligase has been broadened.

### Material and Methods

### Synthesis and Characterization of Synthetic Compounds

Unless otherwise stated, all reagents and solvents were purchased from commercial sources (Sigma-Aldrich, Acros Organics, Alfa Aesar, or TCI America) and used without further purification. Reactions were monitored using analytical thin-layer chromatography (0.25 mm silica gel 60 F254 plates, EMD Biochemicals). Desired products were purified on either flash column chromatography with normal phase silica gel or Varian Prostar preparatory reverse phase HPLC with a C-18 column (Varian Microsorb 300-5 C18 Dynamax). Synthetic products were characterized by electro-spray ionization mass spectrometry (Applied Biosystems 200 QTRAP) and by NMR (Bruker DRX-400).

### Mammalian Cell Culture and Transfection

Human embryonic kidney 293T (HEK), COS-7, and Chinese hamster ovary (CHO) cells were cultured as a monolayer in growth media: minimal essential medium (MEM, Mediatech) supplemented with 10% (v/v) fetal bovine serum (PAA Laboratories) at 37° C. and under 5% CO2. HEK and COS-7 cells for imaging were grown on 150 μm thickness glass cover slips pre-treated with 50 μg/ml fibronectin (Millipore). CHO cells for the cell viability assay were grown in plastic 96-well plates (Greiner Bio One). Cells were typically transfected at ˜70% confluence using Lipofectamine 2000 (Life Technologies) according to the manufacturer's instructions, then labeled 16-20 hours after transfection.

For hippocampal neuron cultures, Spague Dawley rat pups were sacrificed at embryonic day 18. Hippocampal tissue was digested with papain (Worthington) and DNaseI (Roche) and plated in MEM+L-glutamine (Sigma) supplemented with 10% (v/v) fetal bovine serum (PAA Laboratories) and B27 (Life Technologies) on glass cover slips pretreated with poly-D-lysine (Sigma) and mouse laminin (Life Technologies). At 3 days in vitro, half of the growth medium was replaced with Neurobasal (Life Technologies) supplemented with B27 and GlutaMAX (Life Technologies). Neuron transfection was performed at 5 days in vitro, using Lipofectamine 2000, using half the amount of the manufacturer's recommended reagent quantity. Cells were labeled and imaged at 12 days in vitro.

### Genetic Constructs

Constructs used in this study are summarized below with important features listed. Complete nucleotide sequences of all constructs can be found at: http://stellar.mit.edu/S/project/tinglabreagents/index.html

| Name | Features | Notes |
|---|---|---|
| LplA in pYFJ16, for E. coli | His₆-LplA | Trp37 mutants generated by QuikChange as previously |
| expression (Los et al., ACS | | reported |
| Chem. Biol. 3: 373-382 | | |
| (2008)) | | |
| ^{W37V}LplA in pcDNA3, for | His₆-FLAG-LplA | FLAG = DYKDDDDK (SEQ ID NO: 7) |
| mammalian expression | | |
| (Griffin et al., Science, | | |
| 281: 269-272 (1998)). | | |
| LAP-LDL receptor in | SS-LAP-HA-LDL receptor | SS = signal sequence |
| pcDNA4/TO | | LAP = GFEIDKVWHDFPA (SEQ ID NO: 5) |
| | | (modified Lys underlined) |
| | | HA = YPYDVPDYA (SEQ ID NO: 8) |
| LAP-neuroligin-1 in pCAG | SS-LAP-neuroligin-1 | SS = signal sequence |
| | | LAP = GFEIDKVWYDLDA (SEQ ID NO: 4) |
| Nuclear LAP-BFP in | His₆-LAP-BFP-NLS | LAP = GFEIDKVWYDLDA (SEQ ID NO: 4) |
| pcDNA3 | | Lys→Ala mutation in LAP prepared by QuikChange |
| | | NLS = nuclear localization signal from Kalderon et al.⁷ |
| LAP-β-actin | HA-LAP-β-actin | LAP = GFEIDKVWYDLDA (SEQ ID NO: 4) |
| | | HA = YPYDVPDYA (SEQ ID NO: 8) |
| Vimentin-LAP | Vimentin-C-myc-LAP | C-myc = EQKLISEEDL |
| | | LAP = GFEIDKVWYDLDA (SEQ ID NO: 4) |

### Fluorescence Microscopy

Cells placed in Tyrode's buffer or Dulbecco's phosphate buffered saline were imaged using a Zeiss AxioObserver.Z1 inverted confocal microscope with a 40× or 63× oil-immersion objective. The spinning disk confocal head was manufactured by Yokogawa. The following excitation sources and filter sets were used:

| | Laser excitation | Emission | Dichroic |
|---|---|---|---|
| Fluorophore | (nm) | (nm) | (nm) |
| BFP | 405 | 438/30 | 450 |
| Fluorescein/GFP | 491 | 525/30 | 502 |
| Tetramethylrhodamine | 561 | 605/20 | 585 |
| Alexa Fluor 647 | 647 | 680/30 | 660 |

Images were acquired and processed using SlideBook software version 5.0 (Intelligent Imaging Innovations).

### Synthesis of Trans-Cyclooctene Probes

### rel-(1R-4E-pR)-cyclooct-4-ene-1-yl (4-nitrophenyl)carbonate

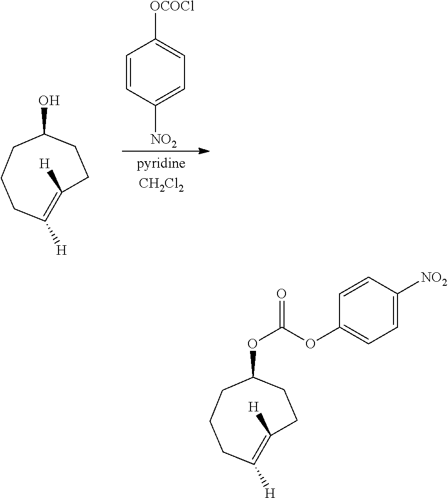

The title compound was synthesized using an adaptation of our previously reported protocol8. To a stirring solution of rel-(1R-4E-pR)-cyclooct-4-enol9 (0.732 g, 5.79 mmol) in anhydrous methylene chloride (100 mL) was added pyridine (1.20 mL, 14.5 mmol). A solution of 4-nitrophenylchloroformate (1.286 g, 6.38 mmol) in methylene chloride (20 mL) was added at room temperature and the resulting solution allowed to stir for 30 minutes. To the reaction was added NH4Cl (aq), and the layers were separated. The aqueous layer was extracted twice with methylene chloride. The organic layers were combined, dried with MgSO4, filtered, and concentrated onto silica gel using a rotary evaporator. Purification by column chromatography (5% ethyl acetate/hexanes) yielded 1.25 g (74%) of the title compound as a pale yellow solid.

mp 74-75° C. ¹H NMR (400 MHz, C6D6, δ): 7.66 (app d, J=9.7 Hz, 2H), 6.74 (app d, J=9.7 Hz, 2H), 5.29-5.12 (m, 2H), 4.40-4.35 (m, 1H), 2.13-1.98 (m, 4H), 1.86-1.73 (m, 2H), 1.71-1.57 (m, 3H), 1.40-1.31 (m, 1H). 13C-NMR (100 MHz, C6D6, δ): 155.3 (u), 152.0 (u), 145.1 (u), 134.5 (dn), 132.7 (dn), 124.8 (dn), 121.2 (dn), 85.7 (dn), 40.5 (u), 38.2 (u), 33.9 (u), 32.2 (u), 30.9 (u). IR (CHCl3, cm-1): 3105, 3007, 2928, 2859, 1756, 1594, 1526, 1348 1261 1219, 993. Elem. Anal. Calcd: 61.85; C, 4.81; N, 5.88; H. Found: 61.99; C, 4.74; N, 5.94 H.

### rel-(1R-4E-pR)-cyclooct-4-ene-1-yl-N-butyric acid carbamate (TCO1)

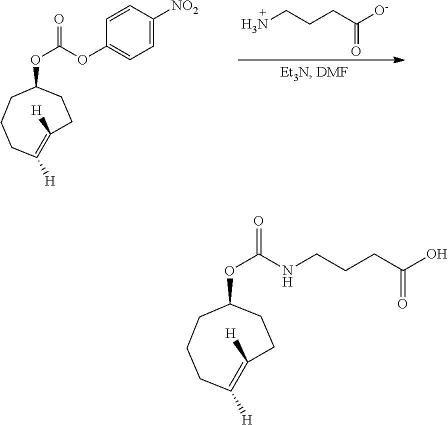

A round bottomed flask was charged with rel-(1R-4E-pR)-cyclooct-4-ene-1-yl (4-nitrophenyl) carbonate (30.0 mg, 0.103 mmol). The flask was evacuated and refilled with N2. Anhydrous dimethylformamide (0.5 mL) was added, followed by triethylamine (44 μL, 0.31 mmol). 4-Aminobutyric acid (15.8 mg, 0.153 mmol) was added in a single portion. The flask was wrapped in foil and the reaction was allowed to stir for 22 h at room temperature. The reaction solution was diluted with water, and extracted three times with ethyl acetate. The aqueous layer was then acidified with 6% aq. acetic acid, and extracted three times with methylene chloride. The organic layers were combined and washed twice with water. The organic layer was dried with MgSO4, filtered, and concentrated onto silica gel using a rotary evaporator. Purification by column chromatography (0-3% methanol/methylene chloride) yielded 9.9 mg (40%) of TCO1 as a colorless oil.

¹H-NMR (400 MHz, CD3OD): 5.62-5.54 (m, 1H), 5.50-5.42 (m, 1H), 4.35-4.14 (m, 1H), 3.09 (t, J=6.9 Hz, 2H), 2.36-2.25 (m, 5H), 2.04-1.88 (m, 4H), 1.77-1.66 (m, 4H), 1.62-1.53 (m, 1H). 13C-NMR (100 MHz, CD3OD, δ): 177.2 (u), 158.9 (u), 136.3 (dn), 133.9 (dn), 81.8 (dn), 55.0 (u), 42.3 (u), 41.3 (u), 39.8 (u), 35.3 (u), 33.6 (u), 32.2 (u), 26.5 (u). IR (CHCl3, cm-1): 3448, 3408, 3007, 2938, 2859, 1707, 1648, 1510, 1442, 1255, 994. ESI-MS (+) calculated for C26H42N2NaO8, [2M+Na]: 533.3. found: 533.3.

### rel-(1R-4E-pR)-cyclooct-4-ene-1-yl-N-pentanoic acid carbamate (TCO2)

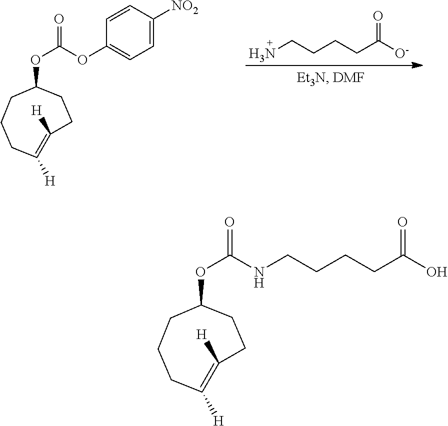

A round bottomed flask was charged with rel-(1R-4E-pR)-cyclooct-4-ene-1-yl (4-nitrophenyl) carbonate (101 mg, 0.347 mmol). The flask was evacuated and refilled with N2. Anhydrous dimethylformamide (1.7 mL) was added, followed by triethylamine (0.140 mL, 1.03 mmol). 5-aminopentanoic acid (60.6 mg, 0.517 mmol) was added in a single portion. The reaction was stirred for 20 hrs at room temperature. The reaction solution was diluted with water, and extracted twice with ethyl acetate. The aqueous layer was then acidified with 6% aq. acetic acid, and extracted three times with methylene chloride. The organic layers were combined and washed twice with water. The organic layer was dried with MgSO₄, filtered, and concentrated onto silica gel using a rotary evaporator. Purification by column chromatography (0-3% methanol/methylene chloride) yielded 64 mg (69%) of TCO₂ as a colorless oil.

1H NMR (400 MHz, CD3OD, δ): 5.65-5.57 (m, 1H), 5.53-5.46 (m, 1H), 4.39-4.28 (m, 1H), 3.10 (t, J=7.0 Hz, 2H), 2.40-2.29 (m, 5H), 2.07-1.90 (m, 4H), 1.80-1.69 (m, 2H), 1.65-1.57 (m, 3H), 1.55-1.47 (m, 2H). 13C NMR (100 MHz, CD3OD, δ): 176.0 (u), 157.3 (u), 134.7 (dn), 132.4 (dn), 80.2 (dn), 40.8 (u), 39.8 (u), 38.3 (u), 33.8 (u), 33.1 (u), 32.1 (u), 30.7 (u), 29.0 (u), 21.8 (u). IR (CHCl3, cm-1): 3453, 3390, 3007, 2928, 2859, 1706, 1658, 1515, 1445, 1236, 995. ESI-MS (+) calculated for C28H46N2NaO8, [2M+Na]: 561.3. found: 561.2.

### (rel-1R,8S,9R,4E)-Bicyclo[6.1.0]non-4-ene-9-ylmethyl-N-butyric acid carbamate (TCO3)

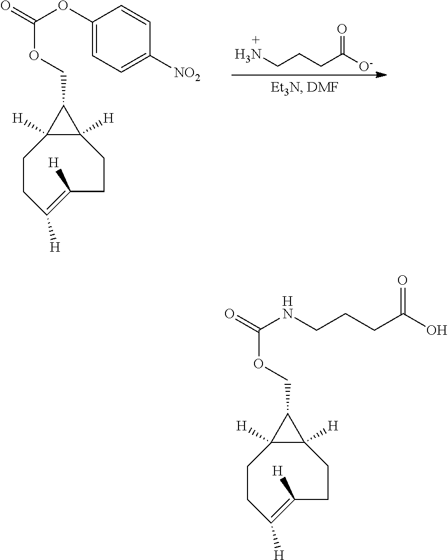

A round bottomed flask was charged with (1R,8S,9R,4E)-bicyclo[6.1.0]non-4-ene-9-ylmethyl (4-nitrophenyl) carbonate10 (39.6 mg, 0.126 mmol). The flask was evacuated and refilled with N₂. Anhydrous dimethylformamide (0.6 mL) was added, followed by triethylamine (53 μL, 0.38 mmol). 4-aminobutyric acid (19.4 mg, 0.189 mmol) was added in a single portion. The flask was wrapped in foil and the reaction was stirred for 18 h at room temperature. The reaction solution was diluted with water, and extracted three times with ethyl acetate. The aqueous layer was then acidified with 6% aq. acetic acid and extracted three times with methylene chloride. The organic layers were combined and washed twice with water. The organic layer was dried with MgSO₄, filtered, and concentrated onto silica gel using a rotary evaporator. Purification by column chromatography (0-3% methanol/methylene chloride) yielded 10 mg (28%) of TCO3 as a colorless oil. The 1^{H} NMR showed the title compound to be a ˜6:1 mixture of carbamate rotamers, on the basis of intergration of the peaks at 3.96-3.85 ppm.

¹H-NMR (400 MHz, CD3OD): 5.89-5.81 (m, 1H), 5.16-5.07 (m, 1H), 3.96-3.85 (d, J=6.5 Hz, 2H), 3.12 (t, J=6.5 Hz, 2H), 2.36-2.14 (m, 6H), 1.96-1.85 (m, 2H), 1.80-1.71 (m, 2H), 0.94-0.83 (m, 1H), 0.66-0.52 (m, 2H), 0.49-0.38 (m, 2H). 13C-NMR (100 MHz, CD3OD, δ): 174.1 (u), 156.4 (u), 136.2 (dn), 129.2 (dn), 67.3 (u), 38.0 (u), 36.7 (u), 31.7 (u), 30.7 (u), 29.1 (u), 25.6 (u), 23.4 (u), 23.1 (dn), 20.3 (dn), 19.2 (dn). IR(CHCl3, cm-1): 3449, 3292, 2997, 2928, 2859, 1708, 1658, 1515, 1447, 1255, 1014. ESI-MS (+) calculated for C30H47N2O8, [2M+H]: 563.3. found: 562.9.

### Synthesis of Tetrazine Tz2

### 3-Nitro-2-[-(trifluoromethyl)benzoyl]hydrazide (1)

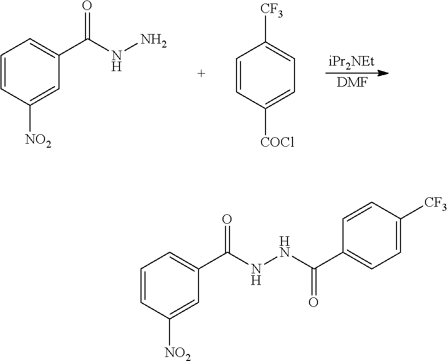

The following is a modification of the procedure of Blackman10. A stirring solution of 3-nitrobenzhydrazide (1.0 g, 5.5 mmol) and diisopropylethylamine (1.4 g, 11 mmol) in DMF (10 mL) was cooled to 0° C. under a nitrogen atmosphere. To this cold solution was slowly added 4-(trifluoromethyl)benzoyl chloride. The reaction mixture was allowed to stir for 3 h at rt. The mixture was diluted with 40 ml saturated bicarbonate solution and a solid was collected by filtration. The solid was rinsed with distilled water, suction dried, and rinsed then with hexane to give 1.6 g (84%) of the product as a pale yellow solid. The properties of the title compound matched those reported by Blackman10, which are listed here: mp 223-225° C.

¹H NMR (DMSO-d6, 400 MHz, δ): 11.0 (s, 1H), 10.9 (s, 1H), 8.76 (t, J=2.2 Hz, 1H), 8.47 (dd, J=8.3 Hz, 2.4 Hz, 1H), 8.37 (dd, J=7.9 Hz, 2.4 Hz, 1H), 8.13 (d, J=8.3 Hz, 2H), 7.94 (d, J=8.3 Hz, 2H), 7.87 (t, J=7.6 Hz, 1H). 13C NMR (DMSO-d6, 100 MHz, □): 164.7 (u), 163.8 (u), 147.9 (u), 136.1 (u), 133.8 (dn), 133.7 (u), 131.7 (u) [q, 2J(CF)=35.2 Hz], 130.5 (dn), 128.4 (u), 126.6 (dn), 125.7 (dn) [q, 3J(CF)=4.0 Hz], 123.9 (u) [q, 1J(CF)=272 Hz], 122.2 (dn). HRMS (ESI+) [M+H] calcd. for C15H9F3N3O4 354.0702. found 354.0705.

### N′-(chloro(4-(trifluoromethyl)phenyl)methylene)-3-nitrobenzohydrazonoyl chloride (2)

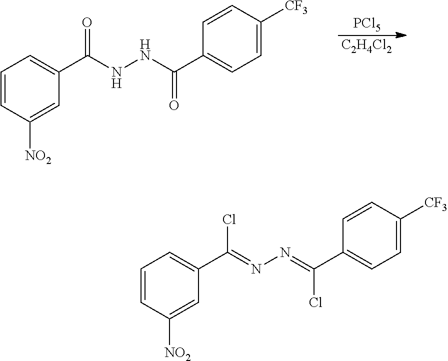

The following is a modification of the procedure of Blackman10. A solution of 3-nitro-2-[-(trifluoromethyl)benzoyl]hydrazide (0.80 mg, 2.3 mmol) and anhydrous dichloroethane (15 mL) in round bottom flask was equipped with a stirbar and a reflux condenser, and PCl5 (1.6 g, 7.7 mmol) was added to the stirring solution under nitrogen atmosphere. The reaction mixture was heated to reflux for 24 h. The reaction mixture was cooled to rt and slowly poured into ice water. The organic layer was separated from aqueous layer. The aqueous layer was extracted with two 15 mL portions of CH₂Cl₂. The organics were combined, washed with saturated aq. NaHCO₃ (15 mL), dried over anhydrous MgSO₄ and concentrated. The residue was purified by column chromatography (gradient of CH₂Cl₂/hexane) to give 0.55 g (62%) of the title compound as yellow solid. The properties of the title compound matched those reported by Blackman10 which are listed here:

mp 78-80° C. 1H NMR (CDCl₃, 400 MHz, δ): 8.93 (t, J=2.0 Hz, 1H), 8.44 (dd, J=8.0 Hz, 1.9 Hz, 1H), 8.38 (dd, J=8.3 Hz, 2.3 Hz, 1H), 8.23 (d, J=8.3 Hz, 2H), 7.71 (d, J=8.4 Hz, 2H), 7.66 (t, J=8.1 Hz, 1H). 13C NMR (CDCl3, 100 MHz, δ): 148.4 (u), 143.6 (u), 142.2 (u), 136.4 (u), 135.1 (u), 133.9 (dn), 133.6 (u)[q, 2J(CF)=33.4 Hz], 129.8 (dn), 128.9 (dn), 126.4 (dn) [q, 3J(CF)=4.0 Hz], 125.6 (dn) 123.6 (u) [q, 1J(CF)=274 Hz], 123.5 (dn). HRMS (ESI+) [M+H] calcd. for C15H9F3N3O2Cl2 390.0024. found 390.0064.

### 3-(3-nitrophenyl)-6-[4-(trifluoromethyl)phenyl]-s-tetrazine (3)

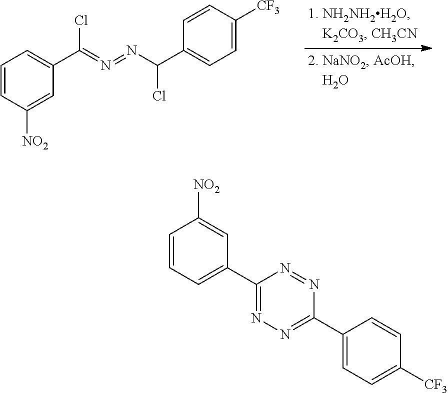

The following is a modification of the procedure of Blackman10. A round bottomed flask was charged with N′-(chloro(4-(trifluoromethyl)phenyl)methylene)-3-nitrobenzohydrazonoyl chloride (0.530 g, 1.36 mmol) and acetonitrile (10 mL), and was equipped with a reflux condenser. Hydrazine hydrate (0.068 mg, 1.36 mmol) was added, and the mixture was heated to reflux behind a blast shield for 1 h. Potassium carbonate (375 mg, 2.72 mmol) was added, and the mixture was heated to reflux for 24 h. Hydrazine hydrate (408 mg, 8.16 mmol) was added, and the mixture was heated to reflux for an additional hour. The mixture was cooled to rt, and diluted with CH₂Cl₂. The organics were washed with brine, dried over anhydrous MgSO4, and concentrated. The crude residue was dissolved in acetic acid (4 mL) at 0° C. The solution was stirred, and a solution of NaNO₂ (0.690 g, 10.0 mmol) in water (1 mL) was added dropwise. The mixture was allowed to stir for 3 h, and was then diluted with CH₂Cl₂ (50 mL). The organics were washed with sat. aq. NaHCO₃ (2×30 mL), dried over anhydrous magnesium sulfate and concentrated. The residue was the purified by column chromatography (gradient CH₂Cl₂ in hexane) to give 3 (260 mg, 55%) as pink solid. Anal. calculated for C₁₅H₈F₃N₅O₂: C, 51.88; H, 2.32; N, 20.17. Found: C, 51.48; H, 2.41; N, 19.81. The properties of the title compound matched those reported by Blackman10, which are listed here:

mp 217-219° C. ¹H NMR (CDCl3, 400 MHz, δ): 9.54 (t, J=2.0 Hz, 1H), 9.01 (dd, J=7.8 Hz, 1.6 Hz, 1H), 8.81 (d, J=8.3 Hz, 2H), 8.51 (dd, J=8.3 Hz, 2.3 Hz, 1H), 7.89 (d, J=8.3 Hz, 2H), 7.84 (t, J=8.1 Hz, 1H). 13C NMR (CDCl3, 100 MHz, δ): 164.7 (u), 163.4 (u), 147.9 (u), 136.1 (u), 133.8 (dn), 133.7 (u), 131.5 (u) [q, 2J(CF)=34.5 Hz], 130.5 (dn), 126.6 (dn) [q, 3J(CF)=4.0 Hz], 125.6 (dn) 123.6 (u) [q, 1J(CF)=272 Hz], 122.2 (dn). HRMS (ESI) [M+]+ calcd. for C15H8F3N5O2 347.0630. found 347.0622.

### 3-(3-aminophenyl)-6-[4-(trifluoromethyl)phenyl]-s-tetrazine (4)

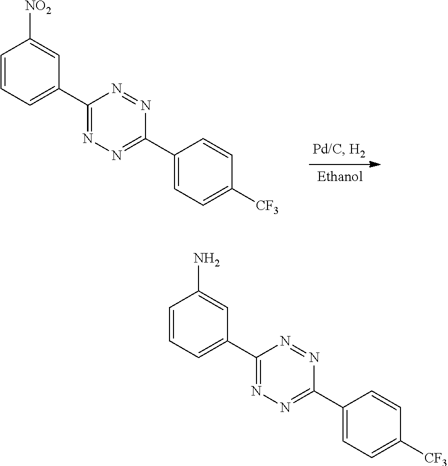 [SMILES: O=[N+]([O-])c1cccc(-c2nnc(-c3ccc(C(F)(F)F)cc3)nn2)c1 ; Nc1cccc(-c2nnc(-c3ccc(C(F)(F)F)cc3)nn2)c1]

A round bottom flask was charged with 10% Pd/C (100 mg), ethanol (15 mL) and 3-(3-nitrophenyl)-6-[4-(trifluoromethyl)phenyl]-s-tetrazine (247 mg, 0.712 mmol) under nitrogen atmosphere. The mixture was allowed to stir, and the flask was purged with hydrogen. Stirring continued under hydrogen (balloon pressure) for 12 h. The reaction mixture was diluted with methanol (25 mL), filtered, concentrated and purified by column chromatography to give the title compound (120 mg, 53%) as a red solid, mp 214-216° C. The properties of the title compound matched those reported by Blackman10, which are listed here:

¹H NMR (DMSO-d6, 400 MHz, δ): 8.71 (d, J=8.3 Hz, 2H), 8.06 (d, J=8.8 Hz, 2H), 7.81 (t, J=2.0 Hz, 1H) 7.71 (m, 1H), 7.32 (t, J=7.4 Hz, 1H), 6.89 (dd, J=8.5 Hz, 1.9 Hz, 1H), 5.5 (m, 2H). 13C NMR (CDCl3, 100 MHz, δ): 163.7 (u), 162.4 (u), 149.6 (u), 135.9 (u), 132.0 (u), 131.9 (u) [q, 2J(C—F)=34.5 Hz], 130.0 (dn), 128.2 (dn), 126.3 (dn) [q, 3J(CF)=4.0 Hz], 121.0 (u) [q, 1J(CF)=273 Hz], 118.2 (dn), 115.2 (dn), 112.4 (dn). HRMS (ESI) [M+H]+ calcd. for C15H11F3N5 318.0967. found 318.0966.

### 5-oxo-5-(3-(6-4-(trifluoromethyl)phenyl)-1,2,4,5-tetrazin-3-yl)phenylamino)pentanoic acid (5)

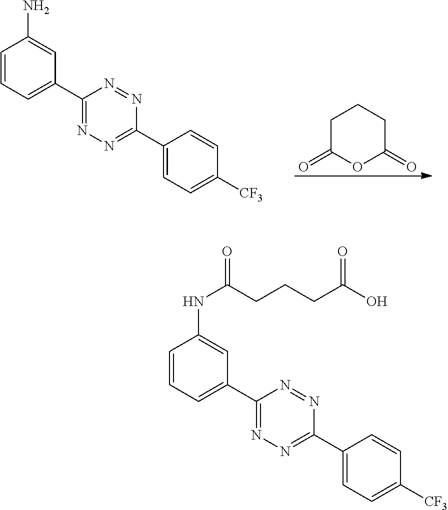 [SMILES: Nc1cccc(-c2nnc(-c3ccc(C(F)(F)F)cc3)nn2)c1 ; O=C1CCCC(=O)O1 ; O=C(O)CCCC(=O)Nc1cccc(-c2nnc(-c3ccc(C(F)(F)F)cc3)nn2)c1]

The following is a modification of the procedure of Blackman10. A 2 dram vial was charged with 3-(3-aminophenyl)-6-[4-(trifluoromethyl)phenyl]-s-tetrazine (100 mg, 0.315 mmol), glutaric anhydride (180 mg, 1.58 mmol) and THF (2 mL). The vial was flushed with nitrogen, capped, and heated with stirring at 80° C. for 4 h. The mixture was cooled to rt, centrifuged, and the supernatant decanted. The solid that was obtained was suspended in CH₂Cl₂ sonicated, centrifuged, supernatant decanted and dried to give the title compound (120 mg, 88%) as a pink solid. The properties of the title compound matched those reported by Blackman10, which are listed here:

mp 246-248° C. ¹H NMR (DMSO-d6, 400 MHz, δ): 10.3 (s, 1H), 8.92 (t, J=1.8 Hz, 1H), 8.74 (d, J=8.2 Hz, 2H), 8.23 (dd, J=7.8 Hz, 1.8 Hz, 1H) 8.09 (d, J=8.2 Hz, 2H), 7.92 (dd, J=8.2 Hz, 2.3 Hz, 1H), 7.63 (t, J=8.2, 1H), 2.43 (t, J=7.1 Hz, 2H), 2.31 (t, J=7.4 Hz, 2H), 1.85 (quin., J=7.0 Hz, 2H); 13C NMR (CDCl3, 100 MHz, δ): 174.3 (u), 171.3 (u), 163.5 (u), 162.6 (u), 140.4 (u), 135.9 (u), 132.0 (u), 132.0 (u) [q, 2J(C—F)=34.5 Hz], 130.1 (dn), 128.4 (dn), 126.4 (dn) [q, 3J(CF)=4.0 Hz], 124.2 (u)[q, 1J(CF)=275 Hz], 123.1 (dn), 122.6 (dn), 118.0 (dn), 35.5 (u) 33.1 (u), 20.4 (u).). HRMS (ESI) [M+H]+ calcd. for C20H16F3N5O3 432.1283. found 432.1283.

### tert-butyl (2-(5-oxo-5-((3-(6-(4-(trifluoromethyl)phenyl)-1,2,4,5-tetrazin-3-yl)phenyl)amino)pentanamido)ethyl)carbamate

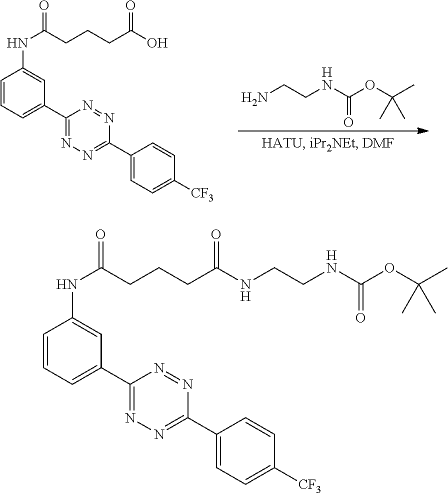 [SMILES: O=C(O)CCCC(=O)Nc1cccc(-c2nnc(-c3ccc(C(F)(F)F)cc3)nn2)c1 ; CC(C)(C)OC(=O)NCCN ; CC(C)(C)OC(=O)NCCNC(=O)CCCC(=O)Nc1cccc(-c2nnc(-c3ccc(C(F)(F)F)cc3)nn2)c1]

A 2 dram vial was swept with nitrogen, and sequentially charged with 5-oxo-5-(3-(6-4-(trifluoromethyl)phenyl)-1,2,4,5-tetrazin-3-yl)phenylamino)pentanoic acid (75 mg, 0.17 mmol), HATU (172 mg, 0.46 mmol) and a solution of tert-butyl (2-aminoethyl)carbamate (70 mg, 0.44 mmol) in anhydrous DMF (2 mL). The vial was capped, and the resulting mixture stirred for 20 h. The mixture was then diluted with CH₂Cl₂ (10 mL) and centrifuged. Residue was thrice suspended in CH₂Cl₂ (10 mL) sonicated, centrifuged, decanted supernatant and dried to give the title compound (70 mg, 70%) as a poorly soluble pink solid.

¹H NMR (DMSO-d6, 400 MHz, δ): 10.3 (s, 1H), 8.94 (t, J=2.0 Hz, 1H), 8.74 (d, J=7.8 Hz, 2H), 8.23 (dd, J=7.8 Hz, 2.0 Hz, 1H) 8.09 (d, J=8.7 Hz, 2H), 8.01-7.83 (m, 2H), 7.63 (t, J=7.8, 1H), 6.83 (br, s, 1H), 3.15-3.05 (m, 2H), 3.05-2.90 (m, 2H), 2.42-2.34 (m, 2H), 2.22-2.09 (m, 2H), 1.94-1.77 (m, 2H), 1.38 (s, 9H). LRMS (ESI) [M+Na]+ calcd. for C27H30F3N7O4 596. found 596.

### N1-(2-aminoethyl)-N-5-(3-(6-(4-(trifluoromethyl)phenyl)-1,2,4,5-tetrazin-3-yl)phenyl)glutaramide trifluoroacetic acid (Tz2)

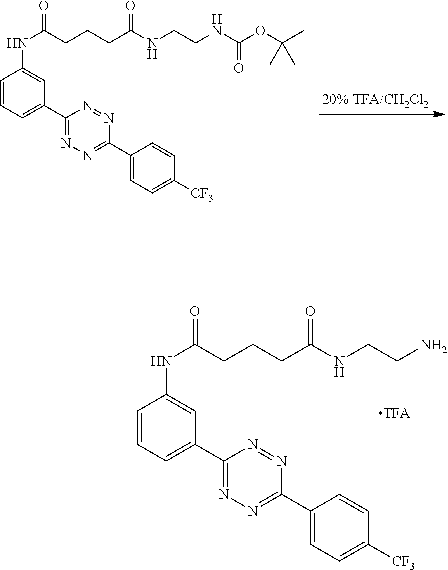 [SMILES: CC(C)(C)OC(=O)NCCNC(=O)CCCC(=O)Nc1cccc(-c2nnc(-c3ccc(C(F)(F)F)cc3)nn2)c1 ; NCCNC(=O)CCCC(=O)Nc1cccc(-c2nnc(-c3ccc(C(F)(F)F)cc3)nn2)c1]

A 2 dram vial containing tert-butyl (2-(5-oxo-5-((3-(6-(4-(trifluoromethyl)phenyl)-1,2,4,5-tetrazin-3-yl)phenyl)amino)pentanamido)ethyl)carbamate (50 mg, 0.87 mmol) was flushed with nitrogen. A solution of 20% trifluoroacetic acid in CH₂Cl₂ (2 mL) was added, and the resulting mixture stirred for 2 h at rt. The mixture was concentrated to give 56 mg (92%, presuming a bis-TFA salt) of Tz2 as red solid. ¹H NMR (DMSO-d6, 400 MHz, δ): 10.3 (s, 1H), 8.93 (t, J=2.2 Hz, 1H), 8.74 (d, J=8.3 Hz, 2H), 8.24 (dd, J=7.8 Hz, 2.0 Hz, 1H) 8.09 (d, J=8.3 Hz, 2H), 8.03 (t, J=5.0 Hz, 1H), 7.92 (m, 1H), 7.72 (br, s 3H), 7.63 (t, J=7.8, 1H), 3.33-3.22 (m, 2H), 2.91-2.78 (m, 2H), 2.40 (t, J=7.8 Hz, 2H), 2.20 (t, J=7.8 Hz, 2H), 1.87 (quint, J=7.8 Hz, 2H). 13C NMR (DMSO-d6, 100 MHz, δ): 173.1 (u), 171.8 (u), 164.0 (u), 163.1 (u), 140.8 (u), 136.4 (u), 132.6 (u) [q, 2J(CF)=32.3 Hz], 132.6 (u) 130.5 (dn), 128.8 (dn), 126.9 (dn) [q, 3J(CF)=3.6 Hz], 124.5 (u) [q, 1J(CF)=281 Hz], 123.6 (dn), 122.9 (dn), 118.4 (dn), 36.9 (u) 36.2 (u), 35.1 (u), 21.4 (u). Peaks due to trifluoroacetate counterion were observed at: 158.6 (u) [q, 2J(CF)=36.2 Hz], 116.4 (u) [q, 1J(CF)=289 Hz]. LRMS (ESI) [M+H]+ calcd. for C22H23F3N7O2 474. found 474.

### Synthesis of Tetrazine-Fluorophore Conjugates

### Aminobenzyltetrazine carboxyfluorescein (Tz1-fluorescein)

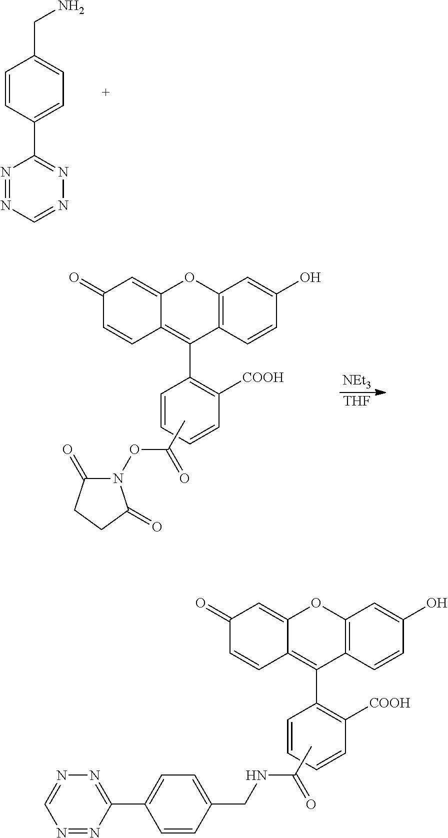

Tetrazine benzylamine (Tz1) was synthesized as previously described11. To a dried flask equipped with a stir bar was added Tz1 (10.7 mg, 0.057 mmol) in 5 mL anhydrous THF followed by 5-(and 6-)carboxyfluorescein, succinimidyl ester (NHS-fluorescein; 13.2 mg, 0.028 mmol, Thermo Scientific) and Et3N (11.9 μL, 0.085 mmol). The mixture was stirred overnight at room temperature under N₂ atmosphere. The solvent was removed under reduced pressure and the resulting solid was purified by normal phase silica gel column chromatography with 17% MeOH in CH₂Cl₂+0.1% (v/v) TFA. The eluate was dried under vacuum, then further purified by HPLC on a C-18 column (10-90% acetonitrile over 30 min. linear gradient). The product eluted at 19 min. and was freeze-dried to give Tz1-fluorescein as a dark orange solid. TLC Rf=0.38 (17% v/v MeOH in CH₂Cl₂ [symbol] +0.1% v/v TFA). ESI (+) calculated for [M−H]−: 544.13. found: 544.02.

### Aminobenzyltetrazine carboxyfluorescein diacetate (Tz1-fluorescein diacetate)

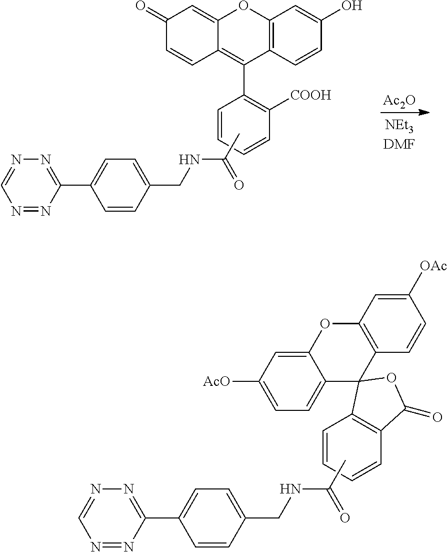

To a dried flask equipped with a stir bar was added Tz1-fluorescein (2 mg, 0.0037 mmol) in 2 mL anhydrous DMF, 3 eq. acetic anhydride, 5 eq. Et3N. The mixture was stirred at room temperature under N₂ atmosphere for 2 hours, during which time the reaction mixture turned from orange to pink. For workup, the reaction mixture was diluted with 20 volumes of H₂O, and the product was extracted into EtOAc. After drying with sodium sulfate, the EtOAc solvent was removed under reduced pressure to give a dark pink oil. The product was further purified by normal phase silica gel column chromatography (isocratic 100% ethyl acetate) to give a dark pink wax. ESI (+) calculated for [M+H]+: 629.15. found: 629.82.

### Aminobenzyltetrazine tetramethylrhodamine (Tz1-TMR)

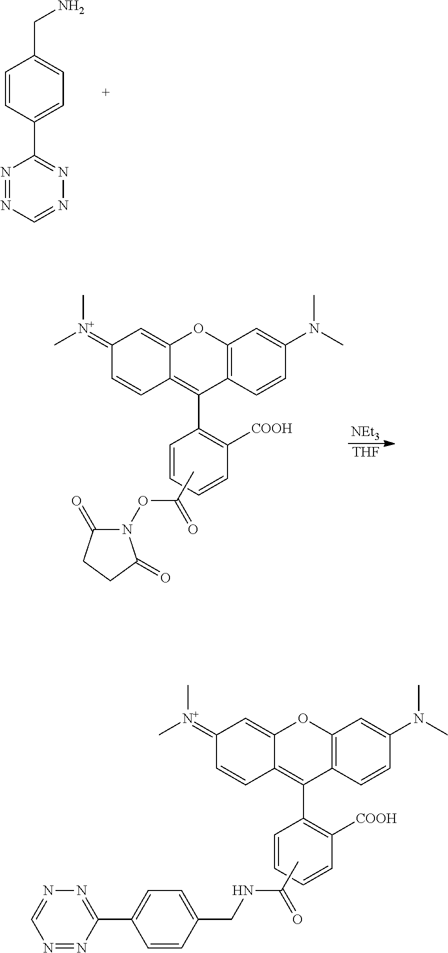

Synthesized under similar conditions as for Tz1-fluorescein, using 5-,6-carboxytetramethylrhodamine, succinimidyl ester (Thermo Scientific). After solvent removal, the resulting solid was purified by HPLC on a C-18 column (10-90% acetonitrile over 30 min. linear gradient). ESI (+) calculated for M+: 600.24. found: 600.30.

### Aminobenzyltetrazine Alexa Fluor 647 (Tz1-Alexa 647)

To a dried glass vial equipped with a stir bar was added 2 mg Tz1 (10.6 μmol), Alexa Fluor 647 carboxylic acid, succinimidyl ester (0.3 mg, Live Technologies), and Et3N (53.0 μmol) in 500 μL anhydrous DMSO. The reaction was stirred overnight at room temperature under N₂ atmosphere. The mixture was diluted with 10 volumes of H2O, then freeze-dried into a dark blue solid. The solid was purified by HPLC on a C-18 column (10-90% acetonitrile over 20 min. linear gradient). The product eluted at 8 min. and was again freeze-dried to give a dark blue solid.

### Trifluoromethyl bisaryltetrazine amine, carboxyfluorescein conjugate (Tz2-fluorescein)

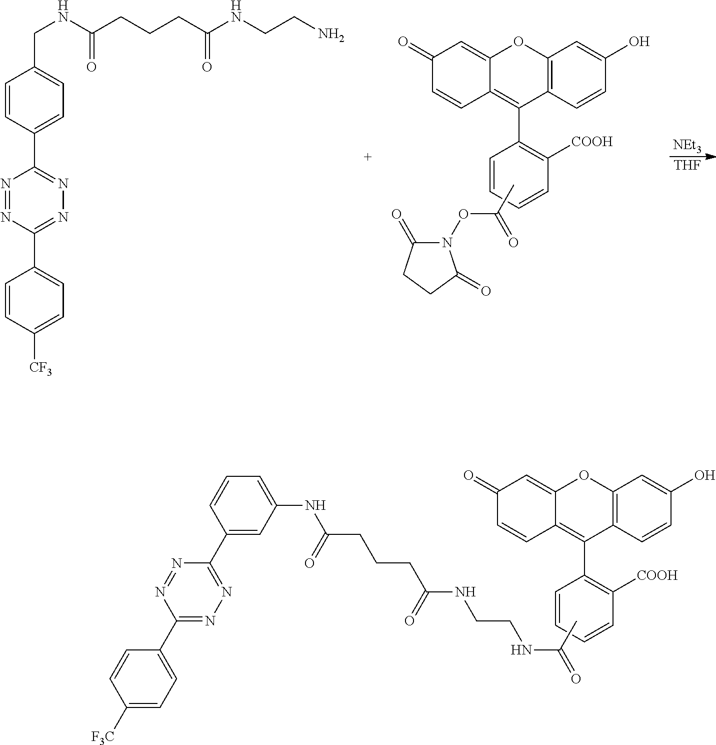

To a dried flask equipped with a stir bar was added Tz2 (20 mg, 0.034 mmol) in 1 mL anhydrous DMF followed by NHS-fluorescein (16 mg, 0.034 mmol) and Et3N (24 μL, 0.17 mmol). The mixture was stirred overnight at room temperature under N₂ atmosphere. The solvent was removed under reduced pressure and the product was purified on normal phase silica gel column chromatography with 5-15% (v/v) MeOH in CH₂Cl₂. The eluate was dried under vacuum, then further purified by HPLC on a C-18 column (10-90% acetonitrile over 30 min. linear gradient). The product eluted at 21 min. and was freeze-dried to give Tz2-fluorescein as a dark orange solid. TLC Rf=0.40 (10% v/v MeOH in CH₂Cl₂). ESI (+) calculated for [M+H]+: 832.4. found: 832.23.

### Trifluoromethyl bisaryltetrazine amine, carboxyfluorescein diacetate conjugate (Tz2-fluorescein diacetate)

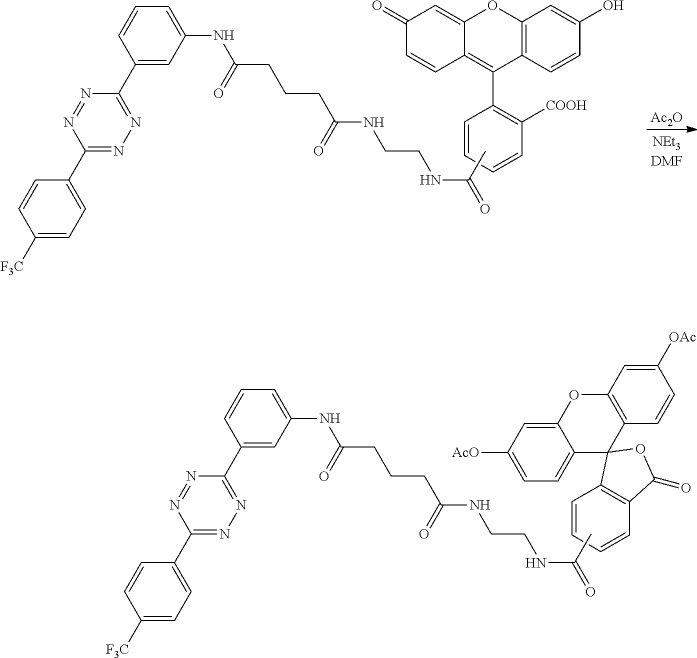

2 mg (0.0024 mmol) Tz2-fluorescein was used to synthesize Tz2-CFDA in the same protocol as for Tz1-CFDA. The extracted product was further purified by normal phase silica gel column chromatography (isocratic 100% ethyl acetate) to give a dark pink wax. ESI (+) calculated for [M+H]+: 916.25. found: 916.44.

HPLC Assay for In Vitro LplA-Mediated Trans-Cyclooctene Probe Ligation onto LAP Reactions were assembled with 250 nM (or 1 μM W37VLplA for Supporting FIG. 1A), 200 μM LAP (GFEIDKVWYDLDA), 500 μM trans-cyclooctene (TCO1, TCO2, or TCO3), 2 mM ATP, and 5 mM Mg(OAc)₂ in Dulbecco's phosphate buffered saline with 10% (v/v) glycerol and incubated at 30° C. for 30 min.

LplA protein was purified as previously described2 and stored at −80° C. in 20 mM Tris-HCl, pH 7.5 supplementated with 10% v/v glycerol. Reactions were quenched with 30 mM EDTA (final concentration) and resolved by HPLC (Varian ProStar) on a C-18 column using a linear gradient of 25-60% acetonitrile in H2O (with 0.1% v/v trifluoroacetic acid) over 14 minutes. Species were detected at 210 nm absorbance. Peaks corresponding to LAP and its trans-cyclooctene adducts were confirmed by ESI mass spectrometry. The extent of conversion was calculated from ratios of peak areas, neglecting minor extinction coefficient changes to LAP due to trans-cyclooctene ligation.

Live Cell Surface Fluorescence Labeling with Dye Washout

HEK cells were rinsed twice with Tyrode's buffer (145 mM NaCl, 1.25 mM CaCl2, 3 mM KCl, 1.25 mM MgCl₂, 0.5 mM NaH₂PO₄, 10 mM glucose, 10 mM HEPES, pH 7.4), then treated with 5 μM W37VLplA, 100 μM TCO₂, 1 mM ATP and 1 mM Mg(OAc)₂ in the same buffer for 15 minutes at room temperature. Cells were rinsed 3 times before further treatment with 100 nM Tz1-fluorescein in Tyrode's buffer for 5 minutes at room temperature. Imaging was performed live after another 2 rinses. LAP-LDL receptor and nuclear cyan fluorescent protein marker were transfected at a 1:1 ratio, with altogether 400 ng plasmid per 1 cm² culture.

Hippocampal neurons were labeled in the same way, except that the TCO2 ligation step was shortened to 10 minutes and performed at 37° C. 100 nM Tz1-Alexa 647 was used. LAP-neuroligin-1 and Homer1b-GFP were transfected at a 1:1 ratio, with altogether 2 μg plasmid per 2 cm² culture. It was routinely observed that the Tz1-Alexa 647 conjugate bound non-specifically to cellular debris in a trans-cyclooctene independent manner, contributing some punctate background in imaging. This problem can be alleviated by having healthy neuron cultures with minimal debris.

Live Cell Surface Fluorogenic Labeling without Dye Washout

HEK cells grown in a monolayer on #1.5 Lab-Tek II chambered coverglass (Nalge Nunc International) were treated with TCO2. After 5 rinses with Tyrode's buffer, the chamber was placed on the microscope objective covered with 200 μL of the same buffer. Image acquisition sequence was initiated immediately after 200 μL of 100 nM Tz1-fluorescein in Tyrode's buffer was added to the chamber, and briefly mixed by pipeting. Final concentration of Tz1-fluorescein was therefore 50 nM after mixing. LAP-LDL receptor and a mCherry fluorescent protein transfection marker were transfected at a 1:1 ratio, with altogether 400 ng plasmid per 1 cm² culture.

To quantify the imaging signal/noise ratio, 17 cells with obvious surface fluorescence (by eye) at the 180 sec. time point were chosen and separate masks created automatically by the Slidebook software over the fluorescent rims. The averaged pixel intensity was defined as “signal”. To measure noise, 10 cells with no obvious surface fluorescence (by eye) at the 180 sec. time point were chosen, and rectangular masks created manually over the interiors of these cells. The averaged (over all 10 masks) pixel intensity was defined as “noise”. Both “signal” and “noise” had a background subtraction from averaged pixel intensity corresponding to non-cellular regions.

Live Intracellular Fluorescence Labeling with Dye Washout

HEK cells were rinsed once with MEM, then treated with 200 μM TCO2 in the same medium for 30 min. at 37° C. Cells were rinsed twice, then left in complete medium (MEM with 10% v/v fetal bovine serum) for a further 30 min. at 37° C. to allow excess unligated TCO₂ to wash out of cells. 500 nM Tz1-fluorescein diacetate or 1 μM Tz1-TMR in MEM was then added to cells for 5 min. at 37° C. Cells were then rinsed twice with complete medium and kept at 37° C. for excess dye to wash out. Complete medium was replaced twice more at 20 and 40 minutes later to improve washout. Cells were imaged live after altogether 2 hours in complete medium. HEK cells were transfected with 300 ng nuclear LAP-blue fluorescent protein and 50 ng W37VLplA per 1 cm² culture.

COS-7 cells expressing cytoskeletal proteins were labeled similarly to HEK cells, except that 100 μM TCO2 was used, Tz1-fluorescein diacetate loading concentration was reduced to 100 nM, and tetrazine-dye washout time was reduced to 1 hour before cells were imaged live. COS-7 cells were transfected with 200 ng LAP-actin or 200 ng vimentin-LAP along with 50 ng W37VLplA per 1 cm² culture.

Measurement of Kcat for In Vitro W37VLplA Mediated Ligation of TCO2 and Lipoate onto LAP

Reactions were assembled with 500 μM TCO₂ or lipoic acid, 500 μM LAP (GFEIDKVWYDLDA), 2 mM ATP, 5 mM Mg(OAc)₂ and 250 nM W37VLplA and kept in a 30° C. waterbath. After 5, 10, 15 and 20 minutes, an aliquot was drawn from the reaction vial, quenched with 30 mM EDTA (final concentration) and the product quantified by HPLC as in Table 3. The plot of product concentration against time was fitted to a linear line whose slope corresponds to the initial velocity. The value of kcat was calculated from the Michaelis-Menten equation Vmax=(kcat)([Enzyme]) at substrate-saturating conditions. Measurements were performed in triplicate.

Measurement of Tetrazine-Dye Fluorescence Turn-on after Diels-Alder Cycloaddition

Tetrazine-fluorophore conjugates were dissolved in Dulbecco's phosphate buffered saline, pH 7.4 at approximately 100 nM concentration. Solutions with >100-fold excess TCO1 in DMSO or DMSO vehicle alone added were transferred into an opaque, flat-bottom 96-well plate (Greiner Bio One) and their fluorescence emission scanned with a Safire Tecan fluorescence microplate reader. Excitation was fixed at 430 nm for fluorescein, 530 nm for TMR, and 610 nm for Alexa 647. Fold-changes in fluorescence turn-on are reported at respective fluorescence emission maximum wavelengths.

### Measurement of In Vitro Second-Order Diels-Alder Cycloaddition Rate Constant Between LAP-TCO2 and Tetrazine-Fluorescein Conjugates

LAP-TCO₂ adduct was prepared by mixing 500 μM LAP with 1 mM TCO₂, 2 μM W37VLplA, 2 mM ATP, and 5 mM Mg(OAc)₂ in Dulbecco's phosphate buffered saline (DPBS), pH 7.4 supplemented with 10% v/v glycerol. Ligation reaction was allowed to proceed at 30° C. for 4 hours to maximize ligation yield. The mixture was then resolved by preparatory HPLC on a C-18 column (25-45% acetonitrile over 30 min. linear gradient, supplemented with 0.1% v/v trifluoroacetic acid), where the product eluted at 19 min. and its identity confirmed by ESI mass spectrometry. The eluate was freeze-dried into a white powder and dissolved in DPBS for subsequent measurements.

To measure second-order rate constant by pseudo-first-order approximation, 100 μL Tz1- or Tz2-fluorescein (100 nM in DPBS) was loaded into an opaque, flat-bottom 96-well plate (Greiner Bio One), then mixed with 100 μL LAP-TCO₂ (3.3 μM in DPBS). The fluorescence intensity at 520 nm was immediately recorded at 9-second intervals until the reaction reached completion in approximately 5 minutes. The fluorescence intensity was then converted to [tetrazine-fluorescein], assuming that initial fluorescence corresponded to 50 nM and final fluorescence corresponded to 0 nM tetrazine-fluorescein. The plot of ln [tetrazine-fluorescein] against time was fitted to a linear line whose slope corresponds to the pseudo-first order rate constant, which was then converted to the second-order rate constant. Measurements were performed in triplicate.

### Comparing Diels-Alder Cycloaddition, Copper Catalyzed Azide-Alkyne Cycloaddition (CuAAC), and Copper Free “Click” Chemistries for Cell Surface Fluorescence Labeling

HEK cells were rinsed twice with Tyrode's buffer, then treated with 1 mM ATP, 5 mM Mg(OAc)₂, and either 10 μM W37VLplA/100 μM TCO₂ (for subsequent Diels-Alder staining) or 10 μM wild-type LplA/100 μM 8-azidooctanoic acid (for subsequent CuAAC and strain-promoted cycloaddition staining)2 in the same buffer for 30 min. at room temperature. These were previously determined, by subsequent lipoic acid pulse labeling, to give almost quantitative yield of 8-azidooctanoic acid ligation. Cells were then rinsed and treated with Tz1-Alexa 647, alkyne-Alexa 647 with 50 μM CuSO4/2.5 mM sodium ascorbate/250 μM THPTA ligand12 (a gift from Chayasith Uttamapinant), or DIBO-Alexa 647 (Life Technologies) in Tyrode's buffer for 3 minutes at room temperature and imaged live after further rinsing. HEK cells were transfected with LAP-LDL receptor and nuclear cyan fluorescent protein marker in a 1:1 ratio, with altogether 400 ng per 1 cm² culture.

Determination of Cell Viability after Cell Surface Fluorescence Labeling by Diels-Alder Cycloaddition and CuAAC

HEK cells grown in flat-bottom 96-well plates (Greiner Bio One) were transfected and treated similarly to those in Supporting FIG. 6A, except that the LplA concentration was reduced to 1 μM, and the TCO₂/8-azidooctanoic acid ligation and fluorescence staining steps were changed to 15 minutes and 5 minutes, respectively. Afterward, 100 μL of premixed CellTiter-Glo reagent (Promega) was added into each well. The plate was shaken in a 30° C. orbital shaker for 10 minutes and the luminescence from each well was recorded by a SPECTRAmax dual-scanning microplate spectrofluorometer. Measurements were performed in triplicate.

### Quantification of Labeling Signal/Noise Ratio for Tz1- and Tz2-Fluorescein Diacetate

Masks over the nuclear regions were generated automatically in the Slidebook software by gating the BFP fluorescence. 24 gates of a wide range of BFP intensities over 3 fields of view for each condition were randomly chosen. The fluorescein intensities within these gates were defined as “signal”. Rectangular gates in the perinuclear regions of these chosen cells were drawn manually and their corresponding fluorescein intensities defined as “noise”. Both “signal” and “noise” were background-adjusted from the averaged fluorescence intensity in non-cellular regions.

Determination of Tz1-Fluorescein Diacetate Labeling Specificity by Polyacrylamide Gel Electrophoresis and Fluorescein in-Gel Fluorescence Imaging

HEK cells grown in 6-well plates (Greiner Bio One) were transfected with 3 μg nuclear LAP-blue fluorescent protein and 500 ng W37VLplA, then treated with TCO2 followed by Tz1-fluorescein diacetate in the same way as for FIG. 3B, except that the dye washout in complete medium at 37° C. was lengthened to 4 hours. Cells were then rinsed twice with DPBS and scraped off the surface. Cells were lysed by 3 rounds of freezing and thawing in hypotonic lysis buffer (1 mM HEPES, 5 mM MgCl2, pH 7.5) supplemented with protease inhibitor cocktail (Sigma Aldrich) and phenylmethanesulfonyl fluoride. The lysate was clarified by centrifuging at 10,000 g for 5 min. at 4° C. and the supernatant resolved on a 12% SDS polyacrylamide gel. Fluorescein in-gel fluorescence was imaged on a FUJIFILM FLA-9000 gel imager with a 473 nm laser using a blue long-pass filter. After fluorescence imaging the same gel was stained with Coomassie and re-imaged under white light after destaining.

### Visualization of Actin Filaments and Vimentin Intermediate Filaments by Tz1-TMR Labeling and Immunofluorescence Staining

HeLa cells grown on glass coverslips were transfected and labeled with Tz1-TMR in the same way as described above. Cells were then fixed with 3.7% (v/v) formaldehyde in DPBS for 15 min. at room temperature and subsequently permeabilized with methanol for 5 min. at −20° C. Samples were blocked with 0.5% (w/v) casein in DPBS for 4 hours at room temperature, then treated with a 1:300 dilution of rabbit-anti-HA antibody (Life Technologies) or mouse-anti-C-myc antibody (Life Technologies) followed by a 1:300 dilution of goat-anti-rabbit or goat-anti-mouse antibody Alexa Fluor 647 conjugate (Life Technologies) for 15 min. each step in the blocking buffer.

### Results

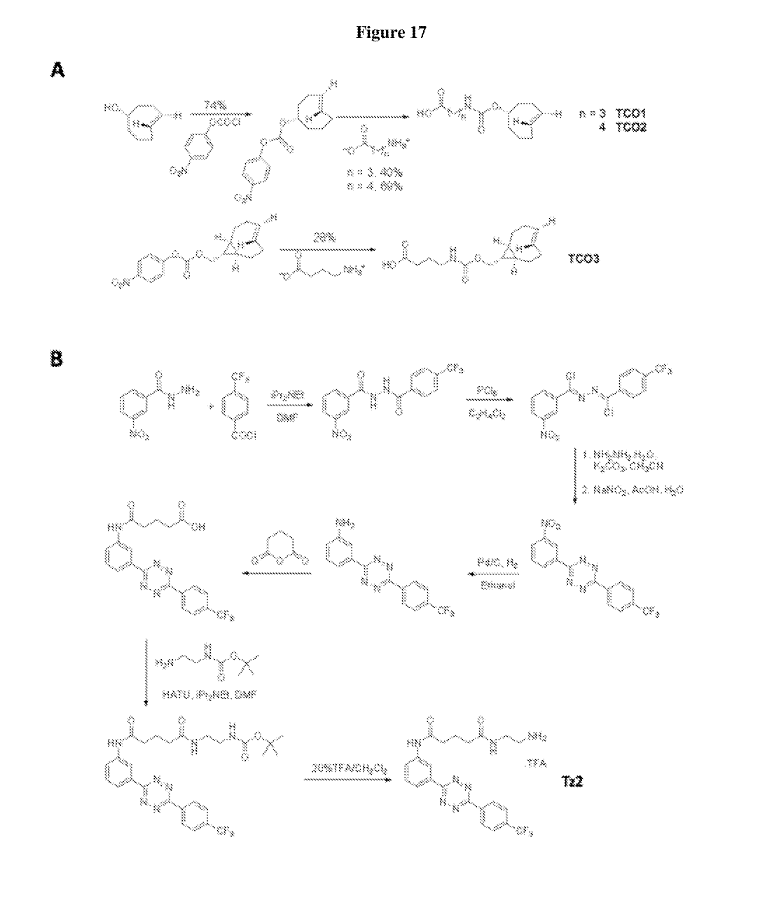

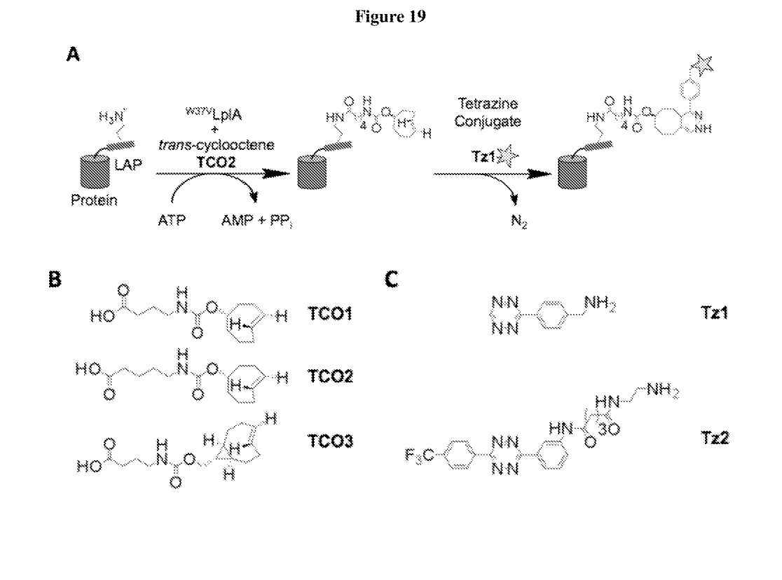

Three types of reactions were considered for the chemoselective derivatization: copper-catalyzed azide-alkyne cycloadditions (CuAAC) (Wang, et al., J. Am. Chem. Soc., 125:3192-3193 (2003)), strain-promoted azide-cycloalkyne cycloadditions (Agard, et al., J. Am. Chem. Soc., 127:11196 (2005)), and inverse-electron-demand Diels-Alder cycloadditions of tetrazines and trans-cyclooctenes (Blackman, et al., J. Am. Chem. Soc., 130:13518-13519 (2008)). An exemplary synthesis scheme of trans-cyclooctenes is shown in FIG. 17. CuAAC is restricted to the cell surface due to its dependence on toxic Cu(I) (Rostovtsev, et al., Angew. Chem., Int. Ed., 41:2596-2599 (2002)). PRIME was previously used in conjunction with strain-promoted cycloaddition for fluorescent labeling of cell surface proteins (Fernandez-Suarez, et al., Nat. Biotechnol., 25:1483-1487 (2007)). The slow kinetics of this reaction (k=10-3 to 1 M-1 s-1)13, however, limited our overall labeling yield and hence the achievable signal-to-noise ratio for imaging. Both CuAAC (k up to 104 M-1 s-1/M copper)14 and the Diels-Alder cycloaddition (k up to 104 M-1 s-1) (Devaraj, et al., Angew. Chem., Int. Ed., 48:7013-7016 (2009)) are much faster. The Diels-Alder reaction is also compatible in principle with the cell interior, although the only previous demonstration was intracellular labeling of a taxol derivative (Devaraj, et al., Angew. Chem., Int. Ed., 49:2869-2872 (2010)). Due to both its speed and potential for intracellular compatibility, the Diels-Alder cycloaddition shown in FIG. 19 was chosen for this study.

To utilize this chemistry, we first needed to choose between having LplA ligate the tetrazine or the trans-cyclooctene. We noted that the trans-cyclooctene moiety would be less bulky and therefore require less re-engineering of LplA. This is because tetrazine itself is unstable in aqueous solution, and must be stabilized by conjugation to one or more aromatic rings (Balcar, et al., Tetrahedron Lett., 24:1481-1484 (1983)), making the overall moiety quite large. Additionally, tetrazines quench the fluorescence of some covalently attached fluorophores, until reaction with trans-cyclooctene16. To allow for the possibility of fluorogen-ic labeling, we opted to conjugate the fluorophore to tetrazine.

Based on our experience, LplA prefers substrates with 3-4 linear methylenes linking the carboxylate and the bulky feature1. We therefore synthesized three trans-cyclooctene substrates for LplA: TCO1, TCO2, and TCO3, with structures shown in FIG. 19B and syntheses enabled by our photochemical flow method (Scheme 1) (Royzen, et al., J. Am. Chem. Soc., 130:3760-3761 (2008)). See also FIG. 17. TCO1 and TCO2 differ only in the length of their aliphatic linkers, while TCO3 has a cyclopropane ring fusion, which adds strain and accelerates the cycloaddition up to 160-fold19. We prepared a panel of LplA mutants and screened for their ability to ligate these three TCOs onto LAP using an HPLC assay (Table 3). Not surprisingly, wild-type LplA was unable to ligate any of the three substrates efficiently. Our other LplAs each harbored a single mutation at Trp37, a gatekeeper residue that has given us access to various unnatural substrates in the past (Uttamapinant, et al., Proc. Natl. Acad. Sci. U.S.A., 107:10914-10919 (2010); Baruah, et al., Angew. Chem., Int. Ed., 47:7018-7021 (2008)). We tested the Trp37→Gly mutant, with the active site maximally enlarged, as well as Trp37→Ile and →Val mutants that carve out a smaller, hydrophobic hole. We found that TCO2 scored significantly better than the other probes, and was best paired with the Val mutant (designated W37VLplA, Table 3).

Enzyme-dependent ligation was confirmed by negative controls omitting ATP or W37VLplA, and by mass spectrometry. We estimated the Michaelis-Menten kcat of TCO2 ligation onto LAP to be 0.34±0.02 s⁻¹, comparable to the fastest unnatural probe that we have reported to date (aryl azide: 0.31±0.04 s-1)20, and only 2-fold slower than ligation of the natural substrate lipoic acid by the same enzyme.

TABLE 3
Ligation efficiencies of lipoic acid ligase variants
with the three trans-cyclooctene substrates (TCO1-3)

| | Ligase | | | |
|---|---|---|---|---|
| Probe | Wild-type | Trp37→Gly | Trp37→Ile | Trp37→Val |
| TCO1 | 0.5 ± 0.0 | — | 0.5 ± 0.4 | — |
| TCO2 | 2.6 ± 0.9 | 52.6 ± 1.1 | 77.8 ± 1.6 | 100.0 ± 8.3 |
| TCO3 | 6.5 ± 1.0 | 22.6 ± 2.0 | 6.0 ± 0.6 | 6.6 ± 0.6 |

The relative abilities of wild-type and mutant ligases to ligate TCO1-3 onto LplA acceptor peptide (LAP) were measured by an HPLC assay after 30 min. reaction time. Average, normalized product percentages from triplicate measurements are shown. (—) indicates no detectable product. Errors, ±1 s. d.

We next focused on the design and syntheses of the tetrazine-fluorophore conjugates. The previously reported tetrazine structure Tz1 (FIG. 19C) reacts rapidly with trans-cyclooctenes, and has been used for small-molecule labeling in the cellular context (Devaraj, et al., Angew. Chem., Int. Ed., 49:2869-2872 (2010)). However, the lack of a second aryl substitution on Tz1 leaves it susceptible to non-specific reactions with cellular nucleophiles and dienophiles. Following the design of Blackman21, we synthesized an alternative 3,6-diaryl-s-tetrazine, Tz2 (FIG. 19C; synthesis shown in FIG. 17). Structures closely related to Tz2 had been shown to be unusually stable toward amines and thiols (Blackman, Thesis, University of Delaware, Newark, Del. (2011)). The electron withdrawing p-trifluoromethyl substituent of Tz2 augments the reactivity toward trans-cyclooctenes.

Both Tz1 and Tz2 were conjugated to fluorescein. Upon reaction with excess trans-cyclooctene, we measured 13.4- and 16.7-fold increases in fluorescein emission, respectively, in agreement with previous reports describing similar dyes16. We also measured second order rate constants for reaction with TCO2-ligated LAP, and found values of 5000±700 and 380±40 M-1 s-1 for Tz1-fluorescein and Tz2-fluorescein, respectively.

We proceeded to test cell surface fluorescence labeling. Here, nucleophiles are less abundant than inside cells, so we utilized only the faster tetrazine probe, Tz1-fluorescein. LAP-tagged low density lipoprotein receptor (LAP-LDL receptor) was expressed in human embryonic kidney 293T cells (HEK cells). We externally supplied 5 μM W37VLplA, 100 μM TCO2 and ATP for 15 minutes. We rinsed off the excess re-agents, then stained the cells with 100 nM Tz1-fluorescein for 5 minutes and observed specific labeling on transfected cells after further brief rinsing. Negative controls with ATP omitted, wild-type LplA, or inactive LAP mutant all eliminated the labeling signal.

Furthermore, we found it possible to perform fluorogenic labeling of LAP-LDL receptor, using 50 nM Tz1-fluorescein and without rinsing. Fluorescence signal accumulated specifically on transfected cells, with signal-to-noise ratios saturating after approximately 3 minutes, and with minimal background signal from the surrounding excess Tz1-fluorescein.

To extend cell surface labeling to other colors and cell types, we conjugated Tz1 to Alexa 647, a brighter fluorophore suitable for single molecule fluorescence detection and super-resolution imaging (Jones, et al., Nat. Methods, 8:499-505 (2011)). Tz1-Alexa 647 was used to label LAP-tagged neuroligin-1 on the surface of rat neurons with high specificity and minimal apparent toxicity.

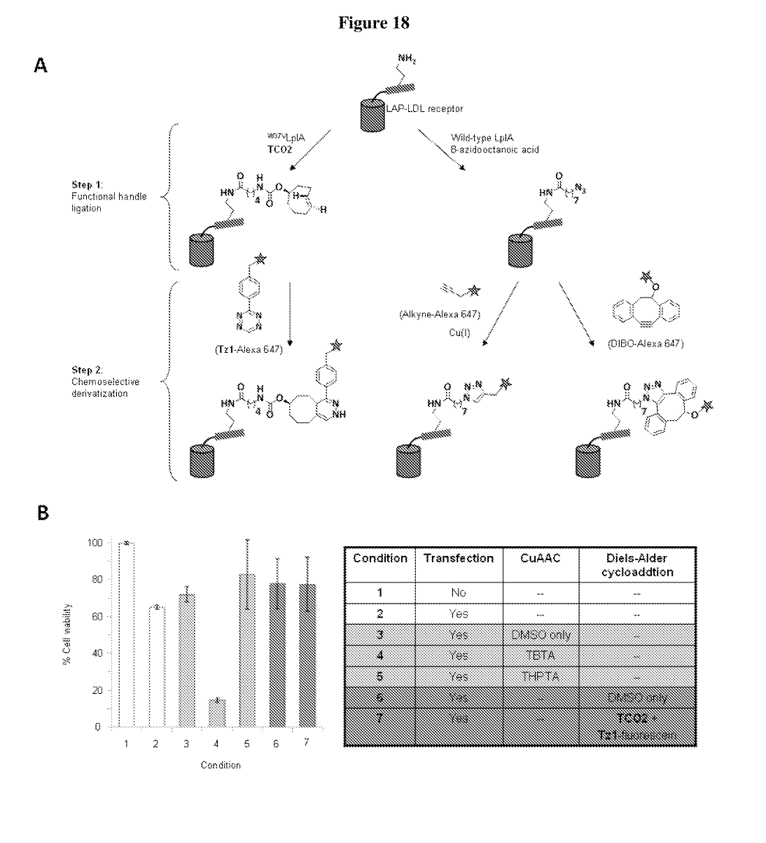

We directly compared Diels-Alder cycloaddition with two other bioorthogonal labeling chemistries that are compatible with the cell surface: CuAAC and strain-promoted azide-alkyne cycloaddition. We found that under otherwise identical conditions, Diels-Alder cycloaddition gave specific signal at 10 nM of Tz1-Alexa 647, while other methods required at least 1 μM of dye to achieve a similar signal-to-noise ratio. These results demonstrate that the Diels-Alder cycloaddition is much more sensitive while retaining similar specificity. FIG. 18A. Additionally, using an assay of cellular ATP content, we found that labeling by the Diels-Alder cycloaddition was not toxic, in contrast to CuAAC with TBTA ligand23 (although CuAAC with a new generation ligand, THPTA24, was considerably less toxic). FIG. 18B.

Negative charges on fluorescein and Alexa 647 prevent their tetrazine conjugates from crossing cell membranes. To label intracellular proteins, we first prepared cell-permeable derivatives, Tz1- and Tz2-fluorescein diacetate. Upon entering the cell interior, endogenous esterases hydrolyze the acetyl groups and release the intact tetrazine-fluorescein conjugate. For initial experiments, we expressed nuclear-localized, LAP-tagged blue fluorescent protein (nuclear LAP-BFP), as well as cytoplasmic W37VLplA inside HEK cells. The We synthesized another cell-permeable, red-shifted fluorophore conjugate, Tz1-tetramethylrhodamine (Tz1-TMR), and proceeded to optimize the cellular labeling conditions for both this conjugate and Tz1-fluorescein diacetate. Following the optimized protocol shown below, we observed labeling signal specific to the nuclei of transfected cells, despite the presence of LplA in both the cytosol and the nucleus.

| 200 μM TCO2 | Washout | 100 nM − 1 μM | Washout |
|---|---|---|---|
| 30 min. | 30 min. | Tz1-dye, 5 min. | 1-2 hours |

Negative controls with TCO2 omitted, wild-type LplA, or an inactive LAP mutant abolished labeling signals. We also examined the labeling specificity by lysing cells after Tz1-fluorescein diacetate treatment, and imaging the fluorescence of the lysate after gel separation. Supporting FIG. 8 shows that a single protein corresponding to the size of LAP-BFP was selectively labeled over the endogenous proteome.

We were unable to achieve fluorogenic labeling inside cells because high fluorescence signal was observed inside untransfected cells as well as cells free of TCO2 treatment, immediately upon loading of both Tz1-fluorophore conjugates. We were, however, able to wash away off-target dyes after 2 hours. In COS-7 cells, where the required dye washout time was shorter, we also successfully labeled actin filaments (LAP-β-actin) and intermediate filaments (vimentin-LAP) with high specificity. It was observed that actin and vimentin filaments labeled by Tz1-TMR co-localized perfectly with filaments detected by immunofluorescence staining in the same cells, indicating that the labeling was specific.

In summary, results from this study show that the tetrazine-trans-cyclooctene Diels-Alder cycloaddition is highly efficient for the fluorescence labeling of cell surface proteins and sufficiently bioorthogonal for labeling of intracellular proteins. We utilized this fast chemistry for the extension of PRIME to a panel of useful fluorophores, including tetramethylrhodamine and Alexa 647, while retaining a level of specificity comparable to direct fluorophore ligation by PRIME1. This method is generally applicable to different proteins in various cell types.

On the cell surface, we achieved fluorogenic labeling using tetrazine-fluorescein, but failed to accomplish fluorogenic labeling with Alexa 647 because its red-shifted fluorescence emission was not significantly quenched when conjugated to Tz1. Inside the cell, we observed a tradeoff between the reactivity and stability of two different tetrazine structures. It is suggested that, while monoaryl-substituted Tz1 is significantly more reactive than diaryl Tz2 toward trans-cyclooctene, the former is also more prone to cross-reactivity with endogenous nucleophiles or dienophiles. This study therefore illustrates the need for next-generation tetrazines that are less kinetically hindered by protective substitutions, and more able to quench the fluorescence of red dyes.

### Example 3

### Fluorophore Targeting to Cellular Proteins Via Enzyme-Mediated Azide Ligation and Strain-Promoted Cycloaddition

Methods for fluorophore targeting to cellular proteins can allow imaging with dyes that are smaller, brighter, and more photostable than fluorescent proteins. Here, we extend LplA-based labeling to green- and red-emitting fluorophores by employing a two-step targeting scheme. First, we found that the W37I mutant of LplA catalyzes site-specific ligation of 10-azidodecanoic acid to LAP in cells, in nearly quantitative yield after 30 minutes. Second, we evaluated a panel of five different cyclooctyne structures, and found that fluorophore conjugates to aza-dibenzocyclooctyne (ADIBO) gave the highest and most specific derivatization of azide-conjugated LAP in cells. However, for targeting of hydrophobic fluorophores such as ATTO 647N, the hydrophobicity of ADIBO was detrimental, and superior targeting was achieved by conjugation to the less hydrophobic monofluorinated cyclooctyne (MOFO). Our optimized two-step enzymatic/chemical labeling scheme was used to tag and image a variety of LAP fusion proteins in multiple mammalian cell lines with diverse fluorophores including fluorescein, rhodamine, Alexa Fluor 568, ATTO 647N, and ATTO 655.

### Methods

### In Vitro Azide Ligation

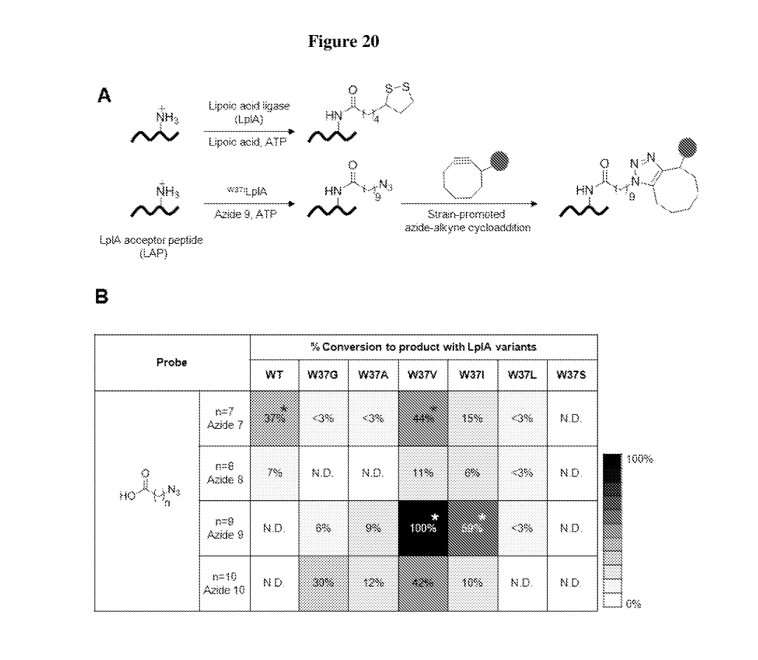

For the screen in FIG. 20B, reactions containing 100 nM LplA enzyme, 20 μM alkyl azide probe, 600 μM LAP peptide (sequence: H2N-GFEIDKVWYDLDA-CO₂H; SEQ ID NO:4), 2 mM ATP, and 2 mM magnesium acetate in 25 mM Na2HPO4 pH 7.2 were incubated at 30° C. for 20 minutes. Reactions were quenched with 40 mM EDTA (ethylenediaminetetraacetic acid, final concentration). Percent conversion to LAP-azide adduct was determined by HPLC with a C18 reverse phase column, recording absorbance at 210 nm. Elution conditions were 30-60% acetonitrile in water with 0.1% trifluoroacetic acid over 20 minutes at 1.0 mL/min flow rate. The percent conversion was calculated from the ratio of LAP-azide to sum of (unmodified LAP+LAP-azide). Reactions containing 1 μM LplA enzyme, 500 μM azide 9, and 300 μM LAP peptide were incubated at 30° C. for 2 hours. To determine kinetic measurements, reactions containing 100 nM W37ILplA, 25-700 μM azide 9, and 600 μM LAP peptide were incubated at 30° C., before quenching at various time points with EDTA.

### Mammalian Cell Culture and Transfection

HEK, HeLa, and COS-7 cells were cultured in Modified Eagle medium (MEM; Cellgro) supplemented with 10% v/v fetal bovine serum (FBS; PAA Laboratories). All cells were maintained at 37° C. under 5% CO2. For imaging, cells were plated on 5 mm×5 mm glass cover slips placed within wells of a 48-well cell culture plate (0.95 cm2 per well) 12-16 hours prior to transfection. HEK cells were plated on glass pre-coated with 50 μg/mL fibronectin (Millipore) to increase adherence. In general, cells were transfected with 200 ng W37ILplA plasmid and 400 ng LAP fusion plasmid using Lipofectamine 2000 (Invitrogen) at 50-70% confluency. For FIGS. 2B and S2B, WTLplA and W37VLplA plasmids were introduced at 20 ng rather than 200 ng, to give comparable expression levels to W37ILplA (at 200 ng), since the former express much more strongly.

### General Protocol for Intracellular Protein Labeling

16-20 hours after transfection, mammalian cells were incubated in complete media (10% FBS in MEM) containing 200 μM azide 9 for 1-2 hours at 37° C. To wash out excess azide 9, cells were rinsed three times with fresh, pre-warmed complete media every 30 minutes for 1-1.5 hours in total. Cells were then incubated with FBS-free MEM containing 10 μM cyclooctyne-fluorophore conjugate for 10 minutes at 37° C., followed by rinsing three times with MEM over 5 minutes. Thereafter, cells were switched to fresh, pre-warmed complete media, and the media was changed every 30 minutes-1 hour, for 1.5-8 hours at 37° C., prior to imaging. We have not observed any morphological changes in the cells during the washout period. For ATTO 647N and ATTO 655 conjugates, because of the intense brightness of the fluorophores, these were loaded at 1 μM rather than 10 μM.

### Cell Imaging

Cells were imaged in Dulbecco's phosphate buffered saline (DPBS) on glass coverslips at room temperature. For confocal imaging, we used a ZeissAxioObserver inverted microscope with a 60× oil-immersion objective, outfitted with a Yokogawa spinning disk confocal head, a Quadband notch dichroic minor (405/488/568/647), and 405 (diode), 491 (DPSS), 561 (DPSS), and 640 nm (diode) lasers (all 50 mW). BFP (excitation 405 nm; emission 445/40 nm), YFP/fluorescein/Oregon Green 488 (excitation 491 nm; emission 528/38 nm), Alexa Fluor 568/TMR/X-rhodamine (excitation 561 nm; emission 617/73 nm), and Alexa Fluor 647/ATTO 647N/ATTO 655 (excitation 640 nm, emission 700/75 nm) images were acquired using Slidebook 5.0 software (Intelligent Imaging Innovations). Acquisition times ranged from 100 milliseconds to 3 seconds. Fluorophore intensities in each experiment were normalized to the same intensity ranges.

### General Synthetic Methods

All reagents were the highest grade available and purchased from Sigma-Aldrich, Anaspec, Thermal Scientific, TCI America, Alfa Aesar, or Life Technologies and used without further purification. Anhydrous solvents were drawn from Sigma-Aldrich SureSeal bottles. Analytical thin layer chromatography was performed on 0.25 mm silica gel 60 F254 plates and visualized under short or long wavelength UV light, or after staining with bromocresol green or ninhydrin. Flash column chromatography was carried out using silica gel (ICN SiliTech 32-63D). Mass spectrometric analysis was performed on an Applied Biosystems 200 QTRAP mass spectrometer using electrospray ionization. HPLC analysis and purification were performed on a Varian Prostar Instrument equipped with a photo-diode-array detector. A reverse-phase Microsorb-MV 300 C18 column (250×4.6 mm dimension) was used for analytical HPLC. NMR spectra were recorded on a Bruker AVANCE 400 MHz instrument.

### Synthesis of Alkyl Azide Probes

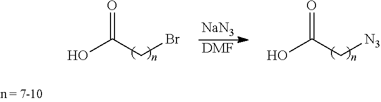

To a solution of the corresponding bromoalkanoic acid (˜1 g, 5 mmol) in 10 mL N,N-dimethylformamide (DMF) was added sodium azide (˜0.5 g, 7.5 mmol). The mixture was allowed to stir at room temperature overnight. The progress of the reaction was monitored by thin layer chromatography (1:2 hexanes:ethyl acetate) followed by bromocresol green stain. Upon completion, DMF was removed under reduced pressure. The resulting residue was re-dissolved in 15 mL of 1 M HCl and extracted with ethyl acetate (3×15 mL). The organic layer was dried over magnesium sulfate, then filtered. After removal of ethyl acetate in vacuo, the crude product was purified by silica gel chromatography (solvent gradient 0-15% ethyl acetate in hexanes) to afford the corresponding azidoalkanoic acid as clear or pale yellow oil. Yields ranged from 50-70%.

Characterization of n=7 azide (8-azidooctanoic acid). 1H NMR (CDCl3): 11.87 (s, 1H) 3.20 (t, 2H, J=6.9), 2.28 (t, 2H, J=7.5), 1.56 (m, 5H), 1.33 (m, 5H). ESI-MS calculated for [M−H]−: 184.11; observed 183.66.

Characterization of n=8 azide (9-azidononanoic acid). 1H NMR (CDCl3) 3.22 (t, 2H, J=6.9), 2.30, (t, 2H, J=7.5), 1.60 (m, 5H), 1.29 (m, 7H). ESI-MS calculated for [M−H]−: 198.12; observed 198.65.

Characterization of n=9 azide (10-azidodecanoic acid). 1H NMR (CDCl3): 3.23 (t, 2H, J=6.9), 2.28 (t, 2H, J=7.5), 1.53 (m, 5H), 1.31 (m, 9H). ESI-MS calculated for [M−H]−: 212.14; observed 212.28.

Characterization of n=10 azide (11-azidoundecanoic acid) 1H NMR (CDCl3) 3.27 (t, 2H, J=7.1), 2.39, (t, 2H, J=7.5), 1.65 (m, 5H), 1.20 (m, 11H). ESI-MS calculated for [M−H]−: 226.16; observed 226.12.

### Synthesis of ADIBO- and DIBO-Fluorophore Conjugates

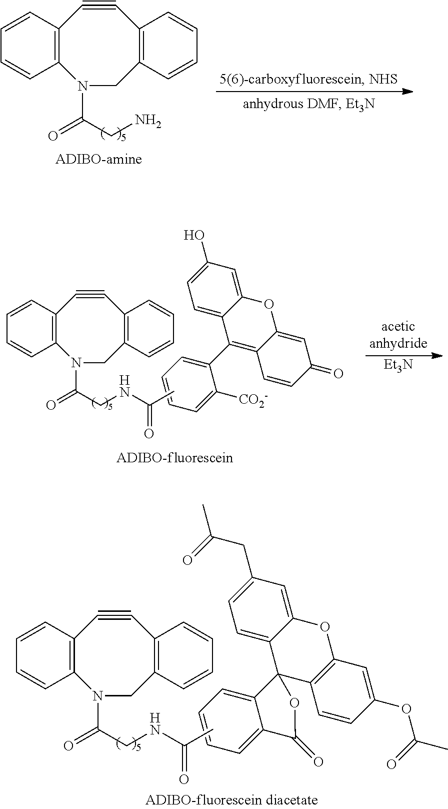

### ADIBO-Fluorescein Diacetate

The synthesis of aza-dibenzocyclooctyne-amine (ADIBO-amine) has been previously described.1 To a solution of ADIBO-amine (3 mg, 9 μmol) in anhydrous DMF (500 μL) was added triethylamine (Et3N, 3.8 μL, 27 μmol) and 5,6-carboxyfluorescein succinimidyl ester (NHS) (9.9 μmol, AnaSpec). The reaction was allowed to proceed for 10 hr at room temperature. Solvent was then removed under reduced pressure. The residue was subsequently dissolved in acetic anhydride (200 μL, 2.1 mmol) and allowed to stir for 30 min

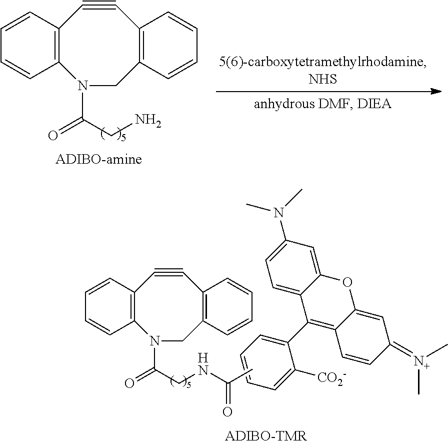

at room temperature. The color of the solution changed from bright yellow to colorless during the course of the reaction. Excess acetic anhydride was removed under reduced pressure. The resulting residue was purified by silica gel chromatography using 0-5% methanol in dichloromethane to afford ADIBO-fluorescein diacetate (Rf=0.5 in 10% methanol in dichloromethane). Purified product was analyzed on HPLC which showed single peak with absorbance at 210 nm. Estimated yield for two steps is ˜60%. ESI-MS for ADIBO-fluorescein diacetate: calculated for [M+H]+: 761.24; observed 760.74.

### ADIBO-TMR

To a solution of ADIBO-amine (70 mg, 0.46 mmol) in anhydrous DMF (2 mL) was added N,N-diisopropylethylamine (DIEA, 0.12 mL, 0.69 mmol) and 5,6-carboxytetramethylrhodamine (TMR) succinimidyl ester (NHS) (100 mg, 0.23 mmol, Sigma-Aldrich). The reaction was allowed to proceed for 12 hr at room temperature. Solvent was then removed under reduced pressure. Conjugate was purified by silica gel chromatography using 0-2% methanol in chloroform to provide dark red crystalline. The purity of the product was checked by HPLC. ESI-MS for ADIBO-TMR: calculated [M+H]+: 688.27; observed 688.8.

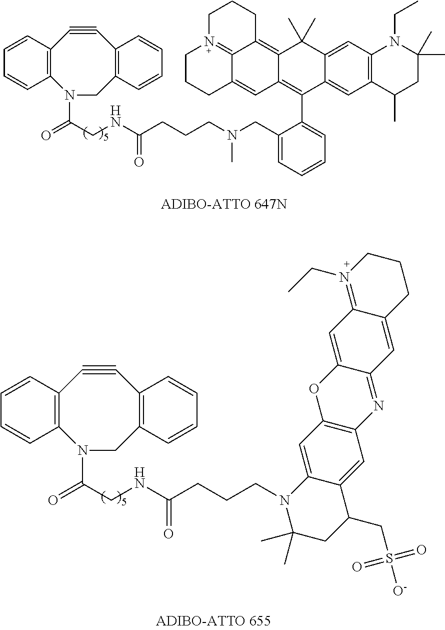

### ADIBO-ATTO 647N, ADIBO-ATTO 655

ADIBO conjugates to ATTO 647N and ATTO 655 were synthesized in a similar manner from ADIBO-amine1. ATTO 647 NHS ester (Sigma-Aldrich) and ATTO 655 NHS ester (Sigma Aldrich) were used. Conjugates were purified by silica gel chromatography using 0-2% methanol in dichloromethane. ESI-MS for ADIBO-ATTO 647N: calculated [M+H]+: 946.56; observed 946.29. ESI-MS for ADIBO-ATTO 655: calculated [M+H]+: 827.34; observed 827.51.

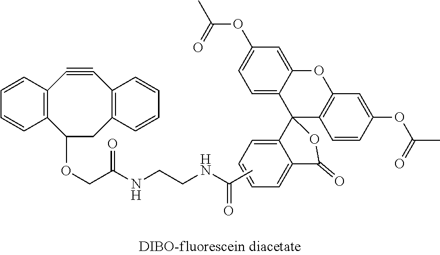

### DIBO-Fluorescein Diacetate

DIBO-fluorescein diacetate was synthesized in an analogous manner to ADIBO-fluorescein diacetate, from commercial DIBO-amine (Invitrogen) and fluorescein NHS ester (AnaSpec). The conjugate was purified by silica gel chromatography using 0-5% methanol in dichloromethane. ESI-MS for DIBO-fluorescein diacetate: calculated [M+H]+: 763.22; observed 763.86.

### DIBO-Oregon Green Diacetate DIBO-Oregon

Green 488 diacetate was a gift from Kyle Gee (Life Technologies).

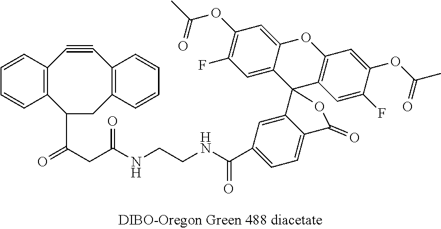 [SMILES: CC(=O)Oc1cc2c(cc1F)C1(OC(=O)c3ccc(C(=O)NCCNC(=O)CC(=O)C4Cc5ccccc5C#Cc5ccccc54)cc31)c1cc(F)c(OC(C)=O)cc1O2]

### Synthesis of MOFO-, DIMAC-, and DIFO-Fluorophore Conjugates

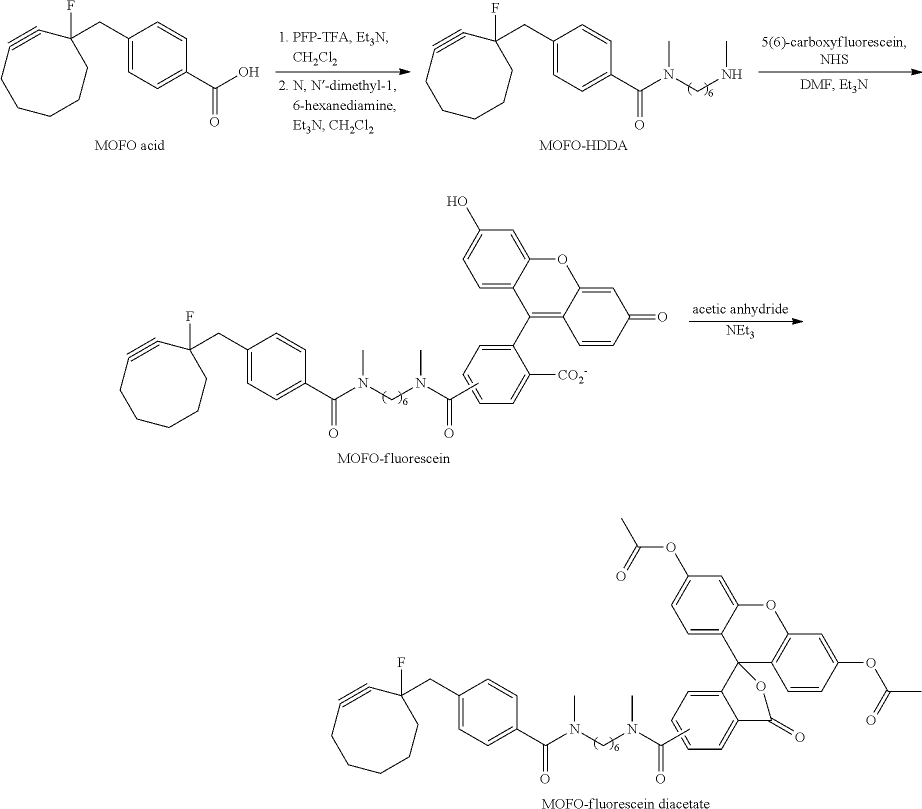

### MOFO-Fluorescein Diacetate

To a solution of MOFO cyclooctyne acid² (5 mg, 19 μmol) in 500 μL, anhydrous dichloromethane was added pentafluorophenyl trifluoroacetate (PFP-TFA, 9.8 μL, 57 μmol) and Et3N (8 μL, 57 μmol). The reaction was allowed to proceed for 2 hr at room temperature. N,N′-dimethyl-1,6-hexanediamine (HDDA, 114 μmol) was then added to the reaction mixture, which was allowed to stir for 5 hr at room temperature. Solvent was removed under reduced pressure. The reaction mixture was purified by silica gel chromatography (10-15% methanol in dichloromethane) to afford MOFO-N,N′-dimethyl-1,6-hexanediamine (MOFO-HDDA). MOFO-HDDA was dissolved in anhydrous DMF (300 μL), and 5(6)-carboxyfluorescein, succinimidyl ester (9.8 mg, 20.9 μmol) and Et3N (8 μL, 57 μmol) were added to the mixture, which was allowed to stir for 10 hr at room temperature. Solvent was removed under reduced pressure. The residue was dissolved in a small amount of acetic anhydride (<200 μL) and allowed to stir for 30 min at room temperature. After removal of acetic anhydride under reduced pressure, the reaction mixture was purified by silica gel chromatography (solvent gradient 0-5% methanol in dichloromethane) to afford MOFO-fluorescein diacetate (Rf=0.4 in 10% methanol in dichloromethane). Estimated overall yield for four steps, 30-40%. ESI-MS for MOFO-fluorescein diacetate: calculated [M+H]+: 829.34; observed 829.44.

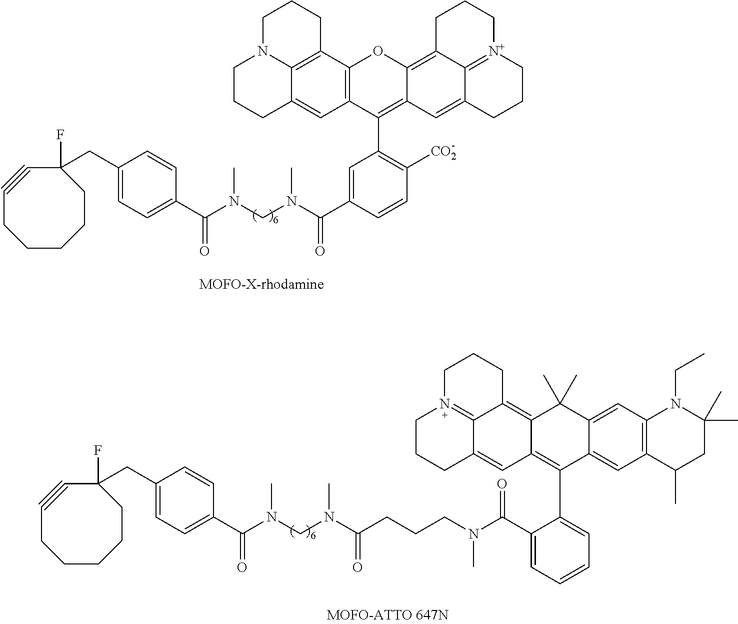

### MOFO-X-Rhodamine

MOFO-ATTO 647N MOFO-HDDA was synthesized as described above, then conjugated to 5(6)-X-rhodamine NHS ester (Anaspec, 5(6)-ROX, SE) or ATTO 647N NHS ester (Sigma-Aldrich). Conjugates were purified by silica gel chromatography using 0-5% methanol in dichloromethane for MOFO-X-rhodamine and 0-2% methanol in dichloromethane for MOFO-ATTO 647N. ESI-MS for MOFO-X-rhodamine: calculated [M+H]+: 903.49; observed 903.72. ESI-MS for MOFO-ATTO 647N: calculated [M+H]+: 1014.66; observed 1014.42.

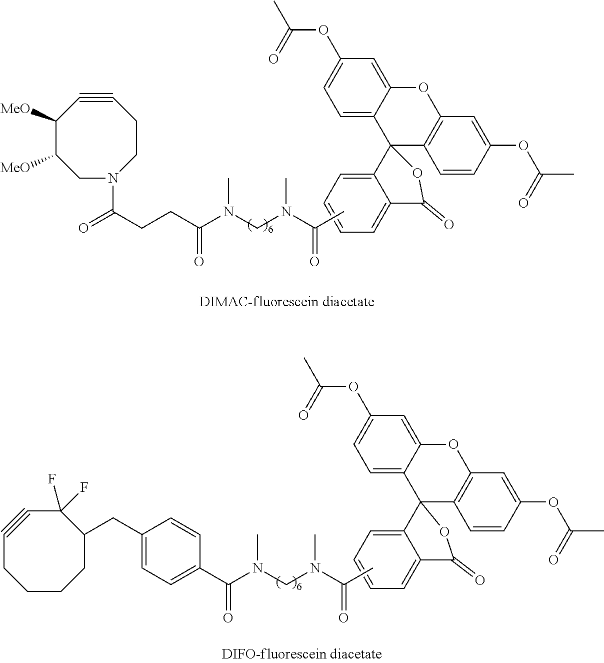

### DIMAC-Fluorescein Diacetate

DIFO-fluorescein diacetate Fluorescein diacetate conjugates to DIMAC3 and DIFO4 were synthesized in a similar manner from their respective acids. Conjugates were purified by silica gel chromatography using 0-5% methanol in dichloromethane. ESI-MS for DIMAC-fluorescein diacetate: calculated [M−H]−: 752.33; observed 752.40. ESI-MS for DIFO-fluorescein diacetate: calculated [M+H]+: 847.33; observed 847.26.

### Plasmids

For bacterial expression of LplA, His6-LplA in pYFJ16. Gautier et al., Chemistry & Biology 15:128-136 (2008). For mammalian expression of LplA, we used His6-FLAG-LplA in pcDNA3. Gautier et al., 2008. For mammalian expression of LAP fusion proteins, we used LAP-β-actin and LAP-MAP2 in Clontech vector, LAP-LDL receptor in pcDNA4, and LAP-neurexin-1β in pNICE. LAP-BFP expression constructs (LAP-BFP, LAP-BFP-NLS, LAP-BFP-CAAX, and LAP-BFP-NES) in pcDNA3 and LAP-mCherry in pcDNA3 were generated from corresponding pcDNA3-LAP-YFP plasmids by replacing YFP with BFP or mCherry, using the BamHI and EcoRI restriction sites. All LplA and LAP point mutants were prepared via QuikChange site-directed mutagenesis. Complete sequences of plasmids used in this study are available at stellar.mit.edu/S/project/tinglabreagents/r02/materials.html.

### Immunofluorescence Staining of LplA

After live cell imaging, cells were fixed with 3.7% formaldehyde in Dulbecco's Phosphate Buffered Saline (DPBS) pH 7.4 for 10 min at room temperature followed by cold precipitation with methanol for 5 min at −20° C., then blocked with 3% BSA in DPBS for 1 hr at room temperature. To visualize FLAG-tagged LplA, cells were incubated with 4 μg/mL mouse monoclonal anti-FLAG antibody (Sigma-Aldrich) in 1% BSA in DPBS for 1 hr at room temperature. Cells were further washed and incubated with 4 μg/mL goat anti-mouse IgG antibody conjugated to Alexa Fluor 568 (Life Technologies) in 1% BSA in DPBS for 1 hr at room temperature, then washed and imaged.

### Kinetic Analysis of Azide 9 Ligation

Reactions were set up as described in the main text. Aliquots were taken and quenched before product conversion exceeded 5%. To calculate initial rates, we determined the amount of product at each time point by generating a calibration curve using purified LAP and LAP-azide 9 mixed at different ratios. This curve correlated the measured ratio of integrated HPLC peak areas to the actual ratio, i.e. adjusted for any differences in extinction coefficient of LAP vs. LAP-azide 9. Initial rates (Vo) were determined at each azide 9 concentration, by plotting the amount of LAP-azide 9 product against time. The slope of the line gives Vo. Vo values were then plotted against azide 9 concentration in FIG. S3C, and Origin 8.5.1 was used to fit the curve to the Michaelis-Menten equation Vo=Vmax[azide 9]/(Km+[azide 9]). From the Vmax, kcat was calculated using Vmax=kcat[E]total. Measurements of Vo values at each azide 9 concentration were performed in triplicate.

### Analysis of Azide 7 and Azide 9 Ligation Yields in Cells

HEK cells were plated into wells of a 12-well culture plate (4 cm² per well) 18 hr prior to transfection and grown to 60% confluency. For azide 7 ligation, cells were transfected with 50 ng WTLplA and 1000 ng pcDNA3-LAP-YFP. For azide 9 ligation, cells were transfected with 500 ng W37ILplA and either 1000 ng pcDNA3-LAP-YFP or pcDNA3-LAP(K⋄A)-YFP using Lipofectamine 2000 (Life Technologies). The LplA:LAP plasmid ratios are identical to the conditions used for imaging. 18 hr after transfection, cells were incubated in growth media (MEM supplemented with 10% FBS) containing 200 μM azide 7 or azide 9 for 30 min or 1 hr at 37° C. Excess azide probe was washed out over 1 hr. Cells were then harvested and lysed in 500 μL hypotonic lysis buffer (1 mM HEPES pH 7.5, 5 mM MgCl₂, 1 mM PMSF (Thermal Scientific, phenylmethanesulfonyl fluoride), 1 mM protease inhibitor cocktail (Sigma-Aldrich)), frozen at −20° C., thawed at room temperature, then mixed by vortexing for 2 min. This freeze-thaw-vortex cycle was repeated three times. Cells were then centrifuged at 13,000 rpm for 2 min, and the supernatant was analyzed on a 12% polyacrylamide native gel without SDS (5 μL lysate per lane) at constant 200 V. Prior to Coomassie staining, in-gel fluorescence of YFP was visualized on a FUJIFILM FLA-9000 instrument using LD473 laser and Long Pass Blue (LPB) filter. A repeat of the experiment in FIG. 2C gave ligation yields of 67% for WTLplA (50 ng plasmid)+azide 7, and 89% for W37ILplA (500 ng plasmid)+azide 9.

Analysis of Two-Step Ligation Yield after Strain Promoted Cycloaddition in Cells

HEK cells plated into wells of a 12-well culture plate (4 cm² per well) were transfected with 500 ng pcDNA3-W37ILplA and 1000 ng pcDNA3-LAP-mCherry using Lipofectamine 2000 (Life Technologies). Azide 9 labeling and washout were performed in the same manner as in FIG. S4A. After excess azide 9 washout, cells were incubated in MEM containing 10 μM DIBO-biotin (Life Technologies) for 10 min at 37° C. Thereafter, cells were further washed for 2.5 hr to remove excess DIBO-biotin. Cells were then harvested and lysed in the same manner as described above. The cell lysate was incubated with 5 μM of streptavidin for 1 hr at 4° C., then analyzed on a 12% SDS-polyacrylamide gel at constant 200 V, under conditions known to preserve biotin-streptavidin binding as well as streptavidin's subunit association.⁶ Prior to Coomassie staining, in-gel fluorescence of mCherry was visualized on FUJIFILM FLA-9000 instrument using SHG532 laser and Long Pass Green (LPG) filter.

Cell Fixation after Live Cell DIMAC-Fluorescein and DIFO-Fluorescein Labeling

After live cell imaging, cells were fixed with 3.7% formaldehyde in DPBS pH 7.4 for 10 min at room temperature followed by cold precipitation with methanol for 5 min at −20° C. Cells were then washed with DPBS several times over 10 min, before imaging.

Cell Surface and Intracellular Labeling with Commercial DIBO Conjugates

### DIBO-Alexa Fluor 647 Cell Surface Labeling

HEK cells plated on glass coverslips in wells of a 48-well cell culture plate (0.95 cm² per well) were transfected with 100 ng pcDNA4-LAP-LDL receptor or 400 ng pNICE-LAP-neurexin-1β using Lipofectamine 2000. At 18 hr after transfection, cells were washed three times with MEM. Enzymatic ligation of azide 9 on the cell surface was performed in MEM with 5 μM W37ILplA, 500 μM azide 9, 2 mM ATP and 2 mM magnesium acetate for 20 min at room temperature (to minimize internalization of cell-surface proteins). After washing three times with MEM, cells were incubated with 10 μM DIBO-Alexa Fluor 647 in MEM for 10 min at room temperature. Cells were then washed three times with MEM and imaged live.

### DIBO-Biotin Cell Surface and Intracellular Labeling

DIBO-biotin cell surface labeling was performed in the same manner as DIBO-Alexa Fluor 647 cell surface labeling, described above. After DIBO-biotin incubation, cells were washed three times with DPBS and fixed with 3.7% formaldehyde in DPBS pH 7.4 for 10 min at room temperature, followed by cold precipitation with methanol for 5 min at −20° C. Fixed cells were then blocked with 1% casein in DPBS for 1 hr at room temperature. To visualize specific labeling, cells were stained with streptavidin conjugated to Alexa Fluor 568 or Alexa Fluor 647 in 0.5% casein in DPBS for 5 min at room temperature, followed by washing three times with DPBS and imaging.

For DIBO-biotin intracellular labeling, HEK cells plated on glass coverslips in wells of a 48-well cell culture plate (0.95 cm² per well) were transfected with 400 ng pcDNA3-LAP-BFP-NLS and 200 ng pcDNA3-W37ILplA. Azide 9 labeling/washout and DIBO-biotin labeling/washout were performed in the same manner as in FIG. S4B. After DIBO-biotin washout, cells were fixed and stained with streptavidin-Alexa Fluor 568, as described above.

### Quantitative Analysis of Fluorophore-Cyclooctyne Labeling Specificity

Cells with signal at least 3-fold greater than autofluorescence from untransfected cells in the cyclooctyne channel were selected by hand for analysis. For each of these cells, one region in the cytosol (representing background) and one region in the nucleus (representing specific signal) were manually circled. The background-corrected mean fluorescence intensity was determined for both regions using SlideBook. Excel was used to plot the nuclear versus cytosolic fluorescence intensity for each cell. Since ATTO 647N labeling signal was low, we selected for analysis cells with signal at least 2-fold greater than autofluoresence from untransfected cells in the ATTO 647N channel.

### Quantitative Analysis of MOFO-Fluorescein Labeling of LAP-BFP Using Four LplA Mutant/Azide Substrate Pairs

Cells with fluorescein signal at least 2-fold greater than autofluorescence from untransfected cells, and BFP signal at least 5-fold greater than autofluorescence were selected by hand for analysis. For each of these cells, the entire area of the cell representing signal was circled. SlideBook was used to calculate the mean intensities in both channels. The background-corrected mean fluorescein intensity was plotted against the background-corrected mean BFP intensity using Excel.

### Quantitative Analysis of LplA Mutant Expression Levels in Cells

Cells with Alexa Fluor 568 signal at least 1.5-fold great than background (area without any cell) were selected by hand for analysis. For each of these cells, the entire area of the cell representing signal was circled. SlideBook was used to calculate the mean intensities in the channel. The background-corrected mean Alexa Fluor 568 intensity was plotted using Excel.

### Other Protocols

LplA and mutants were expressed and purified as previously described. Uttamapinant, et al., Proc. Natl. Acad. Sci. U.S.A., 107:10914-10919 (2010). The 13-amino acid LAP peptide (H2N-GFEIDKVWYDLDA-CO2H)₇ was synthesized by the Tufts University Peptide Synthesis Core Facility and purified to >96% homogeneity.

### Results

### Screening for the Best Alkyl Azide Ligase

To generalize PRIME for targeting of diverse fluorophore structures, our first challenge was to develop a method to efficiently and specifically ligate a functional group handle to LAP fusion proteins inside living cells. Previously we reported that wild-type LplA can catalyze the conjugation of 8-azidooctanoic acid (“azide 7”) to LAP with a k_{cat} of 6.66 min⁻¹ and Kₘ of 127 μM (Fernandez-Suarez, et al., Nature Biotechnology, 25:1483-1487 (2007)). This works well for cell surface labeling, where the azide probe can be added at high concentrations and then excess unligated probe can be easily washed away. For intracellular labeling, however, it is more difficult to thoroughly wash away excess unused probe. It is therefore preferable to deliver the azide probe at lower concentrations so that less residual azide remains after the ligation reaction, to minimize interference with the subsequent [3+2] cycloaddition. To use lower azide concentrations without sacrificing azide ligation yield, we needed to engineer the LplA-catalyzed azide ligation reaction to improve its kinetic properties.

Previous work has shown that Trp37 in the lipoic acid binding pocket serves as a “gatekeeper” residue, and its mutation to smaller side-chains allows LplA to recognize a variety of unnatural substrates. Uttamapinant, et al., Proc. Natl. Acad. Sci. U.S.A., 107:10914-10919 (2010), Puthenveetil, et al., J. Am. Chem. Soc., 131:16430-16438 (2009); Jin, et al., Chembiochem, 12:65-70 (2011); Cohen, et al., Biochemistry, 50:8221-8225 (2011); and Baruah, et al., Angew. Chem., Ind. Ed., 47:7018-7021 (2008), all of which are incorporated by reference herein. To identify an improved LplA/azide pair, we prepared a panel of LplA Trp37 mutants—W37G, A, V, I, L, and S—and screened them against a panel of alkyl azide substrates of various lengths (FIG. 20B). An HPLC assay was used to determine the percent conversion of LAP into LAP-azide conjugate, using 20 μM probe for 20 minutes (FIG. 20B). We found that wild-type LplA and ^{W37V}LplA were the best ligases for the shortest azide 7 probe. For the longer probes, wild-type LplA was no longer effective, and ^{W37V}LplA and ^{W37I}LplA mutants were best. The four best ligase/probe pairs are starred in FIG. 20B.

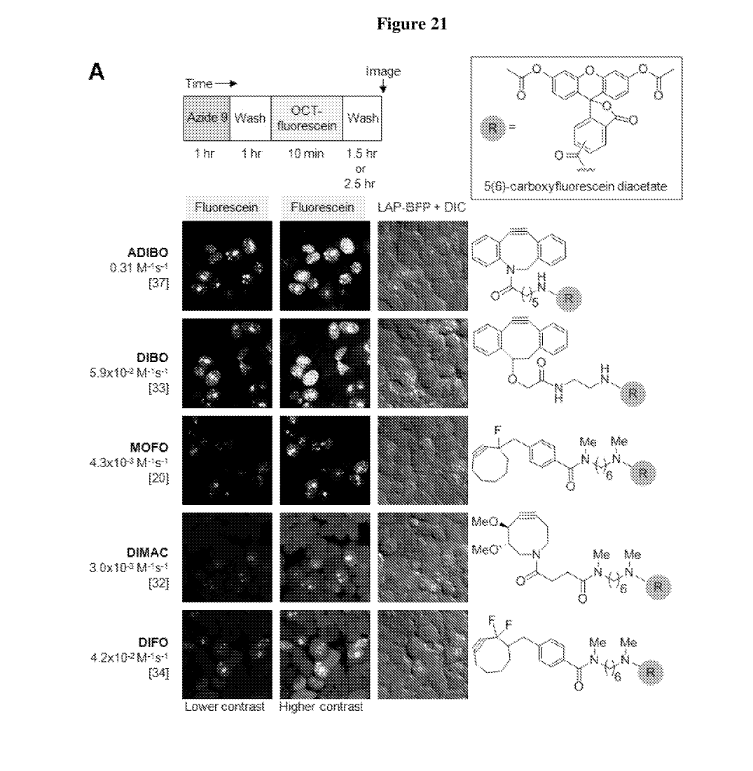

To differentiate between these top four ligase/azide pairs, we tested their performance in living cells. Human Embryonic Kidney (HEK) cells were transfected with plasmids for each LplA mutant and LAP-BFP (Blue Fluorescent Protein). Azide 9 was added to the cells for 1 hour. We empirically optimized the washout time required to fully remove excess azide, using cyclooctyne-fluorescein retention as a readout, and found that 1 hour was adequate. Therefore excess azide 7 and azide 9 were each washed from cells for 1 hour, before addition of the monofluorinated cyclooctyne-fluorophore conjugate, MOFO-fluorescein diacetate (structure in FIG. 21) to derivatize the azide-LAPs. The labeling protocol was as follows: incubation with Azide 7 or Azide 9 for 1 hr, wash for 1 hr, incubation with MOFO-fluorescein for 10 minutes, wash again for 2 hr, and then imaging. After 10 minutes incubation and 2 hours of washing to remove excess fluorophore, cells were imaged.

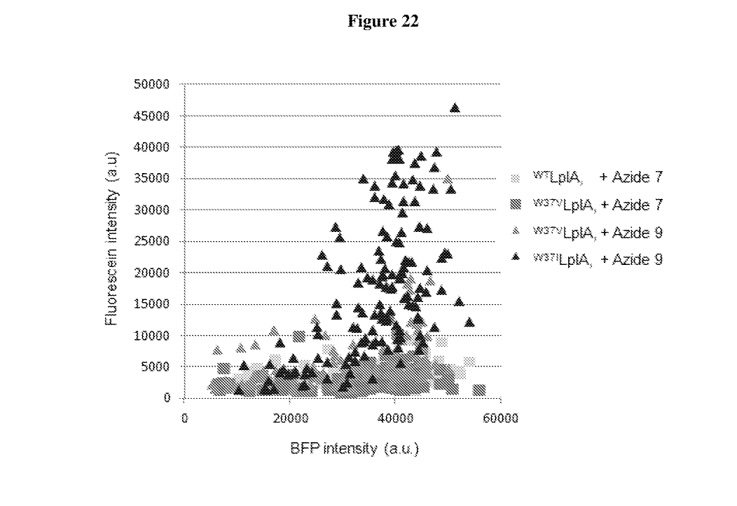

Specific labeling of LAP-BFP was observed in all four combinations, but the highest signal-to-background ratio was obtained for the ^{W37I}LplA/azide 9 pair. Note the substantial improvement in signal intensity (−4-fold greater on average) compared to the wild-type LplA/azide 7 pair previously used for cell surface protein labeling. Fernandez-Suarez, et al., Nature Biotechnology, 25:1483-1487 (2007). These differences are quantified in FIG. 22, in which fluorescein intensity is plotted against LAP-BFP expression level for >100 cells for each condition. Anti-FLAG immunofluorescence staining to detect FLAG-tagged LplA in cells showed that ligase mutant expression levels are all comparable under our experimental conditions.

We also used a gel shift assay as a separate readout of azide ligation yield inside cells. HEK cells were prepared expressing LAP-YFP (Yellow Fluorescent Protein) and either wild-type LplA or ^{W37I}LplA. Azide 7 or azide 9 was added for 30 minutes or 1 hour, before washing and cell lysis. The yield of azide ligation to LAP-YFP was determined by shift on a native polyacrylamide gel. The unmodified fusion protein, visualized by YFP fluorescence, runs at an apparent molecular weight of ˜42 kD. Upon modification, the positively charged lysine of LAP converts into a neutral amide, and the apparent molecular weight of the fusion protein shifts down to ˜40 kD. Based on densitometry, we found that the ^{WT}LplA/azide 7 pair gave 73% ligation yield after 1 hour labeling in cells, whereas the ^{W37I}LplA/azide 9 pair gave nearly quantitative ligation after only 30 minutes of azide 9 incubation. Based on these data, and the cell imaging results, we selected ^{W37I}LplA/azide 9 as our best ligase/azide pair.

### Characterization of Our Azide 9 Ligase, ^{W37I}LplA

We proceeded to fully characterize our best azide ligation reaction. ^{W37I}LplA-catalyzed ligation of azide 9 onto purified LAP peptide was observed in an HPLC analysis. The identity of the LAP-azide 9 product peak was confirmed by mass spectrometry. Negative control reactions with ATP omitted or wild-type LplA in place of ^{W37I}LplA were also analyzed and showed no product formation. We also used HPLC to quantify product amounts in order to measure k_{cat} and Kₘ values. The Michaelis-Menten plot obtained from the results showed a k_{cat} of 3.62 min⁻¹ and a Kₘ of 35 μM for azide 9 ligation catalyzed by ^{W37I}LplA. Compared to our previously reported azide 7 ligation catalyzed by wild-type LplA. (Fernandez-Suarez, et al., 2007), this Kₘ is 4-fold lower. The k_{cat} is 1.8-fold reduced, giving an overall 2-fold improvement in k_{cat}/Kₘ.

### Comparison of Cyclooctyne Structures

Next, we focused on the optimization of the azide derivatization chemistry in cells. Numerous bioorthogonal ligation reactions have been reported to derivatize alkyl azides, including the Staudinger ligation (Schilling, et al, Chem. Soc. Rev., 40:4840-4871 (2011), and copper-catalyzed (del Amo, et al., J. Am. Chem. Soc., 132:16893-16899 (2010) as well as strain-promoted (Sletten, et al., Accounts of Chemical Research null (2011) [3+2] azide-alkyne cycloadditions. Of these, copper-catalyzed [3+2] cycloaddition is the fastest, but copper(I) is toxic to cells (Sletten, et al., 2011) and not easily delivered into the cytosol, where it also could become sequestered by endogenous thiols. On the other hand, copper-free, strain-promoted cycloaddition has been successfully demonstrated inside living cells (Beatty, et al., Chembiochem, 11:2092-2095 (2010); Beatty, et al., Chembiochem n/a (2011); Plass, et al., Angew. Chem., Int. Ed., 50:3878-3881 (2011)), and on the surface of cells within living animals (Baskin, et al., Proc. Natl. Acad. Sci. U.S.A., 104:16793-16797 (2007); Laughlin, et al., Science 320:664-667 (2008); Chang, et al., Proc. Natl. Acad. Sci. U.S.A., 107:1821-1826 (2010)). For this reason, we selected cyclooctyne-fluorophore conjugates to derivatize LAP-azide.

Numerous cyclooctyne structures have been developed by our labs (Agard, et al., Acs Chem. Biology, 1:644-648 (2006); Kuzmin, et al., Bioconjugate Chemistry, 21:2076-2085 (2010); Sletten, et al., Org. Lett., 10:3097-3099 (2008); Ning, et al., Angew. Chem., Int. Ed., 47:2253-2255 (2008); Codelli, et al., J. Am. Chem. Soc., 130:11486-11493 (2008); Jewett, et al., J. Am. Chem. Soc., 132:3688-+(2010); Sanders, et al., J. Am. Chem. Soc., 133:949-957 (2011)) and other labs (Debets, et al., Chem. Commun., 46:97-99 (2010); Stockmann, et al., Chem. Sci., 2:932-936 (2011). These structures vary in terms of ring strain and electron deficiency, which in turn influence reactivity toward azides and endogenous cellular molecules, such as thiols (Beatty, et al., Chembiochem, 11:2092-2095 (2010)). In addition, more hydrophilic cyclooctyne structures have been developed (Sletten, et al., Org. Lett., 10:3097-3099 (2008)) to reduce the extent of nonspecific hydrophobic binding to cells. Because it was not clear which cyclooctyne structure(s) would be the best for our purpose, we selected a panel of five structures, derivatized each with 5(6)-carboxyfluorescein diacetate (FIG. 21A), and compared the performance of these conjugates for LAP-azide labeling inside living cells.

FIG. 21A shows that, for labeling of LAP-BFP-NLS (NLS is a nuclear localization signal) in HEK cells, ADIBO- and DIBO-fluorescein diacetate conjugates give the highest signal, consistent with their superior second-order rate constants (0.31 M⁻¹ s⁻¹ and 5.9×10⁻² M⁻¹ s⁻¹, respectively. Sanders, et al., J. Am. Chem. Soc., 133:949-957 (2011); and Debets, et al., Chem. Commun., 46:97-99 (2010)). Surprisingly, significant nonspecific labeling was seen with DIMAC, even in untransfected cells, despite its more hydrophilic structure (Sletten, et al., Org. Lett., 10:3097-3099 (2008)). Most of this nonspecific signal can be washed away after cells are fixed, suggesting that it arises from non-specific binding to cellular structures. DIFO also gave background, which unlike DIMAC, persisted to some extent after cell fixation; this may reflect covalent addition of endogenous cellular nucleophiles such as glutathione, which has previously been observed (Beatty, et al., Chembiochem, 11:2092-2095 (2010); Chang, et al., Proc. Natl. Acad. Sci. U.S.A., 107:1821-1826 (2010)). Lowering the DIFO-fluorescein diacetate concentration by 10-fold to 1 μM, and shortening the labeling time to 40 seconds reduced the background somewhat, but it was still higher than the background seen with ADIBO and DIBO. Previous studies have shown that DIFO and DIMAC in live mice (Chang, et al., Proc. Natl. Acad. Sci. U.S.A., 107:1821-1826 (2010)) both bind to mouse liver serum albumin, likely via hydrophobic as well as covalent interactions.

Labeling with MOFO-fluorescein diacetate was specific, like with ADIBO and DIBO, although the signal was lower, presumably because of lower azide reactivity (k=4.3×10⁻³ M⁻¹ s⁻¹) (Agard, et al., Acs Chem. Biology, 1:644-648 (2006)). We quantitatively analyzed the signal-to-background ratios resulting from cellular labeling with ADIBO, DIBO, and MOFO, by calculating the cytosolic to nuclear signal intensity ratios for >50 cells from each condition. Because the LAP fusion is nuclear-localized, a nuclear fluorescein signal represents specific labeling, whereas cytosolic fluorescein signal represents nonspecific labeling. FIG. 21B shows that while absolute signals are ˜4-fold higher with ADIBO and DIBO compared to MOFO, the signal-to-background ratios are comparable for all three cyclooctynes. We hypothesize that MOFO gives lower background because it is not as hydrophobic as ADIBO and DIBO. This is supported by the fact that shorter dye washout time is required for MOFO (1.5 hours) compared to ADIBO and DIBO (2.5 hours).

On the basis of these results, we selected ADIBO and DIBO for most of our cellular protein labeling experiments. However, as shown later, due to ADIBO's hydrophobicity, we find that MOFO is a better option when working with very hydrophobic fluorophores such as ATTO 647N.

Intracellular Protein Labeling with Azide 9 Ligase and ADIBO Fluorescein

Having optimized both the azide ligase and the cyclooctyne, we proceeded to characterize two-step labeling inside cells, and explore its generality. HEK cells expressing ^{W37I}LplA and LAP-BFP were labeled with azide 9 for 1 hour followed by ADIBO-fluorescein diacetate. We empirically optimized the ADIBO-fluorophore loading concentration and washout time. More specifically, various amounts of ADIBO-fluorescein (2.5 μM, 5 μM, 10 μM, 20 μM, and 40 μM) were loaded into untransfected COS-7 cells for 10 min at 37° C. and various washout times were tested, ranging from 0 to 5 hr. Fluorescein images were shown with DIC overlay. Since cycloaddition yield in cells increases with cyclooctyne concentration, we determined the highest concentration that we could load, and yet cleanly washout in a reasonable period of time. We found that 10 μM of loaded ADIBO-fluorescein diacetate, followed by 2.5 hours of washout, was optimal.

It was found that HEK cells expressing LAP-BFP were labeled with fluorescein, whereas neighboring untransfected cells were not labeled. Negative controls with azide 9 omitted, LAP mutated, or a catalytically inactive LplA mutant, ^{w37I/K133R}LplA (Fujiwara, et al., J. Bio. Chem., 285:9971-9980 (2010)), did not show fluorescein labeling.

We also tested labeling of different LAP fusion proteins, including LAP-BFP fusions with nuclear export sequence of prenylation tag, or nuclear localization signal, LAP-β-actin, and LAP-MAP2 (microtubule-associated protein 2). Using the two-step protocol shown in FIG. 20A, we successfully labeled LAP in the nucleus, cytosol, and plasma membrane, as well as LAP fusions to actin and MAP2. These experiments were performed in multiple mammalian cell lines—HEK, HeLa, and COS-7-demonstrating the versatility of the method.

### Extension to Diverse Fluorophore Structures

To test our method with other fluorophores, we prepared ADIBO conjugates to tetramethylrhodamine (TMR), ATTO 647N, and ATTO 655. ADIBO-TMR and ADIBO-ATTO 655 both gave specific labeling, but ADIBO-ATTO 647N produced a high level of nonspecific binding. This may be due to the more hydrophobic structure of ATTO 647N (structure shown above). Even by itself, without any cyclooctyne conjugate, we have found that ATTO 647N gives a high level of nonspecific cell staining, primarily in the mitochondria, which is known to concentrate positively-charged hydrophobic dyes. A comparison of LAP-BFP-NLS labeling with ADIBO- and MOFO-conjugates to ATTO 647N showed that MOFO-ATTO 647N gives much more specific labeling than ADIBO-ATTO 647N, likely because the total hydrophobicity of the conjugate is reduced. This ultimately permitted us to perform MOFO-ATTO 647N labeling of LAP-β-actin in live COS-7 cells.

We also tested the effect of varying the linker structure between MOFO and ATTO 647N in an attempt to further reduce the labeling background. The N,N′-dimethyl-1,6-hexanediamine (HDDA) linker that we used for most fluorophore conjugates in this work was replaced by a more hydrophilic polyethylene glycol (PEG) linker. For labeling of LAP-BFP-NLS, no significant reduction in staining background was observed with MOFO-PEG-ATTO 647N, suggesting that the cyclooctyne and fluorophore moieties dominate the hydrophobic properties of the probe.

Results obtained from this study showed live cell labeling of multiple LAP fusion proteins with a diverse palette of fluorophores spanning from fluorescein to ATTO 647N. ADIBO was used for the more hydrophilic dyes such as fluorescein, TMR and ATTO 655. DIBO, which is structurally similar to ADIBO, is used for Oregon Green 488. MOFO is used for the more hydrophobic dyes, X-rhodamine and ATTO 647N.

### Cell Surface Labeling and Measurement of Two-Step Ligation Yield in Cells

In addition to intracellular labeling, we performed cell surface labeling using commercially available cyclooctyne-probe conjugates DIBO-Alexa Fluor 647 or DIBO-biotin. LAP-tagged LDL receptor and neurexin-1β were labeled on the surface of HEK cells, by adding purified ^{W37I}LplA, azide 9, and ATP to the cell medium for 20 minutes. Thereafter, LAP-azide was derivatized using either membrane-impermeant DIBO-Alexa Fluor 647, or DIBO-biotin. The DIBO-biotin was visualized by staining with streptavidin-Alexa Fluor conjugates. Specific, azide-dependent cell surface labeling was seen in all cases.

Because DIBO-biotin is membrane-permeant, it is also possible to perform this labeling inside cells, although biotinylated LAP proteins can only be detected after membrane permeabilization and streptavidin staining. Intracellular labeling was observed in HEK cells co-expressing LAP-BFP-NLS and ^{W37I}LplA. After azide ligation, DIBO-biotin was added for 10 minutes, before washing, fixation, and detection with streptavidin-Alexa Fluor 568.

We used two-step intracellular azide 9/DIBO-biotin labeling to measure our overall LAP labeling yield. After performing labeling using the protocol in FIG. 20A, HEK cells were lysed, incubated with excess streptavidin protein to bind biotinylated LAP-mCherry fusion protein, and the lysate was analyzed by gel. In-gel mCherry fluorescence imaging shows that LAP-mCherry runs at the expected molecular weight (27 kD) in negative control samples in which azide 9 or streptavidin were omitted. However, 21% of LAP-mCherry was found to be shifted up to ˜80 kD, reflecting binding by streptavidin. We conclude that under the labeling conditions described above, the two-step labeling yield in cells is approximately 20%.

### Discussion

We have developed methodology for targeting of diverse fluorophore structures to recombinant cellular proteins modified by a 13-amino acid peptide tag (LAP2). The targeting is accomplished first by enzyme-mediated alkyl azide ligation, and then by strain-promoted cycloaddition with a fluorophore-conjugated cyclooctyne. To develop the method, we systematically optimized the azide ligation reaction through screening of lipoic acid ligase mutants and alkyl azide variants. We then evaluated five different cyclooctyne structures differing in reactivity, selectivity, and extent of non-specific binding to cells, using a live-cell fluorescein targeting assay. Our final, optimized two-step labeling scheme was used to target a diverse panel of fluorophores ranging from fluorescein to ATTO 647N, to a variety of LAP fusion proteins in multiple mammalian cell lines.

Our comparison of cyclooctynes in cells yielded observations that should prove useful even beyond the context of PRIME and enzyme-mediated targeting, due to the numerous and diverse applications to which cyclooctynes are being applied (Beatty, et al., 2010; Beatty, et al., 2011); Plass, et al., 2011; Baskin, et al., Proc. Natl. Acad. Sci. U.S.A., 104:16793-16797 (2007); Laughlin, et al., Science 320:664-667 (2008); Chang, et al., 2010; Jayaprakash, et al., Org. Lett,. 12:5410-5413 (2010); and Bostic, et al., Chem. Commun. (2012)). One of the earliest cyclooctynes, MOFO (monofluorinated) Agard, et al., Acs Chem. Biology, 1:644-648 (2006), performed well inside cells, giving signal to background ratios consistently >5:1 in the context of fluorescein targeting to nuclear LAP. This same cyclooctyne was used for cell surface LplA-mediated labeling in our previous study (Fernandez-Suarez, et al., 2007). In next-generation cyclooctynes, fusion to benzene rings increased ring strain and hence second-order rate constant. Not surprisingly, we found that these cyclooctynes, ADIBO and DIBO, gave ˜4-fold higher absolute signal in cells, compared to MOFO, probably due to increased yield of cycloaddition product. However, the increase in signal was accompanied by an increase in background, likely due to the greater hydrophobicity and hence non-specific binding of these dyes. Consequently, the signal-to-background ratios were comparable for ADIBO, DIBO, and MOFO-fluorescein conjugates. When we extended the cyclooctyne comparison to other fluorophores, we found that ADIBO and DIBO conjugates to well-behaved hydrophilic fluorophores such as fluorescein and Oregon Green gave satisfactory labeling, but when we tried to target very hydrophobic fluorophores such as ATTO 647N, the combined hydrophobicity of the dye and the cyclooctyne (ADIBO) precluded successful labeling, due to high non-specific binding. This was alleviated by using the less hydrophobic MOFO instead. Thus MOFO-ATTO 647N but not ADIBO-ATTO 647N was used to label and image actin in living COS-7 cells. Our study illustrates the need for new cyclooctyne probes that combine high reactivity (as displayed by ADIBO) with low hydrophobicity/non-specific binding (as displayed by MOFO). Alternatively, fluorogenic cyclooctynes (Jewett, et al., Org. Lett., 13:5937-5939 (2011)) would be extremely helpful, hiding non-specific binding, and producing fluorescence only upon specific reaction with azide-conjugated LAP.

Several of the fluorophores targeted using LplA and strain-promoted cycloaddition in this study have exemplary properties that make them attractive alternatives to fluorescent proteins. For instance, X-rhodamine is a bright and photostable fluorophore commonly used for speckle imaging of actin (Lim, et al., Experimental Cell Research, 316:2027-2041 (2010)). ATTO 647N is one of the best fluorophores of any kind for both STED (stimulated emission depletion) (Mueller, et al., Biophysical Journal, 101:1651-1660 (2011); Westphal, et al., Science, 320:246-249 (2008)) and STORM-type (Dempsey, et al., Nat. Meth. 8:1027-1036 (2011)) super-resolution microscopies, due to its intense brightness, photostability, and photoswitching properties. On the cell surface, we targeted Alexa Fluor 647, an excellent fluorophore that has been used for countless ensemble and single molecule imaging experiments (van de Linde, et al., J. Structural Bio., 164:250-254 (2008); Heilemann, et al., Angew. Chem., Int. Ed., 47:6172-6176 (2008); Jones, et al., Nature Methods, 8:499-U96 (2011)). If methods can be developed to deliver sulfonated fluorophores—which include the cyanine dyes and Alexa Fluors—across cell membranes (Pauff, et al., Org. Lett., 13:6196-6199 (2011)), then these too should be targetable to specific cellular proteins using the LplA method.

In this work, we focus on the use of strain-promoted cycloaddition to accomplish two-step fluorophore targeting, but the availability of new and/or improved bio-orthogonal ligation chemistries opens up alternative possibilities. In separate work, we demonstrate two-step fluorophore targeting using LplA in combination with Diels Alder cycloaddition between a trans-cyclooctene and tetrazine (Liu, et al., J. Am. Chem. Soc. 134(2):792-795, 2012)). The very fast cycloaddition kinetics (k˜10⁴ M⁻¹ s⁻¹) yields substantial improvements in signal to background ratio following intracellular protein labeling. Another interesting advance is in copper-catalyzed Click chemistry. Previously discounted for cellular applications due to copper toxicity, new improvements in copper ligand design and reactive oxygen species scavenging have made it possible to perform Click chemistry on live cell surfaces and even animals. If the toxicity can be further reduced, while preserving the fast kinetics of ligation (currently 10⁴-10⁷ fold greater than strain-promoted cycloaddition (Sletten, et al., 2011), then copper-catalyzed Click chemistry will be quite competitive with other methods for bio-orthogonal derivatization on the cell surface (but not inside cells).

Considered in the context of other protein labeling methods (Wombacher, et al., J. Biophotonics, 4:391-402 (2011) and Sletten, et al., Org. Lett., 10:3097-3099 (2008)), the disadvantages of the approach presented here are the requirement for co-expression of the LplA labeling enzyme, the unavoidable background caused by non-specific binding of cyclooctyne-fluorophore conjugates (albeit low in the case of hydrophilic fluorophores such as fluorescein and Oregon Green), and the signal which is fundamentally limited by the kinetics of strain-promoted cycloaddition chemistry. Considering these factors, the methodology will be most useful as a non-toxic (in contrast to FlAsH⁶) labeling method for abundant proteins, whose fusions to large tags (such as fluorescent proteins, HaloTag (Los, et al., 2008)), or SNAP tag (Gautier, et al., 2008)) perturb function. Actin is a key example.

### Example 4

### Synthesis of 7-Aminocoumarin Via Buchwald-Hartwig Cross Coupling for Specific Protein Labeling in Living Cells

### Methods

### Synthetic Methods

All experiments were conducted using oven-dried glassware under N₂ atmosphere and at ambient temperature (20-25° C.) unless otherwise specified. All other chemicals were purchased from Alfa Aesar or Aldrich and used without further purification. ¹H-NMR, ¹³C-NMR and ¹⁹F-NMR spectra were recorded on a Varian Mercury spectrometer and referenced to the solvent. Chemical shifts are reported as δ values (ppm) referenced to the solvent residual signals: CD₃OD, δ-H 3.31 ppm, δ-C 49.15 ppm; CD₂Cl₂, δ-H 5.32 ppm, δ-C 54.00 ppm; D₂O, δ-H 4.80 ppm; CF₃COOH for ¹⁹F-NMR, δ-F-78.50 ppm. Data for ¹H NMR are reported as follows: chemical shift (δ ppm), multiplicity (s=singlet, brs=broad singlet, d=doublet, t=triplet, q=quartet, m=multiplet), integration, coupling constant J (Hz). High-resolution mass spectra were obtained on a Bruker Daltonics APEXIV 4.7 Tesla Fourier transform mass spectrometer. Flash column chromatography was performed with 70-230 mesh silica gel.

### Synthesis of 7-hydroxycoumarin 2

To a solution of 7-hydroxycoumarin-3-carboxylic acid succinimidyl ester 1 (50 mg, from AnaSpec) in anhydrous DMF (0.5 mL) was added 5-aminovaleric acid (55 mg) and anhydrous triethylamine (0.1 mL). The reaction proceeded for 4 hours at 25° C. in the dark. The mixture was diluted with ethyl acetate (10 mL) and 1 M HCl (10 mL). Layers were separated, and the aqueous layer was extracted with ethyl acetate (15 mL×3). The combined organic layer was washed by water and brine. The organic phase was dried over Na₂SO₄ and concentrated in vacuo. The residue was purified by preparatory thin-layer chromatography (silica gel, 90:5:5 EtOAc:MeOH:acetic acid) to give 2 as yellow solid (48 mg, 98%). High-resolution ESI-MS characterization gave 306.0983 observed; 306.0972 calculated for [M+H]⁺. ¹H-NMR (400 MHz, CD₃OD, 25° C.): 8.75 (s, 1H), 7.66 (d, 1H, J=8.7), 6.87 (dd, 1H, J=2.1, 8.6), 6.76 (d, 1H, J=1.9), 3.54 (m, 2H, CH₂), 2.31 (t, 2H, CH₂), 1.68 (m, 4H, CH₂).

### Synthesis of 7-hydroxycoumarin methyl ester 3

To a solution of 2 (5 mg) in MeOH (1 mL) was added 1 M HCl solution in water (0.1 mL). The reaction proceeded for 24 hours at 25° C. Purification by flash column chromatography (silica gel, 20:80 hexanes:EtOAc) afforded 3 (5 mg, 93%) as a yellow solid. High-resolution ESI-MS characterization 320.1139 observed; 320.1129 calculated for [M+H]⁺. ¹H-NMR (500 MHz, CD₃OD, 25° C.): 8.75 (s, 1H), 7.62 (d, 1H, J=8.6), 6.90 (d, 1H, J=8.6), 6.79 (s, 1H), 3.67 (s, 3H, CH₃), 3.44 (m, 2H, CH₂), 2.39 (t, 2H, CH₂), 1.71 (m, 4H, CH₂). ¹³C-NMR (500 MHz, CD₃OD, 25° C.): δ 175.4, 165.3, 163.1, 157.9, 149.5, 132.5, 115.6, 114.1, 112.5, 103.1, 52.2, 40.7, 34.3, 29.7, 23.1.

### Synthesis of 7-trifluoromethylsulfonylcoumarin methyl ester 4

To a solution of 3 (38 mg, 0.12 mmol) in anhydrous dichloromethane (5 mL) and anhydrous pyridine (0.1 mL) at 0° C. was slowly added trifluoromethanesulfonic anhydride (30 μL, 0.18 mmol). The resulting mixture was stirred at room temperature for 2 h. The reaction was quenched with brine and diluted with ethyl acetate (10 mL). Layers were separated, and the aqueous layer was extracted with ethyl acetate (10 mL×3). The combined organic phase was dried over Na₂SO₄ and concentrated in vacuo to afford 4 (39 mg, 87%) as brown solid. The product was used in the next reaction without further purification. ESI-MS characterization gave 452.0611 observed; 452.0621 calculated for [M+H]⁺. ¹H-NMR (500 MHz, CD₂Cl₂, 25° C.): 8.89 (s, 1H), 7.85 (d, 1H, J=8.7), 7.38 (d, 1H, J=2.1), 7.33 (dd, 1H, J=2.0, 8.7), 3.64 (s, 3H, CH₃), 3.45 (m, 2H, CH₂), 2.35 (t, 2H, CH₂), 1.68 (m, 4H, CH₂). ¹³C-NMR (500 MHz, CD₂Cl₂, 25° C.): δ 174.1, 161.1, 160.9, 155.3, 152.6, 147.25, 132.2, 119.2, 119.1, 117.9, 115.3, 110.7, 51.9, 39.9, 34.0, 29.4, 22.8. ¹⁹F-NMR (300 MHz, CD₂Cl₂, 25° C.): δ−72.98.

### Synthesis of 7-diphenylmethyleneaminocoumarin methyl ester 5

An oven-dried flask was charged with (R)-(+)-BINAP (11 mg, 0.02 mmol), palladium(II) acetate (3 mg, 0.2 mmol), 4 (86 mg, 0.2 mmol) and cesium carbonate (164 mg, 0.5 mmol) and then purged with nitrogen. Benzophenone imine (46 mg, 0.025 mmol) and THF (5 mL) was added and the mixture was stirred at reflux under nitrogen for 4 hours. The mixture was cooled to room temperature, filtered, and concentrated. The yellow residue was purified by column chromatography (silica gel, 95:5→50:50 hexanes:EtOAc) to give 5 (53 mg, 70%) as a yellow solid. ESI-MS characterization gave 483.1932 observed; 483.1914 calculated for [M+H]⁺. ¹H-NMR (500 MHz, CD₃OD, 25° C.): 8.75 (s, 1H), 7.73 (d, 1H, J=8.7), 7.2-7.7 (m, 10H) 6.86 (dd, 1H, J=1.9, 8.6), 6.79 (s, 1H), 3.60 (s, 3H, CH₃), 3.42 (m, 2H, CH₂), 2.37 (t, 2H, CH₂), 1.66 (m, 4H, CH₂). ¹³C-NMR (500 MHz, CD₃OD, 25° C.): δ 174.2, 170.1, 162.2, 158.0, 155.8, 148.3, 130.7, 130.5, 130.1, 129.8, 129.7, 128.8, 119.2, 116.6, 114.7, 108.1, 51.9, 39.7, 34.0, 30.2, 22.8.

### Synthesis of 7-aminocoumarin 6

To a stirring solution of 5 (10 mg, 21 mmol) in 1:1 THF:water (10 mL) was added 1M HCl (0.5 mL). The reaction was stirred at 25° C. for 48 hours, then concentrated in vacuo. The yellow residue was purified by column chromatography (silica gel, 94:5:1 EtOAc:MeOH:NH₄OH) to afford 6 as a light yellow solid (5 mg, 76%). ESI-MS characterization gave 303.0973 observed; 303.0986 calculated for [M−H]⁻. ¹H-NMR (500 MHz, D₂O, 25° C.): 8.30 (s, 1H), 7.36 (d, 1H, J=8.3), 6.66 (d, 1H, J=8.6), 6.40 (s, 1H), 3.36 (m, 2H, CH₂), 2.29 (t, 2H, CH₂), 1.66 (m, 4H, CH₂). ¹³C-NMR (500 MHz, CD₃OD, 25° C.): δ 181.8, 164.6, 163.3, 158.4, 148.9, 132.2, 113.5, 109.7, 109.5, 98.4, 39.8, 38.1, 29.8, 24.5. λₘₐₓ (ε)=380 nm (18,400 M⁻¹ cm⁻¹) in pH 7 phosphate buffer.

Synthesis of 7-aminocoumarin-AM

To a stirring solution of 7-aminocoumarin 6 (3 mg, 9 μmol) in anhydrous acetonitrile (1 mL) was added silver(I) oxide (6 mg, 30 μmol) followed by acetoxymethyl bromide (1.5 μL, 15 μmol). The reaction was stirred at 25° C. for 12 hours, then concentrated in vacuo. The yellow residue was purified by column chromatography (silica gel, 8:1 EtOAc:hexane) to afford 7-aminocoumarin-AM as a light yellow solid (3 mg, 81% yield). ESI-MS characterization gave 377.1348 observed; 377.1343 calculated for [M+H]⁺. ¹H-NMR (300 MHz, CDCl₃, 25° C.): 8.39 (s, 1H), 7.28 (d, 1H, J=8.7), 6.70 (dd, 1H, J=8.6, 2.4), 6.45 (d, 1H, J=2.4), 5.72 (s, 2H), 3.34 (m, 2H, CH₂), 2.31 (t, 2H, CH₂), 2.09 (s, 3H, CH₃), 1.70 (m, 4H, CH₂).

### 7-Aminocoumarin and 7-hydroxycoumarin pH profiles

Fluorescence emission was recorded for 150 μM solutions, using a TECAN Safire Microplate Reader and a plastic transparent-bottomed 384-well plate (Greiner). pH 3-6 buffers were prepared by mixing different ratios of 0.1M acetic acid and 0.1M sodium acetate-trihydrate solutions. pH 7-10 buffers were prepared by mixing different ratios of 0.1M Na₂HPO₄ and either 0.1M HCl (for pH 7-9 buffers) or 0.1M NaOH (for pH 10 buffer). Final pH adjustments in all buffer solutions were made by adding small amount of 1M HCl or 1M NaOH.

### In Vitro 7-Aminocoumarin Ligation Reactions

Reactions were assembled as follows: 2 μM LplA enzyme, 150 μM LAP2 synthetic peptide (sequence: GFEIDKVWYDLDA; see Puthenveetil et al., J. Am. Chem. Soc. 2009, 131 16430-16438), 500 μM 7-aminocoumarin 6 probe, 5 mM ATP, and 5 mM Mg(OAc)₂ in 25 mM Na₂HPO₄ pH 7.2. The reaction mixture was incubated at 30° C. for 2 hours and quenched with EDTA (final concentration 100 mM). The mixture was analyzed on a Varian Prostar HPLC using a reverse-phase C18 Microsorb-MV 100 column (250×4.6 mm). Chromatograms were recorded at 210 nm. We used a 10-minute gradient of 30-60% acetonitrile in water with 0.1% trifluoroacetic acid under 1 mL/minute flow rate. LAP2 had a retention time of 7 minutes; after ligation to 7-aminocoumarin, the retention time increased to 9 minutes.

2 μM ^{W37V}LplA and 500 μM coumarin probe were used in one case. Aliquots from the reaction were collected and quenched with EDTA over 55 minutes. For the other case, 1 μM ^{W37V}LplA and 100 μM coumarin probe were used, and aliquots were collected and quenched over 70 minutes. After HPLC analysis, percent product conversions were calculated by dividing the product peak area by the sum of (product+starting material) peak areas.

### Mass Spectrometric Analysis of Peptides

Starred peaks from FIG. 2C were manually collected and injected into an Applied Biosystems 200 QTRAP mass spectrometer. The flow rate was 3 μL/minute and mass spectra were recorded under the positive-enhanced multi-charge mode.

### Mammalian Cell Culture

Human Embryonic Kidney (HEK) cells were cultured in Dulbecco's modified Eagle medium (DMEM; Cellgro) supplemented with 10% v/v fetal bovine serum (PAA Laboratories). For imaging, cells were plated as a monolayer on glass coverslips. Adherence of HEK cells was promoted by pre-coating the coverslip with 50 μg/mL fibronectin (Millipore). All cells were maintained at 37° C. under 5% CO₂.

### PRIME Cell Surface Labeling

HEK cells were transfected at ˜70% confluency with expression plasmids for LAP4.2^{[16]}-neurexin-1β (400 ng for a 0.95 cm² dish) and H2B-YFP (100 ng) using Lipofectamine 2000 (Invitrogen). 18 hours after transfection, cells were treated with 10 μM ^{W37V}LplA enzyme, 200 μM coumarin probe, 1 mM ATP, and 5 mM Mg(OAc)₂ in cell growth media for 20 minutes at room temperature. After removal of excess labeling reagents by replacing media 2-3 times, cells were immediately imaged, or incubated at 37° C. for 20 minutes to allow cell surface protein turnover.

### PRIME Intracellular Labeling

HEK or HeLa cells were transfected with expression plasmids for ^{W37V}LplA (20 ng) and LAP substrate (LAP2-YFP, LAP2-YFP-NLS, or LAP2-β-actin; 400 ng) using Lipofectamine 2000. 18 hours after transfection, cells were treated with 20 μM 7-aminocoumarin-AM in serum-free DMEM for 10 minutes at 37° C. Excess coumarin probe was removed by washing cells with cell growth media 4 times, for 15 minutes each time. Cells were imaged live thereafter.

### Fluorescence Imaging

Cells were imaged in Dulbecco's Phosphate Buffered Saline (DPBS) in confocal mode. We used a Zeiss Axiovert 200M inverted microscope with a 40× oil-immersion objective. The microscope was equipped with a Yokogawa spinning disk confocal head, a Quad-band notch dichroic mirror (405/488/568/647), and 405 (diode), 491 (DPSS), and 561 nm (DPSS) lasers (all 50 mW). 7-Aminocoumarin (405 laser excitation, 445/40 emission), YFP (491 laser excitation, 528/38 emission), and DIC images were collected using Slidebook software. Fluorescence images in each experiment were normalized to the same intensity ranges. Acquisition times ranged from 10-1000 milliseconds.

### Results

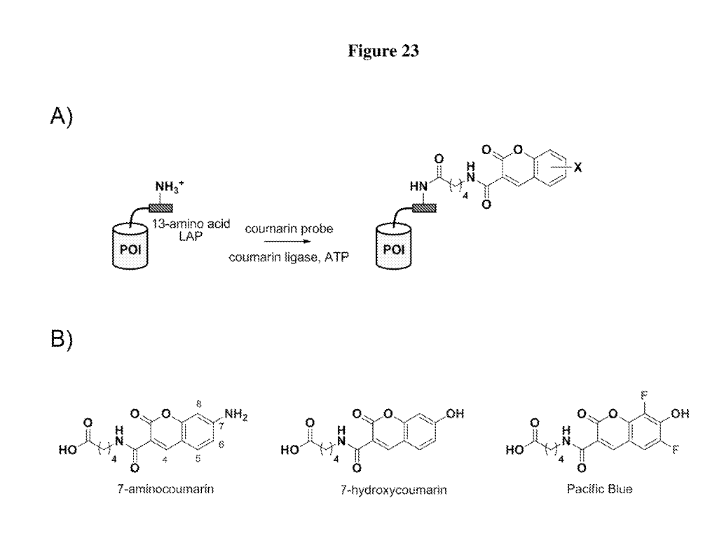

To enable minimally invasive studies of proteins in their native context, it is desirable to tag proteins with small, bright reporter groups. Recently, our lab described PRIME technology (for PRobe Incorporation Mediated by Enzymes) for such tagging (Uttamapinant, 2010; Baruah, et al., 2008; and Fernandez-Suarez, et al., 2007). An engineered variant of Escherichia coli lipoic acid ligase (LplA) is used to covalently attach a fluorescent substrate, such as 7-hydroxycoumarin, onto a 13-amino acid peptide recognition sequence (called LAP, for Ligase Acceptor Peptide) that is genetically fused to a protein of interest (POI). FIG. 23A. The targeting specificity is derived from the extremely high natural sequence specificity of LplA (Cronan, et al., Advances in Microbial Physiology, 50:103-146 (2005)). PRIME was used to label and visualize various LAP-tagged cytoskeletal and adhesion proteins in living mammalian cells.

One limitation of the 7-hydroxycoumarin probe used in our previous study is its pH-dependent fluorescence. The 7-OH substituent has a pKₐ of 7.5 (Sun, et al., Bioorganic & Medicinal Chem. Letters, 8:3107-3110 (1998)), and the fluorophore is only emissive in its anionic form. Proteins labeled by PRIME with 7-hydroxycoumarin (on the extracellular or luminal side) therefore cannot be visualized in acidic compartments of the cell such as the endosome (pH 5.5-6.5; see Demaurex, News in Physiological Sciences 2002, 17 1-5), where >90% of 7-hydroxycoumarin is expected to be neutral and therefore non-fluorescent. This problem prevents the use of 7-hydroxycoumarin for imaging receptor internalization and recycling, for example.

A potential solution is to use 6,8-difluoro-7-hydroxycoumarin (Pacific Blue; see Sun, et al., 1998, and FIG. 23B), which has a reduced 7-OH pKₐ of 3.7. An alternative coumarin structure is 7-aminocoumarin, also shown in FIG. 23B. In contrast to 7-hydroxycoumarin and Pacific Blue, 7-aminocoumarin is expected to be both neutral and highly fluorescent at a wide range of pH values. We also predicted that it would be a substrate for ^{W37V}LplA, since it is sterically similar to 7-hydroxycoumarin and is uncharged at physiological pH.

The synthesis of the 7-aminocoumarin substrate 6 required a novel route, however. Previous synthetic routes to 7-aminocoumarin derivatives have used either Pechmann (Pechmann, Berichte der deutschen chemischen Gesellschaft, 17:929-936 (1884)) or Perkin (Johnson, Organic Reactions, pp. 210-265 (1942)) condensation. The Pechmann reaction condenses aminoresorcinol with β-ketoesters and unavoidably produces 4-alkyl substituted aminocoumarins. Based on our structure-activity studies, a substituent at the 4 position of coumarin is unlikely to be tolerated by LplA. The Perkin reaction condenses aminoresorcinaldehyde with malonic acid and requires N-alkylation to prevent spontaneous Schiff base formation. A resulting N-alkylated aminocoumarin would be considerably larger than 7-hydroxycoumarin and unlikely to be accepted by our coumarin ligase.

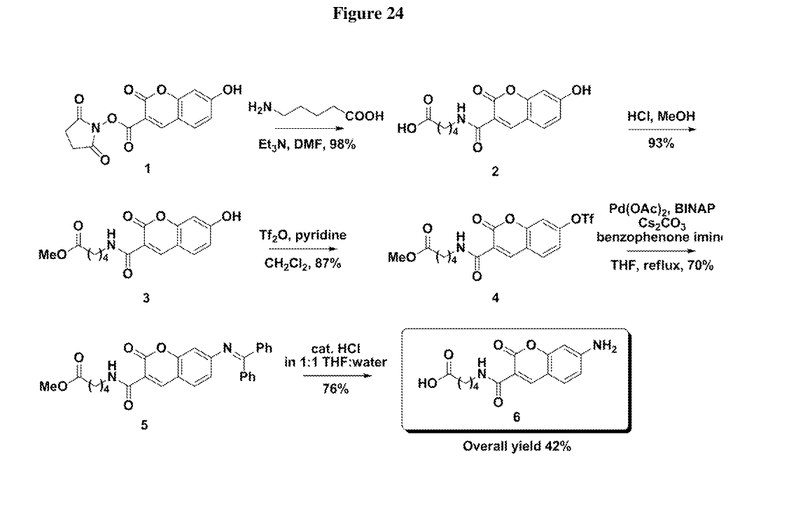

To access the simple, minimally bulky 7-aminocoumarin 6 structure shown in FIG. 23B, we devised a new synthetic route whose key feature is the palladium-catalyzed Buchwald-Hartwig cross coupling (Guram, et al., J. Am. Chem. Soc., 116:7901-7902 (1994); Paul, et al., J. Am. Chem. Soc., 116:5969-5970 (1994)) to convert the 7-OH group of 7-hydroxycoumarin into an unsubstituted primary aniline group. Our synthetic route (Scheme 1 shown in FIG. 24) began with the 7-hydroxycoumarin substrate 2, which was protected as a methyl ester derivative 3. Triflic anhydride and pyridine were used to convert 3 to 7-triflylcoumarin 4 in 87% yield. The Buchwald-Hartwig cross coupling was then performed with benzophenone imine as a surrogate for ammonia (Wolfe, et al., Tetrahedron Letters, 38:6367-6370 (1997). We used a catalytic combination of Pd(OAc)₂, BINAP, and Cs₂CO₃ previously designed to produce high coupling yields for electron-deficient aryl triflates and to reduce triflate hydrolysis (Ahman, et al., Tetrahedron Letters, 38:6363-6366 (1997)). The benzophenone imine-coumarin adduct 5 was obtained after gentle reflux with the catalyst system in THF in 70% yield. Benzophenone imine was then cleaved using acidic hydrolysis, which also hydrolyzed the methyl ester to give the final product, 7-aminocoumarin 6, in 76% yield. The overall yield for five synthetic steps was 42%.

We characterized the photophysical properties of 7-aminocoumarin 6 and compared to the 7-hydroxycoumarin isostere 2. The excitation and emission maxima of 7-aminocoumarin are 380 nm/444 nm, similar to those of 7-hydroxycoumarin (386 nm/448 nm (Sun, et al., Bioorganic & Medicinal Chem. Letters, 8:3107-3110 (1998)). The extinction coefficient of 7-aminocoumarin (18,400 M⁻¹ cm⁻¹) is about half that of 7-hydroxycoumarin (36,700 M⁻¹ cm⁻¹ (Sun, et al., 1998). As expected, 7-aminocoumarin fluorescence is fairly constant across the pH range 3-10, whereas 7-hydroxycoumarin fluorescence drops sharply at pH values <6.5.

We next tested 7-aminocoumarin for ligation by LplA variants. Although ^{W37V}LplA is the best single mutant of LplA for 7-hydroxycoumarin ligation, we previously found that several other LplA single mutants also had coumarin ligation activity (W37I, G, A, S, and L (Uttamapinant, et al., 2010). We therefore tested these LplA variants along with ^{W37V}LplA for 7-aminocoumarin ligation onto LAP. As with 7-hydroxycoumarin, ^{W37V}LplA was still the best among these for ligation of 7-aminocoumarin. An HPLC analysis was performed to monitor this ligation reaction. The starred peak indicated in the HPLC trace was collected and analyzed by mass spectrometry to confirm its identity as the covalent adduct between 7-aminocoumarin and LAP. Negative controls with ATP omitted, or ^{W37V}LplA replaced by wild-type LplA, gave no ligation product.

We compared the kinetics of 7-aminocoumarin and 7-hydroxycoumarin ligation by ^{W37V}LplA. With 500 μM of coumarin probe (likely saturating the ligase active site), 78% LAP was converted to product with 7-aminocoumarin, compared to 46% conversion with 7-hydroxycoumarin, after a 55-minute reaction. A 2-fold difference in reaction extent was also observed at lower coumarin concentration (100 μM) after 70 minutes. At the reaction pH of 7.4, ˜50% of 7-hydroxycoumarin is expected to be in the anionic form, whereas 7-aminocoumarin is neutral. The improved kinetics with 7-aminocoumarin likely reflects preferential binding of ^{W37V}LplA to neutral substrates.

7-aminocoumarin 6 was then used for PRIME labeling in living mammalian cells. Neurexin-1β, a transmembrane neuronal synapse adhesion protein (Craig, et al., Current Opinion in Neurobiology, 17:43-52 (2007)), was fused to LAP at its extracellular N-terminus, and labeled with 7-aminocoumarin and ^{W37V}LplA added to the growth medium. Positive cell imaging signals were observed after 20 minutes of 7-aminocoumarin labeling on Human Embryonic Kidney (HEK) cells expressing LAP-neurexin-1β and a transfection marker (histone 2B fused to yellow fluorescent protein, or H2B-YFP). A point mutation in the LAP sequence (Lys→Ala), or replacement of ^{W37V}LplA with wild-type LplA, eliminated 7-aminocoumarin labeling.

To test the ability of 7-aminocoumarin to visualize neurexin in acidic endosomes, we incubated 7-aminocoumarin-labeled cells at 37° C. for 20 minutes, to allow endocytic internalization of surface pools of neurexin-10. The appearance of internal 7-aminocoumarin puncta in cells was observed after this 20-minute internalization period. In contrast, cells similarly labeled with 7-hydroxycoumarin and then incubated, did not show internal fluorescence, due to quenching of 7-hydroxycoumarin fluorescence in acidic compartments.

We also tested 7-aminocoumarin for intracellular protein labeling. To deliver the probe across the cell membrane, we derivatized the carboxylic acid of 7-aminocoumarin 6 as an acetoxymethyl (AM) ester:

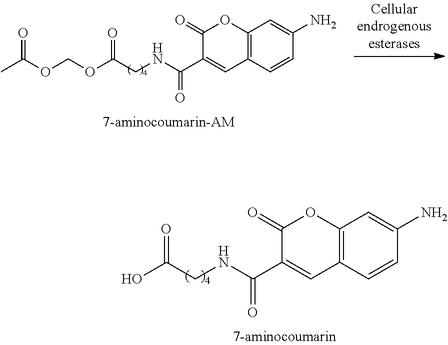

Upon entering cells, the AM ester is cleaved by endogenous esterases (Tsien, Annual Review of Neuroscience, 12:227-253 (1989)), releasing the parent 7-aminocoumarin 6 probe. To perform intracellular protein labeling, HEK cells were transfected with expression plasmids for both the coumarin ligase, ^{W37V}LplA, and a LAP fusion protein. 7-aminocoumarin-AM was incubated with cells for 10 minutes, then media was replaced over 60 minutes to allow endogenous anion transporters to clear excess unconjugated probe from the cytosol (Oh, et al., Pharmaceutical Research, 14:1203-1209 (1997)). Specific labeling was observed in cells expressing LAP-tagged yellow fluorescent protein (LAP-YFP), but not in neighboring untransfected cells. An alanine mutation in LAP sequence abolished 7-aminocoumarin labeling. To illustrate generality, we also labeled LAP-YFP targeted to the nucleus (LAP-YFP-NLS) and LAP fused to cytoskeletal protein β-actin.

In summary, to extend PRIME technology to imaging of proteins in acidic organelles while accommodating the steric and electronic constraints of our engineered coumarin ligase (Uttamapinant, et al., 2010), we have designed a new fluorescent ligase substrate. 7-aminocoumarin was synthesized by a novel route, using palladium-catalyzed Buchwald-Hartwig cross coupling to efficiently convert the 7-OH substituent into a 7-NH₂ substituent. We demonstrated that 7-aminocoumarin could be site-specifically targeted to LAP fusion proteins by the coumarin ligase, both on the cell surface and inside living mammalian cells. PRIME tagging with this new probe represents one step in our ongoing effort to generalize PRIME for labeling of any cellular protein with diverse fluorophore structures.

### Example 5

### Structure-Guided Engineering of a Pacific Blue Fluorophore Ligase for Specific Protein Imaging in Living Cells

Mutation of a gatekeeper residue, tryptophan 37, in E. coli lipoic acid ligase (LplA), expands substrate specificity such that unnatural probes much larger than lipoic acid can be recognized. This approach, however, has not been successful for anionic substrates. Here we report the results of a structure-guided, two-residue screening matrix to discover an LplA double mutant, E20G/W37TLplA, that ligates Pacific Blue as efficiently as W37VLplA ligates 7-hydroxycoumarin. The utility of this Pacific Blue ligase for specific labeling of recombinant proteins inside living cells, on the cell surface, and inside acidic endosomes is demonstrated.

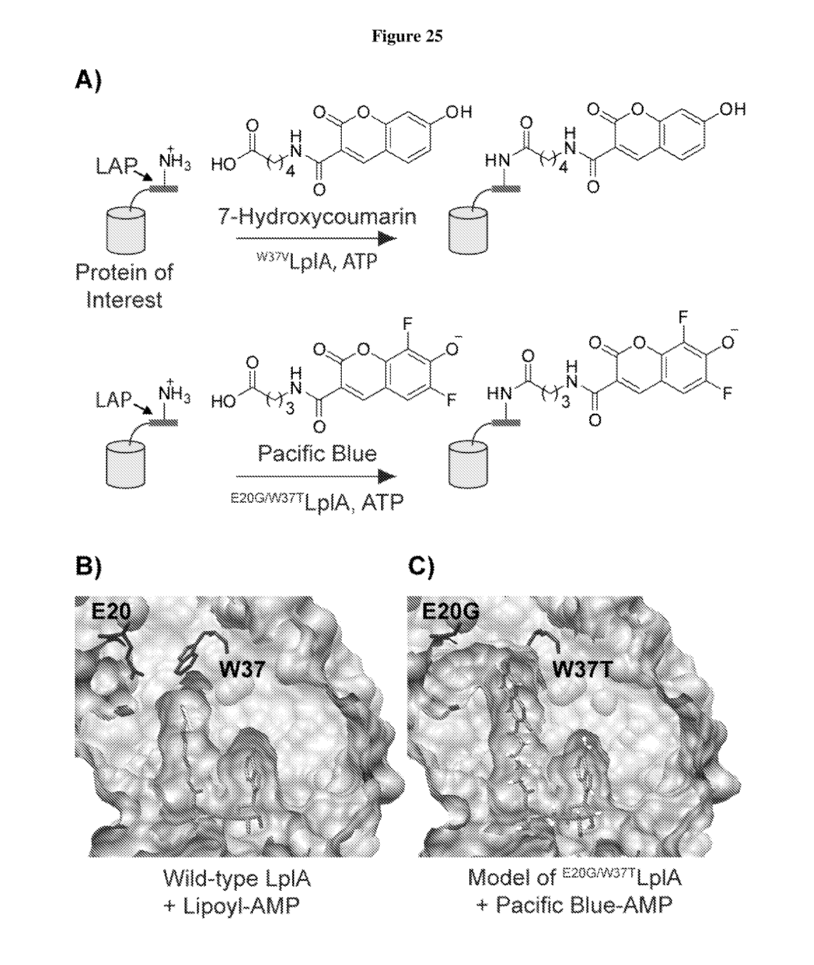

The goal of this work was to use PB as a model compound to explore strategies for engineering new LplA activity, such as recognition of anionic substrates, beyond point mutations at W37. A PB ligase is also a useful alternative to HC ligase for studying proteins in acidic cellular compartments, where HC fluorescence is very low. By performing in vitro screens using a panel of E20 and W37 single and double mutants, we discovered that ^{E20G/W37T}LplA ligates PB with comparable kinetics to ^{W37V}LplA ligation of HC (FIG. 25). We demonstrated the utility of our PB ligase for in vitro, cell surface, and intracellular site-specific protein labeling.

### Materials and Methods

### Plasmids

The LplA-pYJF16 plasmid was used for bacterial expression of LplA. (Uttamapinant, et al., 2010; and Fujiwara, et al., J. Bio. Chem., 285:9971-9980 (2010). The LplA-pcDNA3 plasmid was used for mammalian expression of LplA. For mammalian expression of LAP fusion proteins, LAP-YFP-NLS-pcDNA3, LAP4.2-neurexin-β-pNICE, and vimentin-LAP in Clontech vector were used, and have been described. See, e.g., Uttamapinant, et al., 2010 and Jin, et al., 2011). The LAP sequence used was GFEIDKVWYDLDA (SEQ ID NO:4). For some constructs (neurexin and LDL receptor), an alternative peptide sequence called LAP4.2 was used instead (GFEIDKVWHDFPA; SEQ ID NO:5) (Puthenveetil, et al., 2009). LAP4.2-LDLR-pcDNA4 was generated from HA-LDLR-pcDNA4 (Zou, et al., Acs Chem. Bio., 6:308-313 (2011)) by a two-stage QuikChange to insert the LAP4.2 sequence, and was a gift from Daniel Liu (MIT). The nuclear YFP transfection marker was H2B-YFP and has been described (Howarth, et al., Nature Methods, 5:397-399 (2008)).

All mutants were prepared by QuikChange mutagenesis.

### LplA Expression and Purification

LplA mutants were expressed in BL21 E. coli and purified by His₆-nickel affinity chromatography as previously described. See, e.g., Uttamapinant, et al., 2010.

### In Vitro Screening of LplA Mutants

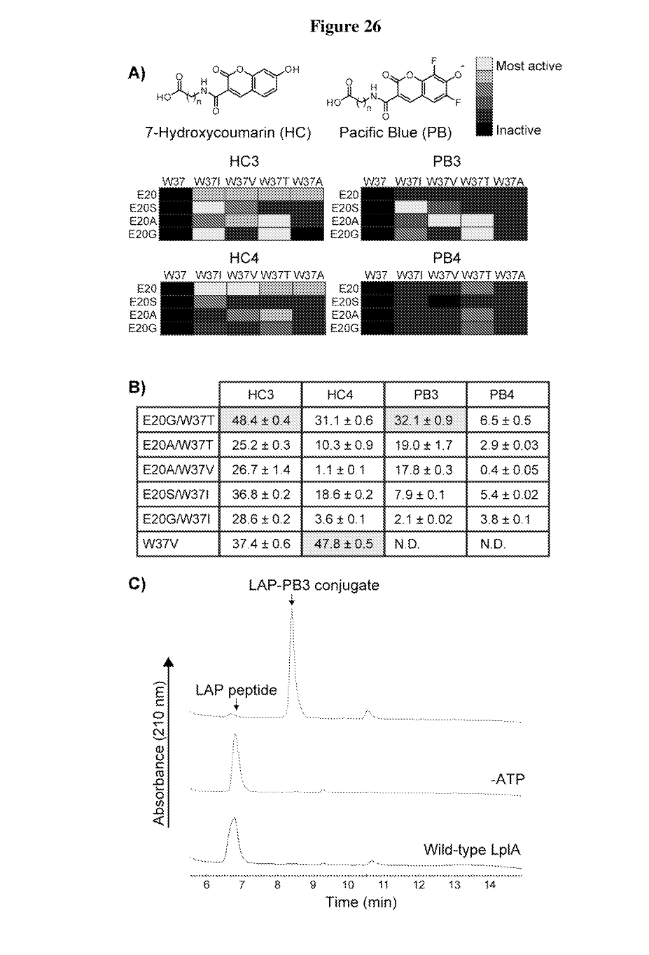

Ligation reactions were assembled as follows for FIG. 26A: 2 μM of purified LplA mutant, 150 μM synthetic LAP peptide (GFEIDKVWYDLDA (SEQ ID NO:4); synthesized by the Tufts Peptide Synthesis Core Facility), 5 mM ATP, 500 μM fluorophore probe, 5 mM magnesium acetate, and 25 mM Na₂HPO₄ pH 7.2 in a total volume of 25 μL. Reactions were incubated for 12 hrs at 30° C.

LplA mutant/probe combinations giving high activity under these conditions were then re-assayed with 10-fold lower probe (50 μM) for 2 hrs.

Product formation was analyzed by Ultra Performance Liquid Chromatography (UPLC) on a Waters Acquity instrument using a reverse-phase BEH C18 column 1.7 μM (1.0×50 mm) with inline mass spectroscopy. Chromatograms were recorded at 210 nm. A gradient of 30 to 70% (acetonitrile+0.05% trifluoroacetic acid) in (water with 0.1% trifluoroacetic acid) over 0.78 min was used.

### Further In Vitro Screening of Top Five LplA Double Mutants

Reactions for the top five LplA double mutants were assembled as above, but with 500 μM probe and a reaction time of 45 min. Reactions were quenched with EDTA to a final concentration of 100 mM. Product formation was analyzed on a Varian Prostar HPLC using a reverse-phase C18 Microsorb-MV 100 column (250×4.6 mm). Chromatograms were recorded at 210 nm. We used a 10-minute gradient of 30-60% acetonitrile in water with 0.1% trifluoroacetic acid under 1 mL/minute flow rate. Percent conversions were calculated by dividing the product peak area by the sum of (product+starting material) peak areas.

### Michaelis-Menten Kinetic Assay

The Michaelis-Menten curve shown in FIG. S4 was generated as previously described.² Reaction conditions were as follows: 2 μM^{E20G/W37T}LplA, 600 μM synthetic LAP peptide, 2 mM magnesium acetate, and 25 mM Na₂HPO₄ pH 7.2.

### Mammalian Cell Culture and Imaging

HEK and HeLa cells were cultured in growth media consisting of Minimum Essential Medium (MEM, Cellgro) supplemented with 10% fetal bovine serum (FBS, PAA Laboratories). Cells were maintained at 37° C. under 5% CO₂. For imaging, HEK cells were grown on glass coverslips pre-treated with 50 μg/mL fibronectin (Millipore) to increase their adherence.

Cells were imaged in Dulbecco's Phosphate Buffered Saline (DPBS) at room temperature. The images in FIGS. 3 and 4 were collected on a Zeiss AxioObserver.Z1 microscope with a 40× oil-immersion objective and 2.5× Optovar, equipped with a Yokogawa spinning disk confocal head containing a Quad-band notch dichroic minor (405/488/568/647 nm). Pacific Blue/coumarin (405 nm laser excitation, 445/40 emission filter), YFP (491 nm laser excitation, 528/38 emission filter), Alexa Fluor 568 (561 nm laser excitation, 617/73 emission filter) and DIC images were collected using Slidebook software (Intelligent Imaging Innovations). Images were acquired for 100 milliseconds to 1 second using a Cascade II:512 camera. Fluorescence images in each experiment were normalized to the same intensity range.

### Cell Surface Labeling

HEK cells were transfected with 200 ng LAP4.2-LDLR-pcDNA4 and 100 ng H₂B-YFP co-transfection marker plasmid, per 0.95 cm² at ˜70% confluency, using Lipofectamine 2000 (Invitrogen). 15 hours after transfection, the growth media was removed, and the cells were washed three times with DPBS. The cells were labeled by applying 100 μM Pacific Blue or hydroxycoumarin probe, 2 μM ligase, 1 mM ATP, and 5 mM Mg(OAc)₂ in DPBS at room temperature for 40 minutes. Cells were then washed three times with DPBS and either imaged immediately or incubated at 37° C. for an additional 30 minutes to allow receptor internalization prior to imaging.

### Intracellular Protein Labeling

HEK cells were transfected at ˜70% confluency with 200 ng of LAP-YFP-NLS-pcDNA3 and 50 ng of FLAG-^{E20G/W37T}LplA-pcDNA3 per 0.95 cm² using Lipofectamine 2000 (Invitrogen). 15 hours after transfection, the growth media was removed, and the cells were washed three times with serum-free MEM. The cells were labeled by applying 20 μM PB3-AM₂ in serum-free MEM at 37° C. for 20 minutes. The cells were then washed three times with fresh MEM. Excess probe was removed by changing the media several times over 40 min.

To visualize LplA expression levels, cells were fixed using 3.7% formaldehyde in PBS pH 7.4 for 10 minutes, followed by methanol at −20° C. for 5 minutes. Fixed cells were washed with DPBS, then blocked overnight with blocking buffer (3% BSA in DPBS with 0.1% Tween-20). Anti-FLAG M2 antibody (Sigma) was added at a 1:300 dilution in blocking buffer for one hour at room temperature. Cells were then washed three times with DPBS before treatment with a 1:300 dilution of goat anti-mouse antibody conjugated to Alexa Fluor 568 (Invitrogen) in blocking buffer for one hour at room temperature. Cells were washed three times with DPBS prior to imaging.

For labeling of vimentin-LAP (FIG. 4B), HeLa cells were transfected with 250 ng vimentin-LAP-Clontech, 50 ng FLAG-^{E20G/W37T}LplA-pcDNA3, and 100 ng H₂B-YFP transfection marker per 0.95 cm² using Lipofectamine 2000. Labeling was performed as above, with an extended 60 minute wash out period to remove excess probe. Cells were then imaged live in DPBS.

We note that, compared to intracellular labeling with hydroxycoumarin, labeling with PB3 generally requires longer washout times, up to 60 minutes in some cases. Shorter wash times result in higher PB background in all cells.

### Probe Synthesis

To synthesize Pacific Blue with the n=3 linker (PB3), Pacific Blue succinimidyl ester (5 mg, 14.7 μmol, Invitrogen) in 120 μL of dry dimethyl sulfoxide (DMSO) was combined with 4-aminobutyric acid (2.9 mg, 28.1 μmol, Alfa Aesar) and triethylamine (TEA, 8 μL, 57.4 μmol). The reaction was allowed to proceed at room temperature overnight in the dark. Purification was performed in batches. 40 μL of crude mixture was diluted into 800 μL of water, and purified by preparatory HPLC (Varian DynamaxMicrosorb 300-5 C18, 250×12.4 mm column). A gradient of 0-100% acetonitrile in water over 20 min was used and detection was performed at 405 nm. Fractions were lyophilized and then dissolved in 50 μL dry dimethylformamide (DMF). PB4 was synthesized in a similar fashion using 5-aminovaleric acid (Alfa Aesar). The syntheses of HC3 and HC4 probes have been described. ¹ ESI-MS [M−H]⁻ for PB3: 326.04 observed, 326.05 calculated (Cronan, et al., (2005) Advances in Microbial Physiology, 50:103-146 (2005) and Uttamapinant, et al., 2010). ¹H NMR for PB3 (D₂O, 300 MHz): 8.58 (d, 1H), 7.23 (dd, 1H), 3.40 (t, 2H), 2.31 (t, 2H), 1.85 (m, 2H). ESI-MS [M−H]⁻ for PB4: 340.08 observed, 340.06 calculated.

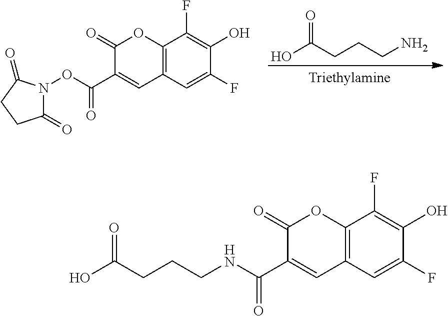 [SMILES: O=C(ON1C(=O)CCC1=O)c1cc2cc(F)c(O)c(F)c2oc1=O ; NCCCC(=O)O ; O=C(O)CCCNC(=O)c1cc2cc(F)c(O)c(F)c2oc1=O]

To synthesize cell-permeable PB3-AM₂, PB3 (0.5 μmol) in 25 μL of DMF was combined with bromomethyl acetate (0.5 μL, 5.1 μmol, Aldrich) and N,N-diisopropylethylamine (DIEA, 1 μL, 5.7 μmol). The reaction was allowed to proceed overnight at room temperature in the dark. 450 μL of water was then added to the reaction mixture, and the product was extracted using 3×800 μL of ethyl acetate. The combined organic layers were concentrated in vacuo to an oil and purified by preparatory-scale silica thin-layer chromatography (2:1 ethyl acetate:hexanes, R_{f} 0.49). The purified PB3-AM₂ was stored in DMSO at −20° C. We have observed that incomplete purification at this step can lead to increased background in cell labeling experiments. ESI-MS [M+H]⁺ for PB3-AM₂: 471.72 observed, 472.11 calculated. ¹H NMR for PB3-AM₂(CDCl₃, 500 MHz): 8.80 (s, 1H), 8.71 (m, 1H), 5.79 (s, 2H), 5.75 (s, 2H), 3.52 (m, 2H), 2.47 (t, 2H), 2.14 (s, 3H), 2.12 (s, 3H), 1.99 (m, 2H).

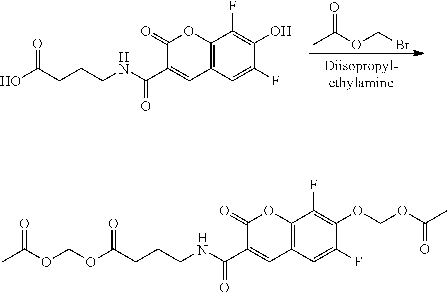

### LplA Modeling

The previously reported structure of E. coli LplA containing lipoyl-AMP (3A7R) was used as a starting point. Uttamapinant, et al., 2010. The energy minimized structure of PB3-AMP conformation was generated using Avogadro with the AMP moiety fixed. Baruah, et 2008. PB3-AMP was then placed into the 3A7R structure with the AMP moieties aligned exactly. E20 and W37 sidechains were changed using the mutate tool in the program Visual Molecular Dynamics. Fernandez-Suarez, et al., 2007.

### UPLC Screening of LplA Single Mutants

In vitro ligation reactions were assembled as described in the main text with the following modifications. 5 μL bacterial lysate containing LplA was used in place of purified enzyme. Total reaction volume was 25 μL. Instead of LAP peptide, 150 μM purified E2p protein (see Uttamapinant, et al., 2010) was used. Reactions proceeded overnight at 30° C. with 500 μM probe. Ultra Performance Liquid Chromatography (UPLC) was used to detect any product formation. All products were confirmed by in-line mass spectrometry.

### Mammalian Cell Lysate Labeling

HEK cells were lysed under hypotonic conditions in 1 mM HEPES pH 7.5 with 5 mM MgCl₂, protease inhibitor cocktail (Calbiochem), and 1 mM phenylmethylsulfonyl fluoride. Three cycles of freeze-thaw with 3 min of vortexing was performed, followed by centrifugation to clear the lysate. To the lysate was added 10 μM purified LAP-YFP protein, 500 nM PB ligase, 500 μM PB3, 5 mM ATP, 5 mM magnesium acetate, and 25 mM sodium phosphate buffer, pH 7.2. Reactions were incubated overnight, then boiled in protein loading buffer for 10 min and analyzed on a 10% SDS-PAGE gel. Coumarin fluorescence was visualized on an Alpha Innotech Chemilmager 5500 instrument.

### Results

### Screening for a Pacific Blue Ligase

Based on the LplA crystal structure (FIG. 25B) (Fujiwara, et al., J. Bio. Chem., 285:9971-9980 (2010)), we decided to focus our engineering efforts on the W37 and E20 positions. We started with a preliminary screen of nineteen W37 point mutants and fourteen E20 point mutants, against four probe structures. These four structures, shown in FIG. 26A, are two Pacific Blue probes with shorter (n=3) and longer (n=4) linkers (PB3 and PB4), and two analogous 7-hydroxycoumarin probes (HC3 and HC4). Some Pacific Blue (PB) ligation product was detected after a 12 hour reaction with W37T, V, I, and A LplA mutants (FIG. S1), so we decided to introduce these mutations into our next screen. Note that the activity of the best point mutant, ^{W37T}LplA, which gave ˜50% conversion to PB ligation product after 12 hours, is too slow for practical utility. For E20, none of the tested point mutants gave product with any of the four probes after 12 hours. Nevertheless, in our next screen, we included E20 mutations to the smaller, neutral sidechains Gly, Ala, and Ser.

Our next library consisted of 7 single mutants (four at W37 and three at E20) and their 12 crossed double mutants, shown in FIG. 26A. Screening was performed using 500 μM probe in an overnight reaction. Any ligase/probe combination with high activity under these conditions was re-assayed using 50 μM probe in a 2 hour reaction. As before, the E20 single mutants had no detectable activity (FIG. 26A). The W37 single mutants were minimally active with both PB probes, although high activity was seen with HC3 and HC4. The best single mutant/probe pair was ^{W37V}LplA with HC4.

The LplA double mutants, however, had interesting patterns of activity with PB. Although none of the mutants ligated PB4 efficiently, PB3 was ligated well by five double mutants (FIG. 26A; re-evaluated quantitatively in FIG. 26B). The best two have the W37T mutation, suggesting that not only size reduction but also polarity increase at this position is beneficial for PB recognition. We noticed that the W37A mutation performed poorly in the context of all double mutants for all 4 probes, perhaps because it destabilizes the binding pocket. The best E20 mutation to pair with W37T was Gly, perhaps because it generates the most space and conformational freedom. Together, our observations suggest that W37 and E20 mutations work synergistically to allow PB uptake: W37 mutations enlarge the binding pocket, while E20 mutations remove repulsive electrostatic interactions (FIG. 25C).

We proceeded to fully characterize our best PB ligase to emerge from this screen, ^{E20G/W37T}LplA. First, HPLC analysis of the ligation reaction was repeated (FIG. 26C), alongside negative controls omitting ATP or replacing PB ligase with wild-type LplA. Second, the kinetic constants for PB3 ligation to LAP were measured by HPLC. Both k_{cat} (0.014±0.001 s⁻¹) and K_{M} (17.5±4.3 μM) values are comparable to those previously determined for HC4 ligation catalyzed by ^{W37V}LplA (k_{cat} 0.019±0.004 s⁻¹ and K_{M} 56±20 μM) Uttamapinant, et al., 2010. Finally, we tested the sequence-specificity of PB3 ligation by labeling a LAP fusion protein within mammalian cell lysate. Only LAP2 was labeled by PB ligase, and not any endogenous mammalian proteins.

Cell Surface Labeling with Pacific Blue Ligase

To test our PB ligase on living cells, we first performed labeling of a cell surface protein. The neuronal adhesion protein neurexin-1β with LAP4.2 (a variant of LAP; see Puthenveetil, et al., 2009) whose sequence is given in the Materials and Methods section above) fused to its extracellular N-terminus was expressed in human embryonic kidney (HEK) cells. Labeling was performed by adding purified PB ligase, PB3 probe, and ATP to the cellular media for 30 min. A ring of PB fluorescence around cells expressing LAP4.2-neurexin was observed, as indicated by the presence of the co-transfection marker, whereas untransfected neighboring cells are not labeled. Negative controls performed with wild type LplA, ATP omitted, or an alanine mutation in LAP resulted in no visible labeling.

A potential advantage of PB ligase over HC ligase is for visualization of proteins in acidic organelles, where HC fluorescence is low due to its pKa of 7.5. To test this experimentally, we used PB ligase or HC ligase to label LAP4.2-LDL receptor (low density lipoprotein receptor) on the surface of HEK cells. After labeling, cells were incubated for 30 min at 37° C. to allow internalization of fluorescently-tagged receptors. PB-tagged LAP4.2-LDL receptor was clearly visible within internalized puncta, whereas HC-tagged LAP4.2-LDL receptor is not. Separate experiments showed that many of the PB-labeled internal puncta overlap with FM4-64, an endosomal marker.

Intracellular Protein Labeling with Pacific Blue Ligase

We tested PB ligase for labeling of intracellular proteins in living mammalian cells. To deliver PB3 across the cell membrane, we first protected the carboxylic acid and 7-hydroxyl groups of PB3 with acetoxymethyl (AM) groups to give PB3-AM₂ (structure shown in the Materials and Methods section above). Endogenous intracellular esterases remove the AM groups to give PB3 inside the cell (Tsien, 1989). HEK cells were co-transfected with plasmids for PB ligase and LAP-YFP-NLS (NLS is a nuclear localization signal; YFP is yellow fluorescent protein). To perform labeling, PB3-AM₂ was incubated with cells for 20 min, then the media was replaced 3 times over 40 min to allow cells to pump out excess, unconjugated probe. The cells were then fixed and anti-FLAG immunostaining was performed to visualize enzyme expression. As expected for specific labeling, PB fluorescence overlaps well with the YFP fluorescence of LAP-YFP-NLS. PB was not seen in neighboring untransfected cells. PB labeling was also absent when wild-type LplA is used in place of PB ligase, or the LAP-YFP-NLS contains a Lys→Ala mutation in the LAP sequence. To illustrate generality, we also performed PB labeling in live cells of vimentin-LAP, an intermediate filament protein and obtained positive imaging results.

### Discussion

In this study, we identified an LplA double mutant capable of recognizing and ligating a charged probe, Pacific Blue. Unlike previous studies where simple enlargement of the binding pocket via a point mutation at W37 was sufficient to allow recognition of large hydrophobic probes, the synergistic effect of mutating both the E20 and W37 positions was required for recognition of Pacific Blue. Guided by the LplA crystal structure, we were able to create a small and focused library of single and double LplA mutants to screen for the desired PB ligation activity. No single mutation had significant activity, but the augmentation of the most active W37 single mutants by E20 mutations resulted in a kinetically efficient PB ligase. We anticipate that these insights into the substrate binding pocket of LplA will prove useful in future engineering efforts. The engineered PB ligase has k_{cat} and K_{M} values similar to those of our previously reported 7-hydroxycoumarin ligase (Uttamapinant, et al., 2010). PB ligase also retained sequence-specificity for LAP over all endogenous mammalian proteins and could therefore be used for specific protein labeling inside and on the surface of living mammalian cells.

With this report, PRIME labeling can now be performed with any of three coumarin probes: Pacific Blue, 7-hydroxycoumarin (Uttamapinant, et al., 2010), or 7-aminocoumarin (AC) (Jin, et al., 2011). The decision of which coumarin to use is dependent on the specific application. HC is the brightest of the three probes, followed by PB and then AC due to its decreased extinction coefficient (Sun, et al., 1998; and Jin, et al., 2011). However as demonstrated here, PB and AC have the added benefit of pH-insensitivity, whereas the pKa of HC makes it unsuitable for imaging in acidic organelles such as endosomes.

### Example 6

### Site-Specific Protein Modification Using Lipoic Acid Ligase and Bis-Aryl Hydrazone Formation

A screen of Trp37 mutants of E. coli lipoic acid ligase (LplA) produced enzymes capable of ligating an aryl-aldehyde or an aryl-hydrazine substrate to LplA's 13-amino acid acceptor peptide (LAP2). Once site-specifically attached to recombinant proteins, aryl-aldehydes could be chemo-selectively derivatized with hydrazine-probe conjugates, and aryl-hydrazines could be derivatized in an analogous manner with aldehyde-probe conjugates. Such two-step labeling was demonstrated for AlexaFluor568 targeting to monovalent streptavidin in vitro, and to neurexin-1β on the surface of living mammalian cells. To further highlight this technique, we also labeled low density lipoprotein receptor on the surface of live cells with fluorescent phycoerythrin protein to allow single molecule imaging and tracking over time.

### Materials and Methods

### Plasmids

For expression of His6-tagged LplA in E. coli, we used the LplA-pYJF16 plasmid. Uttamapinant, et al., 2010. The cloning of LAP-streptavidin-pET21a for bacterial expression is described below:

Monovalent streptavidin containing a single LAP tag was generated starting from the streptavidin-pET21a expression plasmid for the alive subunit. Kent, Chem. Soc. Rev., 38:338-51 (2009) and Howarth, et al., Nature Methods, 3:267-273 (2006). The following primers were used to introduce a LAP tag at the Nterminus using PCR amplification and the amplified fragment was digested and inserted between the NdeI and HindIII restriction sites.

LAP2-Streptavidin-fwd:
(SEQ ID NO: 9)
AAAACATATGGGATTCGAGATCGACAAGGTGTGGT
ACGACCTGGACGCCGGTGCTGAAGCTGGTATCACC
Strep-rev:
(SEQ ID NO: 10)
GTGCGGCCGCAAGCTTTTATTAATG

The LAP-Alkaline Phosphatase construct in FIG. S3 was constructed using the plasmid pQUANTagen(kx) (Yao, et al., J. Am. Chem. Soc. (2012) and Desvaux, et al., Microbiology-Sgm, 153:59-70 (2007)). The LAP tag was introduced between the SalI and SacI restriction sites using the following two annealed primers:

FLAG-LAP2-pQUANTAGEN-fwd:
(SEQ ID NO: 11)
TCGACATGGACTACAAGGATGACGA
CGATAAGGGCTTCGAGATCGACAAGGTGTGGTACGACCTGGACGCCGGAG
CT
FLAG-LAP2-pQUANTAGEN-rev:
(SEQ ID NO: 12)
CCGGCGTCCAGGTCGTACCACACCTTGT
CGATCTCGAAGCCCTTATCGTCGTCATCCTTGTAGTCCATG

For expression of LAP fusion proteins in mammalian cells, we used LAP4.2-neurexin-1β-pNICE (Uttamapinant, et al., 2010) and LAP4.2-LDLR-pcDNA4 (Cohen, et al., Biochemistry, 50:8221-8225 (2011)). Mammalian expression plasmids for BirA-ER, AP-LDLR and H2B-YFP have been described previously. See, e.g., Howarth, et al., Nat. Protoc., 3:534-545 (2008), Zou, et al., ACS Chem. Biol., 6:308-313 (2011), and Howarth, et al., Nat. Methods, 5:397-399 (2008).

### In Vitro Screening for Ald and Hyd Ligation Activity

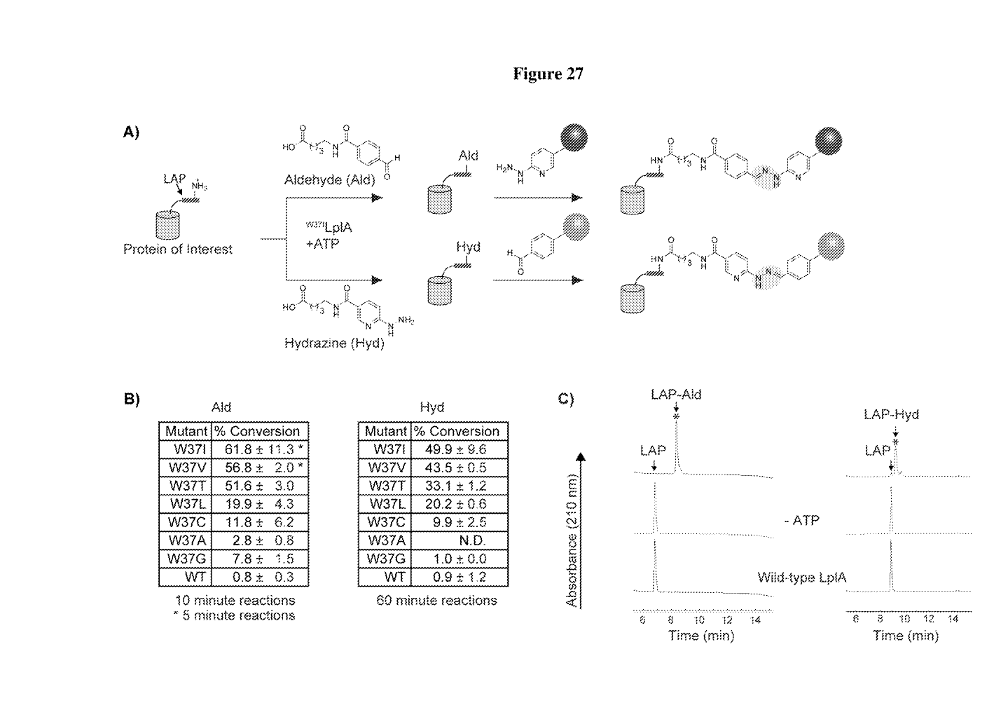

Ligation reactions were assembled as follows: 1 μM of purified LplA mutant (Uttamapinant, et al., 2010), 150 μM synthetic LAP2 peptide (GFEIDKVWYDLDA; SEQ ID NO:4), 5 mM ATP, 500 μM of either Ald or Hyd probe, 5 mM magnesium acetate, and 25 mM Na2HPO4 pH 7.2 in a total volume of 20 μL. Reactions were incubated for 5 to 60 min at 30° C. and then quenched with EDTA to a final concentration of 45 mM. Samples were diluted to a total volume of 80 μL in conjugation buffer (10 mM Na2HPO4, 3.2 mM KH2PO4, 2.7 mM KCl, 140 mM NaCl, pH 5.0) and analyzed on a Varian Prostar HPLC using a reverse-phase C18 Microsorb-MV 100 column (250×4.6 mm). Chromatograms were recorded at 210 nm. For analysis of the aldehyde ligation reaction we used a 10-minute gradient of 30-60% acetonitrile in water with 0.1% trifluoroacetic acid under 1 mL/minute flow rate. For analysis of the hydrazine ligation reaction a gradient of 25-60% over 14 minutes with the same solvents was used. Percent conversions were calculated by dividing the product peak area by the sum of (product+starting material) peak areas. Reactions were performed in triplicate (Ald) or duplicate (Hyd) and the average values are shown. Reactions in FIG. 27C were performed using the conditions above with a 70 minute reaction time for Ald and 120 minute reaction time for Hyd.

### LAP-Monovalent Streptavidin Expression and Purification

Monovalent streptavidin containing a single LAP tag fused to the N-terminus of the “alive” subunit was expressed and purified as previously described (Howarth, et al., 2008). Briefly, the alive (LAP-tagged, His6-tagged) and dead (untagged) subunits of streptavidin were expressed separately in E. coli. The inclusion bodies were solubilized and the alive and dead proteins were combined in a 3:1 ratio. After refolding to obtain a statistical mixture, monovalent streptavidin containing exactly one alive subunit and three dead subunits was purified using gradient nickel affinity chromatography. Monovalency was confirmed using a DNA gel shift assay. LAP-mSA was mixed with 250 bp biotinylated DNA at a 1:1 and 10:1 molar ratio and run on a 1.5% agarose gel. A band corresponding to binding of a single biotinylated DNA was observed. In comparison, wild-type streptavidin under the same conditions binds between 1 to 4 biotinylated DNA molecules.

### In Vitro Labelling of LAP Fusion Proteins

Reactions were assembled using 2 μM LAP-mSA, 500 nM W37ILplA, 5 mM ATP, 100 μM of either Ald or Hyd, 5 mM magnesium acetate, and 25 mM Na2HPO4 pH 7.2 in a total volume of 20 μL. Reactions were incubated at room temperature for 1 hr. Each reaction was then diluted to a volume of 500 μL of PBS and the buffer adjusted to pH 5 using HCl. Thereafter, the solution was concentrated to ˜30 μL using an ultrafiltration concentrator with a MWCO of 5 kDa (Vivaspin 500, GE Healthcare). This was repeated twice in order to fully exchange the buffer and eliminate excess probe. Conjugation was then performed by adding 20 mM aniline and 200 μM of either AlexaFluor568-hydrazide (Invitrogen) or fluorescein-aldehyde (4FB-PEG3-fluorescein, Solulink). Reactions were incubated overnight and analyzed on a 10% SDS-PAGE gel. In gel fluorescence imaging was performed using a Fujifilm FLA-9000.

### Mammalian Cell Culture

HEK and COS-7 cells were cultured in growth media consisting of Minimum Essential Medium (MEM, Cellgro) supplemented with 10% fetal bovine serum (FBS, PAA Laboratories). Cells were maintained at 37° C. under 5% CO₂. For imaging, HEK cells were grown on glass coverslips pre-treated with 50 μg/mL fibronectin (Millipore) to increase their adherence. COS-7 cells were grown in LabTek II chambered coverglass system 8-well plates.

### Microscopy

Cells were imaged in Dulbecco's Phosphate Buffered Saline (DPBS) at room temperature. The confocal images were collected on a Zeiss AxioObserver.Z1 microscope with a 40× oil-immersion objective and 2.5× Optovar. The images were collected in confocal mode using a Yokogawa spinning disk confocal head with a Quad-band notch dichroic mirror (405/488/568/647 nm). YFP (491 nm laser, 528/38 emission filter), AlexaFluor568/Phycoerythrin (561 nm laser, 617/73 emission filter), and Normarski-type DIC images were collected using a Cascade II:512 camera and Slidebook software (Intelligent Imaging Innovations). Fluorescence images in each experiment were normalized to the same intensity range.

TIRF images were acquired on the same microscope using a TIRF slider. YFP (491 nm laser excitation, 525/30 emission filter, 502 nm dichroic mirror), Alexa Fluor 568/Phycoerythrin (561 nm laser excitation, 605/30 emission filter, 585 nm dichroic minor) and Normarski-type DIC images were collected at 100× magnification using Slidebook software (Intelligent Imaging Innovations). Digital images (16 bit) were obtained with a cooled EMCCD camera (QuantEM:512SC, Photometrics) with exposure times between 50 ms and 200 ms.

### Cell Surface Labeling

For some constructs in this work (neurexin-1β and LDLR), an alternative peptide sequence called LAP4.2 (Puthenveetil, et al., 2009) was used (GFEIDKVWHDFPA; SEQ ID NO:5), in order to boost cell surface expression levels. HEK cells were transfected with 200 ng LAP4.2-neurexin-1β and 200 ng H2B-YFP co-transfection marker plasmid, per 0.95 cm2 cells at ˜70% confluency, using Lipofectamine 2000 (Invitrogen). 15 hours after transfection, the growth media was removed, and the cells were washed three times with DPBS with 0.5% casein. Casein was added to DPBS for all washing and labeling steps as a blocking agent and was required to reduce non-specific sticking of the probes. The cells were then labeled by applying 100 μM Ald probe, 1 μM W37ILplA, 1 mM ATP, and 5 mM Mg(OAc)₂ in DPBS with 0.5% casein at 37° C. for 45 minutes. Cells were then washed three times with DPBS with 0.5% casein and treated with 10 mM aniline and 100 μM AlexaFluor568-Hydrazide at 4° C. for 30 min. Cells were washed an additional three times and imaged live. The cell surface labeling was performed in the same fashion with the following changes: labeling was done using Hyd probe for 45 min at room temperature, and the fluorophore conjugation was done using 3 μM PE-Ald (4FB-R PE, Solulink) for 45 min at 4° C.

COS-7 cells were transfected with 200 ng LAP4.2-LDLR and 100 ng H2b-YFP co-transfection marker, only 20 μM Hyd probe was used in the initial labeling, and 0.3 μM PE-Ald with 20 mM aniline for 45 min was used for the fluorophore conjugation.

### Synthesis of Aldehyde (Ald) and Hydrazine (Hyd) Probes

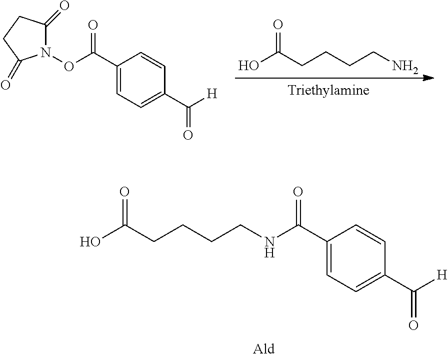 [SMILES: O=Cc1ccc(C(=O)ON2C(=O)CCC2=O)cc1 ; NCCCCC(=O)O ; O=Cc1ccc(C(=O)NCCCCC(=O)O)cc1]

The Ald probe was synthesized by reacting a solution of S-4FB (5 mg, 20.25 μmol, Solulink) in 100 μL of dry dimethyl sulfoxide (DMSO) with 5-aminovaleric acid (4.5 mg, 40 μmol, Alfa Aesar) and triethylamine (TEA, 8.4 μL, 60 μmol). The reaction was allowed to proceed at 30° C. for 4 hrs. Purification was performed by HPLC on a C18 Microsorb-MV 100 column (250×4.6 mm). A gradient of 0-100% acetonitrile in water over 20 min was used and detection was performed at 210 nm. Fractions were lyophilized and then dissolved in 50 μL dry DMSO.ESI-MS [M−H]−Ald: 248.2 observed, 248.09 calculated.

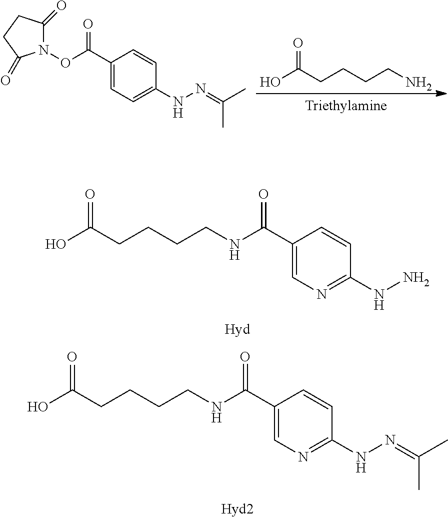

The hydrazine probe was synthesized in similar fashion by reacting S-HyNic (2.5 mg, 8.6 μmol, Solulink) with 5-aminovaleric acid (1.9 mg, 17.2 μmol) and triethylamine (TEA, 3.6 μL, 25.8 μmol) in 43 μL of dry DMSO. The products were purified via HPLC as described above. Purified products Hyd and Hyd2 were obtained. We note that both the hydrazine (Hyd) and ketone protected hydrazone (Hyd2) probe were capable of ligation by W37ILplA. Our measured values of Hyd ligation were done using purified Hyd probe to avoid potential complications to the analysis resulting from a mixture of products.

ESI-MS [M+H]+Hyd: 253.2 observed, 253.13 calculated. Hyd2: 293.2 observed, 293.16 calculated.

### Mass Spectrometric Analysis of Probe-LAP Conjugates

Starred peaks were manually collected and injected into an Applied Biosystems 200 QTRAP mass spectrometer.

### Measurement of Kcat Values for Ald and Hyd Ligation

Values of kcat for W37ILplA ligation of the Ald and Hyd probes onto LAP peptide were determined by measuring the initial reaction rates by HPLC. The conditions used were as follows: 1 μM W37ILplA, 600 μM LAP, 500 μM of Ald or Hyd, 2 mM magnesium acetate, and 25 mM sodium phosphate buffer, pH 7.2. Each initial rate was measured in triplicate and the average value reported. The error shown represents ±1 s.d. The equation kcat=Vmax/[E] was used to determine the kcat value.

Ald Ligation Vmax=19.7±0.7 μM/min; kcat=0.33±0.01 s-1

Hyd Ligation Vmax=1.25±0.16 μM/min; kcat=0.021±0.003 s-1

### Cell Surface Labeling of Biotinylated Cell Surface Receptor

Monovalent streptavidin-AF568 conjugate (mSA-AF568) was prepared as described herein. Briefly, the reaction was assembled using 7.5 μM LAP-mSA, 1 μMW37ILplA, 1 mM Ald, 5 mM ATP, 5 mM magnesium acetate, and 25 mM Na2HPO4 pH 7.2 in a total volume of 50 μL. Reactions were allowed to react at room temperature for 3 hr before ultrafiltration. Conjugation was performed by adding 20 mM aniline and 500 μM of AlexaFluor568-hydrazide and reacting overnight at 4° C. Ultrafiltration was repeated in order to remove unreacted AlexaFluor568-hydrazide. HEK cells were transfected with 200 ng BirA-ER, 200 ng AP-LDLR and 100 ng H2b-YFP co-transfection marker plasmid, per 0.95 cm2 at ˜70% confluency, using Lipofectamine 2000 (Invitrogen). After 4 hrs, the media was replaced with complete media containing 10 μM biotin. 15 hours after transfection, the growth media was removed, and the cells were washed three times with DPBS with 0.5% casein. The mSA-AF568 conjugate described above was diluted 1:50 in DPBS with 0.5% casein and added to the cells for 10 minutes at 4° C. Cells were washed three times and imaged.

### Mammalian Lysate Labeling

HEK cells were lysed under hypotonic conditions in 1 mM HEPES pH 7.5 with 5 mM MgCl2, protease inhibitor cocktail (Calbiochem), and 1 mM phenylmethylsulfonyl fluoride. Three cycles of freeze-thaw with 3 min of vortexing was performed, followed by centrifugation to clear the lysate. Samples were then stored at −80° C. Lysate samples were incubated with 10 μM LAP-YFP, 500 nM W37ILplA, 100 μM Ald or Hyd, 5 mM ATP, 5 mM magnesium acetate, and 25 mM sodium phosphate buffer, pH 7.2 overnight. The pH was then adjusted to 5 and 10 mM aniline and 200 μM of either AF568-Hyd or Fluorescein-Ald were added. After 1 hr, samples were boiled in protein loading buffer for 10 min and analyzed on a 10% SDS-PAGE gel. In gel fluorescence imaging was done on a Fujifilm FLA-9000.

### PE Intensity Distribution Analysis

Multiple images of PE labeling of LAP4.2-LDLR on the cell surface of COS cells randomly spread onto a glass slide were captured. Individual PE particles in each frame were identified using Insight3 software (developed by Prof. Xiaowei Zhuang's group at Harvard) and the average intensity of each was exported. Histograms of intensity distribution were generated using a bin size of 50.

Transfected COS cells expressing LAP4.2-LDL receptor were labeled using the conditions described above. Fluorescence was shown over a period of 60 s using TIRF. In order to reduce photobleaching of the PE probe, the imaging buffer was supplemented with an oxygen scavenger system that consisted of 5.6% (w/v) glucose oxidase, 0.4% (w/v) catalase, and 10% (w/v) glucose. Frames were captured at a rate of 1 per second, with an exposure time of 200 ms.

### Results

E. coli LplA catalyzes highly sequence-specific lipoic acid conjugation to a 13-amino acid recognition sequence, LAP2 (Puthenveetil, et al., 2009). We have previously shown that mutation of the lipoic acid binding pocket can confer the ability to ligate a range of unnatural substrate structures, including 7-hydroxycoumarin (Uttamapinant, et al., 2010), an aryl azide photocrosslinker (Baruah, et al., 2008), and trans-cyclooctene (Liu, et al., J. Am. Chem. Soc. (2011)). To test if mutants of LplA could accept arylaldehyde and aryl hydrazine substrates, we synthesized the two structures shown in FIG. 27A, in addition to analogs with one less methylene. These four substrates were screened against wild-type LplA and the seven mutants shown in FIG. 27B. We have previously observed that the W37 position, which is located at the end of the lipoic acid binding tunnel, acts as a “gatekeeper” residue whose mutation allows LplA to accept substrates whose size and shape differ greatly from lipoic acid. We tested a small panel of W37 mutants which have previously shown activity for unnatural probe ligation. Uttamapinant, et al., 2010; Liu, et al., 2011; and Jin, et al., 2011. No activity was detected with any of the LplA mutants with the shorter aldehyde and hydrazine substrates. However, the longer aryl aldehyde (“Ald”) shown in FIG. 27A was recognized and ligated to the LAP peptide by several of the W37 mutants, with W37ILplA having the highest activity (FIG. 27B). Using 1 μM W37ILplA, 500 μM Ald probe, and 150 μM LAP peptide, the reaction proceeded to 62% completion in 5 minutes (FIG. 27B).

We found that the aryl hydrazine (“Hyd”) probe was also ligated by many of the LplA mutants, but not as efficiently as the aryl aldehyde (“Ald”). Interestingly, the relative activity of the W37 mutants for the Hyd probe was similar to that with the Ald probe, with W37ILlpA again having the highest activity. However, the overall activity with the Hyd probe was lower than that for the Ald probe, reacting to 50% completion using W37ILplA over 60 min. We determined the kcat values for W37ILplA-catalyzed attachment of the Ald and Hyd probes to LAP peptide. Ald ligation had a kcat of 0.33±0.01 s-1 while Hyd ligation had a kcat of 0.021±0.003 s-1. Both ligations required ATP and could not be catalysed by did not proceed using wild-type LplA (FIG. 27C). Identities of product peaks were confirmed by mass spectrometry.

In Vitro Protein Labeling with LplA and Bis-Aryl Hydrazone Formation

We proceeded to test whether our LplA-mediated protein tagging method could be used for specific modification of proteins in vitro. We first turned our attention to streptavidin, a protein used ubiquitously in biotechnology due to its extremely high affinity and specificity for the small-molecule biotin. The ability to form site-specific conjugates of streptavidin to reporters such as fluorophores, enzymes (e.g., horse radish peroxidase, alkaline phosphatase) and phycoerthyrin could be extremely beneficial for enhancing activity and hence performance in applications ranging from ELISA and western blotting to live cell imaging.

We prepared streptavidin protein displaying a single LAP tag by utilizing our previously described monovalent streptavidin technology (Howarth, et al., 2006). Monovalent streptavidin was prepared by refolding one equivalent of wild-type streptavidin (“alive”, A) with three equivalents of “dead” (non-biotin-binding, D) streptavidin. The resulting mixture of heterotetramers was then purified by gradient nickel affinity chromatography to isolate the species with exactly one wild-type subunit and three dead subunits, i.e., a single biotin binding site in the context of a tetrameric protein. We genetically fused the 13-amino acid LAP2 tag to the N-terminus of the wild-type subunit. Therefore, the resulting purified monovalent streptavidin (mSA) had a single LAP tag on the functional biotin-binding subunit of the tetrameric protein.

Labeling with W37ILplA was performed with either Ald or Hyd substrate for 1 hr. After labeling, the crude mixtures were combined with either AlexaFluor568-hydrazide (AF568-Hyd) orfluorescein-aldehyde to selectively derivatize Ald or Hyd, respectively. Reactions were performed in the presence of 20 mM aniline catalyst at pH 5.0, overnight at room temperature. Specific conjugation of AF568-Hyd to Ald-functionalized mSA-LAP, and specific conjugation of fluorescein-aldehyde to Hyd-functionalized mSA-LAP were observed. Importantly, negative controls with ATP omitted from the first step, or wild-type LplA used in place of W37ILplA, showed no labeling.

To test if these site-specific mSA-LAP-fluorophore conjugates were active and functional, we used them to perform labeling and imaging of biotinylated cell surface proteins. HEK cells were transfected with plasmids for acceptor peptide (AP)-tagged low density lipoprotein receptor (LDLR) and endoplasmic reticulum (ER)-targeted biotin ligase. Previous work has shown that such conditions result in site-specific biotinylation of the AP tag in the ER lumen by biotin ligase (Howarth, et al., 2008). These cells were then treated with the mSA-LAP-AlexaFluor568 conjugate described above. Specific fluorescence labeling was seen in transfected cells expressing AP-LDLR and the nuclear yellow fluorescent protein (YFP) transfection marker. Labeling was not seen when the AP tag was mutated, excess biotin was added to quench mSA, or cells were not transfected. Hence, the results obtained from this study demonstrates that the mSA-fluorophore conjugate prepared by LplA and bis-aryl hydrazone formation was functional for live cell labeling and imaging.

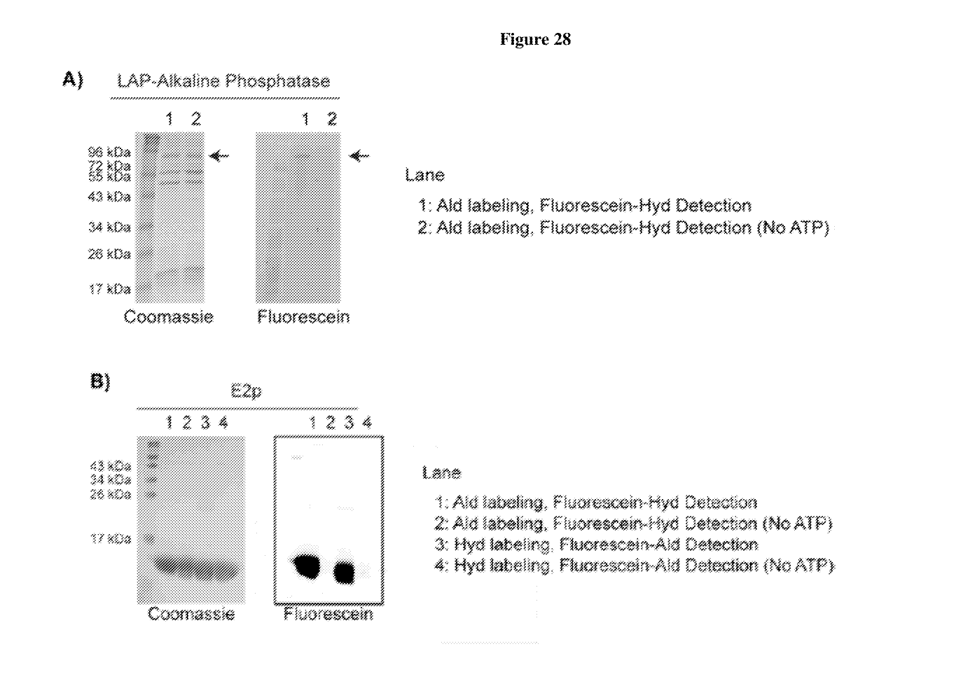

To illustrate generality, we performed similar labeling of two other proteins. One is alkaline phosphatase, an enzyme frequently attached to antibodies and streptavidin and used to generate a chromogenic signal in ELISA assays. We prepared a LAP fusion to the N-terminus of alkaline phosphatase, labeled with LplA and Ald, and then derivatized with fluorescein-Hyd. The results show that this labeling was effective and dependent on ATP. FIG. 28. The second protein we labeled was E2p, a 9 kDa domain of pyruvate dehydrogenase, one of LplA's natural protein substrates in E. coli (Green, et al., Biochem. J., 309:853-862 (1995)). FIG. 28 shows successful conjugation of fluorescein-Ald to Hyd-labeled E2p protein, as well as the reverse scheme.

A major benefit of the LplA protein labeling strategy is the exceptional sequence specificity of LplA. Hence, we explored the ability of our two-step labeling protocol to specifically conjugate fluorophores to LAP in complex mixtures containing thousands of competing proteins. A labeling experiment with a LAP-YFP fusion in mammalian cell lysate was performed. AlexaFluor568 and fluorescein are conjugated to LAP-YFP only, and not any endogenous mammalian proteins, using LplA and bis-aryl hydrazone formation. Negative controls with LAP-YFP omitted or wild-type LplA in place of W37ILplA show no labeling.

Cell Surface Protein Labeling with LplA and Bis-Aryl Hydrazone Formation

We next tested our labeling protocol in the context of the living mammalian cell surface. This context tests both the specificity of our labeling scheme, and its biocompatibility. We co-transfected HEK cells with expression plasmids for LAP4.2-neurexin-1β and a nuclear YFP transfection marker. Neurexin-1β is a single transmembrane protein with an extracellular N terminus that functions as a neuronal adhesion protein. LAP4.2 (Puthenveetil, et al., 2009) is a less hydrophobic variant of LAP that frequently gives improved surface targeting compared to LAP fusions as described above. Labeling was performed with W37ILplA, ATP, and 100 μM Ald for 45 min at 37° C. Reagents were washed away, and then 100 μM AF568-Hyd was added together with 10 mM aniline at 4° C. for 30 min. After washing, cells were immediately imaged. The results show that cell surface labeling was specific to transfected cells expressing LAP4.2-neurexin-1β. Negative controls using wild-type LplA, ATP omitted, or a LAP containing an alanine mutation showed no labeling.

Cell Surface Protein Labeling with Phycoerythrin and Single Molecule Imaging

Single molecule imaging is a powerful way to study protein trafficking in cells without losing information through ensemble averaging. Single molecule imaging in the cellular context requires fluorophores that are exceptionally bright and photostable. Quantum dots have excellent fantastic photophysical properties but commercial versions are very large and multivalent (Howarth, et al., 2008). Small organic dyes such as the AlexaFluors and cyanine dyes are much dimmer and require intense illumination to in order to achieve reasonable high signal-to-noise ratios at the single molecule level. However, under these conditions, photobleaching occurs too rapidly and prevents to allow single molecule tracking for longer than a few minutes or even seconds (Altman, et al., Nat. Methods, 9:68-71 (2012)).

For biotechnological applications requiring extreme fluorophore brightness, such as fluorescence activated cell sorting (FACS), phycoerythrin has been used as a much brighter alternative to organic dyes and a smaller and less expensive alternative to QDs. R-phycoerythrin (PE) is a 240 kD protein with a disk shape (of disk-shape, with a diameter of 11 nm×and a thickness of 6 nm), containing 34 embedded phycobilin-type chromophores. It is usually obtained by purification from red algae (Chang, et al., J. Mol. Biol., 262 721-722 (1996)). With an extinction coeffient (ε) of 2.0×106 M-1 cm-1 at 566 nm, and quantum yield (QY) of 0.85, it is >25 times brighter than AlexaFluor 568 (ε=91,300 M-1 cm1 at 568 nm; QY=0.69), which emits at the same waveleng than organic fluorophore with similar emission spectrum.

PE has rarely been explored as a reagent for single molecule imaging. Previously, Irvine, et al. used PE for single timepoint imaging of single peptide molecules binding to label major histocompatibility complex (MHC) on the surface of antigen presenting cells in order to count the copy number of peptide-MHC (Irvine, et al., Nature, 419:845-849 (2002)). We wished to explore the use of our LplA method to target PE to specific cell surface proteins, and to image them at the single molecule level. Since PE can only be practically added to cells at low micromolar concentrations, it is essential that it be targeted using a method with an extremely high second order rate constant. For instance, calculations shows that the yield would be <1% using a targeting method with a rate constant of ˜0.1 M-1 s-1, such as azide-azadibenzocyclooctyne cycloaddition (Yao, et al., 2012 and Desvaux, et al., 2007) after 1 hour of labeling. With its extremely fast kinetics and cell compatibility, the bis-aryl hydrazone conjugation is therefore ideal for this application.

To first see if we could conjugate phycoerythrin selectively, we prepared HEK cells expressing LAP4.2-neurexin-1β, and labeled them with the Hyd probe using W37ILplA. After labeling, cells were washed and treated with 20 mM aniline and PE modified with 4-formylbenzamide (PE-Ald). After 45 min the cells were washed and imaged. Clear labeling was observed in transfected cells. No labeling was seen in negative controls using wild-type LplA, with ATP omitted, or with an alanine mutation in LAP.

To perform single molecule imaging with PE, we next prepared COS7 cells expressing LAP4.2-LDLR on their surfaces. LDLR is a constitutively internalized receptor that promotes the plasma clearance of LDL particles via clathrin-mediated endocytosis pathway. A single-molecule imaging platform for LDLR based on our hydrazine-labeling technique could potentially provide additional insight into the mechanisms of LDLR for targeting LDLR to the clathrin-coated pits for example. We labeled the LDLR using our Hyd probe, followed by treatment with 20 mM aniline and PE-Ald. Individual labeled LDLR molecules appeared as single diffraction-limited spots on the cell surface, imaged by total internal reflection fluorescence (TIRF) microscopy. To confirm that the labeled spots were indeed single receptors and not aggregates, we compared the intensity distribution of >2900 spots on cells to individual PE molecules randomly distributed on glass slides. Similar distributions were observed on glass slides and on cell surfaces. The labeled receptors are also dynamic, as shown in time-lapse imaging experiments captured at a frame rate of 1 fps over a period of 60 s. The brightness of PE molecules offers high signal-to-background ratios unmatched that is unparalleled by organic fluorophores, and photobleaching is reduced because of the lower laser intensity required for illumination.

### Conclusion

In summary, LplA provides a general method for targeting small molecule probes with extremely high specificity to proteins in vitro, in lysate, and in living cells. Bis-aryl hydrazone formation is an extremely fast and biocompatible ligation reaction. By combining these two technologies in this study, we have developed a method to prepare protein-small molecule and protein-protein conjugates with high specificity and great facility. We demonstrated the methodology on monovalent streptavidin, alkaline phosphatase, YFP, LDL receptor and neurexin-1β, preparing conjugates to AlexaFluor568, fluorescein, and the extremely bright fluorescent protein phycoerythrin.

Presently, several methods exist to incorporate the reaction partners for conventional hydrazone/oxime formation, such as alkyl aldehydes viausing the formylglycine generating enzyme (FGE) (Wu, et al., Proc. Natl. Acad. Sci. U.S.A., 106:3000-3005 (2009); and Blanden, et al., Bioconjug. Chem., 22:1954-1961 (2011)) or ketones by via incorporation of the unnatural amino acid p-acetylphenylalanine (Hutchins, et al., Chem. & Biol., 18:299-303 (2011)). In comparison to these methods, our LplA-based labeling takes advantage of the enhanced kinetics and stability of bis-aryl hydrazone formation, and we show that the same LplA mutant can target both the aryl aldehyde reaction partner AND and the hydrazinopyridine reaction partner.

We note that our method may be improved by the use of 4-aminophenylalanine as an alternative to aniline for catalysis, where it may be more gentle on sensitive proteins such as tubulin (Blanden, et al., Bioconjug. Chem., 22:1954-1961 (2011)). Although we have demonstrated specific labeling on the surface of live cells, we note that expansion of this methodology for the labeling of intracellular proteins is likely to be complicated by the presence of endogenous aldehydes in the cell's interior. This study expands the panel of probes that can be ligated by LplA mutants for specific labeling of proteins. In comparison to lipoic acid ligation by wild-type LplA (kcat=0.22 s-1), and 7-hydroxycoumarin ligation by W37VLplA (kcat=0.019 s⁻¹), the measured kcat for Ald ligation (0.33±0.01 s-1) is extremely rapid and among the best for an unnatural probe/LplA mutantligase pair (Uttamapinant, et al., 2010)). The hydrophobic nature of the substrate recognition may also partially explain the ten-fold greater activity of Ald versus Hyd, as where the polar nature of the hydrazine may interfere with binding.

We envision the use of this method for preparation of being used to prepare improved conjugates of streptavidin and antibodies to reporters, particularly enzyme reporters such as peroxidase and alkaline phosphatase, where non-specific chemical conjugation methods could block their active sites and reduce activity. Such reagents could lead to improved sensitivity and reproducibility for ELISAs, western blots, and immunofluorescence staining. Finally, we note that our method showcases the use of phycoerythrin for single molecule imaging of specific proteins in the context of live cells. We believe this should be generalizable and provide an alternative to small organic dyes (due to increased brightness) and QDs (due to smaller size and lower cost).

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method for preparing a protein conjugate, the method comprising:
contacting a fusion protein with a lipoic acid analog in the presence of a lipoic acid ligase polypeptide to produce a protein conjugate in which the lipoic acid analog is linked to the fusion protein,
wherein
the lipoic acid analog is a substrate of the lipoic acid ligase polypeptide and has the following Formula: 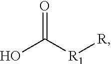
or an ester thereof, wherein R₁ is a branched or unbranched, substituted or unsubstituted C₂-C₁₄ alkyl or alkene, and R is a moiety that comprises
(i) a functional group handle, or
(ii) a directly detectable group;
wherein when R₁ is a C₅-C₁₀ alkyl or alkene, the functional group handle is not an azide, when R₁ is a C₄-C₈ alkyl or alkene, the functional group handle is not an alkyne, when R₁ is C₈-C₁₁ alkyl or alkene, the functional group handle is not a halide, and when R₁ is a C₃-C₄ alkyl, the directly detectable group is not a moiety selected from the group consisting of an aryl azide, a tetrafluorobenzoic derivative, benzophenone, coumarin, or Pacific blue, and
wherein the fusion protein comprises the target protein and an acceptor polypeptide.

2. The method of claim 1, wherein the directly detectable label is not a moiety of aryl azide, diazirine, benzophenone, chloroalkane, fluorobenzoic derivative, coumarin, resorufin, xanthene-type fluorophore, fluorescein, or metal-binding ligand.

3. The method of claim 1, wherein the acceptor polypeptide comprises the amino acid sequence P⁻⁴P⁻³P⁻²P⁻¹P⁰P⁺¹P⁺²P⁺³P⁺⁴P⁺⁵ (SEQ ID NO:2), in which:
P⁻⁴ is a hydrophobic amino acid residue,
P⁻³ is E or D,
P⁻² is any amino acid residue,
P⁻¹ is D, N, E, Y, A, or V,
P⁰ is K,
P⁺¹ is a hydrophobic amino acid residue,
P⁺² is a hydrophobic amino acid residue or S,
P⁺³ is a hydrophobic amino acid residue,
P⁺⁴ is E or D, and
P⁺⁵ is a hydrophobic amino acid residue.

4. The method of claim 3, wherein:
P⁻⁴ is I, V, L, or F,
P⁻² is I,
P⁺¹ is A or V,
P⁺² is an aromatic residue,
P⁺³ is an aliphatic hydrophobic residue or an aromatic hydrophobic residue, or
P⁺⁵ is an aliphatic hydrophobic residue.

5. The method of claim 3, wherein the acceptor polypeptide comprises amino acid sequence selected from the group consisting of:

| | |
|---|---|
| GFEIDKVWYDLDA, | (SEQ ID NO: 4) |
| GFEIDKVFYDLDA, | (SEQ ID NO: 6) |
| GFEIDKVWHDFPA, | (SEQ ID NO: 5) |
| and | |
| DEVLVEIETDKAVLEVPGGEEE | (SEQ ID NO: 3) |

6. The method of claim 1, wherein R is a moiety comprising a functional group handle selected from the group consisting of cyclooctene, trans-cyclooctene, azide, picolyl azide, alkyne, tetrazine, aldehyde, hydrazine, hydrozide, ketone, hydrozylamine, quadricyclane, alkene, diaryltetrazole, phosphine, diene, haloalkane, thiol, allyl sulfide, ether, thiophene, thioether, and alkyl amine.

7. The method of claim 6, further comprising contacting the protein conjugate with a compound that contains a dectable label to produce a labeled protein conjugate.

8. The method of claim 7, wherein the dectable label is selected from the group consisting of benzophenone, diazirine, aryl azide, coumarin, unbelliferone, pacific blue, resorufin, BODIPYs, cyanine, AlexaFluor, ATTO dye, NBD, rhodamine, tetramethylrhodamine, Texas red, Lucifer yellow, Cascade yellow, dansyl, Rose Bengal, and erosin.

9. The method of claim 1, wherein R is a moiety comprising a directly detectable group selected from the group consisting of benzophenone, diazirine, aryl azide, coumarin, unbelliferone, pacific blue, resorufin, BODIPYs, cyanine, AlexaFluor, ATTO dye, NBD, rhodamine, tetramethylrhodamine, Texas red, Lucifer yellow, Cascade yellow, dansyl, Rose Bengal, and erosin.

10. The method of claim 1, wherein the lipoic acid ligase polypeptide is a wild-type lipoic acid ligase or a functional fragment thereof.

11. The method of claim 1, wherein the lipoic acid ligase polypeptide is a functional variant of a wild-type ligase.

12. The method of claim 9, wherein the lipoic acid ligase polypeptide comprises at least one amino acid substitution at a position corresponding to W37 in SEQ ID NO:1.

13. The method of claim 10, wherein the lipoic acid ligase polypeptide is an LplA mutant selected from the group consisting of W37V, W37S, W37I, W37L, W37A, W37G, E20G/W37T, and E20A/F147A/H149G.

14. A method for preparing a protein conjugate, the method comprising:
contacting a fusion protein with a lipoic acid analog in the presence of a lipoic acid ligase polypeptide to produce a protein conjugate in which the lipoic acid analog is linked to the fusion protein,
wherein
the lipoic acid analog is a substrate of the lipoic acid ligase polypeptide and has the following Formula: 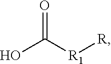
or an ester thereof, wherein R₁ is a branched or unbranched, substituted or unsubstituted C₉-C₁₄ alkyl or alkene, and R is a moiety that comprises a functional group handle or a directly detectable group, and
wherein
the fusion protein comprises the target protein and an acceptor polypeptide.

15. The method of claim 14, wherein the acceptor polypeptide comprises the amino acid sequence P⁻⁴P⁻³P⁻²P⁻¹P⁰P⁺¹P⁺²P⁺³P⁺⁴P⁺⁵ (SEQ ID NO:2), in which:
P⁻⁴ is a hydrophobic amino acid residue,
P⁻³ is E or D,
P⁻² is any amino acid residue,
P⁻¹ is D, N, E, Y, A, or V,
P⁰ is K,
P⁺¹ is a hydrophobic amino acid residue,
P⁺² is a hydrophobic amino acid residue or S,
P⁺³ is a hydrophobic amino acid residue,
P⁺⁴ is E or D, and
P⁺⁵ is a hydrophobic amino acid residue.

16. The method of claim 15, wherein:
P⁻⁴ is I, V, L, or F,
P⁻² is I,
P⁺¹ is A or V,
P⁺² is an aromatic residue,
P⁺³ is an aliphatic hydrophobic residue or an aromatic hydrophobic residue, or
P⁺⁵ is an aliphatic hydrophobic residue.

17. The method of claim 14, wherein the acceptor polypeptide comprises amino acid sequence selected from the group consisting of:

| | |
|---|---|
| GFEIDKVWYDLDA, | (SEQ ID NO: 4) |
| GFEIDKVFYDLDA, | (SEQ ID NO: 6) |
| GFEIDKVWHDFPA, | (SEQ ID NO: 5) |
| and | |
| DEVLVEIETDKAVLEVPGGEEE | (SEQ ID NO: 3) |

18. The method of claim 14, wherein R is a moiety comprising a functional group handle selected from the group consisting of cyclooctene, trans-cyclooctene, azide, picolyl azide, alkyne, tetrazine, aldehyde, hydrazine, hydrozide, ketone, hydrozylamine, quadricyclane, alkene, diaryltetrazole, phosphine, diene, haloalkane, thiol, allyl sulfide, ether, thiophene, thioether, and alkyl amine.

19. The method of claim 18, further comprising contacting the protein conjugate with a compound that comprises a detectable label to produce a labeled protein conjugate.

20. The method of claim 19, wherein the detectable group is selected from the group consisting of benzophenone, diazirine, aryl azide, coumarin, unbelliferone, pacific blue, resorufin, BODIPYs, cyanine, AlexaFluor, ATTO dye, NBD, rhodamine, tetramethylrhodamine, Texas red, Lucifer yellow, Cascade yellow, dansyl, Rose Bengal, and erosin.

21. The method of claim 20, wherein R is a moiety comprising a directly detectable group selected from the group consisting of benzophenone, diazirine, aryl azide, coumarin, unbelliferone, pacific blue, resorufin, BODIPYs, cyanine, AlexaFluor, ATTO dye, NBD, rhodamine, tetramethylrhodamine, Texas red, Lucifer yellow, Cascade yellow, dansyl, Rose Bengal, and erosin.

22. The method of claim 14, wherein the lipoic acid ligase polypeptide is a wild-type lipoic acid ligase or a functional fragment thereof.

23. The method of claim 14, wherein the lipoic acid ligase polypeptide is a functional variant of a wild-type ligase.

24. The method of claim 14, wherein the lipoic acid ligase polypeptide comprises at least one amino acid substitution at a position corresponding to W37 in SEQ ID NO:1.

25. The method of claim 24, wherein the lipoic acid ligase polypeptide is an LplA mutant selected from the group consisting of W37V, W37S, W37I, W37L, W37A, W37G, E20G/W37T, and E20A/F147A/H149G.

26. A method for preparing a protein conjugate, the method comprising:
contacting a fusion protein with a lipoic acid analog in the presence of a lipoic acid ligase polypeptide to produce a protein conjugate in which the lipoic acid analog is linked to the fusion protein, wherein
the lipoic acid analog is a substrate of the lipoic acid ligase polypeptide and has the following Formula: 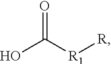
or an ester thereof, wherein R₁ is a branched or unbranched, substituted or unsubstituted C₂-C₁₄ alkyl or alkene, and R is a moiety that comprises a functional group handle or a directly detectable group,
wherein
the fusion protein comprises the target protein and an acceptor polypeptide, and wherein the lipoic acid ligase polypeptide is a truncated mutant of a wild-type lipoic acid ligase, the mutant having a deletion of a C-terminal fragment up to a position corresponding to E256 in SEQ ID NO:1 as compared to the wild-type lipoic acid ligase.

27. The method of claim 26, wherein the acceptor polypeptide comprises the motif P⁻⁴P⁻³P⁻²P⁻¹P⁰P⁺¹P⁺²P⁺³P⁺⁴P⁺⁵ (SEQ ID NO:2), in which:
P⁻⁴ is a hydrophobic amino acid residue,
P⁻³ is E or D,
P⁻² is any amino acid residue,
P⁻¹ is D, N, E, Y, A, or V,
P⁰ is K,
P⁺¹ is a hydrophobic amino acid residue,
P⁺² is a hydrophobic amino acid residue or S,
P⁺³ is a hydrophobic amino acid residue,
P⁺⁴ is E or D, and
P⁺⁵ is a hydrophobic amino acid residue.

28. The method of claim 27, wherein:
P⁻⁴ is I, V, L, or F,
P⁻² is I,
P⁺¹ is A or V,
P⁺² is an aromatic residue,
P⁺³ is an aliphatic hydrophobic residue or an aromatic hydrophobic residue, or
P⁺⁵ is an aliphatic hydrophobic residue.

29. The method of claim 26, wherein the acceptor polypeptide comprises amino acid sequence selected from the group consisting of:

| | |
|---|---|
| GFEIDKVWYDLDA, | (SEQ ID NO: 4) |
| GFEIDKVFYDLDA, | (SEQ ID NO: 6) |
| GFEIDKVWHDFPA, | (SEQ ID NO: 5) |
| and | |
| DEVLVEIETDKAVLEVPGGEEE | (SEQ ID NO: 3) |

30. The method of claim 22, wherein R is a moiety comprising a functional group handle is selected from the group consisting of cyclooctene, trans-cyclooctene, azide, picolyl azide, alkyne, tetrazine, aldehyde, hydrazine, hydrozide, ketone, hydrozylamine, quadricyclane, alkene, diaryltetrazole, phosphine, diene, haloalkane, thiol, allyl sulfide, ether, thiophene, thioether, and alkyl amine.

31. The method of claim 30, further comprising contacting the protein conjugate with a compound that comprises a detectable label to produce a labeled protein product.

32. The method of claim 31, wherein the detectable label is selected from the group consisting of benzophenone, diazirine, aryl azide, coumarin, unbelliferone, pacific blue, resorufin, BODIPYs, cyanine, AlexaFluor, ATTO dye, NBD, rhodamine, tetramethylrhodamine, Texas red, Lucifer yellow, Cascade yellow, dansyl, Rose Bengal, and erosin.

33. The method of claim 22, wherein R is a moiety comprising a directly detectable group selected from the group consisting of benzophenone, diazirine, aryl azide, coumarin, unbelliferone, pacific blue, resorufin, BODIPYs, cyanine, AlexaFluor, ATTO dye, NBD, rhodamine, tetramethylrhodamine, Texas red, Lucifer yellow, Cascade yellow, dansyl, Rose Bengal, and erosin.

34. The method of claim 26, wherein the truncated mutant comprises at least one amino acid substitution at a position corresponding to W37 in SEQ ID NO:1.
